(19) United States
(12) Patent Application Publication
(10) Pub. No.: US 2026/0258445 A1
(43) Pub. Date: Sep. 3, 2026

(54) RECOMBINANT ADENO-ASSOCIATED VIRAL VECTOR FOR GENE DELIVERY

(71) Applicant: Abeona Therapeutics Inc., Cleveland, OH (US)

(72) Inventors: Timothy J. MILLER, New York, NY (US); Linas PADEGIMAS, Mayfield, OH (US)

(21) Appl. No.: 19/345,117

(22) Filed: Sep. 30, 2025

Related U.S. Application Data
(63) Continuation of application No. 17/299,434, filed on Dec. 4, 2019, now Pat. No. 12,454,701.
(60) Provisional application No. 62/775,871, filed on Dec. 5, 2018.
(60) Provisional application No. 62/801,195, filed on Feb. 5, 2019.
(60) Provisional application No. 62/863,126, filed on Jun. 18, 2019.
(60) Provisional application No. 62/914,856, filed on Oct. 14, 2019.

(51) Int. Cl. C12N 15/86; A61K 48/00; C07K 14/005; C07K 14/47; C07K 14/705; C12N 9/26; C12N 9/40

(52) U.S. Cl. CPC C12N 15/86; A61K 48/0058; C07K 14/005; C07K 14/4712; C07K 14/705; C12N 9/2411; C12N 9/2465; C12Y 302/0102; C12Y 302/01022; C12N 2750/14122; C12N 2750/14143; C12N 2750/14171

(57) ABSTRACT

Provided herein are recombinant AAV vectors, AAV viral vectors, and capsid proteins for improved gene therapy, and methods for their manufacture and use.

### CROSS REFERENCE TO RELATED APPLICATIONS

This application is a continuation of U.S. patent application Ser. No. 17/299,434, filed Jun. 3, 2021, which is a U.S. National Phase application, filed under 35 U.S.C. § 371 (c), of International Application No. PCT/US2019/064396, filed Dec. 4, 2019, which claims priority to U.S. Provisional Application Nos. 62/775,871, filed Dec. 5, 2018; 62/801,195, filed Feb. 5, 2019; 62/863,126 filed Jun. 18, 2019, and 62/914,856 filed Oct. 14, 2019, each of which is incorporated by reference herein in its entirety for all purposes.

### INCORPORATION BY REFERENCE OF SEQUENCE LISTING

The content of the sequence electronic listing (ABEO_002_05US_SeqList_ST26.xml; Size: 414,040 bytes; and Date of Creation: Sep. 29, 2025) is herein incorporated by reference in its entirety.

### BACKGROUND

Adeno-associated viral vectors are promising delivery vectors for gene therapy. However, their therapeutic efficacy is undermined by the vectors' delivery efficiency or limited tissue tropisms. Therefore, there is an urgent need for new AAV vectors with a better therapeutic potential.

### SUMMARY

The present disclosure relates generally to the field of gene therapy and in particular, to recombinant adeno-associated viral (AAV) vector particles (also known as AAV viral vectors) with novel capsid proteins, their manufacture, and their use to deliver transgenes to treat or prevent a disease or disorder.

### BRIEF DESCRIPTION OF THE DRAWINGS

FIG. 1 Strategy for AIM capsid library construction.

FIG. 2A Comparison of transduction efficiency in tissue culture. HEK 293 cells were plated in a 96-well plate at 50,000 cells per well. Cells were transduced with capsids containing AAV9-GFP or AAV214-GFP virus at an MOI of 5E+5. Images were taken 45 hours post transduction.

FIG. 2B Comparison of transduction efficiency in different tissues of mouse dosed with 2E+11 viral genomes (vg) of AAV9 or AAV214 viruses.

FIG. 2C Comparison of transduction efficiency and expression levels in brain of mice dosed with 2E+11 vg of AAV9 or AAV214 viruses.

FIG. 3A Scanning laser ophthalmoscopy imaging of mouse retinas after AAV administration. Wild-type C57BL/6J mice were administered the labeled AAV serotype by both subretinal (right eye) and intravitreal (left eye) injection. 1 μL of AAV vector at 5E+12 vg/mL (5E+9 vg/eye) was injected for both methods of administration and animals were imaged after 10 days with a HRA2 Spectralis Scanning Laser Ophthalmoscope (Heidelberg Engineering, Carlsbad, CA). Images where cataract prevented sufficient observation were omitted from figure.

FIG. 3B IHC analysis of mouse eye dosed with AAV204-GFP intravitreally.

FIG. 4 compares GFP expression mediated by AAV204 or AAV9 transduction in primates by RT-qPCR. The dotted line corresponds to background calculated as-RT mean plus 2 or 4 standard deviations.

FIGS. 5A-5F shows AAV204-mediated eye expression. FIG. 5A shows transduction spread mediated by intravitreal injection of AAV204, mostly peripheral and some foveal. FIG. 5B shows (by GFP expression) transduction of a variety of retinal cells including photoreceptors and RPE cells in primates after intravitreal injection of AAV204 virus. FIG. 5C shows massive photoreceptor and RPE transduction in the macula where most cones (photoreceptors responsible for color vision) were concentrated.

FIGS. 5D-5F show expression of GFP from AAV204 driven by the VMD2 (vitelliform macular degeneration-2 promoters), which is a cell-specific promoter for the RPE. SLO imaging was performed at Day 14 (FIG. 5D) and Day 28 (FIG. 5E) after intra-vitreal injection of the 2.5×10¹² viral genomes (vg) vector. FIG. 5F shows GFP expression and nuclei (DAPI) in the periphery at Day 28.

FIG. 6 shows IHC analysis of NHP eye explant transduction performed ex vivo.

FIG. 7 Neutralizing antibody quantitation strategy. Absence of luminescence indicated that target AAV was bound by neutralizing antibodies.

FIG. 8 demonstrates different immunogenicity of AAV204 and AAV9. Neutralizing antibody titer was obtained using an in-house developed process. Either AAV9-Luc or pA-AAV204-Luc was incubated with various dilutions of serum at an MOI of 25,000. After incubation, the virus/serum mixture was transferred to wells containing 20,000 Lec2 cells and incubated with the cells for 24 hours, after which time luminescence was measured and compared to a control value from cells transduced with only virus at the same MOI.

FIG. 9 shows comparison of lung transduction using AAV204 or AAV6 (benchmark of AAV lung transduction) via intratracheal delivery.

FIGS. 10A-10C shows functionality of CFTRAR and full-length expression cassette by FLIPR assay (FIG. 10A) and dose response (FIG. 10B) to AAV204 packed CFTRΔR expression cassette treatment. FIG. 10C shows a comparison of CFTRΔR versus full-length codon-optimized CFTR expression with respect to membrane potential assayed by FLIPR. 293 cells were transduced with AAV204 expressing the proteins were monitored for changes in fluorescence. Baseline was read for 1 minute and then 50 μM forskolin was added to the cells.

FIGS. 11A-11C shows AAV204 ability to transduce cultivated CF patient cells (FIG. 11A), CFTRΔR expression and proper localization in cell membrane (FIG. 11B), and CFTRΔR expression restores CFTR current in human CF patient cells (FIG. 11C).

FIG. 12A shows the in vivo effect of administering AAV204 particles comprising CFTR transgene nasal to mouse models of cystic fibrosis. FIG. 12B shows effect of AAV204/CFTRΔR treatment (by increase of nasal membrane potential) in different CF patient cells.

FIG. 13 shows the bio distribution of the AAV9-CLN3 and AAV214-CLN3 vectors in CLN3Δex7/8 mice model 30 days after intravenous administration of viral particles.

FIG. 14 shows the expression of AAV9-CLN3 and AAV214-CLN3 vectors in CLN3 Aex7/8 mice brain tissues as measured by RT-qPCR.

FIG. 15 shows the immunoblot of GLA expression in transduced HEK293 cells.

FIG. 16 shows the enzymatic activities (supraphysiological) of GLA in plasma, brain, liver, spinal cord, heart, kidney, and eye in C57BL/6 mice after AAV administration.

FIG. 17 shows the enzymatic activities of GAA in brain, bicep, diaphragm and liver in C57BL/6 mice after AAV administration by intravenous injection.

FIGS. 18A-18B shows the immunoblot (FIG. 18A) and enzymatic (FIG. 18B) analysis of recombinant hGAA expressed in HEK293 cells by transfection.

FIGS. 19A-19E shows the enzymatic activities of GAA in plasma (FIG. 19A), brain (FIG. 19B), bicep (FIG. 19C), diaphragm (FIG. 19D) or liver (FIG. 19E) in C57BL/6 mice after AAV administration. FIG. 19F shows glycogen levels in GAA−/− mice treated IV with AAV capsid. Data is presented as % of glycogen found in the GAA−/− mice. Decreased glycogen shows restoration of GAA functionality by AAV9- and AAV214-mediated expression of codon-optimized GAA enzyme.

FIG. 20 shows an alignment of the VP1 amino acid sequences from AAV204 (SEQ ID NO: 2) and AAV6 (SEQ ID NO: 63).

FIG. 21 shows an alignment of the VP1 protein amino acid sequences from AAV214 (SEQ ID NO: 3); AAV214A (SEQ ID NO: 30), AAV214AB (SEQ ID NO: 84), AAV214e (SEQ ID NO: 31), AAV214e8 (SEQ ID NO: 32), AAV214e9 (SEQ ID NO: 33), AAV214e10 (SEQ ID NO: 34), ITB204_45 (SEQ ID NO: 49), AAV9 (SEQ ID NO: 71) and AAV8 (SEQ ID NO: 67).

FIG. 22 shows an alignment of the VP2 protein amino acid sequences from AAV214 (SEQ ID NO: 35); AAV214A (SEQ ID NO: 36), AAV214AB (SEQ ID NO: 85), AAV214e (SEQ ID NO: 37), AAV214e8 (SEQ ID NO: 38), AAV214e9 (SEQ ID NO: 39), AAV214e10 (SEQ ID NO: 40), ITB204_45 (SEQ ID NO: 50), AAV9 (SEQ ID NO: 72) and AAV8 (SEQ ID NO: 68).

FIG. 23 shows an alignment of VP3 protein amino acid sequences from AAV214 (SEQ ID NO: 41); AAV214A (SEQ ID NO: 42), AAV214AB (SEQ ID NO: 86), AAV214e (SEQ ID NO: 43), AAV214e8 (SEQ ID NO: 44), AAV214e9 (SEQ ID NO: 45), AAV214e10 (SEQ ID NO: 46), ITB204_45 (SEQ ID NO: 51), AAV9 (SEQ ID NO: 73) and AAV8 (SEQ ID NO: 69).

FIG. 24A shows expression of a GFP transgene delivered by an AAV110 vector particle and an AAV9 vector particle in muscle following intramuscular administration. The upper panel shows left and right legs in white light showing the overall tissue structure. The lower panel shows GFP fluorescence.

FIG. 24B provides a quantitative analysis of the fluorescence obtained in FIG. 24A for the AAV110 particle (ITCord1.10) compared to AAV9.

FIG. 25A-25C illustrates expression, relative to AAV9, of a transgene delivered by the AAV110 particle (ITCord1.10) following intramuscular administration. The data shows that AAV110 expression in muscle is particularly high.

FIG. 26A shows immunohistochemistry of muscle tissue to detect GFP expression following intramuscular administration of AAV110 and AAV9 particles expressing GFP. The FIG. shows GFP expressed from AAV9 vector particles (lower left panel), GFP expressed from AAV110 particles (lower right panel) and control muscle (upper panel). Tissue was stained with anti-GFP antibody.

FIG. 26B. IM Delivered AAV214 Transduces a Larger Muscle Area Than AAV9. Whole rat muscle (biceps femoris) was analyzed for GFP or mCherry expression by immunohistochemistry 10 days post-IM injection. Fixed and frozen sections were probed with GFP and mCherry pAb. AAV214 displayed a significantly larger transduction area in comparison to AAV9 which was largely confined to the upper portion of the muscle consistent with the injection site.

FIG. 27 shows a bioluminescence image showing luciferase, expressed as a transgene and exposed to luciferin. The data was obtained 28 days following AAV214 administration.

FIG. 28 compares muscle expression of SMN-1 protein delivered intravenously in AAV214 and AAV9.

FIG. 29 shows expression of GFP in heart and in bicep as a transgene mediated by variants of the AAV214 versus AAV9. The y-axis shows the log 10 value of virus copy number per microgram of genomic DNA.

FIG. 30 shows a diagram of the VP1, VP2, and VP3 capsid proteins. The VP1- and VP2-specific portions are indicated along with the VP3 portion, which is identical to the VP3 protein produced. The amino acid sequence of AAV214 VP3 (SEQ ID NO:41) is shown and variable regions I-IX are indicated. The full VP1 protein amino acid sequence for AAV214 is provided as SEQ ID NO:3.

FIGS. 31A-31C illustrate AAV214-treated animals demonstrate reduced generation of AAV9 neutralizing antibodies. FIG. 31A shows Animals dosed by IM with either AAV9 or AAV214 were assayed for neutralizing antibodies against AAV9. Analysis was performed by measuring the ability of animal serum to inhibit the transduction of an AAV9.luciferase vector into the permissive cell type, Lec2. Three days post-transduction, cells were assayed for luciferase activity. Each group consisted of 2 or 3 rats for either control, AAV9 or AAV214. FIGS. 31B and 31C show cross-reactivity to AAV9 and AAV204. FIG. 31B shows the development of various AAV neutralizing antibodies after AAV9 challenge. FIG. 31C shows the development of various AAV neutralizing antibodies after AAV204 challenge.

### DETAILED DESCRIPTION

Some embodiments according to the present disclosure will be described more fully hereinafter. Aspects of the disclosure may, however, be embodied in different forms and should not be construed as limited to the embodiments set forth herein. Rather, these embodiments are provided so that this disclosure will be thorough and complete, and will fully convey the scope of the invention to those skilled in the art. The terminology used in the description herein is for the purpose of describing particular embodiments only and is not intended to be limiting.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the present application and relevant art and should not be interpreted in an idealized or overly formal sense unless expressly so defined herein.

Unless the context indicates otherwise, it is specifically intended that the various features of the invention described herein can be used in any combination. Moreover, the disclosure also contemplates that In embodiments, any feature or combination of features set forth herein can be excluded or omitted. To illustrate, if the specification states that a complex comprises components A, B and C, it is specifically intended that any of A, B or C, or a combination thereof, can be omitted and disclaimed singularly or in any combination.

Unless explicitly indicated otherwise, all specified some embodiments, features, and terms intend to include both the recited embodiment, feature, or term and biological equivalents thereof.

### INCORPORATION BY REFERENCE

All references, articles, publications, patents, patent publications, and patent applications cited herein are incorporated by reference in their entireties for all purposes. However, mention of any reference, article, publication, patent, patent publication, and patent application cited herein is not, and should not, be taken as an acknowledgment or any form of suggestion that they constitute valid prior art or form part of the common general knowledge in any country in the world.

### Definitions

The practice of the present technology will employ, unless otherwise indicated, conventional techniques of organic chemistry, pharmacology, immunology, molecular biology, microbiology, cell biology and recombinant DNA, which are within the skill of the art. See, e.g., Sambrook, Fritsch and Maniatis, Molecular Cloning: A Laboratory Manual, 2nd edition (1989); Current Protocols In Molecular Biology (F. M. Ausubel, et al. eds., (1987)); the series Methods in Enzymology (Academic Press, Inc.): PCR 2: A Practical Approach (M. J. MacPherson, B. D. Hames and G. R. Taylor eds. (1995)), Harlow and Lane, eds. (1988) Antibodies, a Laboratory Manual, and Animal Cell Culture (RI. Freshney, ed. (1987)).

As used in the description of the invention and the appended claims, the singular forms “a,” “an” and “the” are intended to include the plural forms as well, unless the context clearly indicates otherwise.

As used herein, the term “comprising” is intended to mean that the compositions and methods include the recited elements, but do not exclude others. As used herein, the transitional phrase “consisting essentially of” (and grammatical variants) is to be interpreted as encompassing the recited materials or steps and those that do not materially affect the basic and novel characteristic(s) of the recited embodiment. Thus, the term “consisting essentially of” as used herein should not be interpreted as equivalent to “comprising.” “Consisting of” shall mean excluding more than trace elements of other ingredients and substantial method steps for administering the compositions disclosed herein. Aspects defined by each of these transition terms are within the scope of the present disclosure.

All numerical designations, e.g., pH, temperature, time, concentration, and molecular weight, including ranges, are approximations which are varied (+) or (−) by increments of 1.0 or 0.1, as appropriate, or, alternatively, by a variation of +/−15%, 10%, 5%, 2%. It is to be understood, although not always explicitly stated, that all numerical designations are preceded by the term “about”. It also is to be understood, although not always explicitly stated, that the reagents described herein are merely exemplary and that equivalents of such are known in the art. The term “about,” as used herein when referring to a measurable value such as an amount or concentration and the like, is meant to encompass variations of 20%, 10%, 5%, 1%, 0.5%, or even 0.1% of the specified amount.

The terms “acceptable,” “effective,” or “sufficient” when used to describe the selection of any components, ranges, dose forms, etc. disclosed herein intend that said component, range, dose form, etc. is suitable for the disclosed purpose.

Also, as used herein, “and/or” refers to and encompasses any and all possible combinations of one or more of the associated listed items, as well as the lack of combinations when interpreted in the alternative (“or”).

Unless specifically recited, the term “host cell” includes a eukaryotic host cell, including, for example, fungal cells, yeast cells, higher plant cells, insect cells and mammalian cells. Non-limiting examples of eukaryotic host cells include simian, bovine, porcine, murine, rat, avian, reptilian and human, e.g., HEK293 cells and 293T cells.

The term “isolated” as used herein refers to molecules or biologicals or cellular materials being substantially free from other materials.

As used herein, the terms “nucleic acid sequence” and “polynucleotide” are used interchangeably to refer to a polymeric form of nucleotides of any length, either ribonucleotides or deoxyribonucleotides. Thus, this term includes, but is not limited to, single-, double-, or multi-stranded DNA or RNA, genomic DNA, cDNA, DNA-RNA hybrids, or a polymer comprising, consisting essentially of, or consisting of purine and pyrimidine bases or other natural, chemically or biochemically modified, non-natural, or derivatized nucleotide bases.

A “gene” refers to a polynucleotide containing at least one open reading frame (ORF) that is capable of encoding a particular polypeptide or protein. A “gene product” or, alternatively, a “gene expression product” refers to the amino acid sequence (e.g., peptide or polypeptide) generated when a gene is transcribed and translated.

As used herein, “expression” refers to the two-step process by which polynucleotides are transcribed into mRNA and/or the process by which the transcribed mRNA is subsequently translated into peptides, polypeptides, or proteins. If the polynucleotide is derived from genomic DNA, expression may include splicing of the mRNA in a eukaryotic cell.

“Under transcriptional control” is a term well understood in the art and indicates that transcription of a polynucleotide sequence, usually a DNA sequence, depends on its being operatively linked to an element that contributes to the initiation of, or promotes, transcription. “Operatively linked” intends that the polynucleotides are arranged in a manner that allows them to function in a cell. In one aspect, this invention provides promoters operatively linked to the downstream sequences.

The term “encode” as it is applied to polynucleotides refers to a polynucleotide which is said to “encode” a polypeptide if, in its native state or when manipulated by methods well known to those skilled in the art, it can be transcribed to produce the mRNA for the polypeptide and/or a fragment thereof. The antisense strand is the complement of such a nucleic acid, and the encoding sequence can be deduced therefrom.

The term “promoter” as used herein means a control sequence that is a region of a polynucleotide sequence at which the initiation and rate of transcription of a coding sequence, such as a gene or a transgene, are controlled. Promoters may be constitutive, inducible, repressible, or tissue-specific, for example. Promoters may contain genetic elements at which regulatory proteins and molecules such as RNA polymerase and transcription factors may bind. Non-limiting exemplary promoters include Rous sarcoma virus (RSV) LTR promoter (optionally with the RSV enhancer), a cytomegalovirus (CMV) promoter, an SV40 promoter, a dihydrofolate reductase promoter, a β-actin promoter, a phosphoglycerol kinase (PGK) promoter, a U6 promoter, an H1 promoter, a ubiquitous chicken β-actin hybrid (CBh) promoter, a small nuclear RNA (U1a or U1b) promoter, an MeCP2 promoter, an MeP418 promoter, an MeP426 promoter, a minimal MeCP2 promoter, a VMD2 promoter, an mRho promoter or an EFI promoter.

Additional non-limiting exemplary promoters provided herein include, but are not limited to EF1a, Ubc, human β-actin, CAG, TRE, Ac5, Polyhedrin, CaMKIIa, Gal1, TEF1, GDS, ADH1, Ubi, and α-1-antitrypsin (hAAT). It is known in the art that the nucleotide sequences of such promoters may be modified in order to increase or decrease the efficiency of mRNA transcription. See, e.g., Gao et al. (2018) Mol. Ther.: Nucleic Acids 12:135-145 (modifying TATA box of 7SK, U6 and H1 promoters to abolish RNA polymerase III transcription and stimulate RNA polymerase II-dependent mRNA transcription). Synthetically-derived promoters may be used for ubiquitous or tissue specific expression. Further, virus-derived promoters, some of which are noted above, may be useful in the methods disclosed herein, e.g., CMV, HIV, adenovirus, and AAV promoters. In embodiments, the promoter is used together with an enhancer to increase the transcription efficiency. Non-limiting examples of enhancers include an interstitial retinoid-binding protein (IRBP) enhancer, an RSV enhancer or a CMV enhancer.

An enhancer is a regulatory element that increases the expression of a target sequence. A “promoter/enhancer” is a polynucleotide that contains sequences capable of providing both promoter and enhancer functions. For example, the long terminal repeats of retroviruses contain both promoter and enhancer functions. The enhancer/promoter may be “endogenous” or “exogenous” or “heterologous.” An “endogenous” enhancer/promoter is one which is naturally linked with a given gene in the genome. An “exogenous” or “heterologous” enhancer/promoter is one which is placed in juxtaposition to a gene by means of genetic manipulation (i.e., molecular biological techniques) such that transcription of that gene is directed by the linked enhancer/promoter. Non-limiting examples of linked enhancer/promoter for use in the methods, compositions and constructs provided herein include a PDE promoter plus IRBP enhancer or a CMV enhancer plus U1a promoter. It is understood in the art that enhancers can operate from a distance and irrespective of their orientation relative to the location of an endogenous or heterologous promoter. It is thus further understood that an enhancer operating at a distance from a promoter is thus “operably linked” to that promoter irrespective of its location in the vector or its orientation relative to the location of the promoter.

The term “protein”, “peptide” and “polypeptide” are used interchangeably and in their broadest sense to refer to a compound of two or more subunits of amino acids, amino acid analogs or peptidomimetics. The subunits may be linked by peptide bonds. In another aspect, the subunit may be linked by other bonds, e.g., ester, ether, etc. A protein or peptide must contain at least two amino acids and no limitation is placed on the maximum number of amino acids which may comprise, consist essentially of, or consist of a protein's or peptide's sequence. As used herein the term “amino acid” refers to either natural and/or unnatural or synthetic amino acids, including glycine and both the D and L optical isomers, amino acid analogs and peptidomimetics.

As used herein, the term “signal peptide” or “signal polypeptide” intends an amino acid sequence usually present at the N-terminal end of newly synthesized secretory or membrane polypeptides or proteins. It acts to direct the polypeptide to a specific cellular location, e.g. across a cell membrane, into a cell membrane, or into the nucleus. In embodiments, the signal peptide is removed following localization. Examples of signal peptides are well known in the art. Non-limiting examples are those described in U.S. Pat. Nos. 8,853,381, 5,958,736, and 8,795,965. In embodiments, the signal peptide can be an IDUA signal peptide.

The terms “equivalent” or “biological equivalent” are used interchangeably when referring to a particular molecule, biological material, or cellular material and intend those having minimal homology while still maintaining desired structure or functionality. Non-limiting examples of equivalent polypeptides include a polypeptide having at least about 60%, at least about 65%, at least about 70%, at least about 75%, at least about 80%, at least about 85%, at least about 90%, at least about 95% identity or at least about 99% identity to a reference polypeptide (for instance, a wild-type polypeptide); or a polypeptide which is encoded by a polynucleotide having at least about 70%, at least about 75%, at least about 80%, at least about 85%, at least about 90%, at least about 95% identity, at least about 97% sequence identity or at least about 99% sequence identity to the reference polynucleotide (for instance, a wild-type polynucleotide).

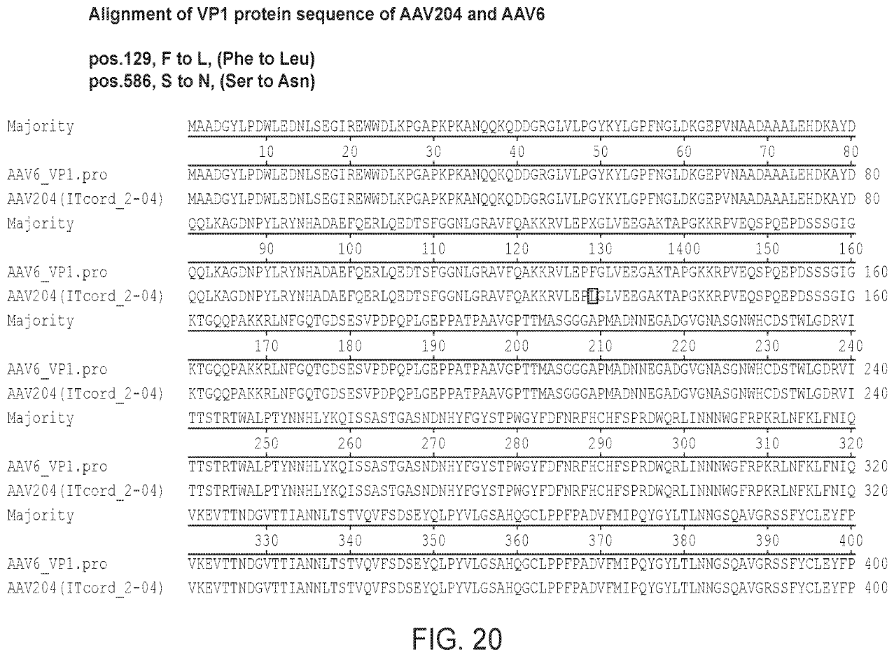

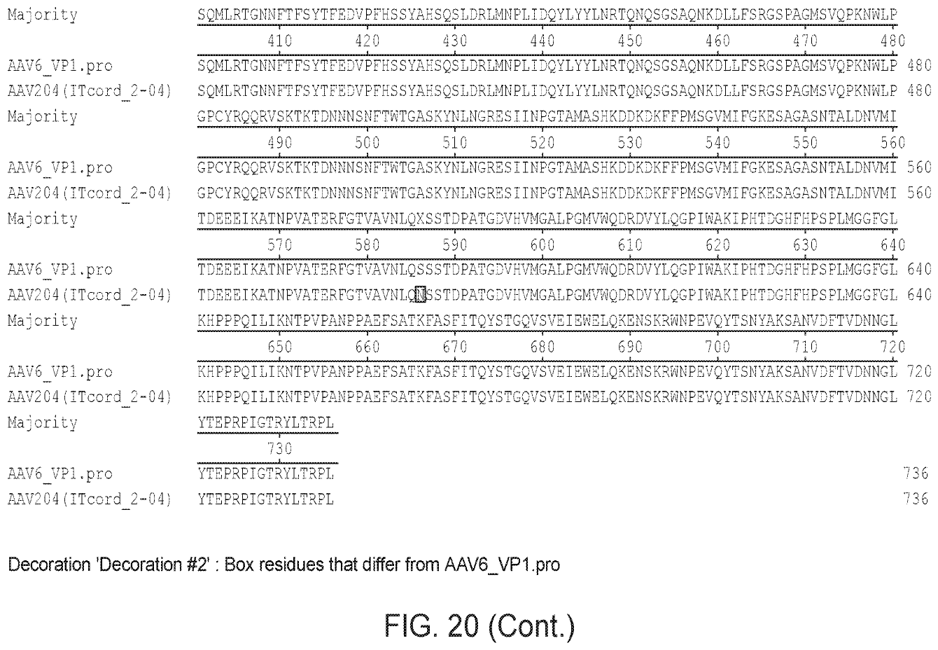

“Homology” or “identity” or “similarity” refers to sequence similarity between two peptides or between two nucleic acid molecules. Percent identity can be determined by comparing a position in each sequence that may be aligned for purposes of comparison. When a position in the compared sequence is occupied by the same base or amino acid, then the molecules are identical at that position. A degree of identity between sequences is a function of the number of matching positions shared by the sequences. “Unrelated” or “non-homologous” sequences share less than 40% identity, less than 25% identity, with one of the sequences of the present disclosure. Alignment and percent sequence identity may be determined for the nucleic acid or amino acid sequences provided herein by importing said nucleic acid or amino acid sequences into and using ClustalW (available at https://genome.jp/tools-bin/clustalw/). For example, the ClustalW parameters used for performing the protein sequence alignments found herein (e.g., FIGS. 20-23) were generated using the Gonnet (for protein) weight matrix. In embodiments, the ClustalW parameters used for performing nucleic acid sequence alignments using the nucleic acid sequences found herein are generated using the ClustalW (for DNA) weight matrix.

As used herein, amino acid modifications may be amino acid substitutions, amino acid deletions or amino acid insertions. Amino acid substitutions may be conservative amino acid substitutions or non-conservative amino acid substitutions. A conservative replacement (also called a conservative mutation, a conservative substitution or a conservative variation) is an amino acid replacement in a protein that changes a given amino acid to a different amino acid with similar biochemical properties (e.g., charge, hydrophobicity or size). As used herein, “conservative variations” refer to the replacement of an amino acid residue by another, biologically similar residue. Examples of conservative variations include the substitution of one hydrophobic residue such as isoleucine, valine, leucine or methionine for another; or the substitution of one charged or polar residue for another, such as the substitution of arginine for lysine, glutamic acid for aspartic acid, glutamine for asparagine, and the like. Other illustrative examples of conservative substitutions include the changes of: alanine to serine; asparagine to glutamine or histidine; aspartate to glutamate; cysteine to serine; glycine to proline; histidine to asparagine or glutamine; lysine to arginine, glutamine, or glutamate; phenylalanine to tyrosine, serine to threonine; threonine to serine; tryptophan to tyrosine; tyrosine to tryptophan or phenylalanine; and the like.

As used herein, the term “vector” refers to a nucleic acid comprising, consisting essentially of, or consisting of an intact replicon such that the vector may be replicated when placed within a cell, for example by a process of transfection, infection, or transformation. It is understood in the art that once inside a cell, a vector may replicate as an extrachromosomal (episomal) element or may be integrated into a host cell chromosome. Vectors may include nucleic acids derived from retroviruses, adenoviruses, herpesvirus, baculoviruses, modified baculoviruses, papovaviruses, or otherwise modified naturally-occurring viruses. Exemplary non-viral vectors for delivering nucleic acid include naked DNA; DNA complexed with cationic lipids, alone or in combination with cationic polymers; anionic and cationic liposomes; DNA-protein complexes and particles comprising, consisting essentially of, or consisting of DNA condensed with cationic polymers such as heterogeneous polylysine, defined-length oligopeptides, and polyethyleneimine, in some cases contained in liposomes; and the use of ternary complexes comprising, consisting essentially of, or consisting of a virus and polylysine-DNA.

With respect to general recombinant techniques, vectors that contain both a promoter and a cloning site into which a polynucleotide can be operatively linked are well known in the art. Such vectors are capable of transcribing RNA in vitro or in vivo, and are commercially available from sources such as Agilent Technologies (Santa Clara, Calif) and Promega Biotech (Madison, Wis.). In order to optimize expression and/or in vitro transcription, it may be necessary to remove, add or alter 5′ and/or 3′ untranslated portions of cloned transgenes to eliminate extra, potential inappropriate alternative translation initiation codons or other sequences that may interfere with or reduce expression, either at the level of transcription or translation. Alternatively, consensus ribosome binding sites can be inserted immediately 5′ of the start codon to enhance expression.

A “viral vector” is defined as a recombinantly produced virus or viral particle that contains a polynucleotide to be delivered into a host cell, either in vivo, ex vivo or in vitro. Examples of viral vectors include retroviral vectors, AAV vectors, lentiviral vectors, adenovirus vectors, alphavirus vectors and the like. Alphavirus vectors, such as Semliki Forest virus-based vectors and Sindbis virus-based vectors, have also been developed for use in gene therapy and immunotherapy. See, e.g., Schlesinger and Dubensky (1999) Curr. Opin. Biotechnol. 5:434-439 and Ying, et al. (1999) Nat. Med. 5 (7): 823-827.

As used herein, the term “recombinant expression system” or “recombinant vector” refers to a genetic construct or constructs for the expression of certain genetic material formed by recombination.

A “gene delivery vehicle” is defined as any molecule that can carry inserted polynucleotides into a host cell. Examples of gene delivery vehicles are liposomes, micelles biocompatible polymers, including natural polymers and synthetic polymers; lipoproteins; polypeptides; polysaccharides; lipopolysaccharides; artificial viral envelopes; metal particles; bacteria; viruses, such as baculoviruses, adenoviruses and retroviruses; bacteriophage, cosmid, plasmid, and fungal vectors; and other recombination vehicles typically used in the art which have been described for expression in a variety of eukaryotic and prokaryotic hosts, and may be used for gene therapy as well as for simple protein expression. Liposomes that also comprise, consist essentially of, or consist of a targeting antibody or fragment thereof can be used in the methods disclosed herein. In addition to the delivery of polynucleotides to a cell or cell population, direct introduction of the proteins described herein to the cell or cell population can be done by the non-limiting technique of protein transfection, alternatively culturing conditions that can enhance the expression and/or promote the activity of the proteins disclosed herein are other non-limiting techniques.

A polynucleotide disclosed herein can be delivered to a cell or tissue using a gene delivery vehicle. “Gene delivery,” “gene transfer,” “transducing,” and the like as used herein, are terms referring to the introduction of an exogenous polynucleotide (sometimes referred to as a “transgene”) into a host cell, irrespective of the method used for the introduction. Such methods include a variety of well-known techniques such as vector-mediated gene transfer (by, e.g., viral infection/transfection, or various other protein-based or lipid-based gene delivery complexes) as well as techniques facilitating the delivery of “naked” polynucleotides (such as electroporation, “gene gun” delivery and various other techniques used for the introduction of polynucleotides). The introduced polynucleotide may be stably or transiently maintained in the host cell. Stable maintenance typically requires that the introduced polynucleotide either contains an origin of replication compatible with the host cell or integrates into a replicon of the host cell such as an extrachromosomal replicon (e.g., a plasmid) or a nuclear or mitochondrial chromosome. A number of vectors are known to be capable of mediating transfer of genes to mammalian cells, as is known in the art and described herein.

A “plasmid” is a DNA molecule that is typically separate from and capable of replicating independently of the chromosomal DNA. In many cases, it is circular and double-stranded. Plasmids provide a mechanism for horizontal gene transfer within a population of microbes and typically provide a selective advantage under a given environmental state. Plasmids may carry genes that provide resistance to naturally occurring antibiotics in a competitive environmental niche, or, alternatively, the proteins produced may act as toxins under similar circumstances. It is known in the art that while plasmid vectors often exist as extrachromosomal circular DNA molecules, plasmid vectors may also be designed to be stably integrated into a host chromosome either randomly or in a targeted manner, and such integration may be accomplished using either a circular plasmid or a plasmid that has been linearized prior to introduction into the host cell.

“Plasmids” used in genetic engineering are called “plasmid vectors”. Many plasmids are commercially available for such uses. The gene to be replicated is inserted into copies of a plasmid containing genes that make cells resistant to particular antibiotics, and a multiple cloning site (MCS, or polylinker), which is a short region containing several commonly used restriction sites allowing the easy insertion of DNA fragments at this location. Another major use of plasmids is to make large amounts of proteins. In this case, researchers grow bacteria or eukaryotic cells containing a plasmid harboring the gene of interest, which can be induced to produce large amounts of proteins from the inserted gene.

In aspects where gene transfer is mediated by a DNA viral vector, such as an adenovirus (Ad) or adeno-associated virus (AAV), a vector construct refers to the polynucleotide comprising, consisting essentially of, or consisting of the viral genome or part thereof, and a transgene.

The term “adeno-associated virus” or “AAV” as used herein refers to a member of the class of viruses associated with this name and belonging to the genus Dependoparvovirus, family Parvoviridae. Adeno-associated virus is a single-stranded DNA virus that grows only in cells in which certain functions are provided by a co-infecting helper virus. General information and reviews of AAV can be found in, for example, Carter, 1989, Handbook of Parvoviruses, Vol. 1, pp. 169-228, and Berns, 1990, Virology, pp. 1743-1764, Raven Press, (New York). It is fully expected that the same principles described in these reviews will be applicable to additional AAV serotypes characterized after the publication dates of the reviews because it is well known that the various serotypes are quite closely related, both structurally and functionally, even at the genetic level. (See, for example, Blacklowe, 1988, pp. 165-174 of Parvoviruses and Human Disease, J. R. Pattison, ed.; and Rose, Comprehensive Virology 3:1-61 (1974)). For example, all AAV serotypes apparently exhibit very similar replication properties mediated by homologous rep genes; and all bear three related capsid proteins such as those expressed in AAV2. The degree of relatedness is further suggested by heteroduplex analysis which reveals extensive cross-hybridization between serotypes along the length of the genome; and the presence of analogous self-annealing segments at the termini that correspond to “inverted terminal repeat sequences” (ITRs). The similar infectivity patterns also suggest that the replication functions in each serotype are under similar regulatory control. Multiple serotypes of this virus are known to be suitable for gene delivery; all known serotypes can infect cells from various tissue types. At least 11 sequentially numbered AAV serotypes are known in the art. Non-limiting exemplary serotypes useful in the methods disclosed herein include any of the 11 serotypes, e.g., AAV2, AAV8, AAV9, or variant serotypes, e.g., AAV-DJ and AAV PHP.B. The AAV particle comprises, consists essentially of, or consists of three major viral proteins: VP1, VP2 and VP3. In embodiments, the AAV refers to the serotype AAV1, AAV2, AAV4, AAV5, AAV6, AAV7, AAV8, AAV9, AAV10, AAV11, AAV12, AAV13, AAVPHP.B, or AAVrh74.

An “AAV vector” as used herein refers to a vector comprising, consisting essentially of, or consisting of one or more heterologous nucleic acid (HNA) sequences and one or more AAV inverted terminal repeat sequences (ITRs). Such AAV vectors can be replicated and packaged into infectious viral particles when present in a host cell that provides the functionality of rep and cap gene products; for example, by transfection of the host cell. In embodiments, AAV vectors contain a promoter, at least one nucleic acid that may encode at least one protein or RNA, and/or an enhancer and/or a terminator within the flanking ITRs that is packaged into the infectious AAV particle. The encapsidated nucleic acid portion may be referred to as the AAV vector genome. Plasmids containing AAV vector may also contain elements for manufacturing purposes, e.g., antibiotic resistance genes, etc., but these are not encapsidated and thus do not form part of the AAV particle.

As used herein, the term “viral capsid” or “capsid” refers to the proteinaceous shell or coat of a viral particle. Capsids function to encapsidate, protect, transport, and release into the host cell a viral genome. Capsids are generally comprised of oligomeric structural subunits of protein (“capsid proteins”). As used herein, the term “encapsidated” means enclosed within a viral capsid. The viral capsid of AAV is composed of a mixture of three viral capsid proteins: VP1, VP2, and VP3. The mixture of VP1, VP2 and VP3 contains 60 monomers that are arranged in a T=1 icosahedral symmetry in a ratio of 1:1:10 (VP1:VP2:VP3) or 1:1:20 (VP1:VP2:VP3) as described in Sonntag F et al., (June 2010). “A viral assembly factor promotes AAV2 capsid formation in the nucleolus”. Proceedings of the National Academy of Sciences of the United States of America. 107 (22): 10220-5, and Rabinowitz J E, Samulski R J (December 2000). “Building a better vector: the manipulation of AAV virions”. Virology. 278 (2): 301-8, each of which is incorporated herein by reference in its entirety.

An “AAV virion” or “AAV viral particle” or “AAV viral vector” or “AAV vector particle” or “AAV particle” refers to a viral particle composed of at least one AAV capsid protein and an encapsidated polynucleotide AAV vector. Thus, production of AAV vector particle necessarily includes production of AAV vector, as such a vector is contained within an AAV vector particle.

As used herein, the term “helper” in reference to a virus or plasmid refers to a virus or plasmid used to provide the additional components necessary for replication and packaging of any one of the AAV vectors disclosed herein. The components encoded by a helper virus may include any genes required for virion assembly, encapsidation, genome replication, and/or packaging. For example, the helper virus or plasmid may encode necessary enzymes for the replication of the viral genome. Non-limiting examples of helper viruses and plasmids suitable for use with AAV constructs include pHELP (plasmid), adenovirus (virus), or herpesvirus (virus). In embodiments, the pHELP plasmid may be the pHELPK plasmid, wherein the ampicillin expression cassette is exchanged with a kanamycin expression cassette; pHELPK has the sequence shown in SEQ ID NO: 92.

As used herein, a packaging cell (or a helper cell) is a cell used to produce viral vectors. Producing recombinant AAV viral vectors requires Rep and Cap proteins provided in trans as well as gene sequences from Adenovirus that help AAV replicate. In some aspects, Packaging/helper cells contain a plasmid is stably incorporated into the genome of the cell. In other aspects, the packaging cell may be transiently transfected. Typically, a packaging cell is a eukaryotic cell, such as a mammalian cell or an insect cell.

As used herein, a reporter protein is a detectable protein that is operably linked to a promoter to assay the expression (for example, tissue specificity and/or strength) of the promoter. In aspects, a reporter protein may be operably linked to a polypeptide. In aspects, reporter proteins may be used in monitoring DNA delivery methods, functional identification and characterization of promoter and enhancer elements, translation and transcription regulation, mRNA processing and protein: protein interactions. Non-limiting examples of a reporter protein are β-galactosidase; a fluorescent protein, such as, Green Fluorescent Protein (GFP) or Red Fluorescent Protein (RFP); luciferase; glutathione S-transferase; and maltose binding protein.

A “composition” is intended to mean a combination of active polypeptide, polynucleotide or antibody, and another compound or composition, inert (e.g., a detectable label) or active (e.g., a gene delivery vehicle).

A “pharmaceutical composition” is intended to include the combination of an active polypeptide, polynucleotide or antibody with a carrier, inert or active such as a solid support, making the composition suitable for diagnostic or therapeutic use in vitro, in vivo or ex vivo.

As used herein, the term “pharmaceutically acceptable carrier” encompasses any of the standard pharmaceutical carriers, such as a phosphate buffered saline solution, water, and emulsions, such as an oil/water or water/oil emulsion, and various types of wetting agents. The compositions also can include stabilizers and preservatives. For examples of carriers, stabilizers and adjuvants, see Martin (1975) Remington's Pharm. Sci., 15th Ed. (Mack Publ. Co., Easton).

A “subject” of diagnosis or treatment is a cell or an animal such as a mammal, or a human. A subject is not limited to a specific species and includes non-human animals subject to diagnosis or treatment and those subject to infections or animal models, including, without limitation, simian, murine, rat, canine, or leporid species, as well as other livestock, sport animals, or pets. In embodiments, the subject is a human.

The term “tissue” is used herein to refer to tissue of a living or deceased organism or any tissue derived from or designed to mimic a living or deceased organism. The tissue may be healthy, diseased, and/or have genetic mutations. The biological tissue may include any single tissue (e.g., a collection of cells that may be interconnected), or a group of tissues making up an organ or part or region of the body of an organism. The tissue may comprise, consist essentially of, or consist of a homogeneous cellular material or it may be a composite structure such as that found in regions of the body including the thorax which for instance can include lung tissue, skeletal tissue, and/or muscle tissue. Exemplary tissues include, but are not limited to those derived from liver, lung, thyroid, skin, pancreas, blood vessels, bladder, kidneys, brain, biliary tree, duodenum, abdominal aorta, iliac vein, heart and intestines, including any combination thereof.

As used herein, “treating” or “treatment” of a disease in a subject refers to (1) preventing the symptoms or disease from occurring in a subject that is predisposed or does not yet display symptoms of the disease; (2) inhibiting the disease or arresting its development; or (3) ameliorating or causing regression of the disease or the symptoms of the disease. As understood in the art, “treatment” is an approach for obtaining beneficial or desired results, including clinical results. For the purposes of the present technology, beneficial or desired results can include one or more, but are not limited to, alleviation or amelioration of one or more symptoms, diminishment of extent of a condition (including a disease), stabilized (i.e., not worsening) state of a condition (including disease), delay or slowing of condition (including disease), progression, amelioration or palliation of the condition (including disease), states and remission (whether partial or total), whether detectable or undetectable.

As used herein the term “effective amount” intends to mean a quantity sufficient to achieve a desired effect. In the context of therapeutic or prophylactic applications, the effective amount will depend on the type and severity of the condition at issue and the characteristics of the individual subject, such as general health, age, sex, body weight, and tolerance to pharmaceutical compositions. In the context of gene therapy, In embodiments the effective amount is the amount sufficient to result in regaining part or full function of a gene that is deficient in a subject. In other some embodiments, the effective amount of an AAV viral particle is the amount sufficient to result in expression of a gene in a subject. In embodiments, the effective amount is the amount required to increase galactose metabolism in a subject in need thereof. The skilled artisan will be able to determine appropriate amounts depending on these and other factors.

In embodiments, the effective amount will depend on the size and nature of the application in question. It will also depend on the nature and sensitivity of the target subject and the methods in use. The skilled artisan will be able to determine the effective amount based on these and other considerations. The effective amount may comprise, consist essentially of, or consist of one or more administrations of a composition depending on the embodiment.

As used herein, the term “administer” or “administration” intends to mean delivery of a substance to a subject such as an animal or human. Administration can be effected in one dose, continuously or intermittently throughout the course of treatment. Methods of determining the most effective means and dosage of administration are known to those of skill in the art and will vary with the composition used for therapy, the purpose of the therapy, as well as the age, health or gender of the subject being treated. Single or multiple administrations can be carried out with the dose level and pattern being selected by the treating physician or in the case of pets and other animals, treating veterinarian.

### AAV Structure and Function

AAV is a replication-deficient parvovirus, the single-stranded DNA genome of which is about 4.7 kb in length, including two 145-nucleotide inverted terminal repeat (ITRs). There are multiple serotypes of AAV. The nucleotide sequences of the genomes of the AAV serotypes are known. For example, the complete genome of AAV-1 is provided in GenBank Accession No. NC_002077; the complete genome of AAV-2 is provided in GenBank Accession No. NC 001401 and Srivastava et al., J. Virol., 45:555-564 (1983); the complete genome of AAV-3 is provided in GenBank Accession No. NC_1829; the complete genome of AAV-4 is provided in GenBank Accession No. NC_001829; the AAV-5 genome is provided in GenBank Accession No. AF085716; the complete genome of AAV-6 is provided in GenBank Accession No. NC_001862; at least portions of AAV-7 and AAV-8 genomes are provided in GenBank Accession Nos. AX753246 and AX753249, respectively; the AAV-9 genome is provided in Gao et al., J. Virol., 78:6381-6388 (2004); the AAV-10 genome is provided in Mol. Ther., 13 (1): 67-76 (2006); and the AAV-11 genome is provided in Virology, 330 (2): 375-383 (2004). The sequence of the AAV rh.74 genome is provided in U.S. Pat. No. 9,434,928, incorporated herein by reference in its entirety. U.S. Pat. No. 9,434,928 also provide the sequences of the capsid proteins and a self-complementary genome. In one aspect, the genome is a self-complementary genome. Cis-acting sequences directing viral DNA replication (rep), encapsidation/packaging and host cell chromosome integration are contained within the AAV ITRs. Three AAV promoters (named p5, p19, and p40 for their relative map locations) drive the expression of the two AAV internal open reading frames encoding rep and cap genes. The two rep promoters (p5 and p19), coupled with the differential splicing of the single AAV intron (at nucleotides 2107 and 2227), result in the production of four rep proteins (rep 78, rep 68, rep 52, and rep 40) from the rep gene. Rep proteins possess multiple enzymatic properties that are ultimately responsible for replicating the viral genome.

The cap gene is expressed from the p40 promoter and encodes the three capsid proteins, VP1, VP2, and VP3. Alternative splicing and non-consensus translational start sites are responsible for the production of the three related capsid proteins. More specifically, after the single mRNA from which each of the VP1, VP2 and VP3 proteins are translated is transcribed, it can be spliced in two different manners: either a longer or shorter intron can be excised, resulting in the formation of two pools of mRNAs: a 2.3 kb- and a 2.6 kb-long mRNA pool. The longer intron is often preferred and thus the 2.3-kb-long mRNA can be called the major splice variant. This form lacks the first AUG codon, from which the synthesis of VP1 protein starts, resulting in a reduced overall level of VP1 protein synthesis. The first AUG codon that remains in the major splice variant is the initiation codon for the VP3 protein. However, upstream of that codon in the same open reading frame lies an ACG sequence (encoding threonine) which is surrounded by an optimal Kozak (translation initiation) context. This contributes to a low level of synthesis of the VP2 protein, which is actually the VP3 protein with additional N terminal residues, as is VP1, as described in Becerra S P et al., (December 1985). “Direct mapping of adeno-associated virus capsid proteins B and C: a possible ACG initiation codon”. Proceedings of the National Academy of Sciences of the United States of America. 82 (23): 7919-23, Cassinotti P et al., (November 1988). “Organization of the adeno-associated virus (AAV) capsid gene: mapping of a minor spliced mRNA coding for virus capsid protein 1”. Virology. 167 (1): 176-84, Muralidhar S et al., (January 1994). “Site-directed mutagenesis of adeno-associated virus type 2 structural protein initiation codons: effects on regulation of synthesis and biological activity”. Journal of Virology. 68 (1): 170-6, and Trempe J P, Carter B J (September 1988). “Alternate mRNA splicing is required for synthesis of adeno-associated virus VP1 capsid protein”. Journal of Virology. 62 (9): 3356-63, each of which is herein incorporated by reference. A single consensus poly-A site is located at map position 95 of the AAV genome. The life cycle and genetics of AAV are reviewed in Muzyczka, Current Topics in Microbiology and Immunology, 158:97-129 (1992).

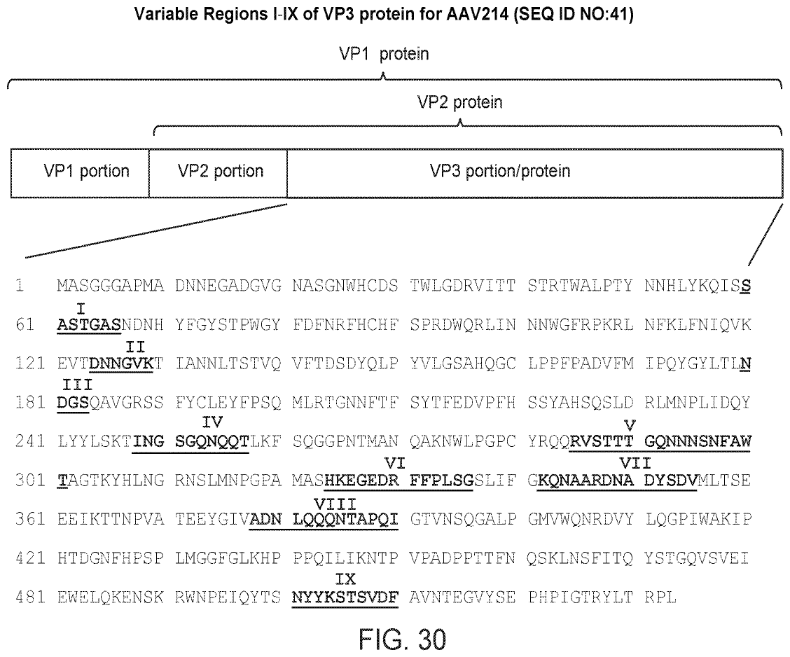

Each VP1 protein contains a VP1 portion, a VP2 portion and a VP3 portion. The VP1 portion is the N-terminal portion of the VP1 protein that is unique to the VP1 protein. The VP2 portion is the amino acid sequence present within the VP1 protein that is also found in the N-terminal portion of the VP2 protein. The VP3 portion and the VP3 protein have the same sequence. The VP3 portion is the C-terminal portion of the VP1 protein that is shared with the VP1 and VP2 proteins. See FIG. 30.

The VP3 protein can be further divided into discrete variable surface regions I-IX (VR-I-IX). Each of the variable surface regions (VRs) can comprise or contain specific amino acid sequences that either alone or in combination with the specific amino acid sequences of each of the other VRs can confer unique infection phenotypes (e.g., decreased antigenicity, improved transduction and/or tissue-specific tropism relative to other AAV serotypes) to a particular serotype as described in DiMatta et al., “Structural Insight into the Unique Properties of Adeno-Associated Virus Serotype 9” J. Virol., Vol. 86 (12): 6947-6958 Jun. 2012, the contents of which are incorporated herein by reference.

AAV possesses unique features that make it attractive as a vector for delivering foreign DNA to cells, for example, in gene therapy. AAV infection of cells in culture is noncytopathic, and natural infection of humans and other animals is silent and asymptomatic. Moreover, AAV infects many mammalian cells allowing the possibility of targeting many different tissues in vivo. Moreover, AAV transduces slowly dividing and non-dividing cells, and can persist essentially for the lifetime of those cells as a transcriptionally active nuclear episome (extrachromosomal element). The AAV proviral genome is inserted as cloned DNA in plasmids, which makes construction of recombinant genomes feasible. Furthermore, because the signals directing AAV replication and genome encapsidation are contained within the ITRs of the AAV genome, some or all of the internal approximately 4.3 kb of the genome (encoding replication and structural capsid proteins, rep-cap) may be replaced with foreign DNA to generate AAV vectors. The rep and cap proteins may be provided in trans. Another significant feature of AAV is that it is an extremely stable and hearty virus. It easily withstands the conditions used to inactivate adenovirus (56° to 65° C. for several hours), making cold preservation of AAV less critical. AAV may even be lyophilized. Finally, AAV-infected cells are not resistant to superinfection.

Multiple studies have demonstrated long-term (>1.5 years) recombinant AAV-mediated protein expression in muscle. See, Clark et al., Hum Gene Ther, 8:659-669 (1997); Kessler et al., Proc Nat. Acad Sc. USA, 93:14082-14087 (1996); and Xiao et al., J Virol, 70:8098-8108 (1996). See also, Chao et al., Mol Ther, 2:619-623 (2000) and Chao et al., Mol Ther, 4:217-222 (2001). Moreover, because muscle is highly vascularized, recombinant AAV transduction has resulted in the appearance of transgene products in the systemic circulation following intramuscular injection as described in Herzog et al., Proc Natl Acad Sci USA, 94:5804-5809 (1997) and Murphy et al., Proc Natl Acad Sci USA, 94:13921-13926 (1997). Moreover, Lewis et al., J Virol, 76:8769-8775 (2002) demonstrated that skeletal myofibers possess the necessary cellular factors for correct antibody glycosylation, folding, and secretion, indicating that muscle is capable of stable expression of secreted protein therapeutics. Recombinant AAV (rAAV) genomes of the invention comprise, consist essentially of, or consist of a nucleic acid molecule encoding a therapeutic protein (e.g., CFTR) and one or more AAV ITRs flanking the nucleic acid molecule. AAV DNA in the rAAV genomes may be from any AAV serotype for which a recombinant virus can be derived including, but not limited to, AAV serotypes AAV-1, AAV-2, AAV-3, AAV-4, AAV-5, AAV-6, AAV-7, AAV-8, AAV-9, AAV-10, AAV-11, AAV-12, AAV-13, AAV PHP.B and AAV rh74. Production of pseudotyped rAAVis disclosed in, for example, WO2001083692. Other types of rAAV variants, for example rAAV with capsid mutations, are also contemplated. See, e.g., Marsic et al., Molecular Therapy, 22 (11): 1900-1909 (2014). The nucleotide sequences of the genomes of various AAV serotypes are known in the art.

### AAV Vector Particles, Capsid Proteins, and AAV Vectors

Provided herein are AAV vector particles, AAV Vectors, and capsid proteins that have desirable tissue specificity and find use in delivering a variety of therapeutic payloads, including nucleic acids, and proteins useful in the treatment of disease.

### AAV Capsid Proteins

The disclosure provides AAV particles possessing properties of high gene transfer efficiency and increased tissue tropism. AAV vector delivery currently relies on the use of serotype selection for tissue targeting based on the natural tropism of the virus or by the direct injection into target tissues. If systemic delivery is required to achieve maximal therapeutic benefit, then serotype selection is the only available option for tissue targeting combined with tissue specific promoters. Many currently available AAV vectors are thus suboptimal for gene therapy.

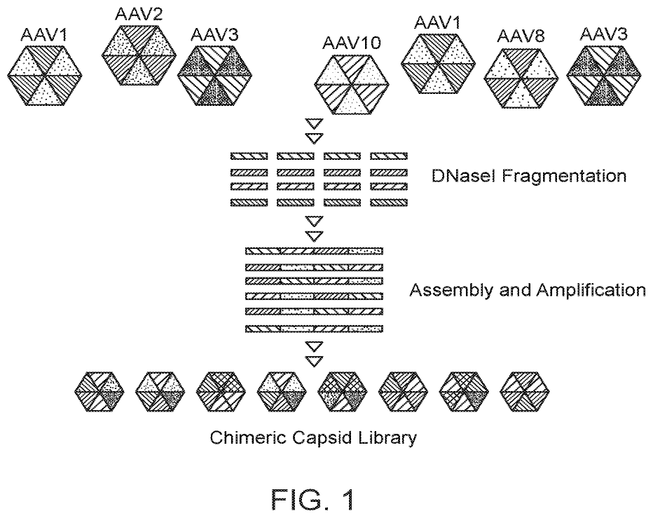

The present disclosure provides AAV capsid protein sequences that confer high gene transfer efficiency and increased tissue specificity on the AAV capsids comprising them. In embodiments, the AAV capsid sequences provided herein were generated using the AAV capsid generating platform shown in FIG. 1.

In embodiments, the VP1 capsid protein comprises any one of the amino acid sequences listed in Table 1, or a sequence having up to 1, 2, 3, 4, 5, 6, 7, 8, 9, or 10 amino acids mutated, deleted or added as compared to, any one of the amino acid sequence listed in Table 1. In aspects, up to 20 amino acids, up to 30 amino acids, or up to 40 amino acids may be mutated, deleted or added compared to these sequences, In embodiments, the VP1 capsid protein is encoded by any one of the nucleic acid sequences listed in Table 1, or a sequence having up to 5, up to 10, up to 30, or up to 60 nucleotide changes to any one of the nucleic acid sequences listed in Table 1.

TABLE 1
VP1 Capsid Proteins

| Amino Acid | NA | AAV |
|---|---|---|
| SEQ ID NO: | SEQ ID NO: | Capsid Name |
| 1 | 98 | AAV 110 |
| 2 | 15 | AAV 204 |
| 3 | 18 | AAV 214 |
| 30 | 19 | AAV 214A |
| 31 | 20 | AAV 214e |
| 32 | 21 | AAV 214e8 |
| 33 | 22 | AAV 214e9 |
| 34 | 23 | AAV 214e0 |
| 49 | 47 | AAV ITB102_45 |
| 84 | 82 | AAV 214AB |

In embodiments, the AAV VP1 protein comprises, consists essentially of, or consists of an amino acid sequence of SEQ ID NOs: 1-3, 30-34, 49 or 84, or a sequence having up to 1, 2, 3, 4, 5, 6, 7, 8, 9, or 10 amino acids different from SEQ ID NOs: 1-3, 30-34, 49 or 84. Also provided are polynucleotides encoding these VP1 proteins. In embodiments, the polynucleotides encoding the VP1 proteins comprise, consist essentially of, or consist of the sequence of SEQ ID NOs: 15, 18-23, 47, 82, and 98 or a sequence having up to 5, up to 10, or up to 30 nucleotide changes to SEQ ID NOs: 15, 18-23, 47, 82, and 98.

In embodiments, the AAV capsid sequence is an AAV-110 capsid protein (SEQ ID NO: 1), AAV204 capsid protein (SEQ ID NO: 2), AAV214 capsid protein (SEQ ID NO: 3) or AAV ITB102_45 capsid protein (SEQ ID NO: 49). In embodiments, the AAV capsid protein is a variant of the AAV214 capsid protein. In embodiments, the AAV capsid protein is AAV214A (SEQ ID NO: 30), AAV-214-AB (SEQ ID NO: 84), AAV214e (SEQ ID NO: 31), AAV214e8 (SEQ ID NO: 32), AAV214e9 (SEQ ID NO: 33) or AAV214e10 (SEQ ID NO: 34).

Sequences for exemplary VP2 and VP3 proteins are provided in Table 2 and Table 3. Given the VP2 and VP3 sequences, the VP1 portions may be determined by alignment with the full, VP1 protein sequence.

TABLE 2
VP2 Capsid Proteins

| Amino Acid | |
|---|---|
| SEQ ID NO: | Name |
| 35 | 214 |
| 36 | 214A |
| 37 | 214e |
| 38 | 214e8 |
| 39 | 214e9 |
| 40 | 214e10 |
| 85 | 214AB |
| 50 | ITB102_45 |

An exemplary nucleic acid for ITB102_45 is SEQ ID NO:47. Exemplary nucleic acids for the other capsid VP2 portions may be derived from the corresponding portions of the VP1 capsid protein nucleic acids.

TABLE 3
VP3 Capsid proteins

| Amino Acid | NA | AAV |
|---|---|---|
| SEQ ID NO: | SEQ ID NO: | Capsid Name |
| 17 | 16 | 204 |
| 41 | 24 | 214 |
| 42 | 25 | 214A |
| 43 | 26 | 214e |
| 44 | 27 | 214e8 |
| 45 | 28 | 214e9 |
| 46 | 29 | 214e10 |
| 86 | 83 | 214AB |
| 51 | 48 | ITB102_45 |

The VP3 proteins of AAV214, AAV214e, AAV214e8, AAV214e9, AAV214e10 have the same amino acid (SEQ ID NO:41) and nucleic acid (SEQ ID NO: 24) sequences.

In embodiments, the AAV VP2 proteins comprise, consist essentially of, or consist of an amino acid sequence of any one of SEQ ID NOs: 35-40, 50 or 85, or a sequence having up to 1, 2, 3, 4, 5, 6, 7, 8, 9, or 10 amino acids different from SEQ ID NOs: 35-40, 50 or 85. Also provided are polynucleotides encoding these VP2 proteins. In embodiments, the polynucleotide encoding the VP2 protein comprises, consists essentially of, or consists of the sequence of SEQ ID NO: 47, or a sequence having up to 5, up to 10, or up to 30 nucleotide changes to SEQ ID NOs: 47.

In embodiments, the AAV VP3 proteins comprise, consist essentially of, or consist of an amino acid sequence of SEQ ID NOs: 17, 41-46, 51, or 86, or a sequence having up to 1, 2, 3, 4, 5, 6, 7, 8, 9, or 10 amino acids different from SEQ ID NOs: 17, 41-46, 51, or 86. Also provided are polynucleotides encoding these VP3 proteins. In embodiments, the polynucleotides encoding the proteins that comprise, consist essentially of, or consist of the sequence of SEQ ID NOs: 16, 24-29, 48 and 83, or a sequence having up to 5, up to 10, or up to 30 nucleotide changes to SEQ ID NOs: 16, 24-29, 48 and 83.

In embodiments, the AAV capsid protein is a chimeric protein. In embodiments, a VP1, VP2, or VP3 portion of the AAV capsid protein disclosed herein may be replaced with a VP1, VP2, or VP3 portion from a different AAV capsid protein disclosed herein.

In embodiments, provided herein is an AAV capsid protein comprising a leucine residue at amino acid 129, an asparagine residue at amino acid 586 and a glutamic acid residue at amino acid 723, wherein amino acid positions in the AAV capsid protein are numbered with respect to amino acid positions in the amino acid sequence of SEQ ID NO: 2. In some cases, the protein comprises the amino acid sequence of SEQ ID NO: 2. In other cases, these amino acids may be introduced into other capsid proteins.

In embodiments, provided herein is an AAV VP1 capsid protein comprising a VP1 portion, a VP2 portion and a VP3 portion, wherein the VP1 portion comprises a leucine (L) residue at amino acid 129, wherein the VP2 portion comprises a threonine (T) or asparagine (N) residue at amino acid 157 and a lysine (K) or serine(S) residue at amino acid 162, and wherein the VP3 portion comprises asparagine (N) residue at amino acid 223, an alanine (A) residue at amino acid 224, a histidine (H) residue at amino acid 272, a threonine (T) residue at amino acid 410, a histidine (H) residue at amino acid 724 and a proline (P) residue at amino acid 734, wherein amino acid positions in the AAV capsid protein are numbered with respect to amino acid positions in the amino acid sequence of SEQ ID NO: 3 (i.e., VP1 capsid subunit numbering).

In embodiments, the VP1 portion further comprises an aspartic acid (D) or alanine (A) residue at amino acid 24, wherein amino acid positions in the AAV capsid protein are numbered with respect to amino acid positions in the amino acid sequence of SEQ ID NO: 3. In embodiments, the VP2 portion further comprises one or more of (i) a proline (P) residue at amino acid 148; (ii) an arginine (R) residue inserted at amino acid 152; (iii) an arginine (R) residue at amino acid 168; (iv) an isoleucine (I) residue at amino acid 189; and (v) a serine(S) residue at amino acid 200, wherein amino acid positions in the AAV capsid protein are numbered with respect to amino acid positions in the amino acid sequence of SEQ ID NO: 3.

In embodiments, one or more variable regions I through IX (see FIG. 30) in the disclosed VP3 portion capsid proteins may be removed and replaced with alternative regions. Suitable alternatives are identified in the table below. The location for these, as well as the identity of additional alternatives may be identified by alignment to SEQ ID NO:41 as shown in FIG. 30. In embodiments, one or more VRs may have an insertion of 1, 2 or 3 amino acids. In embodiments, one or more VRs may have a deletion of 1, 2 or 3 amino acids.

| VR | Sequence |
|---|---|
| I | SASTGAS (SEQ ID NO. 52); |
| | NSTSGGSS (SEQ ID NO. 53); |
| | SSTSGGSS (SEQ ID NO: 87) |
| II | DNNGVK (SEQ ID NO. 54) |
| III | NDGS (SEQ ID NO. 55) |
| IV | INGSGQNQQT (SEQ ID NO. 56) |
| VI | RVSTTTGQNNNSNFAWTA (SEQ ID NO. 57) |
| VII | HKEGEDRFFPLSG (SEQ ID NO. 58); |
| VIII | ADNLQQQNTAPQI (SEQ ID NO. 60); |
| IX | NYYKSTSVDF (SEQ ID NO. 61). |

The disclosure provides nucleic acids encoding any one of the AAV capsid proteins disclosed herein. The disclosure also provides vectors comprising any one of the nucleic acids disclosed herein.

In embodiments, AAV is an AAV9 serotype. Alternative serotypes or modified capsid viruses can be used to optimize neuronal tropism. Alternative vectors include: a modified AAV9 serotype vector for higher neuronal tropism than standard AAV9, e.g., PHP.B that uses a Cre-lox recombination system to identify neuronally targeted vectors. Alternatively, the AAV9 PHP.B has a modified amino acid 498 of VP1 from asparagine to lysine to reduce the liver tropism. Further variants of AAVrh74 that have mutated several amino acids can be used for very broad tissue tropism including the brain.

### AAV Vectors

The AAV vectors supply the nucleic acid that becomes encapsidated into the AAV vector particle including element(s) involved in controlling expression of the nucleic acids in the subject, as well as the ITRs to facilitate encapsidation. In embodiments, the AAV vectors disclosed herein comprise at least one heterologous nucleic acid (HNA) sequence, which, when expressed in a cell of a subject, is effective to treat a disease or disorder. In embodiments, the HNA sequence comprises a transgene. In embodiments, the AAV vectors comprise at least one ITR sequence and at least one transgene. In embodiments, the transgene encodes a therapeutic protein or a therapeutic RNA.

In embodiments, control of transgene expression in the host cell may be regulated by regulatory elements contained within the AAV vector, including promoter sequences, and poly-A sites. In embodiments, the AAV vector may also encode a signal peptide. In embodiments, the AAV vectors have 5′ and 3′ inverted terminal repeats (ITRs). The 5′ ITR is located upstream of a promoter, which in turn is upstream of the transgene. In embodiments, the 5′ and 3′ ITR have the same sequence. In embodiments, they have a different sequence. In embodiments, an AAV vector of the disclosure may comprise, in 5′ to 3′ orientation, a first (5′) ITR, a promoter, a transgene, a poly-A site, and a second (3′) ITR.

In embodiments, the AAV vector has the nucleotide sequence shown in SEQ ID NO: 88 (pA_CF1), SEQ ID NO: 89 (pA_CF3), SEQ ID NO: 90 (pA_CF5), or SEQ ID NO: 91 (pA_CF7). These vectors contain the following components:

| Plasmid | | | | Signal | |
|---|---|---|---|---|---|
| name | Promoter | Intron | Kozak | Peptide | CFTRΔR |
| pA-CF1 | U1a | none | Full | none | CFTRΔR |
| | | | | | Codon |
| | | | | | optimized |
| pA-CF3 | U1a | none | Full | none | CFTRΔR |
| | | | | | codon |
| | | | | | optimized |
| pA-CF5 | H1 mut. | none | Full | none | CFTRΔR |
| | | | | | Codon |
| | | | | | optimized |
| pA-CF7 | H1 mut. | none | Full | none | CFTR |
| | | | | | Codon |
| | | | | | optimized |
| | | | | | (full size) |

In embodiments, the HNA (for example, an HNA comprising a transgene) is operably linked to a constitutive promoter. The constitutive promoter can be any constitutive promoter known in the art and/or provided herein. In embodiments, the constitutive promoter comprises, consists essentially of, or consists of a Rous sarcoma virus (RSV) LTR promoter (optionally with the RSV enhancer), a cytomegalovirus (CMV) promoter, an SV40 promoter, a dihydrofolate reductase promoter, a beta-actin promoter, a phosphoglycerol kinase (PGK) promoter, a U6 promoter, an H1 promoter, a hybrid chicken beta actin promoter, a MeCP2 promotor, an H1 promoter, a U1a promoter, a mMeP418 promoter, a mMeP426 promoter, a minimal MeCP2 promoter, a CAG promoter, or an EF1 promoter. It is known in the art that the nucleotide sequences of such promoters may be modified in order to increase or decrease the efficiency of mRNA transcription. See, e.g., Gao et al. (2018) Mol. Ther.: Nucleic Acids 12:135-145 (modifying TATA box of 7SK, U6 and H1 promoters to abolish RNA polymerase III transcription and stimulate RNA polymerase II-dependent mRNA transcription). In embodiments, the HNA sequence is operably linked to a tissue-specific control promoter, or an inducible promoter. In embodiments, the tissue-specific control promoter is a central nervous system (CNS) cell-specific promoter, a lung-specific promoter, a skin-specific promoter, a muscle-specific promoter, a liver-specific promoter, an eye-specific promoter (e.g., a VMD2, or mRho promoter).

In embodiments, the promoter may comprise, consist essentially of or consist of a polynucleotide having the sequence of SEQ ID NO: 96 (mouse U1 promoter) or a SEQ ID NO: 97 (a H1 promoter). In embodiments, the promoter is an U1a or U1b promoter, EF1 promoter, or CBA (chicken beta-actin). In embodiments, the promoter may comprise, consist essentially of or consist of any one of the nucleic acid sequences listed in Table 5, or a sequence having up to 5, up to 10, or up to 30 nucleotide changes to any one of the nucleic acid sequences listed in Table 5.

TABLE 5

| | Nucleic acid |
|---|---|
| Promoter name | SEQ ID No. |
| Mouse U1a promoter | 152 |
| Polymerase III H1 mutant promoter | 153 |
| Chicken β-actin hybrid promoter CBh | 154 |
| (CBh promoter consists of CMV enhancer, | |
| CBA promoter, first CBA exon and partial | |
| intron) | |
| MeCP2 min promoter sequence | 155 |
| MeCP2 promoter sequence | 156 |
| MeCP418 promoter sequence | 157 |
| MeCP426 promoter sequence | 158 |
| VMD2 promoter | 159 |
| PDE6b promoter | 160 |
| mRho promoter | 161 |
| CMV promoter | 162 |
| UbC promoter | 163 |

In embodiments, the HNA sequence is operably liked to an additional regulatory element. The additional regulatory element can be a woodchuck hepatitis virus post-transcriptional regulatory element (“WPRE”). In embodiments, AAV vectors may comprise regulatory components suitable for growth and culture of the vector in a bacterial host for vector production purposes. For example, the vector may comprise genes for antibiotic resistance, and maintenance of the plasmid in bacteria, as well as associated regulatory elements to control protein expression in bacteria.

In embodiments, the HNA sequence is operably linked to a poly-A site. The polyadenylation site comprises, consists essentially of or consists of an MeCP2 poly-A site, a retinol dehydrogenase 1 (RDH1) poly-A site, a bovine growth hormone (BGH) poly-A site, an SV40 poly-A site, a SPA49 poly-A site, a sNRP-TK65 poly-A site, a sNRP poly-A site, or a TK65 poly-A site. An exemplary SPA49 poly-A sequence is described in Ostedgaard et al., Proc. Nat'l Acad. Sci. USA (Feb. 22, 2005) 102:2952-2957, incorporated herein by reference.

### Heterologous Nucleic Acids (HNA)

The AAV vectors disclosed herein infect and deliver one or more heterologous nucleic acids (HNA) to target tissues. In embodiments, the HNA sequences are transcribed and optionally, translated in the cells of the target tissue.

In some cases, the HNA encodes an antisense RNA, microRNA, siRNA, or guide RNA (gRNA). CRISPR technology has been used to target the genome of living cells for modification. Cas9 protein is a large enzyme that must be delivered efficiently to target tissues and cells to mediate gene repair through the CRISPR system and current CRISPR/Cas9 gene correction protocols suffer from a number of drawbacks. Long-term expression of Cas9 can elicit host immune responses. An additional guide RNA may be delivered via a separate vector due to packaging constraints. In embodiments, the HNA encodes a Cas9 protein or an equivalent thereof.

In embodiments, the HNA comprises a transgene encoding a protein, which may be expressed in cells of a subject to treat a disease or a disorder, resulting from reduced or eliminated activity of the native protein. Thus, in embodiments, the transgene may encode a protein selected from cystic fibrosis transmembrane conductance regulator (CFTR), N-acetyl-alpha-glucosaminidase (NAGLU), N-sulfoglucosamine sulfohydrolase (SGSH), palmitoyl-protein thioesterase 1 (PPT1), survival of motor neuron 1, telomeric (SMN1), alkaline phosphatase, biomineralization associated (ALPL, also known as TNALP), glial cell derived neurotrophic factor (GDNF), glucosylceramidase beta (GBA1), iduronidase alpha-L-(IDUA), cytochrome P450 family 4 subfamily V member 2 (CYP4V2), retinoschisin 1 (RS1), phosphodiesterase 6B (PDE6B), methyl-CpG binding protein 2 (MeCP2), rhodopsin (Rho), or ceroid lipofuscinosis, neuronal, 1 (CLN1).

In embodiments, the transgene encodes a CFTR. In embodiments, the CFTR comprises a mutant sequence, a codon-optimized sequence, and/or a truncated sequence of CFTR. Exemplary suitable CFTR sequences are disclosed in U.S. Patent Pub. No. 20110035819, which is incorporated herein by reference in its entirety. In embodiments, the CFTR comprises a deletion of amino acids 708-759 (“CFTRΔR”). See Ostedgaard et al., Proc. Nat'l Acad. Sci. USA (Feb. 22, 2005) 102:2952-2957, incorporated herein by reference in its entirety.

In embodiments, a transgene comprises, consists essentially of, or consists of a nucleic acid having the sequence of SEQ ID NO: 4 (a codon-optimized CFTRΔR) or SEQ ID NO: 93 (full-length codon optimized CFTR), or a sequence having up to 5, up to 10, or up to 30 nucleotide changes to SEQ ID No. 4 or 93. In embodiments, the transgene encodes a protein that comprises, consists essentially of, or consists of an amino acid having the sequence of SEQ ID NO: 95 (a CFTRΔR) or SEQ IS NO: 94 (full-length CFTR), or a sequence having up to 1, 2, 3, 4, 5, 6, 7, 8, 9, or 10 amino acids different from SEQ ID NO: 94 or 95.

In embodiments, the transgene encodes a CLN3 lysosomal/endosomal transmembrane protein, battenin (CLN3) protein, alpha-galactosidase A (GLA), or acid alpha-glucosidase (GAA).

In embodiments, the GAA protein is encoded by a nucleotide sequence of SEQ ID NOs: 5, 6 or 7, or a sequence having up to 5, up to 10, or up to 30 nucleotide changes to SEQ ID Nos. 5, 6, or 7. In embodiments, the GAA protein comprises an amino acid sequence of SEQ ID NO: 8, or a sequence having up to 1, 2, 3, 4, 5, 6, 7, 8, 9, or 10 amino acids different from SEQ ID NO. 8.

In embodiments, the GLA protein is encoded by a nucleotide sequence SEQ ID NO: 9 or 10, or a sequence having up to 5, up to 10, or up to 30 nucleotide changes to SEQ ID No. 9 or 10. In embodiments, the GLA protein comprises an amino acid sequence of SEQ ID NO: 11, or a sequence having up to 1, 2, 3, 4, 5, 6, 7, 8, 9, or 10 amino acids different from SEQ ID No. 11.

In embodiments, the CLN3 protein is encoded by a nucleotide sequence of SEQ ID NO: 12 or 13, or a sequence having up to 5, up to 10, or up to 30 nucleotide changes to SEQ ID No. 12 or 13. In embodiments, the CLN3 protein comprises an amino acid sequence of SEQ ID NO: 14, or a sequence having up to 1, 2, 3, 4, 5, 6, 7, 8, 9, or 10 amino acids different from SEQ ID No. 14.

In embodiments, the transgene comprises any one of the nucleic acid sequences listed in Table 4, or a sequence having up to 5, up to 10, or up to 30 nucleotide changes to any one of the DNA sequences in Table 4. In embodiments, the transgene encodes any one of the amino acid sequences listed in Table 4, or a sequence having up to 1, 2, 3, 4, 5, 6, 7, 8, 9, or 10 amino acids different from any one of the amino acid sequences listed in Table 4.

TABLE 4

| | Nucleic | Amino | |
|---|---|---|---|
| | acid | acid SEQ | |
| | SEQ | ID Nos. | |
| | ID | encoded | |
| Name of transgene | Nos. | by transgene | Features |
| Sulfoglucosamine | 99 | 134 | Natural |
| sulfohydrolase | | | |
| (SGSH) | | | |
| CO1-SGSH | 100 | 134 | Codon optimized |
| CO1-SGSH-GET | 101 | 135 | Codon optimized + |
| | | | GET |
| CO2-SGSH | 102 | 134 | Codon optimized |
| Ceroid | 103 | 136 | Codon optimized |
| Lipofuscinosis, | | | |
| Neuronal, 1 (CLN1) | | | |
| Survival Motor | 104 | 137 | Natural |
| Neuron 1 (SMN1) | | | |
| CO1-SMN1 | 105 | 137 | Codon optimized |
| CO2-SMN1 | 106 | 137 | Codon optimized |
| Tissue Non-specific | 107 | 138 | Natural, Contains |
| Alkaline | | | D10 tag at C end |
| Phosphatase | | | |
| (TNALP) | | | |
| CO1-TNALP | 108 | 138 | Codon optimized, |
| | | | Contains D10 tag at |
| | | | C end |
| CO2-TNALP | 109 | 138 | Codon optimized, |
| | | | Contains D10 tag at |
| | | | C end |
| Glial Cell Derived | 110 | 139 | Natural, splice |
| Neurotrophic Factor | | | variant 1 |
| (GDNF) | | | |
| Tissue Glucosyl | 111 | 140 | Natural |
| Ceramidase beta | | | |
| (GBA1) | | | |
| CO1-GBA1 | 112 | 140 | Codon optimized |
| CO2-GBA1 | 113 | 140 | Codon optimized |
| Iduronidase alpha- | 114 | 141 | Natural |
| L- (IDUA) | | | |
| CO1-IDUA | 115 | 141 | Codon optimized |
| Cytochrome P450 | 116 | 142 | Natural |
| family 4 subfamily V | | | |
| member 2 (CYP4V2) | | | |
| Retinoschisin 1 | 117 | 143 | Natural |
| (RS1) | | | |
| Phosphodiesterase | 118 | 144 | Natural |
| 6B (PDE6B) | | | |
| Methyl-CpG | 119 | 145 | Natural |
| Binding Protein | | | |
| (MeCP2) | | | |
| N-acetyl-alpha- | 120 | 146 | Natural |
| glucosaminidase | | | |
| (NAGLU) | | | |
| Ceroid | 121 | 14 | Natural |
| Lipofuscinosis, | | | |
| Neuronal 3 (CLN3) | | | |
| CO1-CLN3 | 122 | 14 | Codon optimized |
| Acid Alpha- | 123 | 8 | Natural |
| Glucosidase (GAA) | | | |
| CO1-GAA | 124 | 8 | Codon optimized |
| CO2-GAA | 125 | 8 | Codon optimized |
| CO3-GAA | 126 | 8 | Codon optimized |
| Alpha- | 127 | 148 | Natural |
| Galactosidase A | | | |
| (GLA) | | | |
| CO1-GLA | 128 | 148 | Codon optimized |
| CO1-GLA-GET | 129 | 149 | Codon optimized + |
| | | | GET |
| CO2-GLA | 130 | 148 | Codon optimized |
| CO3-GLA | 131 | 148 | Codon optimized |
| Cystic Fibrosis | 132 | 150 | Codon optimized, |
| Transmembrane | | | Contains R domain |
| Regulator ΔR | | | deletion |
| (CFTRΔR) | | | |
| Cystic Fibrosis | 133 | 151 | Codon optimized, |
| Transmembrane | | | full length |
| Regulator (CFTR) | | | |

In embodiments, the transgene comprises a nucleic acid sequence set forth in any one of SEQ ID Nos. 99-133, or a sequence having up to 5, up to 10, or up to 30 nucleotide changes to any one of SEQ ID Nos. 99-133. In embodiments, the transgene encodes an amino acid sequence set forth in any one of SEQ ID Nos. 134-151, or a sequence having up to 1, 2, 3, 4, 5, 6, 7, 8, 9, or 10 amino acids different from any one of the amino acid sequences SEQ ID Nos. 134-151.

In embodiments, the heterologous nucleic acid encodes a reporter protein; for example, a fluorescent protein.

### Methods of Producing AAV Viral Vectors

A variety of approaches may be used to produce AAV viral vectors. In embodiments, packaging is achieved by using a helper virus or helper plasmid and a cell line. The helper virus or helper plasmid contains elements and sequences that facilitate viral vector production. In another aspect, the helper plasmid is stably incorporated into the genome of a packaging cell line, such that the packaging cell line does not require additional transfection with a helper plasmid.

In embodiments, the cell is a packaging or helper cell line. In embodiments In aspects, the helper cell line is eukaryotic cell; for example, an HEK 293 cell or 293T cell. In embodiments, the helper cell is a yeast cell or an insect cell.

In embodiments, the cell comprises a nucleic acid encoding a tetracycline activator protein; and a promoter that regulates expression of the tetracycline activator protein. In embodiments, the promoter that regulates expression of the tetracycline activator protein is a constitutive promoter. In embodiments, the promoter is a phosphoglycerate kinase promoter (PGK) or a CMV promoter.

A helper plasmid may comprise, for example, at least one viral helper DNA sequence derived from a replication-incompetent viral genome encoding in trans all virion proteins required to package a replication incompetent AAV, and for producing virion proteins capable of packaging the replication-incompetent AAV at high titer, without the production of replication-competent AAV.

Helper plasmids for packaging AAV are known in the art, see, e.g., U.S. Patent Pub. No. 2004/0235174 A1, incorporated herein by reference. As stated therein, an AAV helper plasmid may contain as helper virus DNA sequences, by way of non-limiting example, the Ad5 genes E2A, E4 and VA, controlled by their respective original promoters or by heterologous promoters. AAV helper plasmids may additionally contain an expression cassette for the expression of a marker protein such as a fluorescent protein to permit the simple detection of transfection of a desired target cell.

The disclosure provides methods of producing AAV particles comprising transfecting a packaging cell line with any one of the AAV helper plasmids disclosed herein; and any one of the AAV vectors disclosed herein. In embodiments, the AAV helper plasmid and AAV vector are co-transfected into the packaging cell line. In embodiments, the cell line is a mammalian cell line, for example, human embryonic kidney (HEK) 293 cell line. The disclosure provides cells comprising any one of the AAV vectors and/or AAV particles disclosed herein.

### Pharmaceutical Compositions

The disclosure provides pharmaceutical compositions comprising any one of the AAV vectors, AAV capsids and/or AAV particles described herein. Typically, the AAV particles are administered for therapy.

The pharmaceutical composition, as described herein, may be formulated by any methods known or developed in the art of pharmacology, which include but are not limited to contacting the active ingredients (e.g., viral particles or recombinant vectors) with an excipient or other accessory ingredient, dividing or packaging the product to a dose unit. The viral particles of this disclosure may be formulated with desirable features, e.g., increased stability, increased cell transfection, sustained or delayed release, biodistributions or tropisms, modulated or enhanced translation of encoded protein in vivo, and the release profile of encoded protein in vivo.

As such, the pharmaceutical composition may further comprise saline, lipidoids, liposomes, lipid nanoparticles, polymers, lipoplexes, core-shell nanoparticles, peptides, proteins, cells transfected with viral vectors (e.g., for transplantation into a subject), nanoparticle mimics or combinations thereof. In embodiments, the pharmaceutical composition is formulated as a nanoparticle. In embodiments, the nanoparticle is a self-assembled nucleic acid nanoparticle.

A pharmaceutical composition in accordance with the present disclosure may be prepared, packaged, and/or sold in bulk, as a single unit dose, and/or as a plurality of single unit doses. The amount of the active ingredient is generally equal to the dosage of the active ingredient which would be administered to a subject and/or a convenient fraction of such a dosage such as, for example, one-half or one-third of such a dosage. The formulations of the invention can include one or more excipients, each in an amount that together increases the stability of the viral vector, increases cell transfection or transduction by the viral vector, increases the expression of viral vector encoded protein, and/or alters the release profile of viral vector encoded proteins. In embodiments, the pharmaceutical composition comprises an excipient. Non limiting examples of excipients include solvents, dispersion media, diluents, or other liquid vehicles, dispersion or suspension aids, surface active agents, isotonic agents, thickening or emulsifying agents, preservatives, or combination thereof.

In embodiments, the pharmaceutical composition comprises a cryoprotectant. The term “cryoprotectant” refers to an agent capable of reducing or eliminating damage to a substance during freezing. Non-limiting examples of cryoprotectants include sucrose, trehalose, lactose, glycerol, dextrose, raffinose and/or mannitol.

### Therapeutic Methods

This disclosure provides methods of preventing or treating a disorder, comprising, consisting essentially of, or consisting of administering to a subject a therapeutically effective amount of any one of the pharmaceutical compositions disclosed herein.

In embodiments, the disorder is a CNS disorder, a skin disorder, a lung disorder, a muscle disorder, a liver disorder, or an ophthalmic disease (or a retinal disease). In embodiments, the disorder is cystic fibrosis.

In embodiments, the disorder is hypophosphatasia, amyotrophic lateral sclerosis (ALS), spinal muscular atrophy (SMA), recessive dystrophic epidermolysis bullosa (RDEB), lysosomal storage disorder (including Duchenne's Muscular Dystrophy, and Becker muscular dystrophy), juvenile Batten disease, infantile Batten disease, autosomal dominant disorders, muscular dystrophy, Bietti's Crystalline Dystrophy, retinoschisis (e.g., degenerative, hereditary, tractional, exudative), hemophilia A, hemophilia B, multiple sclerosis, diabetes mellitus, Fabry disease, Pompe disease, neuronal ceroid lipofuscinosis 1 (CLN1), CLN3 disease (or Juvenile Neuronal Ceroid Lipofuscinosis), Gaucher disease, cancer, arthritis, muscle wasting, heart disease, intimal hyperplasia, Rett syndrome, epilepsy, Huntington's disease, Parkinson's disease, Alzheimer's disease, an autoimmune disease, cystic fibrosis, thalassemia, Hurler's Syndrome (MPS IH), Sly syndrome, Scheie Syndrome, Hurler-Scheie Syndrome, Hunter's Syndrome, Sanfilippo Syndrome A (mucopolysaccharidosis IIIA or MPS IIIA), Sanfilippo Syndrome B (mucopolysaccharidosis IIIB or MPS IIIB), Sanfilippo Syndrome C, Sanfilippo Syndrome D, Morquio Syndrome, Maroteaux-Lamy Syndrome, Krabbe's disease, phenylketonuria, spinal cerebral ataxia, LDL receptor deficiency, hyperammonemia, anemia, arthritis, or adenosine deaminase deficiency.

In addition to specific transgenes disclosed herein, known active enzyme sequences may be used as transgenes to deliver functional enzyme activity.

One of the challenges for treating cystic fibrosis is the size restriction in packaging a CFTR gene in a viral particle and the difficulty of delivery of viral particles to lung cells. The AAV particles of this disclosure solve these problems by providing CFTR transgene constructs that are efficiently packaged and have better lung tropism. Therefore, in embodiments, the disclosure provides compositions and methods for treating cystic fibrosis.

In embodiments, the disorder is CLN3 disease. CLN3 disease or Juvenile Neuronal Ceroid Lipofuscinosis is a lysosomal storage disease caused by an autosomal recessively inherited mutation in the CLN3 gene. CLN3 disease is a progressive neurodegenerative disorder in which the central nervous system (CNS) is greatly affected resulting in behavioral issues, vision loss, and other cognitive disabilities.

In embodiments, the disorder is Fabry disease. Fabry disease is an X-linked lysosomal storage disorder caused by a deficiency in alpha-galactosidase A (GLA) activity that results in the accumulation of the glycolipid products, globotriaosylceramide (Gb3) and lyso-Gb3 in the lysosome. Disease presentation is highly heterogeneous but usually includes frequent bouts of peripheral neurotrophic pain, angiokeratomas, reduced sweat production, corneal dystrophy, and gastrointestinal complications. As the disease progresses patients suffer from cardiomyopathy, renal insufficiency and cerebrovascular disease, all of which are the primary causes of reduced life-span in Fabry patients. While males are the most severely affected population of patients with mutations in the GLA gene, it has become increasingly clear that female patients are also frequently symptomatic but are often misdiagnosed. Enzyme replacement therapy (ERT) is currently the only FDA-approved therapy to treat Fabry and requires bi-weekly injections of relatively large quantities of recombinant protein. While ERT reduces the accumulation of Gb3 in the heart, kidney and vasculature it fails to completely treat all symptoms of Fabry, primarily due to its inability to efficiently enter the CNS. Gene therapy strategies have been investigated and while many show great promise in correcting the glycolipid accumulation, most have failed to efficiently enter the CNS and also suffered from an immune response often seen during GLA replacement.

In embodiments, the AAV viral vectors disclosed herein are used to treat Fabry disease in patients, who are unresponsive to ERT, or when ERT fails to address all symptoms. In embodiments, the AAV viral vectors disclosed herein are used to treat Fabry disease in patients who have already been administered ERT.

In embodiments, the disorder is Pompe disease. Pompe disease is a lysosomal storage disorder caused by a deficiency in acid α-glucosidase (GAA) activity that results in the accumulation of glycogen in the lysosome. The disease presents as a form of muscular dystrophy which primarily affects both smooth and striated musculature as well as the central nervous system (CNS), with early mortality. Enzyme replacement therapy (ERT) is currently the only FDA-approved therapy to treat Pompe and requires bi-weekly injections of relatively large quantities of recombinant protein. While ERT significantly reduces the mortality rate of infantile Pompe patients, who typically die by the age of two without therapy, it fails to completely ameliorate all symptoms of Pompe, primarily due to its inability to efficiently enter the CNS and resulting immune responses to the GAA protein. Gene therapy strategies have been investigated and while many show great promise in correcting the glycogen accumulation and other symptoms of Pompe. Most have suffered from the severe immune response seen during GAA replacement. Previous work has demonstrated that hepatic-specific expression can make animals tolerate to the GAA protein and significantly reduce the humoral response.

In embodiments, the AAV viral vectors disclosed herein are used to treat Pompe disease in patients who have already been administered ERT; for example those who are unresponsive to ERT, or when ERT fails to address all their symptoms.

In embodiments, the cancer is a solid cancer; for example, bladder, breast, cervical, colon, rectal, endometrial, kidney, lip, oral, liver, melanoma, mesothelioma, non-small cell lung, non-melanoma skin, ovarian, pancreatic, prostate, sarcoma, small cell lung tumor, or thyroid.

In embodiments, the disorder is an ophthalmic disease. The eye is immune privileged tissue. Only a very small number of viruses is necessary for therapeutic benefit. In embodiments, the ophthalmic disease affects photoreceptor and RPE cells. In some embodiments, the ophthalmic disease comprises, consists essentially of, or consists of retinitis pigmentosa (e.g., autosomal recessive (SPATA7 gene; LRAT gene; TULP1 gene), autosomal dominant (AIPL1 gene), and X-linked (RPGR gene)), eye disorders related to mutations in the bestrophin-1 (BEST-1) gene (e.g., vitelliform macular dystrophy, age-related macular degeneration, autosomal dominant vitreoretinochoroidopathy, glaucoma, cataracts), Leber congenital amaurosis (LCA; aryl-hydrocarbon interacting protein-like 1 (AIPL1) gene), cone-rod dystrophy (CRD; ABCA4 gene), Stargardt's (ABCA4 gene), choroideremia (CHM gene), Usher Syndrome (MYO7A gene; CDH23 gene; USH2A gene; CLRN1 gene), retinoschisis (RS1 gene), Bietti's Crystalline Dystrophy (CYP4V2 gene) or Achromatopsia (CNGA3 gene, CNGB3 gene, GNAT2 gene, PDE6C gene, or PDE6H gene).

In embodiments, the subject is a mammal; for example, a human. In particular aspects, the human is an infant human; for example, under 3 years old, 2 years old, or under 1 year old.

The methods of treatment and prevention disclosed herein may be combined with appropriate diagnostic techniques to identify and select patients for the therapy or prevention. For example, the method of treating or preventing a disorder, for example, cystic fibrosis, disclosed herein may further comprise steps of performing a genetic test to identify a gene mutation or deletion related to the disorder in the subject. In embodiments, the method of treating or preventing a disorder, for example, cystic fibrosis, comprises administering to a subject who has been previously identified as carrying a mutation related to the disorder, or as being at high risk for developing the disorder (for example, based on hereditary factors).

The disclosure provides methods of increasing the level of a protein in a host cell, comprising contacting the host cell with any one of the AAV particles disclosed herein, wherein the AAV particle comprises any one of the AAV vectors disclosed herein, comprising an HNA sequence encoding the protein. In embodiments, the protein is a therapeutic protein. In embodiments, the host cell is in vitro, in vivo, or ex vivo. In embodiments, the host cell is derived from a subject. In embodiments, the subject suffers from a disorder, which results in a reduced level and/or functionality of the protein, as compared to the level and/or functionality of the protein in a normal subject.

In embodiments, the level of the protein is increased to level of about 1×10⁻⁷ ng, about 3×10⁻⁷ ng, about 5×10⁻⁷ ng, about 7×10⁻⁷ ng, about 9×10⁻⁷ ng, about 1×10⁻⁶ ng, about 2×106 ng, about 3×10⁻⁶ ng, about 4×10⁻⁶ ng, about 6×10⁻⁶ ng, about 7×10⁻⁶ ng, about 8×10⁻⁶ ng, about 9×10⁻⁶ ng, about 10×10⁻⁶ ng, about 12×10⁻⁶ ng, about 14×10⁻⁶ ng, about 16×10⁻⁶ ng, about 18×10⁻⁶ ng, about 20×10⁻⁶ ng, about 25×10⁻⁶ ng, about 30×10⁻⁶ ng, about 35×10⁻⁶ ng, about 40×10⁻⁶ ng, about 45×10⁻⁶ ng, about 50×10⁻⁶ ng, about 55×10⁻⁶ ng, about 60×10⁻⁶ ng, about 65×106 ng, about 70×10⁻⁶ ng, about 75×10⁻⁶ ng, about 80×10⁻⁶ ng, about 85×10⁻⁶ ng, about 90×10⁻⁶ ng, about 95×10⁻⁶ ng, about 10×10⁻⁵ ng, about 20×10⁻⁵ ng, about 30×10⁻⁵ ng, about 40×10⁻⁵ ng, about 50×10⁻⁵ ng, about 60×10⁻⁵ ng, about 70×10⁻⁵ ng, about 80×10⁻⁵ ng, or about 90×10⁻⁵ ng in the host cell.

The disclosure provides methods of introducing a gene of interest to a cell in a subject comprising contacting the cell with an effective amount of any one of the AAV viral vector particles disclosed herein, wherein the particle contains any one of the AAV vectors disclosed herein, comprising the gene of interest.

### Dosage and Administration

Methods of determining the most effective means and dosage of administration are known to those of skill in the art and will vary with the composition used for therapy, the purpose of the therapy and the subject being treated. Single or multiple administrations can be carried out with the dose level and pattern being selected by the treating physician. It is noted that dosage may be impacted by the route of administration. Suitable dosage formulations and methods of administering the agents are known in the art. Non-limiting examples of such suitable dosages may be as low as 10⁹ vector genomes to as much as 10¹⁷ vector genomes per administration.

In embodiments of the methods described herein, the number of viral particles (e.g., AAV) administered to the subject ranges from about 10⁹ to about 10¹⁷. In particular some embodiments, about 10¹⁰ to about 10¹², about 10¹¹ to about 10¹³, about 10¹¹ to about 10¹², about 10¹¹ to about 10¹⁴, about 5×10¹¹ to about 5×10¹², or about 10¹² to about 10¹³ viral particles are administered to the subject. For administration to a human eye, a total dose of about 1×10¹⁰ vg/eye may be used, and a total dose of 5×10⁹ vg/eye may be used for a mouse eye. Non-invasive, in vivo imaging techniques can be used to monitor efficacy/safety in animals, which include but are not limited to scanning laser ophthalmoscopy (SLO), optical coherence tomography (OCT), multi-photon microscopy, fluorescein angiography.

In embodiments, the AAV particles repair the gene deficiency in a subject. In embodiments, the ratio of repaired target polynucleotide or polypeptide to unrepaired target polynucleotide or polypeptide in a successfully treated cell, tissue, organ or subject is at least about 1.5:1, about 2:1, about 3:1, about 4:1, about 5:1, about 6:1, about 7:1, about 8:1, about 9:1, about 10:1, about 20:1, about 50:1, about 100:1, about 1000:1, about 10,000:1, about 100,000:1, or about 1,000,000:1. The amount or ratio of repaired target polynucleotide or polypeptide can be determined by any method known in the art, including but not limited to Western blot, Northern blot, Southern blot, PCR, sequencing, mass spectrometry, flow cytometry, immunohistochemistry, immunofluorescence, fluorescence in situ hybridization, next generation sequencing, immunoblot, and ELISA.

In embodiments, the viral particle is introduced to the subject intravenously, intrathecally, intracerebrally, intraventricularly, intranasally, intratracheally, intra-aurally, intra-ocularly, or peri-ocularly, orally, rectally, transmucosally, inhalationally, transdermally, parenterally, subcutaneously, intradermally, intramuscularly, intrapleurally, topically, intralymphatically, intracisternally; such introduction may also be intra-arterial, intracardiac, subventricular, epidural, intracerebral, intracerebroventricular, sub-retinal, intravitreal, intraarticular, intraperitoneal, intrauterine, or any combination thereof. In embodiments, the viral particles are delivered to a desired target tissue, e.g., to the lung, eye, or CNS, as non-limiting examples. In embodiments, delivery of viral particles is systemic. The intracisternal route of administration involves administration of a drug directly into the cerebrospinal fluid of the brain ventricles. It could be performed by direct injection into the cisterna magna or via a permanently positioned tube.

For treating an ophthalmic disease (or an eye disorder) intraocularly, there are multiple modes of administration known to those skilled in the art, including but not limited to: lacrimal gland (LG) administration, topical eye drop, intra-stromal administration to the cornea, intra-cameral administration (anterior chamber), intravitreal administration, sub-retinal administration, systemic administration, or a combination thereof. 80% of genetic eye disorders occur in the photoreceptors. Intravitreal delivery of small volume gene therapies can occur in an out-patient clinic.

Administration of the AAV vector, AAV particle or compositions of this disclosure can be effected in one dose, continuously or intermittently throughout the course of treatment. In embodiments, the AAV vector, AAV particle or compositions of this disclosure are parenterally administered by injection, infusion or implantation.

In embodiments, the AAV particles of this disclosure show enhanced tropism for brain and cervical spine. In embodiments, the viral particles of the disclosure can cross the blood-brain-barrier (BBB). In embodiments, the AAV particles of this disclosure show high retinal tropism by subretinal and intravitreal injections. In embodiments, the AAV particles of this disclosure target multiple eye cell types, such as, for example, cones, rods, and retinal pigment epithelium (RPE). In embodiments, AAV particles of this disclosure escape neutralizing antibodies against natural serotypes, and thus enable potential redosing. In a further aspect, the AAV particles and compositions of the disclosure may be administered in combination with other known treatments for the disorder being treated.

### Kits

The agents, vectors, or compositions described herein may, In embodiments, be assembled into pharmaceutical or diagnostic or research kits to facilitate their use in therapeutic, diagnostic or research applications. In embodiments, the kits of the present disclosure include any one of the modified AAV capsid proteins, AAV vectors, AAV particles, host cells, isolated tissues, compositions, or pharmaceutical compositions as described herein.

In embodiments, a kit further comprises instructions for use. Specifically, such kits may include one or more agents described herein, along with instructions describing the intended application and the proper use of these agents. As an example, In embodiments, the kit may include instructions for mixing one or more components of the kit and/or isolating and mixing a sample and applying to a subject. In embodiments, agents in a kit are in a pharmaceutical formulation and dosage suitable for a particular application and for a method of administration of the agents. Kits for research purposes may contain the components in appropriate concentrations or quantities for running various experiments.

The kit may be designed to facilitate use of the methods described herein and can take many forms. Each of the compositions of the kit, where applicable, may be provided in liquid form (e.g., in solution), or in solid form, (e.g., a dry powder). In certain cases, some of the compositions may be constitutable or otherwise processable (e.g., to an active form), for example, by the addition of a suitable solvent or other species (for example, water or a cell culture medium), which may or may not be provided with the kit. In embodiments, the compositions may be provided in a preservation solution (e.g., cryopreservation solution). Non-limiting examples of preservation solutions include DMSO, paraformaldehyde, and CryoStor® (Stem Cell Technologies, Vancouver, Canada). In embodiments, the preservation solution contains an amount of metalloprotease inhibitors.

In embodiments, the kit contains any one or more of the components described herein in one or more containers. Thus, In embodiments, the kit may include a container housing agents described herein. The agents may be in the form of a liquid, gel or solid (powder). The agents may be prepared sterilely, packaged in a syringe and shipped refrigerated. Alternatively, they may be housed in a vial or other container for storage. A second container may have other agents prepared sterilely. Alternatively, the kit may include the active agents premixed and shipped in a syringe, vial, tube, or other container. The kit may have one or more or all of the components required to administer the agents to a subject, such as a syringe, topical application devices, or IV needle tubing and bag.

It is to be understood that while the invention has been described in conjunction with the above embodiments, the foregoing description and examples are intended to illustrate and not limit the scope of the invention. Other aspects, advantages and modifications within the scope of the invention will be apparent to those skilled in the art to which the invention pertains.

In addition, where features or aspects of the invention are described in terms of Markush groups, those skilled in the art will recognize that the invention is also thereby described in terms of any individual member or subgroup of members of the Markush group.

### EXAMPLES

### Example 1

### Capsid Generating Platform

Some AAV capsid sequences provided herein (e.g., AAV204, AAV110) were generated using the AAV capsid generating platform shown in FIG. 1. Briefly, the platform comprises a DNase I fragmentation step, and an assembly and amplification step, which finally result in the formation of a chimeric capsid library. Other AAV capsid sequences provided herein (e.g., AAV214) were generated by rational design.

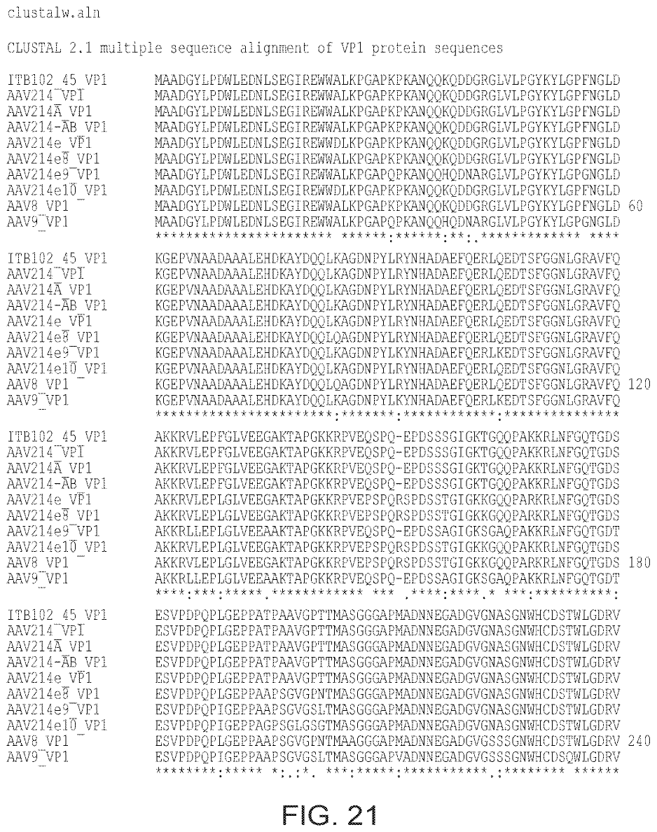

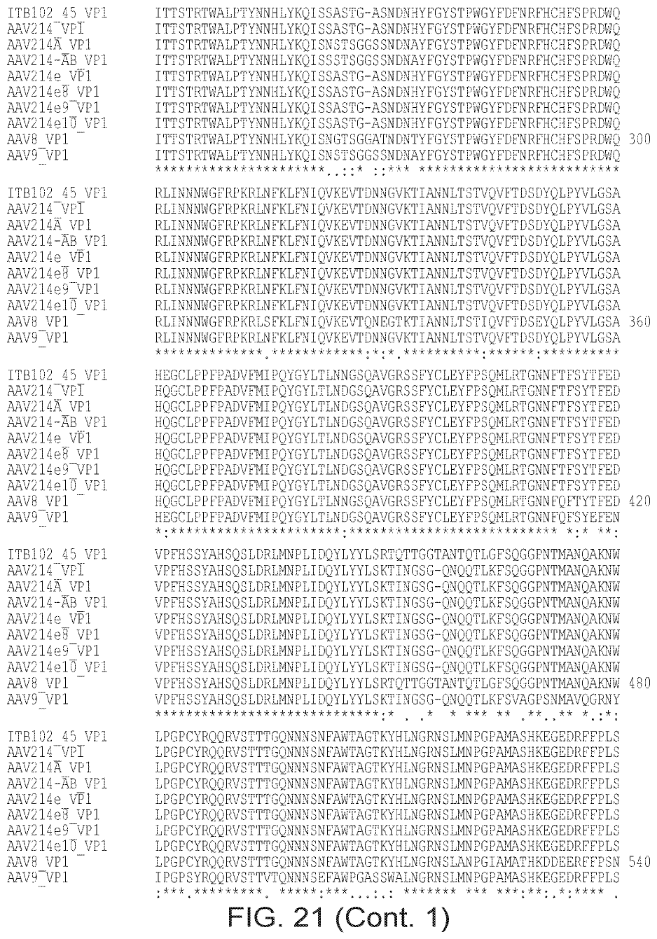

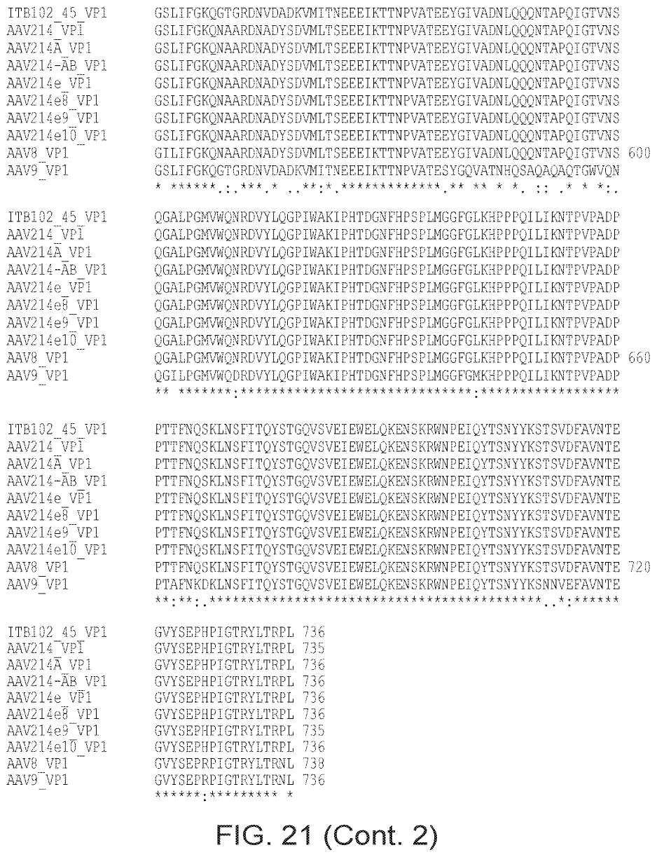

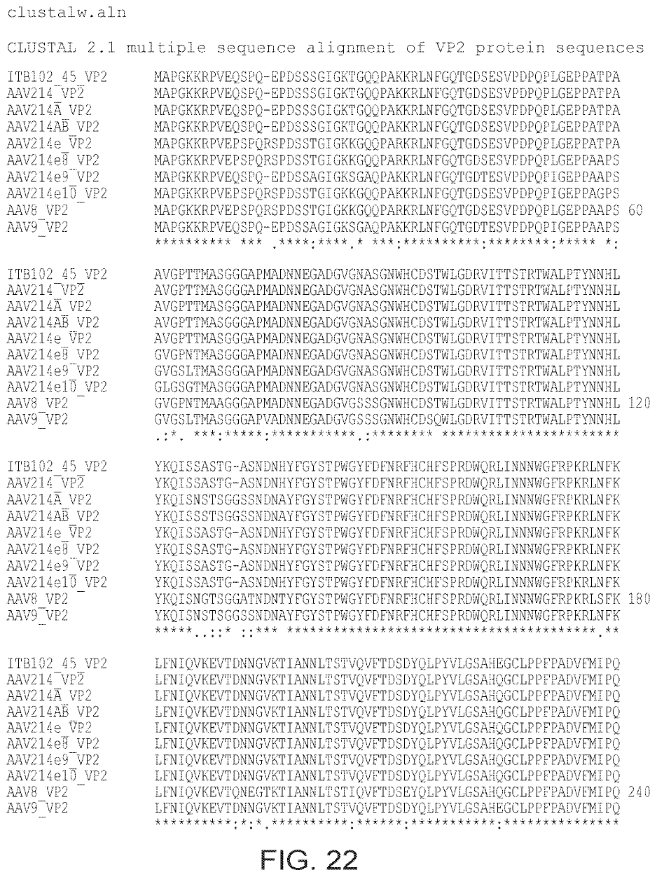

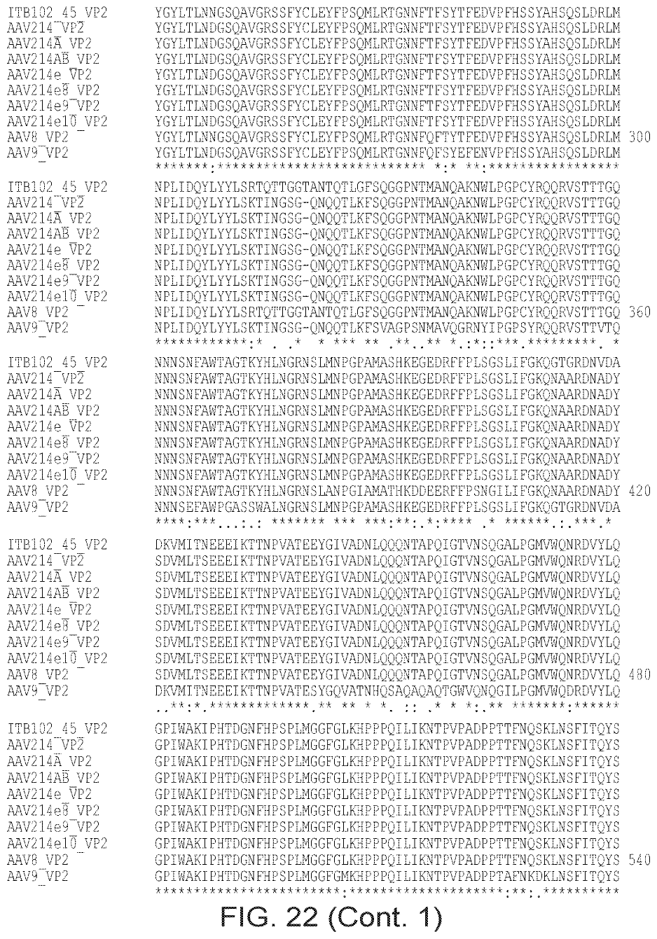

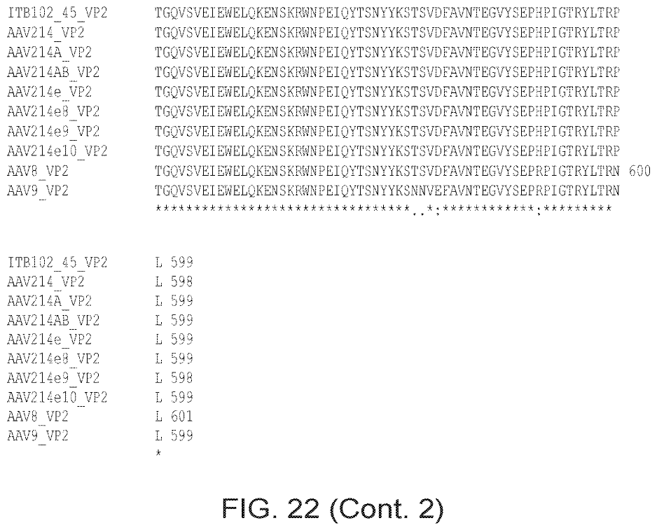

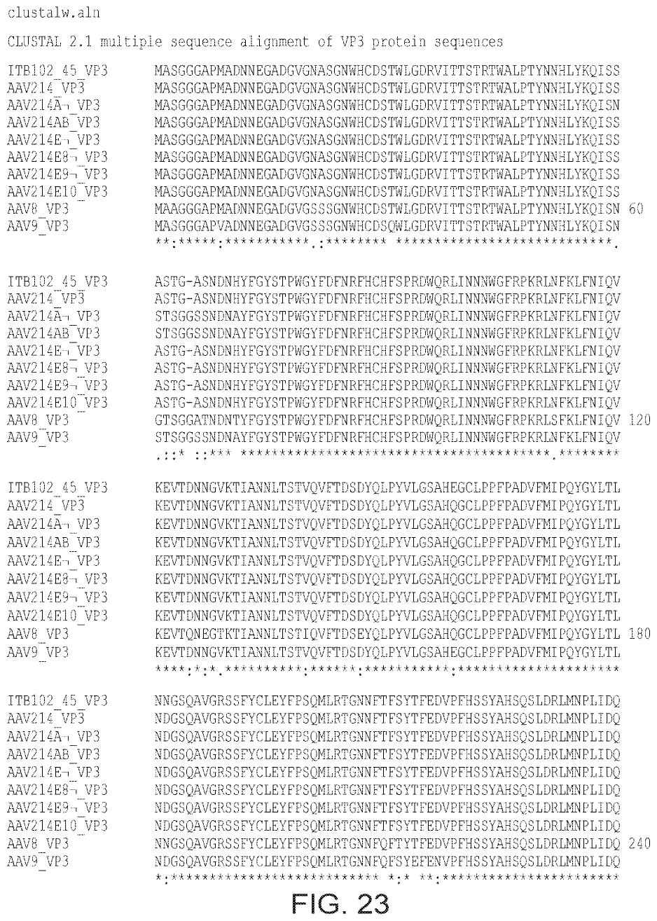

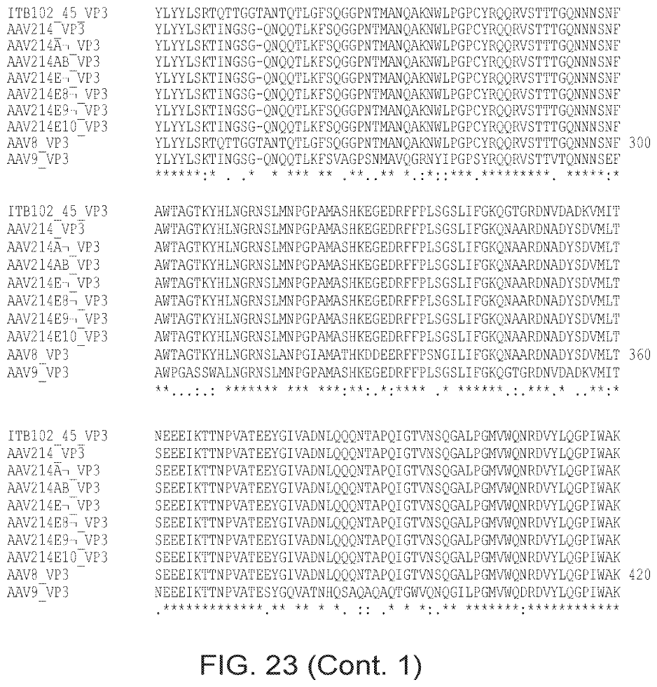

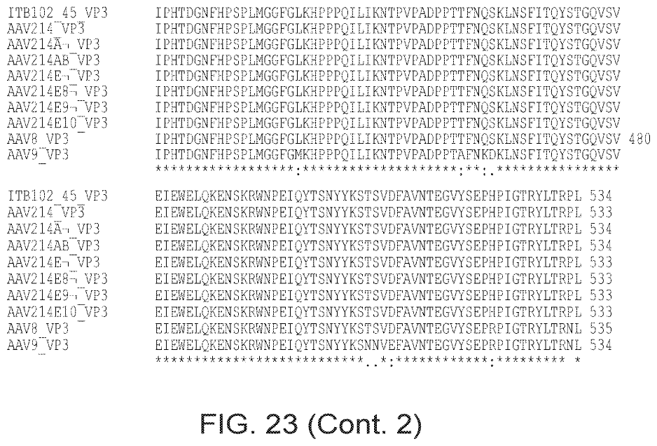

Capsid proteins generated using these methodologies were analyzed by alignment with the amino acid sequences of known AAV capsid proteins. Sequence alignment of VP1 protein sequence from AAV204 (SEQ ID NO: 2) and AAV6 (SEQ ID NO: 63) is shown in FIG. 20. Sequence alignment of VP1 amino acid sequences of AAV 214, AAV 214A, AAV 214e, AAV 214e8, AAV 214e9, AAV 214e10, AAV 214AB, and AAV ITB102_45 is provided in FIG. 21. Sequence alignment of VP2 amino acid sequences of AAV 214, AAV 214A, AAV 214e, AAV 214e8, AAV 214e9, AAV 214e10, AAV 214AB, and AAV ITB102_45 is provided in FIG. 22. Sequence alignment of VP1 amino acid sequences of AAV 214, AAV 214A, AAV 214e, AAV 214e8, AAV 214e9, AAV 214e10, AAV 214AB, and AAV ITB102_45 is provided in FIG. 23.

Viral vectors may be made using a standard triple-transfection method known in the art. Briefly, three separate plasmids expressing, respectively, the viral capsid protein, helper proteins (e.g., the essential viral Rep and Cap proteins), and the transgene of interest are transfected into adherent or suspension 293 cells, and viral particles are later harvested using ultracentrifugation or chromatography followed by diafiltration/ultrafiltration and terminal sterile filtration. See, e.g., Guo et al., Mol. Ther. Methods Clin. Dev., Vol. 13, pp. 40-46 at 44 (November 2018); Wang et al., Human Gene Ther. Methods, Vol. 25, pp. 261-68 at 262; and Gao et al., Human Gene Ther. Methods, Vol. 11, pp. 2079-91, each of which is incorporated herein by reference in their entireties for all purposes.

### Example 2

### Characterization of AAV214 and AAV204 Viral Vectors

The efficiency of transduction of AAV214 or AAV204 vectors in different target tissues was assessed as described below.

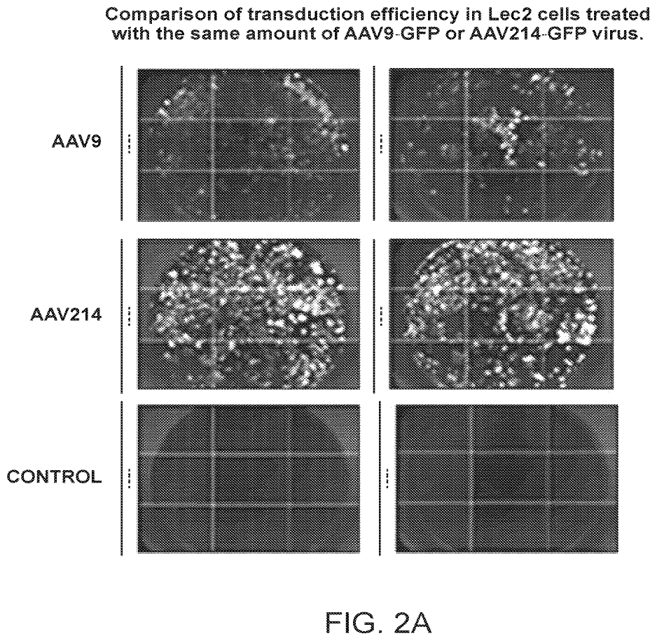

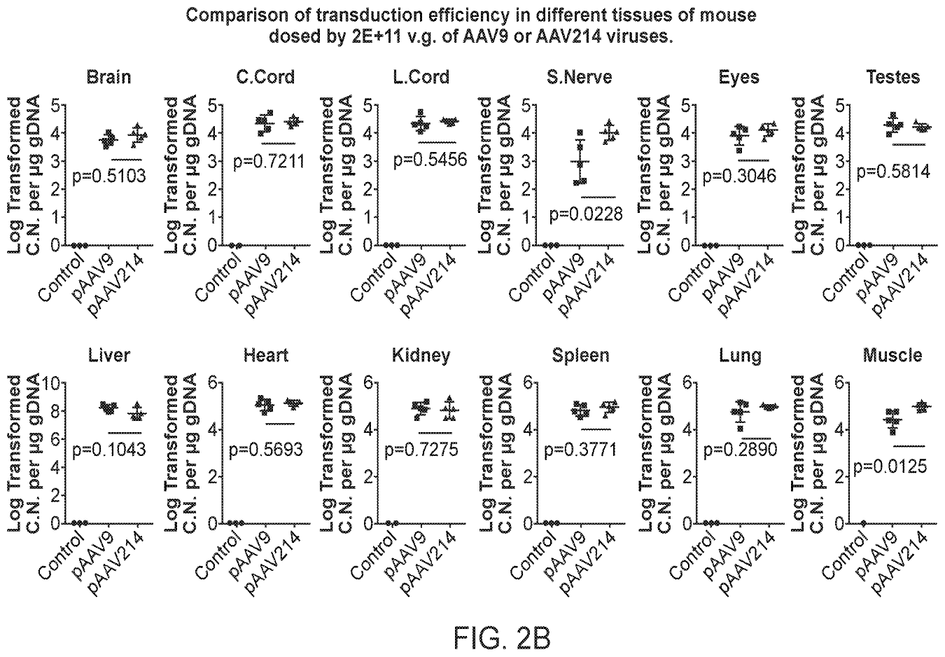

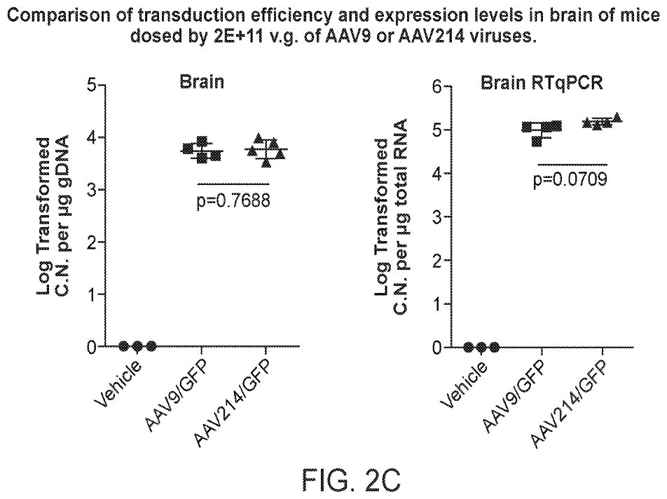

The transduction efficiency of an AAV214 viral vector comprising an EGFP transgene (AAV214-GFP) and an AAV9 viral vector comprising an EGFP transgene (AAV9-GFP) were evaluated in vitro. HEK 293 cells were seeded in a 96-well plate at 50,000 cells per well. Cells were transduced with AAV214-GFP or AAV9-GFP at an MOI of 5E+5. Images taken 45 hours post transduction revealed that transduction efficiency was higher for AAV214-GFP in HEK 293 cells (FIG. 2A). It is noted that “GFP” as used herein refers to EGFP (see, e.g., Zhang et al. (1996) Biochem. Biophys. Res. Commc'n. 227 (3): 707-11) unless otherwise specified.

To test transduction efficiency in vivo, 10-week old C57BL/6 mice were dosed by intravenous (IV) injection of 2E+11 vg of AAV214-GFP or AAV9-GFP in 200 μL of TMN200 (200 mM Tris-HCl, 1 mM MgCl2, 200 mM NaCl and 0.001% Pluronic F68). Thirteen days later, mice were euthanized, tissue samples of internal organs (brain, spinal cord (cervical and lumbar), sciatic nerve, eyes, heart, kidney, liver, lung, testes, spleen and muscle) were collected, and total DNA was isolated and analyzed using an absolute qPCR approach for

GFP gene copy number estimation. Obtained AAV biodistribution data were plotted using Prism software for statistical analysis (GraphPad Software). An unpaired t-test performed on log-transformed data did not reveal a statistically significant difference between AAV9-GFP and AAV214-GFP transduction efficiencies in most tissues tested (p<0.05). However, in the case of sciatic nerve and muscle, the mean value of detected viral DNA copy number per microgram of total DNA isolated from AAV214-GFP dosed animals was higher and statistically significantly different from AAV9-GFP dosed animals (sciatic nerve: 4.1-fold, p=0.0228; muscle: 3-fold, p=0.0125) (FIG. 2B).

The same experiment was repeated with the brain sample split into two halves. One half was used for total DNA isolation and followed by biodistribution analysis using absolute qPCR. The other half was used for total RNA isolation, DNase treatment, conversion into cDNA, and qPCR analysis to quantify EGFP gene expression levels. Obtained AAV biodistribution and transgene expression data were plotted using Prism software for statistical analysis. An unpaired t-test performed on log-transformed data did not reveal a statistically significant difference between AAV9-GFP and AAV214-GFP transduction efficiencies (p=0.7668) or expression levels (p=0.0709) in brain tissue (FIG. 2C).

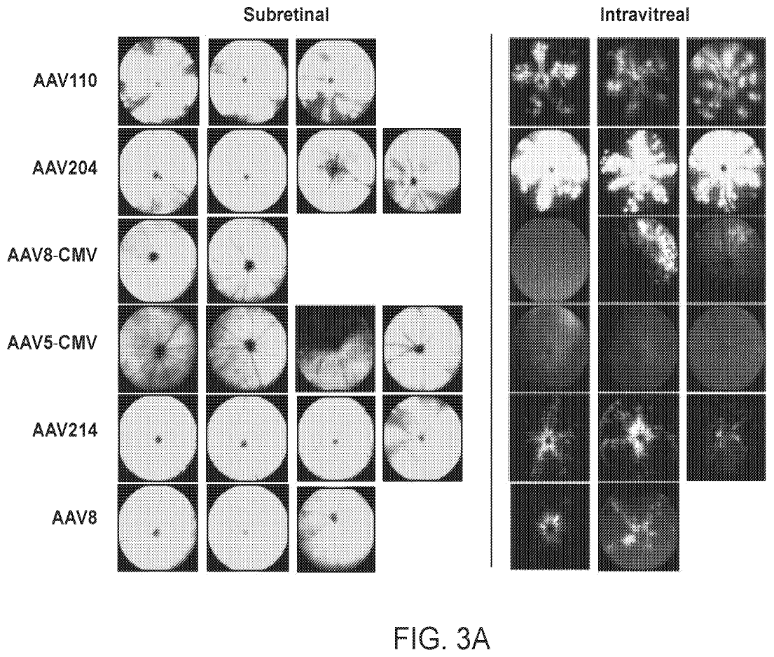

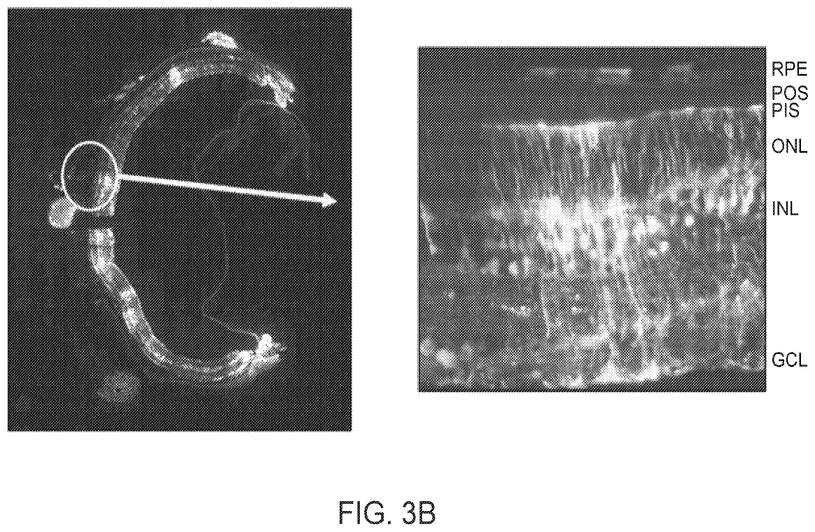

Wild-type C57BL/6J mice were administered a set of AAV viral vectors including AAV204-GFP, AAV110-GFP, and AAV214-GFP by both subretinal (right eye) and intravitreal (left eye) injection. 1 μL of AAV vector at 5E+12 vg/mL (5E+9 vg/eye) was injected for both methods of administration and animals were imaged after 10 days with an HRA2 Spectralis Scanning Laser Ophthalmoscope (Heidelberg Engineering, Carlsbad, CA). Images where cataract prevented sufficient observation were omitted from the analysis. Ophthalmoscopy imaging revealed that all tested viruses were capable of transfecting retinal cells if dosed into the subretinal cavity. However, only AAV204 and AAV110 showed enhanced transduction of retinal cells mediated by intravitreal delivery (FIG. 3A). Immunohistochemistry analysis of AAV204-GFP intravitreally dosed mouse eyes demonstrated GFP expression in various types of retinal cells including photoreceptors, RPE, Müller glia cells, retinal ganglion cells and bipolar cells (FIG. 3B).

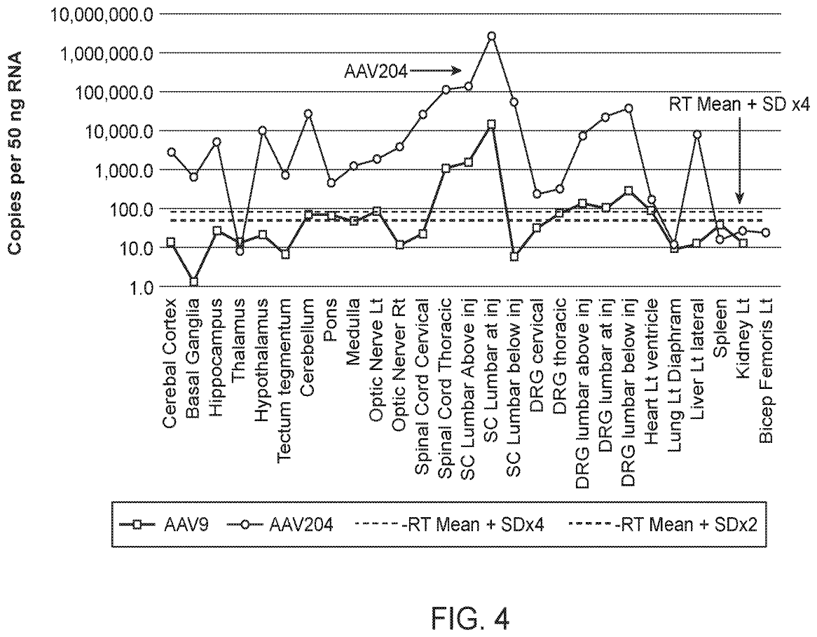

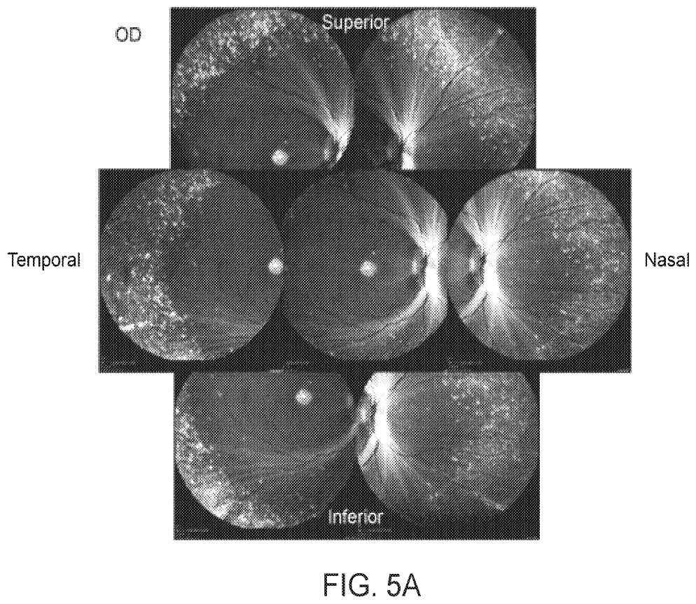

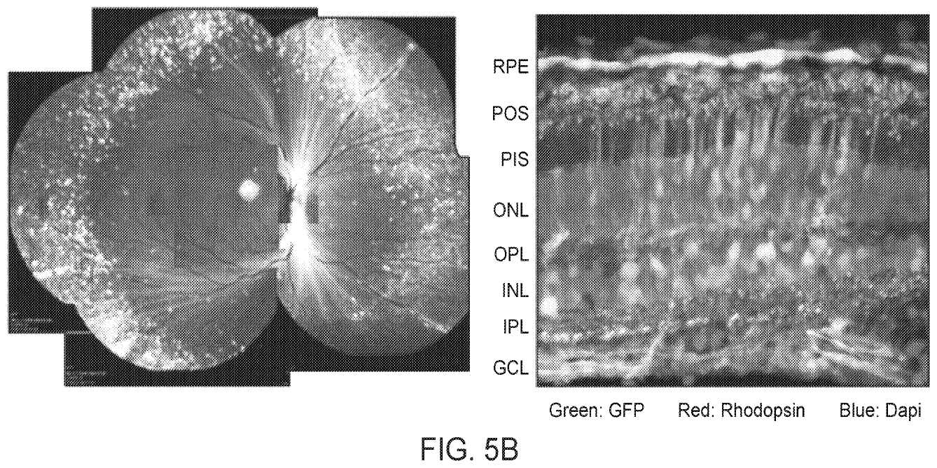

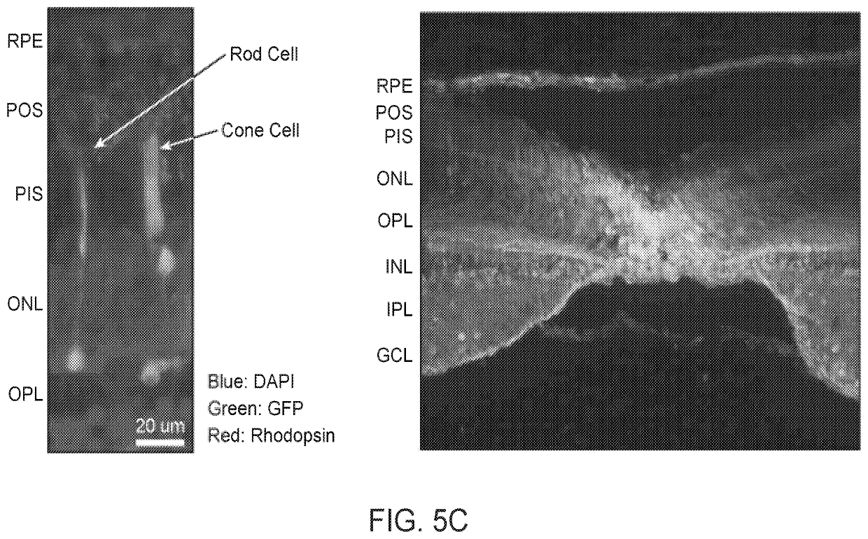

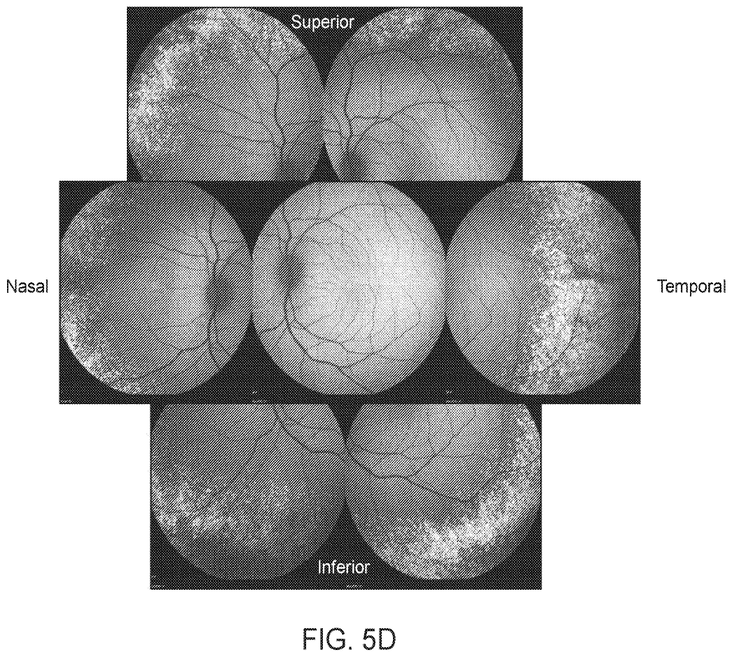

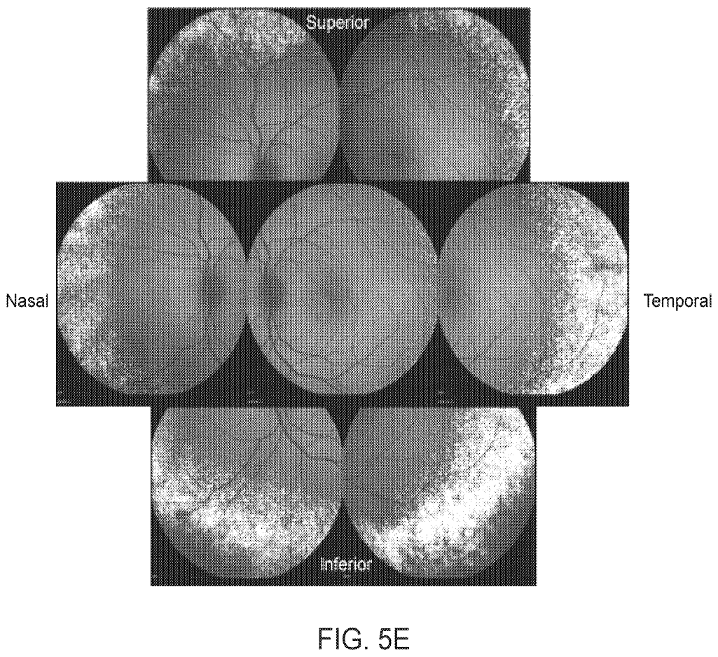

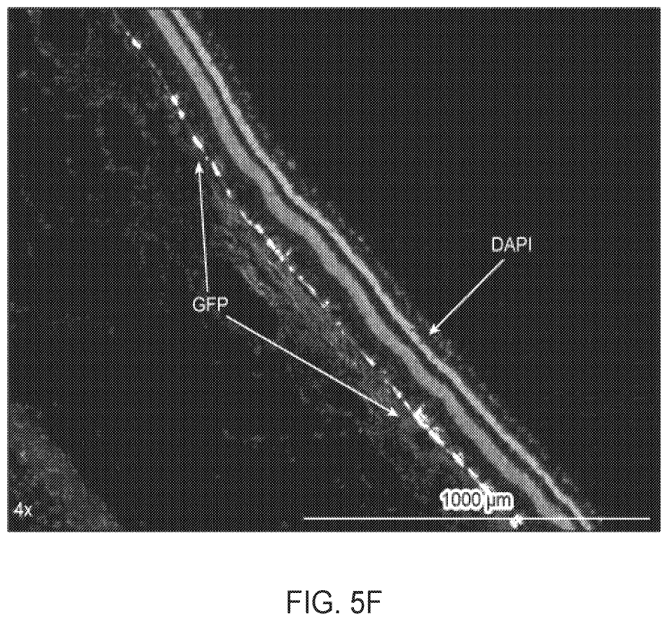

AAV204-GFP and AAV9-GFP were administered by intrathecal (1E+13 vg) and/or intravitreal injection (1.5E+12 vg) to 2.5- to 3-year-old cynomolgus monkeys (Macaca fascicularis) each weighing about 2 kg. After four weeks, animals were euthanized and GFP expression was evaluated by RT-qPCR. Data analysis showed that AAV204-GFP mediated delivery resulted in enhanced GFP expression in most of the tissues that were assayed, including specific areas of the brain and spinal cord. See FIG. 4. The eyes of the intravitreally dosed (1.5E+12 vg of AAV204-GFP per eye) cynomolgus monkeys were evaluated by scanning laser ophthalmoscopy (SLO), sectioned and analyzed for GFP, rhodopsin and genomic DNA using conventional immunochemistry staining methods. As shown in FIGS. 5A and 5B, the administration of AAV204-GFP resulted in significant transduction of the vector in the peripheral retina and the foveal region of the eye. Enhanced expression of GFP delivered by AAV204 was seen in retinal cells including photoreceptors, RPE, bipolar cells and ganglion cells (FIG. 5B). A significant number of rods and cones were transduced in the macula (FIG. 5C).

The AAV204 vector can also be combined with RPE-specific promoters to express proteins specifically. FIGS. 5D-5F show expression of GFP from AAV204 driven by the VMD2 (vitelliform macular degeneration-2) promoter (SEQ ID NO:159). 2.5×10¹² viral genomes (vg) vector were administered intravitreally and expression was monitored at 14 days and at 28 days (sacrifice). Scanning laser ophthalmoscopy (SLO) imaging was performed at Day 14 (FIG. 5D) and Day 28 (FIG. 5E). FIG. 5F shows GFP expression and nuclei (DAPI) in the periphery at Day 28.

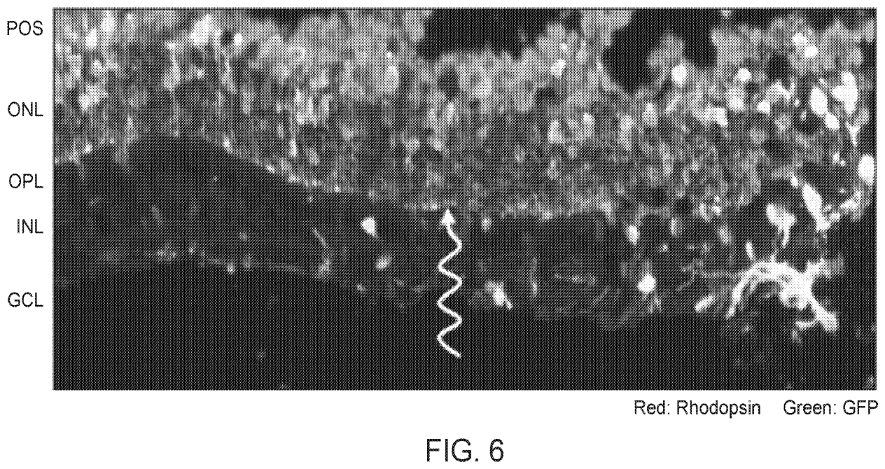

AAV204-GFP was also evaluated in non-human primate explant cultures. Cynomolgus monkey retinas were isolated from eyes within 1 h of the animal being humanely euthanized. Retinas were dissected into ~5×5 mm sections and cultured in transwell insert culture dishes. One day post-isolation, explants were transduced with AAV204-GFP in culture media and incubated for one-week post-transduction. Explants were fixed, embedded and sectioned for standard immunohistochemistry. Sections were stained for GFP (green) and rhodopsin (red) and imaged using fluorescence microscopy. Sections showed significant GFP expression in the photoreceptor layer after AAV204-GFP transduction (FIG. 6).

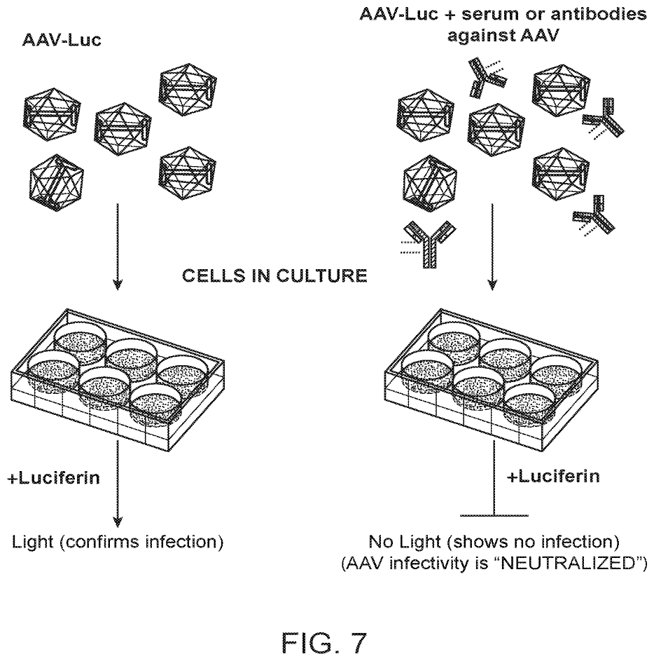

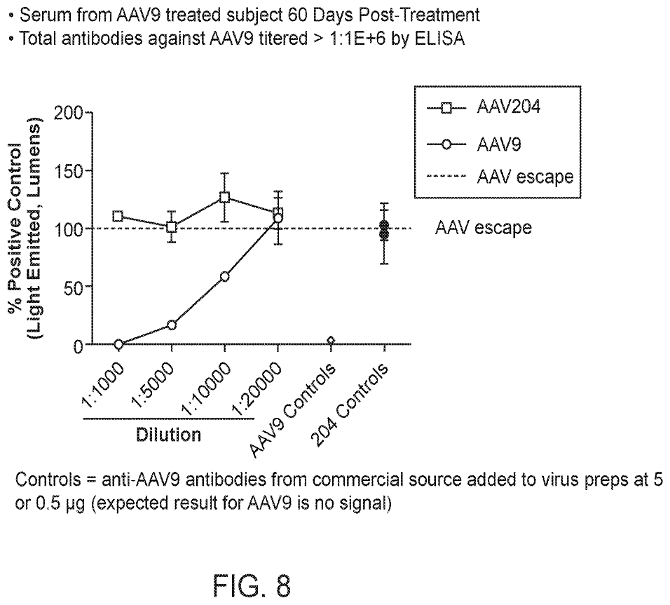

Immunogenicity of the AAV204 vector was evaluated using the neutralizing antibody assay (FIG. 7). Either AAV9-Luc virus comprised of AAV9 capsid and a firefly luciferase expression cassette or AAV204-Luc virus comprised of AAV204 capsid and a firefly luciferase expression cassette was incubated with various dilutions of serum from an AAV9-treated human subject (60 days post-treatment) at MOI of 25,000. After incubation the virus/serum mixture was transferred to wells containing 20,000 Lec2 cells. Serum-treated cells were incubated for 24 hours and then emitted luminescence was measured and compared to a control value from cells transduced with untreated virus at the same MOI. The results showed that AAV204 vector particles had reduced immunogenicity compared to AAV9 vector particles (see FIG. 8).

### Example 3

### Amelioration of Defects Caused by Mutations in the Cystic Fibrosis Transmembrane Conductance Regulator (CFTR) Gene Using a CFTR Transgene Delivered by AAV204 Viral Vector

AAV204 vector particles containing a nucleic acid encoding a codon-optimized CFTR transgene (that is, a CFTRΔR gene comprising nucleic acid sequence set forth in SEQ ID NO: 4, and encoding a protein lacks amino acids 708-759 of full length CFTR) were prepared using an pA-CF3 plasmid (SEQ ID NO: 89) having, from 5′ to 3′, a 5′ ITR, a mouse U1a promoter (SEQ ID NO: 96), the CFTRΔR transgene, a synthetic poly-A sequence (49 bp) and a 3′ ITR. The resulting particles were used to deliver the CFTRΔR transgene into either cells or mice, as described below.

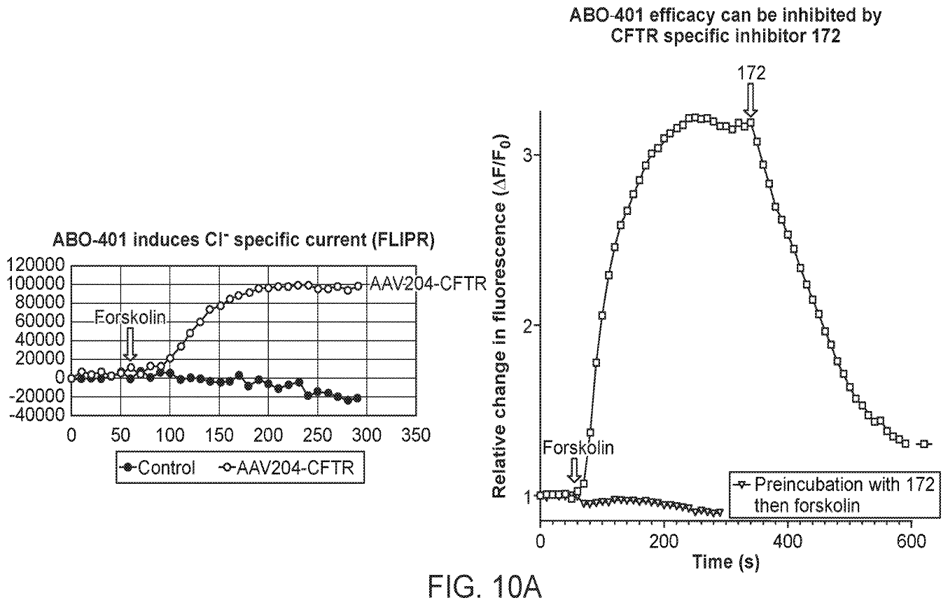

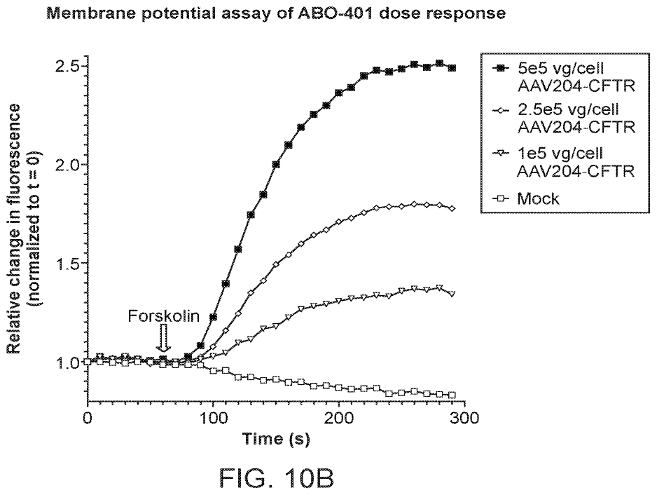

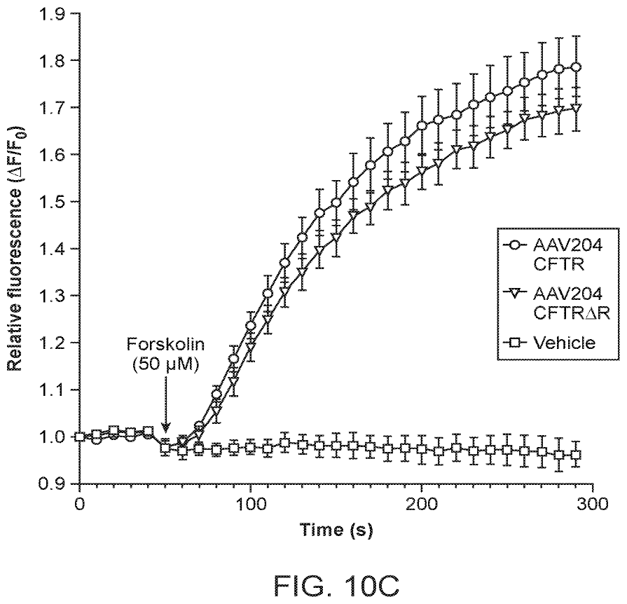

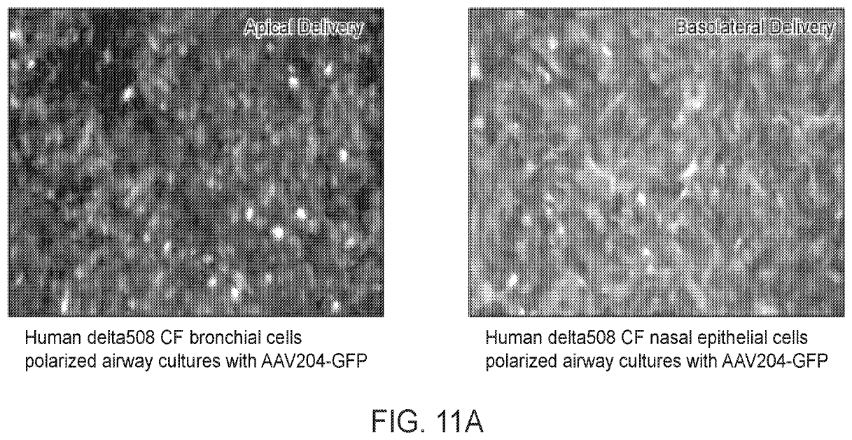

In vitro assays: An AAV204 viral vector comprising the CFTRΔR transgene was used to transduce Lec2 cells. The FLIPR assay was used to measure the functionality of the CFTRΔR transgene delivered by the AAV204-CFTRΔR viral vector. The results showed that human CFTR (hCFTR) ion channel function was restored by stimulation with forskolin, a known opener of the CFTR chloride channel, when the cells were transduced using the AAV204-CFTRΔR viral vector, as compared to cells transfected with a control AAV204 viral vector lacking a transgene. Further, chloride-specific current signal was increased by 3.5-fold compared to baseline in cells transduced using the AAV204-CFTRΔR viral vector as compared to the control. See FIG. 10A, left panel. Pre-incubation with a selective inhibitor of CFTR, CFTRinh-172 (4-[[4-Oxo-2-thioxo-3-[3-trifluoromethyl)phenyl]-5-thiazolidinylidene]methyl]benzoic acid) (Tocris Bioscience) (available at http://tocris.com/products/cftrinh-172_3430) prevented forskolin-induced membrane potential changes in the presence of CFTRΔR. See FIG. 10B. These results demonstrate the functionality of the CFTRΔR expression cassette.

A membrane potential assay was performed to evaluate whether the functionality of the CFTRΔR expression cassette was dependent on the amount of the AAV204-CFTRΔR virus used for transfection. See FIG. 10B. The results showed that the membrane potential changes in the presence of CFTRΔR were indeed dependent on the dose of the virus particle delivering the transgene. We also expressed the full-length CFTR using AAV204 and obtained increased fluorescence in response to forskolin. (FIG. 10C). We also confirmed by western blot that expression from AAV204 results in fully-processed full-length CFTR and CFTRΔR (data not shown). These data confirm that that AAV204 delivery of either protein restores chloride channel function in vitro.

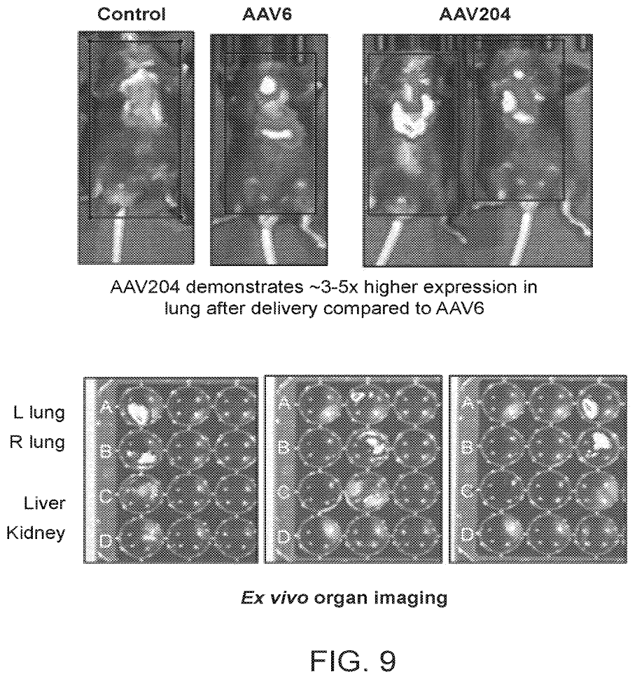

In vivo assays using mouse model: An AAV204 viral vector comprising a luciferase transgene was administered to mice intratracheally. Bioluminescence imaging (BLI) was used to assess the ability of an AAV204 viral vector to transduce lung cells, as reflected by luciferase expression, in comparison to AAV6. FIG. 9, upper panel, shows that luciferase expression mediated by the AAV204 viral vector was about 3.5-fold higher compared to the AAV6 viral vector. FIG. 9, lower panel illustrates expression, shown by ex vivo BLI, in the left and right lungs with little or no expression in the liver or kidney. These results demonstrate that an AAV204 viral vector is capable of promoting enhanced expression of a reporter transgene in specific tissues in mice.

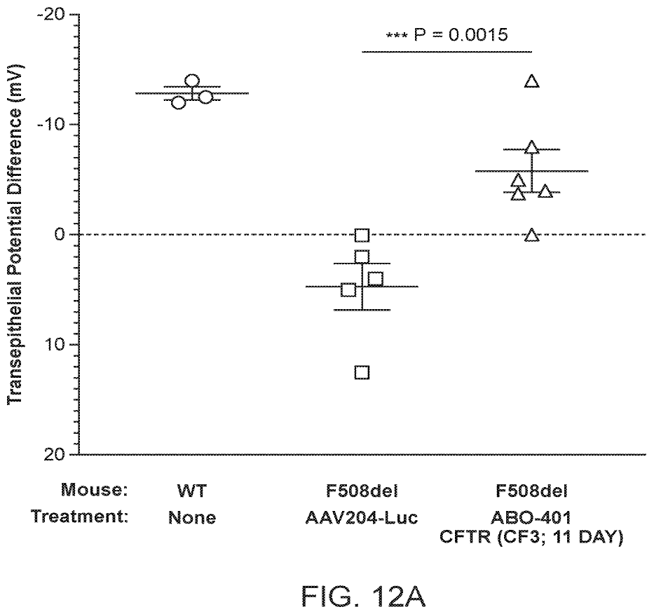

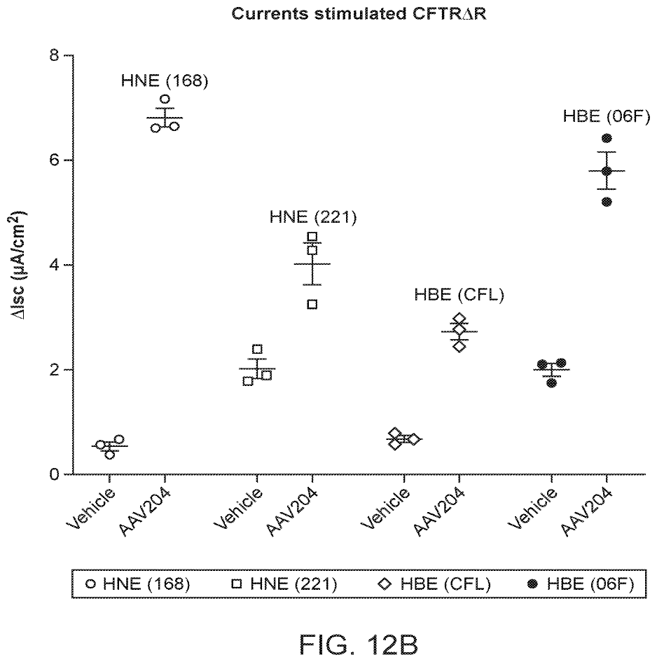

The efficacy of AAV204 vector particles comprising the CFTRΔR transgene was tested in a mouse model of cystic fibrosis referred to as “F508del”. These mice carry a mutant CFTR gene comprising a deletion of a single amino acid, F508, which is the most common CFTR mutation in humans, affecting approximately 90% of CF patients (see, e.g., Park et al., PLOS One (Feb. 10, 2016) 11 (2): e0149131). AAV204 vector particles comprising the CFTRΔR transgene or the luciferase transgene were intranasally administered to wild-type and F508del mice. Nasal potential difference (NPD) was measured to determine the functionality of the CFTRΔR transgene. As shown in FIG. 12A, mice that were administered the AAV204-CFTR vector particles showed corrected forskolin-stimulated current, as compared to mice administered the control vector containing a luciferase transgene (AAV204-Luc).

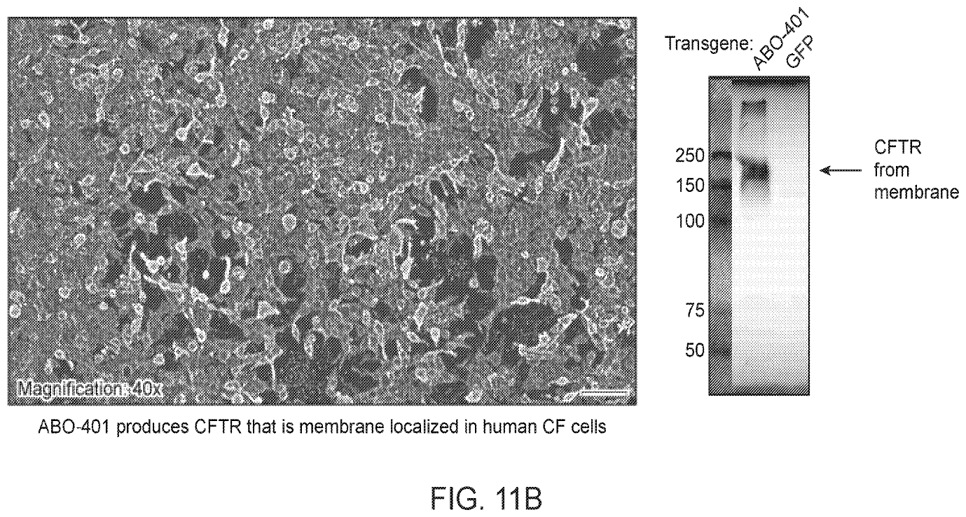

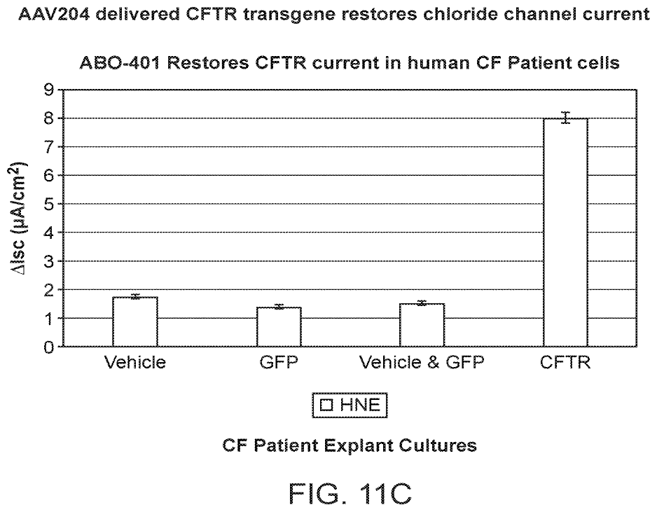

Assays using human patient cells: The ability of AAV204 vector particles comprising the CFTRΔR transgene to mediate delivery of hCFTR into human airway cells isolated from patients suffering from cystic fibrosis, and to correct chloride transport in these cells was evaluated. When AAV204 vector particles comprising GFP transgene were applied to the apical and basolateral compartments, AAV204 transduced human nasal and bronchial epithelial (HNE and HBE) cells isolated from cystic fibrosis patients, and maintained in air-liquid interface cultures. See FIG. 11A. The CFTRΔR protein was membrane-localized in these cells; see FIG. 11B, left panel. FIG. 11B, right panel shows a western blot illustrating membrane localization.

The functionality of AAV204 vector particles comprising the CFTRΔR transgene was evaluated as described below. The results show that CFTR current was restored in explant cultures of human nasal epithelial cells derived from cystic fibrosis patients following transduction of the AAV204 vector comprising the CFTRΔR transgene. See FIG. 11C. It was also tested whether the AAV particles could restore CFTR function in nasal and bronchial cells isolated from human cystic fibrosis patients by measuring changes in transmembrane conductance using an Ussing chamber, which is known to those skilled in the art to measure the movement of ions between the surfaces of polarized epithelium. Briefly, in an Ussing chamber the apical and basolateral surfaces of the epithelium face two separate chambers containing symmetrical salt solutions. Ion transport across the epithelium produces a potential difference between the two chambers. Diffusion forces that would otherwise create a potential difference are actively cancelled out by applying a short-circuit current (I_{sc}) across the epithelium. This allows for the movement of ions by active transport following stimulation, to be measured by changes in this current (Δl_{sc}) and calculation of cystic fibrosis transmembrane conductance, as is well-known in the art. See, e.g., Li et al., J. Cystic Fibrosis (July 2004) 3:123-126; Park et al., PLOS One (Feb. 10, 2016) 11 (2): e0149131. As shown in FIG. 12B, when CFTRΔR was transduced, forskolin-stimulated, CFTRinh-172-inhibited current was restored to 6-7 μA/cm² as compared to vehicle.

In sum, our results show that AAV204 mediates efficient delivery of highly-expressed, functional CFTR, and further, restores CFTR function in cells in vitro, in mouse models and in explant cultures of human patient cells. These results demonstrate the therapeutic potential in cystic fibrosis of AAV204 particles comprising the CFTRΔR transgene.

### Example 4

### Amelioration of Defects Caused by CLN3 Disease Using Optimized CLN3 Transgenes Delivered by AAV214 Vector

An AAV capsid (AAV214) with enhanced tropism for CNS tissue after systemic administration, and an optimized CLN3 (comprising a nucleic acid sequence of SEQ ID NO: 122) transgene cassette to improve biodistribution and expression in CNS and somatic tissues, were developed and tested for functionality. AAV9 was used as a benchmark to assess the tropism of AAV214 and the biodistribution of the optimized CLN3 transgene cassette in a mouse model of juvenile neuronal ceroid lipofuscinosis, which lacks a 1.02 kb segment spanning exons 7 and 8 of CLN3 (CLN3Δex7/8) in a C57BL/6 background. This CLN3 deletion is one that occurs in approximately 85% of mutated CLN3 alleles and recapitulates many disease phenotypes associated with human disease, including motor deficits, glial activation, and progressive accumulation of lysosomal storage material.

TABLE 6
Study design with CLN3^{Δex7/8} mice model

| | | | | | Dose | Dose |
|---|---|---|---|---|---|---|
| | | Treat- | | No. | amount | volume |
| Group | Strain | ment | Route | Animals | (vg/kg) | (ul/animal) |
| 1 | CLN3^{Δex7/8} | vehicle | IV | 3 male | 0 | 200 |
| | | | | 6 female | | |
| 2 | CLN3^{Δex7/8} | AAV9- | IV | 5 male | 2 × 10¹³ | 200 |
| | | CLN3 | | 5 female | | |
| 3 | CLN3^{Δex7/8} | AAV214- | IV | 5 male | 2 × 10¹³ | 200 |
| | | CLN3 | | 5 female | | |

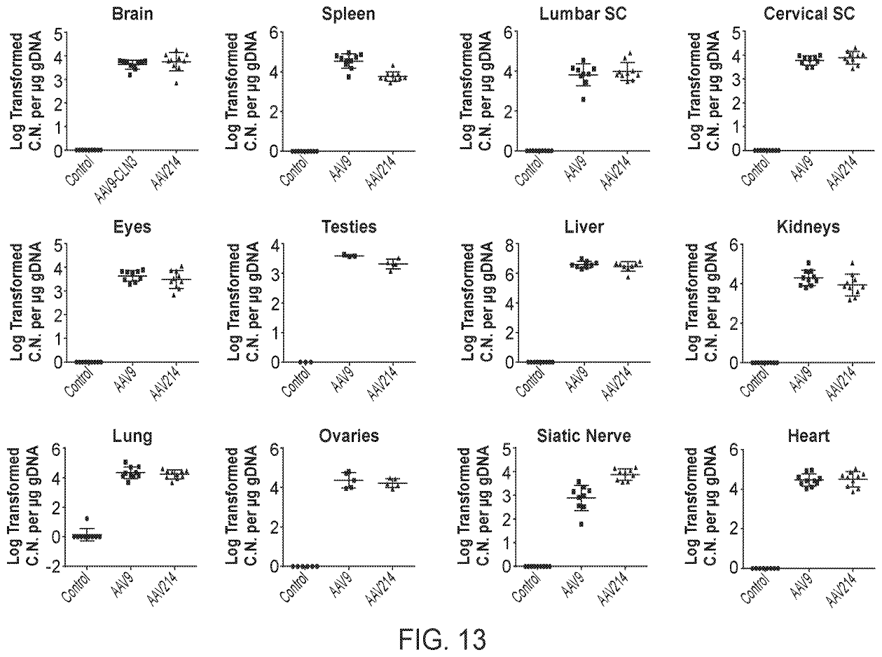

AAV9 and AAV214 viral vectors each comprising the CLN3 transgene (AAV9-CLN3 and AAV214-CLN3, respectively) were intravenously administered to wild type mice at a dosage of 2.0×10¹³ vg/kg (viral vector genomes/kilogram). See Table 6. After 30 days, the animals were humanely sacrificed and tissues were harvested for biodistribution analysis, which evaluates the delivery of the vector particles to several different organs, including the primary regions of the CNS and the spinal cord (cervical and lumbar). A t-test analysis of log-transformed data did not reveal a statistically significant difference between AAV214-CLN3 and AAV9-CLN3 in biodistribution values for most tissues that were tested (see FIG. 13). However, the sciatic nerve demonstrated statistically significantly (p=0.0001) higher (744%) biodistribution using AAV214-CLN3 compared to AAV9, while spleen was better transduced with AAV9-CLN3 (p<0.0001). Current studies assessing expression and dose response over a longer duration indicate that AAV214-CLN3 can be used for effectively delivering the CLN3 expression cassette to CNS tissues via systemic administration.

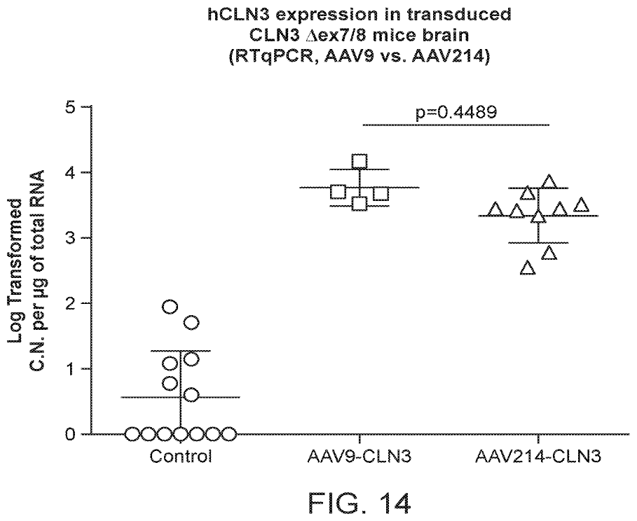

Expression of the CLN3 transgene was assessed by RTqPCR using total RNA isolated from left hemispheres of the brain. One-way ANOVA analysis of log-transformed data revealed no statistically significant difference in mean CLN3 expression values for AAV9-CLN3 versus AAV214-CLN3 dosed animals (p=0.4489). However, both tested viral vectors produced higher CLN3 expression levels than control (p<0.0001; FIG. 14).

In summary, these results showed that the novel AAV214 viral vector comprising an optimized CLN3 expression cassette demonstrates equivalent tropism to AAV9 in the most tissues including CNS if dosed via systemic administration in mouse model of CLN3 disease. These results indicate that the AAV214 vector comprising the optimized CLN3 transgene described herein can be used in prevention and treatment of CLN3 disease.

### Example 5

### Amelioration of Defects Caused by Fabry Disease Using Optimized GLA Transgenes Delivered by AAV214 Vector

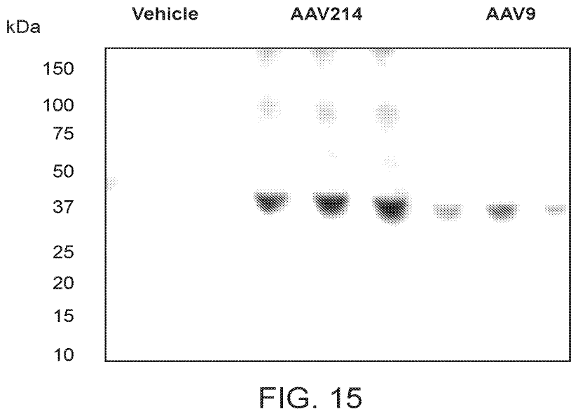

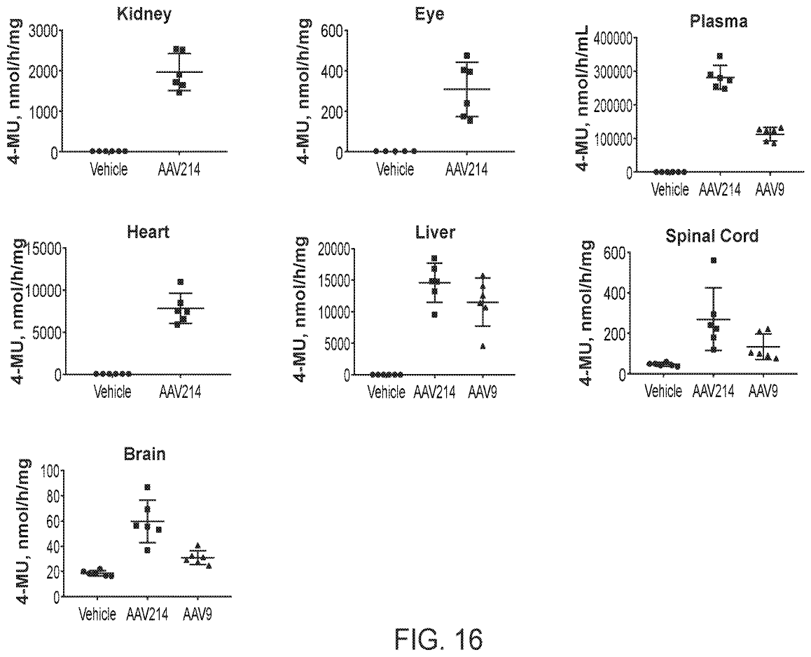

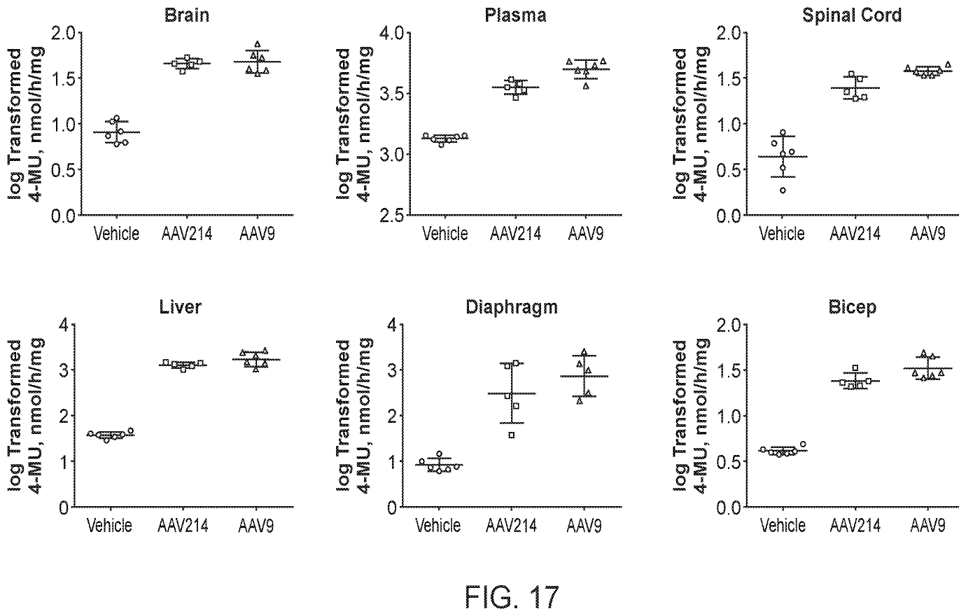

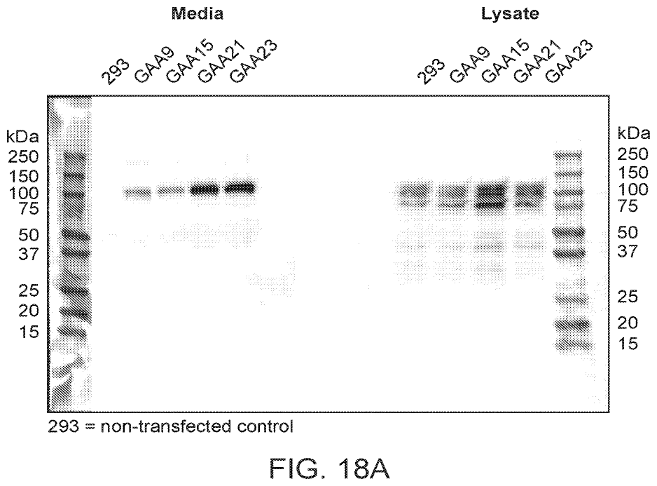

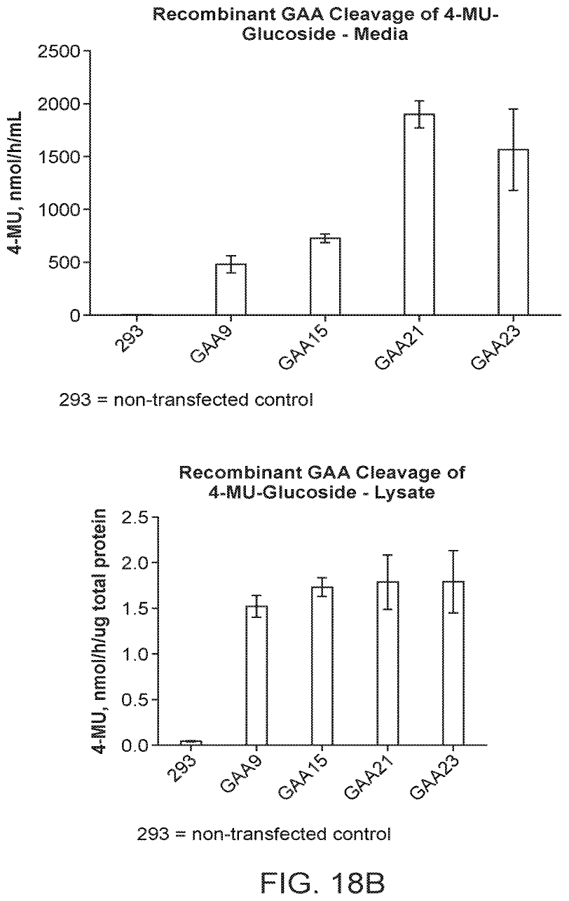

AAV9 and AAV214 viral vectors comprising a CBh promoter, CBA-MVM hybrid intron, natural GLA transgene sequence, and TK65 poly-A site were administered to wild type C57BL/6 mice by IV injection (see Table 7). Expected transgenic GLA protein size in plasma samples was confirmed by immunoblotting (FIG. 15). GLA enzymatic activity was assessed in plasma, brain, spinal cord, heart, kidney, liver and eye. Statistical analysis performed on log-transformed GLA enzyme activity values showed that all AAV214-GLA transduced samples had statistically significantly higher GLA activity compared to control (p<0.0001). GLA enzyme activity was also statistically significantly higher in AAV214-GLA transduced plasma, brain and spinal cord tissues compared to AAV9-GLA (FIG. 16). In summary, analysis of GLA enzymatic activity shows effective transduction of AAV214 constructs into multiple target tissues, particularly CNS tissues, demonstrating therapeutic benefit of the AAV214 vector in subjects with Fabry disease.

TABLE 7
Animal Study Design

| | Mouse | | Dose | Dose Volume | |
|---|---|---|---|---|---|
| Group | Strain | Vector | (vg/kg) | (microliter) | Animals |
| 1 | C57BL6 | Vehicle | 0 | 0 | 6 |
| 2 | C57BL6 | AAV9-hGLA | 1 × 10¹³ | 200 | 6 |
| 3 | C57BL6 | AAV214-hGLA | 1 × 10¹³ | 200 | 6 |

No acute toxic effects from systemic administration of the GLA transgene via an AAV9 or AAV214 viral vector were observed after the 10-day study in wild type animals. No animals treated in this experiment exhibited any adverse effects due to treatment. The effective delivery of AAV9 and AAV214 to target tissues, notably to the CNS, heart and kidney, after systemic administration demonstrates the ability to safely transduce key target tissues associated with Fabry disease.

### Example 6

### Amelioration of Defects Caused by Pompe Disease Using Optimized GAA Transgenes Delivered by AAV214 Vector

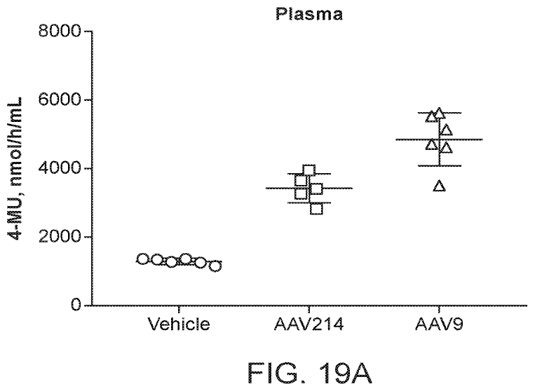

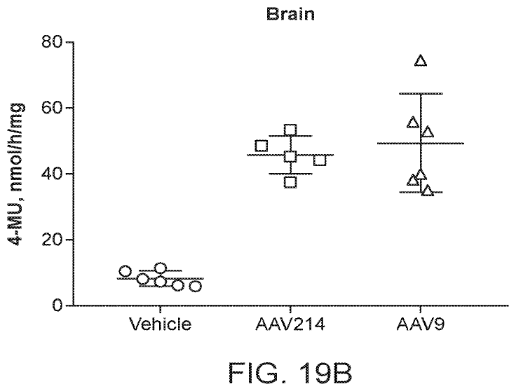

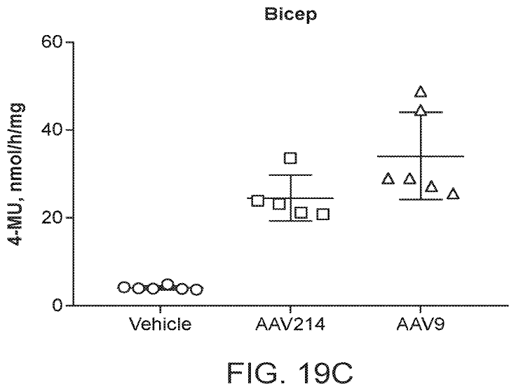

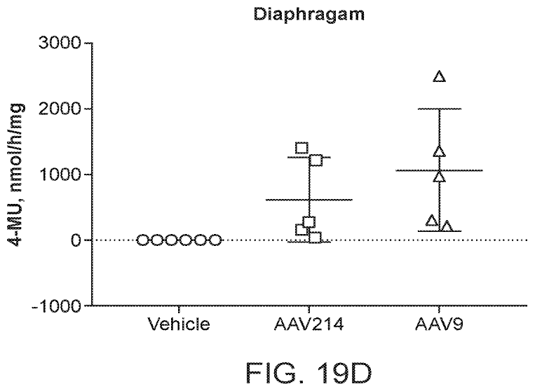

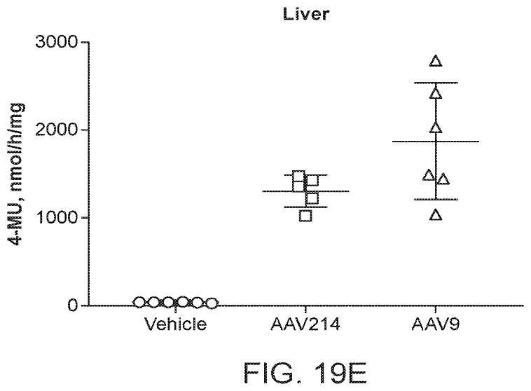

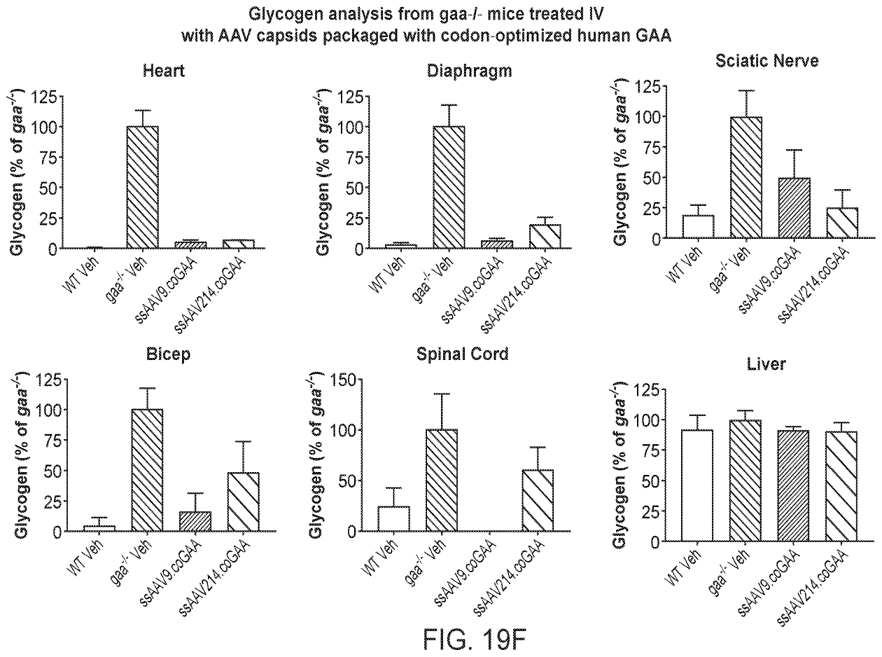

AAV9-GAA and AAV214-GAA vectors comprising a CBh promoter, CBA-MVM hybrid intron, codon optimized GAA transgene sequence, and BGH poly-A site were intravenously dosed by into wild-type C57BL/6 mice (see Table 8). In order to determine whether the transgenes were effectively delivered to the target tissues, GAA enzymatic activity protein was tested in brain, spinal cord, diaphragm, bicep, liver and plasma from the treated mice. One-way ANOVA analysis of log-transformed values of GAA enzymatic activity revealed that all tested tissues of dosed animals had statistically significantly (p<0.002) higher GAA activity compared to control animals. No statistically significant difference in enzyme activity was shown between AAV214 and AAV9 dosed tissues, except for in plasma, where AAV9 had slight advantage (p=0.0018) (FIGS. 19A-E). Analysis of GAA enzymatic activity confirmed effective transduction of AAV214 constructs into multiple target tissues, including an ability to cross the blood brain barrier, and transduction to tissues important for treating Pompe disease, such as, biceps and diaphragm (FIG. 19). These results suggest that a single intravenous injection of an AAV214 viral vector comprising an optimized GAA expression cassette as described herein may be sufficient to achieve delivery of the corrected GAA transgene to the target tissues. No acute toxic effects from the systemic administration of the GAA transgene via the AAV9 or AAV214 vector were observed after the 10-day study in wild type animals.

FIG. 19F shows repair of the underlying molecular pathology by AAV-delivered GAA. Glycogen analysis from gaa−/− mice treated intravenously with AAV capsids packaged with codon-optimized human GAA. Glycogen content was measured indirectly by release of glucose following amyloglucosidase treatment. Free glucose was measured with Infinity Glucose Reagent and analyzed on a SpectraMax i3×. Data is presented as % of gaa−/− vehicle control treated animals. The data shows the reduction in glycogen levels obtained by AAV-delivered GAA. Glycogen clearance was observed in all target tissues with AAV214 performing as effectively as AAV9.

These data confirm that systemic delivery of AAV9 and AAV214, notably with muscle and peripheral nervous system (PNS) expression, demonstrates the ability to both safely transduce key target tissues associated with Pompe disease and to restore GAA functionality.

TABLE 8
Animal Study Design

| | Mouse | | Dose | Dose Vol. | |
|---|---|---|---|---|---|
| Group | Strain | Vector | (vg/kg) | (microliters) | Animals |
| 1 | C57BL6 | Vehicle | 0 | 0 | 6 |
| 2 | C57BL6 | AAV9-hGAA | 1 × 10¹³ | 200 | 6 |
| 3 | C57BL6 | AAV214-hGAA | 1 × 10¹³ | 200 | 5 |

### Example 7

### AAV110 Vector Particles Show Highly Specific Muscle Tropism

AAV110 particles were prepared using the pAAV110 plasmid (also referred as ITCord1.10 plasmid) encoding the AAV110 capsid proteins. An AAV110-GFP viral vector comprising a CBh promoter, CBA-MVM hybrid intron, EGFP transgene sequence, and BGH poly-A site were administered (1×10¹¹ vg total, equivalent to 5×10¹² vg/kg) into each leg (biceps femoris) in a single injection in C57Bl/6 wild type mice. Another group of animals was administered an equivalent amount of AAV9-GFP viral vector for comparison.

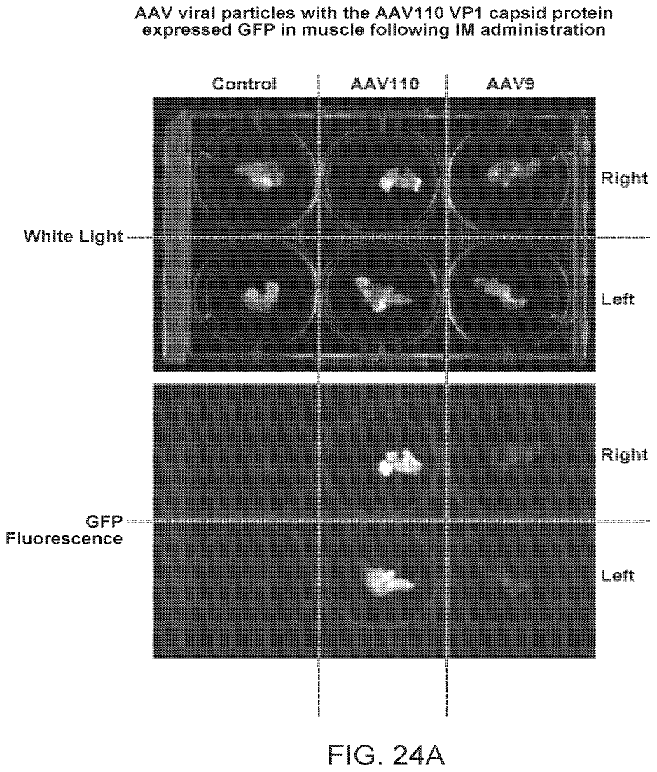

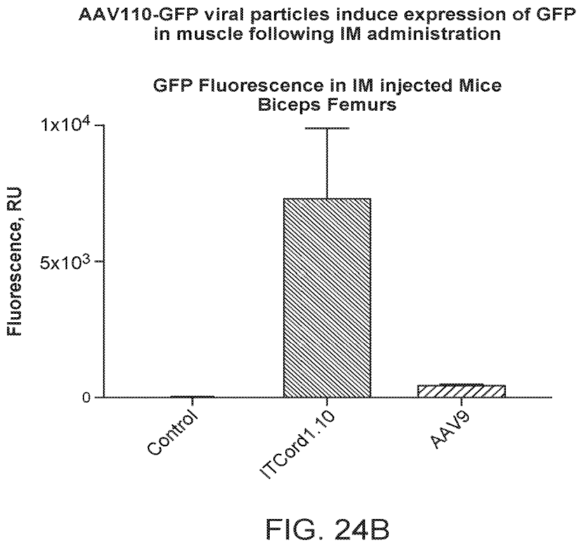

GFP expression was evaluated by imaging the leg muscle for fluorescence. FIG. 24A. The data showed the both right and left legs administered the AAV110-GFP viral vector expressed a high level of GFP, establishing muscle tropism for the AAV110 capsid. In contrast, AAV9-GFP vector particles provided substantially less muscle expression (FIG. 24B).

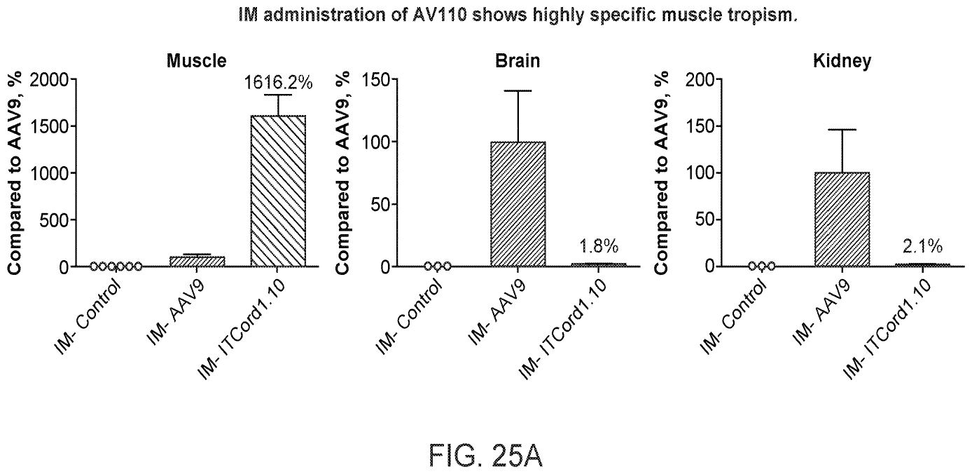

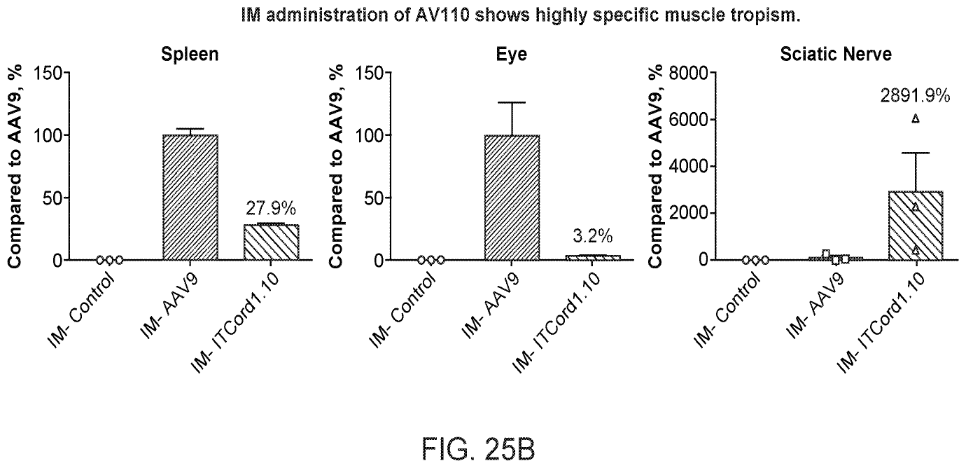

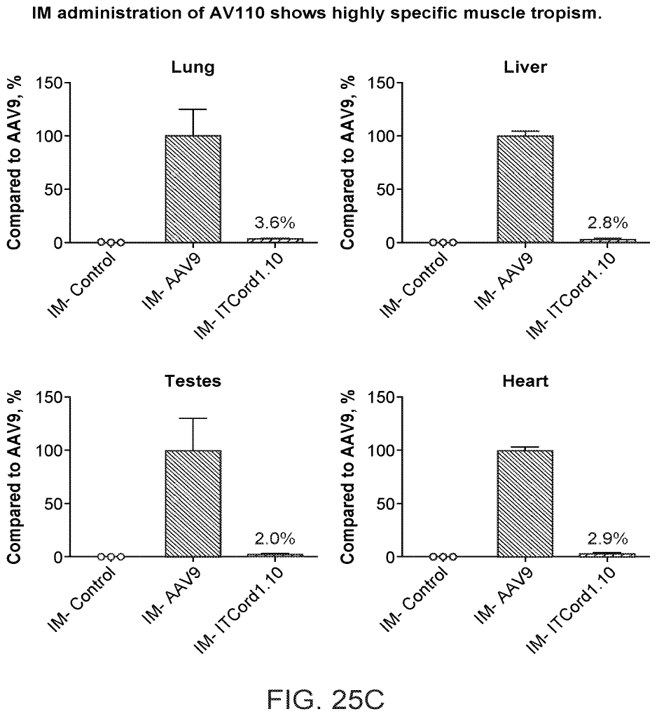

To assess the GFP transgene distribution in other tissues induced by intramuscular dosing of AAV110-GFP or AAV9-GFP we examined transgene biodistribution (BD) in a panel of organs. See FIG. 25. The data confirms that AAV110 transduction mostly happens in muscle, as well as in the sciatic nerve and spleen. In contrast to AAV9, AAV110 shows little to no biodistribution in brain, kidney, eye, lung, heart, liver and testes. In each case, BD was about 3% or less than that obtained with AAV9 intramuscular delivery of transgene.

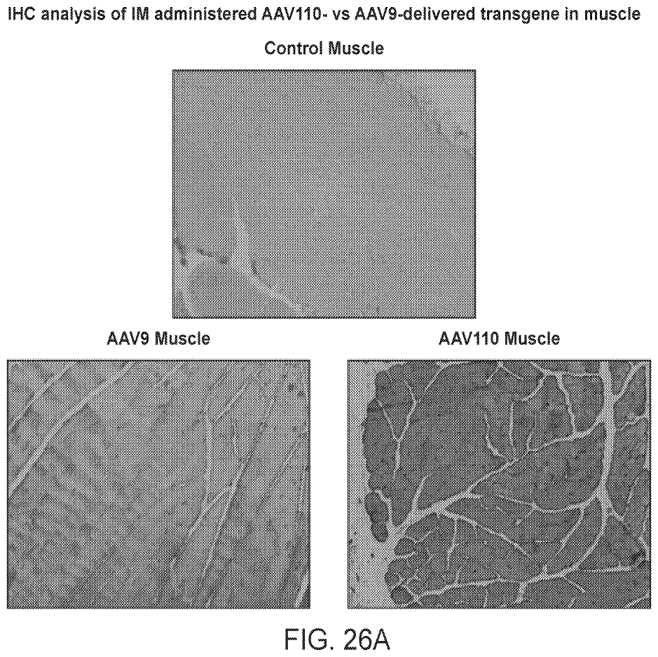

Immunohistochemistry analysis of muscle tissue confirmed high levels of GFP expression in muscle with AAV110 and less with AAV9 (FIG. 26). This data confirms superior muscle tropism and expression by AAV110.

### Example 8

AAV214 Vector Particles Provide High Level Expression in Muscle after IM and IV Administration

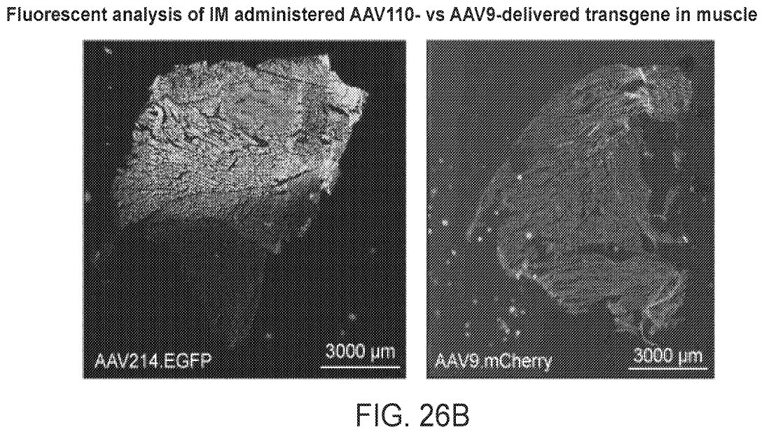

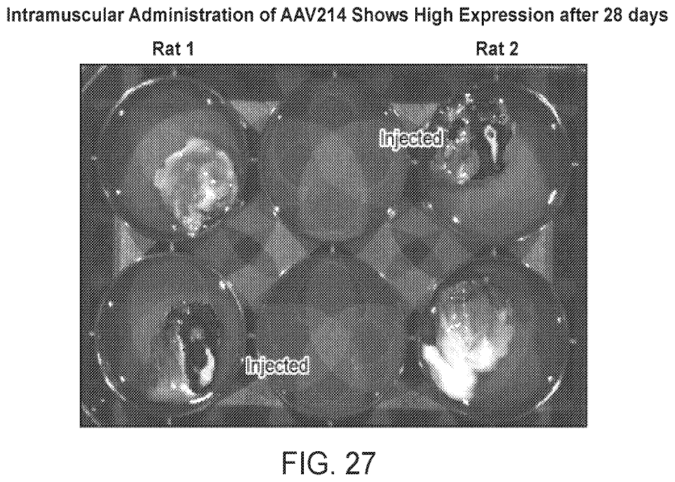

An AAV viral vector comprising the AAV214 capsid proteins and a luciferase expression cassette driven by U1a promoter (AAV214-Luc) was generated. We administered AAV214-Luc to the right leg of adult wild-type rats at a dose of 5×10¹² vg/kg in each muscle in a total volume of 0.1 mL per muscle. The left leg was untreated. To measure expression, we exposed the muscles to luciferin and measured emitted light. The data in the table below shows that injected muscle but not untreated muscle shows high expression 28 days after administration. FIG. 27 shows luciferase activity in the tissue, indicating activity of the expressed enzyme.

| | Total Flux [photons/second] | | |
|---|---|---|---|
| ID | pre-luciferin | 0 min post-luciferin | 5 min post-luciferin |
| Rat 1-left | 8.95 × 10⁴ | 5.21 × 10⁴ | 6.06 × 10⁴ |
| Rat 1-right | 1.75 × 10⁶ | 6.52 × 10⁶ | 1.24 × 10⁷ |
| Rat 2-right | 5.65 × 10⁶ | 9.12 × 10⁶ | 1.11 × 10⁷ |
| Rat 2-left | 1.32 × 10⁵ | 9.34 × 10⁶ | 1.46 × 10⁷ |

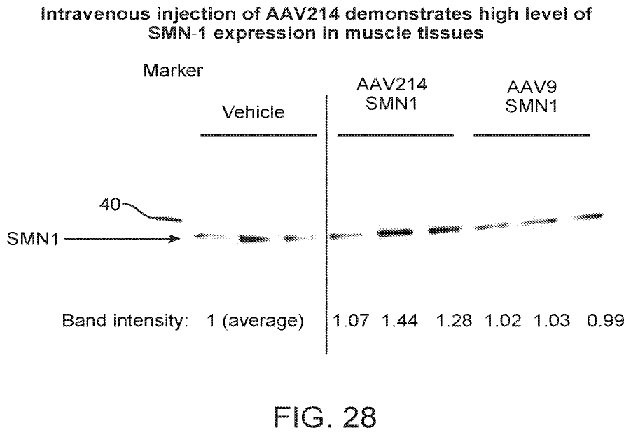

Similar results were obtained with a different transgene, codon optimized SMN-1 (survival of motor neuron 1) (which is defective in spinal muscle atrophy (SMA)) driven by a CBh promoter. We compared the ability of intravenously-administered AAV214-SMN1 viral vector particles with AAV9-SMN1 vector particles to express SMN-1 when administered intravenously. FIG. 28 shows expression of SMN1 in Tibialis anterior muscle tissue following infection of juvenile wild-type mice. The data illustrate that AAV214 vector particles provide improved expression with an increase from at least 10 to 30%, relative to vehicle and suitable for muscle transduction when delivered intravenously.

Comparison of AAV214 and AAV9 viral vectors to transduce muscle tissue showed that IM delivered AAV214 is able to transduce a larger muscle area than AAV9. Whole rat muscle (biceps femoris) was analyzed for GFP or mCherry expression by immunohistochemistry 10 days post-IM injection. Fixed and frozen sections were probed with GFP and mCherry pAb. AAV214 displayed a significantly larger transduction area in comparison to AAV9, which was largely confined to the upper portion of the muscle consistent with the injection site. (FIG. 26B).

### Example 9

AAV Vector Particles Containing Capsid Proteins Derived from AAV214 Exhibit High Expression in Muscle after IV Dosing

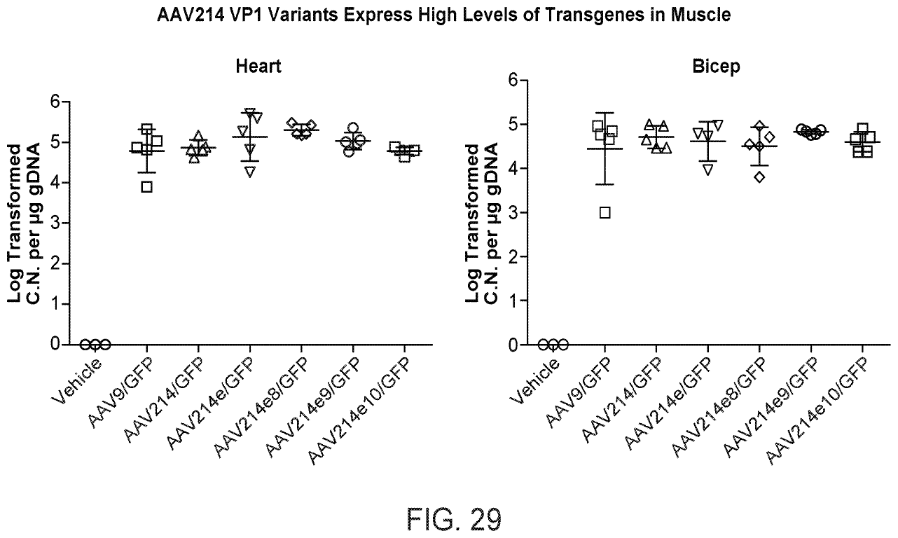

AAV214 VP1 proteins were modified by exchanging their N-terminus with amino acid sequences from known AAV serotypes (AAV8, AAV9, AAVrh10), thereby producing the variants shown in the table below. AAV viral vector particles were then prepared with each of newly-derived capsid proteins, essentially as described in Example 1, and their ability to transduce muscle following intravenous administration was assessed. We found that each viral vector conferred good muscle transduction in leg and heart (FIG. 29). One-way ANOVA analysis of log transformed biodistribution data did not reveal a statistically significant difference (p>0.05) in mean biodistribution values for the viral vectors tested.

| VP1 Amino Acid | VP1 Nucleic Acid | AAV |
|---|---|---|
| SEQ ID NO: | SEQ ID NO: | Capsid Name |
| 31 | 20 | AAV 214e |
| 32 | 21 | AAV 214e8 |
| 33 | 22 | AAV 214e9 |
| 34 | 23 | AAV 214e10 |

### Example 10

### Capsid-Induced Cross-Neutralizing Antibody Production

AAV204 and AAV214 have limited or very low cross-reactivity with AAV9 nAbs and low possibility of inducing nAbs production cross-reacting with AAV9.

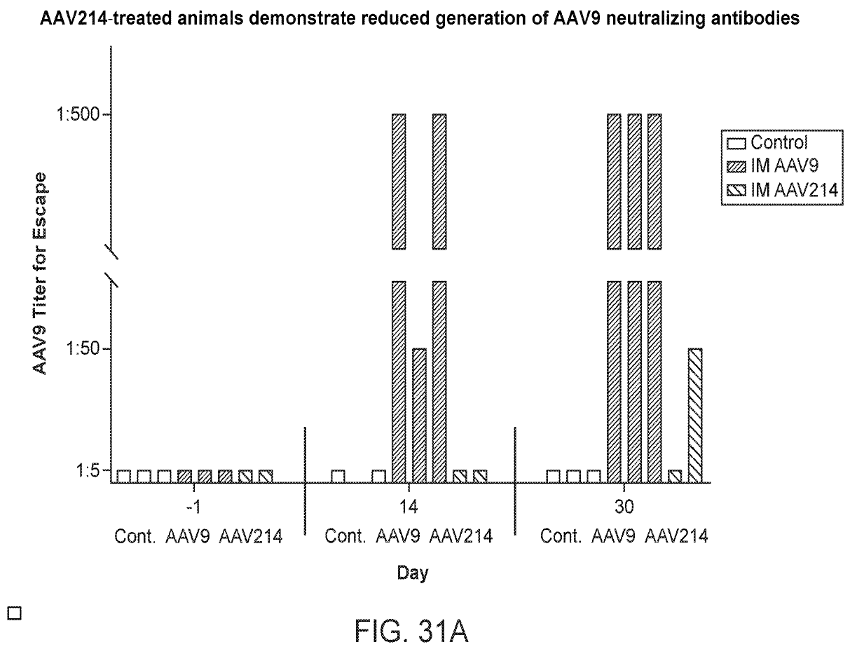

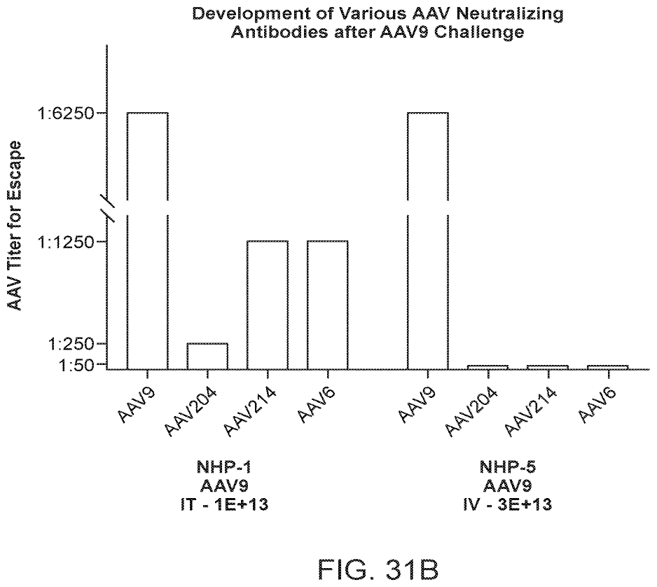

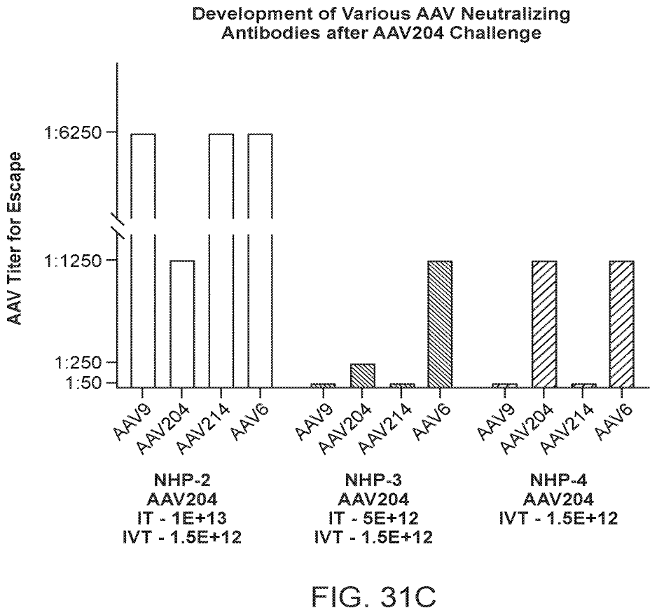

We tested the ability of animals dosed by IM with either AAV9 or AAV214 to produce neutralizing antibodies against AAV9. (FIG. 31A). Analysis was performed by measuring the ability of animal serum to inhibit the transduction of an AAV9.luciferase vector into the permissive cell type, Lec2. Three days post-transduction cells were assayed for luciferase activity. Each group consisted of 2 or 3 rats for either control, AAV9 or AAV214. Advantageously, animals injected with AAV214 by IM do not show a cross-reactive immune response to AAV9, which could allow for a larger patient population due to inclusion of patients with pre-existing immunity to AAV9, either naturally occurring or due to previous dosing.

Similar data were obtained in non-human primates (NHPs). Both AAV9 intrathecally (IT) and intravenously (IV) dosed NHPs developed nAbs against AAV9 (FIG. 31B). The IT dosed animal serum showed much higher cross-reactivity to other tested viruses (AAV204, AAV214 and AAV6). However, we believe that dosing route doesn't have significant impact on nAbs development differences because two animals dosed with AAV204 by intrathecal plus intravitreal route (IT+IV) also revealed similar differences in cross reactivity (FIG. 31C; see NHP-2 and NHP-3). Moreover, intravitreally (IVT) only AAV204 dosed animal (FIG. 31C; see NHP-4) showed similar cross reactivity as IT+IVT treated animal (NHP-3). The differences in the developed cross reactivity might be explained by the identity of the AAV capsid protein epitope against of which nAbs were produced. Both AAV9 dosed animal serum samples showed low reactivity to AAV204 (FIG. 31B), and two of three AAV204 treated animals demonstrated very low reactivity to AAV9 and AAV214 suggesting high chance of compatibility (FIG. 31C). In contrast, AAV6 revealed high cross reactivity in all AAV204 dosed animals (FIGS. 31B and C).

### Alternative Embodiments

1. A polynucleotide encoding an adeno-associated viral (AAV) capsid protein that comprises an amino acid sequence with at least 70% identity to SEQ ID NO: 3, 30-34, 49 or 84 or the use of a polynucleotide encoding an adeno-associated viral (AAV) capsid protein that comprises an amino acid sequence with at least 70% identity to SEQ ID NO: 1.
2. The polynucleotide of embodiment 1, wherein the amino acid sequence has at least 80% identity to SEQ ID NO: 3, 30-34, 49 or 84.
3. The polynucleotide of embodiment 1, wherein the amino acid sequence has at least 90% identity to SEQ ID NO: 3, 30-34, 49 or 84.
4. The polynucleotide of embodiment 1, wherein the amino acid sequence has at least 95% identity to SEQ ID NO: 3, 30-34, 49 or 84.
5. The polynucleotide of embodiment 1, wherein the amino acid sequence has at least 99% identity to SEQ ID NO: 3, 30-34, 49 or 84.
6. The polynucleotide of embodiment 1, wherein the amino acid sequence comprises SEQ ID NO: 3, 30-34, 49 or 84.
7. The polynucleotide of any one of embodiments 1-6, wherein the amino acid sequence comprises a VP1, VP2, and VP3 portion of the AAV capsid protein and wherein the VP3 portion has the sequence of SEQ ID NO:41.
8. The polynucleotide of embodiment 7, wherein the the AAV capsid protein is at least 70%, 80%, 90%, or 99% identical to SEQ ID NO: 3, 30-34, 49 or 84.
9. The polynucleotide of any one of embodiments 1-8, wherein the polynucleotide is contained within a plasmid, a bacterial artificial chromosome, a yeast artificial chromosome a phage, or a viral vector.
10. A host cell comprising the polynucleotide of any one of embodiments 1-8.
11. An AAV capsid protein comprising an amino acid sequence with at least 70%, 80%, 90%, or 99% identity with SEQ ID NO: 3, 30-34, 49 or 84.
12. An AAV capsid protein comprising an amino acid sequence having the sequence of SEQ ID NO:2, 3, 30-34, 49 or 84.
13. An AAV viral vector comprising
   (i) an AAV capsid protein of embodiment 11 or 12, and
   (ii) an AAV vector.
14. The AAV viral vector of embodiment 13, wherein the AAV vector comprises a heterologous nucleic acid.
15. The AAV viral vector of embodiment 14, wherein the heterologous nucleic acid is a transgene.
16. The AAV viral vector of embodiment 13 to 15, wherein the transgene encodes a cystic fibrosis transmembrane conductance regulator (CFTR), a CLN3 protein, an alpha-galactosidase A (GLA), or an acid alpha-glucosidase (GAA).
17. The AAV viral vector of embodiment 15, wherein the transgene comprises a sequence with at least 70%, 80%, 90%, or 99% identity with any one of SEQ ID NOs: 5, 6, 7, 9, 10, 12, and 13.
18. The AAV viral vector of embodiment 15, wherein the transgene encodes a protein comprising an amino acid sequence with at least 70%, 80%, 90%, or 99% identity with any one of SEQ ID NOs: 4, 5, 8, 11, and 14.
19. The AAV viral vector of embodiment 13 to 18, wherein the heterologous nucleic acid is operably linked to a promoter.
20. The AAV viral vector of embodiment 19, wherein the promoter is a tissue-specific control promoter, or a constitutive promoter.
21. The AAV viral vector of embodiment 20, wherein the promoter is a constitutive promoter which is a a Rous sarcoma virus (RSV) LTR promoter (optionally with the RSV enhancer), a cytomegalovirus (CMV) promoter, an SV40 promoter, a dihydrofolate reductase promoter, a beta-actin promoter, a phosphoglycerol kinase (PGK) promoter, a U6 promoter, an H1 promoter, a CAG promoter, a hybrid chicken beta-actin promoter, an MeCP2 promoter, an EF1 promoter, a ubiquitous chicken β-actin hybrid (CBh) promoter, a U1a promoter, a U1b promoter, an MeCP2 promoter, an MeP418 promoter, an MeP426 promoter, a minimal MeCP2 promoter, a VMD2 promoter, an mRho promoter, EF1a promoter, Ubc promoter, human β-actin promoter, TRE promoter, Ac5 promoter, Polyhedrin promoter, CaMKIIa promoter, Gal1 promoter, TEF1 promoter, GDS promoter, ADH1 promoter, Ubi promoter, or α-1-antitrypsin (hAAT) promoter.
22. The AAV viral vector of embodiment 10, wherein the promoter is a tissue-specific control promoter, which is a central nervous system (CNS) cell-specific promoter, a lung-specific promoter, a skin-specific promoter, a muscle-specific promoter, a liver-specific promoter, or an eye-specific promoter.
23. The AAV viral vector of embodiment 14, wherein the heterologous nucleic acid encodes an mRNA, siRNA, gRNA, or microRNA.
24. The AAV viral vector of embodiment 14, wherein the heterologous nucleic acid encodes a polypeptide.
25. The AAV viral vector of embodiment 14, wherein the heterologous nucleic acid encodes a cystic fibrosis transmembrane conductance regulator (CFTR), a CLN3 protein, an alpha-galactosidase A (GLA), or an acid alpha-glucosidase (GAA).
26. The AAV viral vector of embodiment 25, wherein the heterologous nucleic acid encodes a CFTR.
27. The AAV viral vector of embodiment 26, wherein the CFTR comprises or consists of an amino acid sequence encoded by SEQ ID NO: 4.
28. The AAV viral vector of embodiment 15, wherein the heterologous nucleic acid encodes a protein comprising an amino acid sequence with at least 70%, 80%, 90%, or 99% identity with any one of SEQ ID NOs; 5, 8, 11, and 14.
29. The AAV viral vector of embodiment 15, wherein the heterologous nucleic acid comprises a sequence with at least 70%, 80%, 90%, or 99% identity with any one of SEQ ID NOs: 4, 5, 6, 7, 9, 10, 12, and 13.
30. The AAV viral vector of embodiment 18, wherein the heterologous nucleic acid encodes a reporter protein.
31. A method of introducing a gene of interest to a cell in a subject, comprising contacting the cell with an effective amount of an AAV viral vectors of any one of embodiments 13-30.
32. The method of embodiment 31, wherein the AAV viral vector is introduced to the subject orally, rectally, transmucosally, inhalationally, transdermally, parenterally, intravenously, subcutaneously, intradermally, intramuscularly, intrapleurally, intracerebrally, intrathecally, intracerebrally, intraventricularly, intranasally, intra-aurally, intra-ocularly, peri-ocularly, topically, intralymphatically, intracistemally, intrathecally, or intra-vitreally.
33. The method of embodiment 31 or 32, wherein the subject is a mammal.
34. The method of embodiments 31 to 33, wherein the subject is human.
35. The method of embodiment 31 to 34, wherein the cell is a somatic cell.
36. The method of embodiment 35, wherein the somatic cell is a nerve cell, a retinal cell, a muscle cell, an epithelial cell, a lung cell, a liver cell, a stem cell, or a skin cell.
37. A pharmaceutical composition comprising the polynucleotide of any one of embodiments 1-8, the AAV capsid protein of embodiment 11 or 12, or the AAV viral vector of any one of embodiments 13-30.
38. A method of treating a disorder, comprising administering to a subject a therapeutically effective amount of the pharmaceutical composition of embodiment 37.
39. The method of embodiment 38, wherein the disorder is a CNS disorder, a skin disorder, a lung disorder, a muscle disorder, a liver disorder, or a retinal disorder.
40. The method of embodiment 38 or 39, wherein the disorder is amyotrophic lateral sclerosis (ALS), spinal muscular atrophy (SMA), Fabry disease, Pompe disease, CLN3 disease (or Juvenile Neuronal Ceroid Lipofuscinosis), recessive dystrophic epidermolysis bullosa (RDEB), juvenile Batten disease, autosomal dominant disorder, muscular dystrophy, hemophilia A, hemophilia B, multiple sclerosis, diabetes mellitus, Gaucher disease, cancer, arthritis, muscle wasting, heart disease, intimal hyperplasia, epilepsy, Huntington's disease, Parkinson's disease, Alzheimer's disease, cystic fibrosis, thalassemia, Hurler's Syndrome, Sly syndrome, Scheie Syndrome, Hurler-Scheie Syndrome, Hunter's Syndrome, Sanfilippo Syndrome A (mucopolysaccharidosis IIIA or MPS IIIA), Sanfilippo Syndrome B (mucopolysaccharidosis IIIB or MPS IIIB), Sanfilippo Syndrome C, Sanfilippo Syndrome D, Morquio Syndrome, Maroteaux-Lamy Syndrome, Krabbe's disease, phenylketonuria, Batten's disease, spinal cerebral ataxia, LDL receptor deficiency, hyperammonemia, arthritis, macular degeneration, retinitis pigmentosa, ceroid lipofuscinosis, neuronal, 1 (CLN1), or adenosine deaminase deficiency.
41. The method of embodiment 38, wherein the disorder is spinal muscular atrophy (SMA), recessive dystrophic epidermolysis bullosa (RDEB), Fabry disease, Pompe disease, CLN3 disease (or Juvenile Neuronal Ceroid Lipofuscinosis), MPS IIIA, MPS IIIB, juvenile Batten disease, and Duchenne muscular dystrophy (DMD), or Becker muscular dystrophy.
42. The method of embodiment 38, wherein disorder is cancer and the cancer is bladder cancer, breast cancer, cervical cancer, colon cancer, rectal cancer, endometrial cancer, kidney cancer, lip cancer, oral cancer, liver cancer, melanoma, mesothelioma, non-small cell lung cancer, nonmelanoma skin cancer, oral cancer, ovarian cancer, pancreatic cancer, prostate cancer, sarcoma, small cell lung cancer, or thyroid cancer.
43. The method of embodiment 37 to 42, wherein the subject is a mammal.
44. The method of embodiment 43, wherein the subject is human.
45. A kit comprising the polynucleotide of any one of embodiments 1-8, the cell of embodiment 10, the AAV capsid protein of embodiment 12 or 13, and/or the AAV viral vector of any one of embodiments 13-30.
46. An AAV packaging system, comprising the
   polynucleotide of any one of embodiments 1-8 and a helper cell.
47. The AAV package system of embodiment 46, wherein the helper cell is a yeast cell, a mammalian cell, or an insect cell
48. A nucleic acid encoding an AAV capsid protein, the AAV capsid protein comprising a leucine residue at amino acid 129, an asparagine residue at amino acid 586 and a glutamic acid residue at amino acid 723, wherein amino acid positions in the AAV capsid protein are numbered with respect to amino acid positions in the amino acid sequence of SEQ ID NO: 2.
49. The nucleic acid of embodiment 48, wherein the encoded AAV capsid amino acid sequence is at least 95% identical to the amino acid sequence of SEQ ID NO: 2.
50. The nucleic acid of embodiment 48, wherein the encoded AAV capsid amino acid sequence is at least 99% identical to the amino acid sequence of SEQ ID NO: 2
51. The nucleic acid of embodiment 48, wherein the nucleic acid sequence is at least 99% identical to the nucleotide sequence of SEQ ID NO: 15.
52. The nucleic acid of embodiment 48, wherein the nucleic acid sequence is 100% identical to the nucleotide sequence of SEQ ID NO: 15.
53. A vector comprising the nucleic acid of embodiment 48 to 52.
54. An AAV capsid protein encoded by the nucleic acid of embodiment 48 to 52.
55. The AAV capsid protein of embodiment 54, wherein the protein comprises the amino acid sequence of SEQ ID NO: 2.
56. An AAV viral vector comprising the AAV capsid protein encoded by the nucleic acid of embodiment 54 or 55 and an AAV vector, wherein the AAV vector comprises a heterologous nucleic acid.
57. The AAV viral vector of embodiment 56, wherein the heterologous nucleic acid is operably linked to a constitutive promoter.
58. The AAV viral vector of embodiment 56 or 57, wherein the heterologous nucleic acid encodes a polypeptide.
59. The AAV viral vector of embodiment 56 or 57, wherein heterologous nucleic acid encodes an antisense RNA, microRNA, or RNAi.
60. The AAV viral vector of embodiment 56, wherein the AAV capsid protein comprises the amino acid sequence of SEQ ID NO: 2.
61. A nucleic acid encoding an AAV capsid protein comprising a VP1 portion, a VP2 portion and a VP3 portion, wherein the VP3 portion comprises variable regions (VR) I to IX wherein:
(a) VR-II comprises amino acid sequence DNNGVK (SEQ ID NO. 54);
(b) VR-III comprises amino acid sequence NDGS (SEQ ID NO. 55);
(c) VR-IV comprises amino acid sequence INGSGQNQQT (SEQ ID NO. 56);
(d) VR-V comprises amino acid sequence RVSTTTGQNNNSNFAWTA (SEQ ID NO. 57);
(e) VR-VI comprises amino acid sequence HKEGEDRFFPLSG (SEQ ID NO. 58);
(f) VR-VII comprises amino acid sequence KQNAARDNADYSDV (SEQ ID NO. 59);
(g) VR-VIII comprises amino acid sequence ADNLQQQNTAPQI (SEQ ID NO. 60); and
(h) VR-IX comprises amino acid sequence NYYKSTSVDF (SEQ ID NO. 61).
62. The nucleic acid of embodiment 61, wherein the VR-I region comprises SASTGAS (SEQ ID NO. 52).
63. The nucleic acid of embodiment 61, wherein the VR-I region comprises NSTSGGSS (SEQ ID NO. 53) or SSTSGGSS (SEQ ID NO. 87).
64. The nucleic acid of embodiment 61 to 63, wherein the VP3 portion further comprises one or more of:
(i) an asparagine (N) at amino acid 223;
(ii) an alanine (A) residue at amino acid 224;
(iii) a threonine (T) residue at amino acid 410;
(iv) a histidine residue at amino acid 724; and
(v) a proline (P) residue at amino acid 734, wherein amino acid positions in the AAV capsid protein are numbered with respect to amino acid positions in the amino acid sequence of SEQ ID NO: 3.
65. The nucleic acid of embodiment 61 to 64, wherein the encoded AAV capsid amino acid sequence is at least 95% identical to the amino acid sequence of SEQ ID NO: 3, 30, 31, 32, 33, 34, 49 or 84.
66. The nucleic acid of embodiment 61 to 65, wherein the encoded AAV capsid amino acid sequence is at least 99% identical to the amino acid sequence of SEQ ID NO: 3, 30, 31, 32, 33, 34, 49 or 84.
67. The nucleic acid of embodiment 61 to 66, wherein the nucleic acid sequence is at least 99% identical to the nucleotide sequence selected from SEQ ID NO: 18, 19, 20, 21, 22, 23, 47, 82 or 98.
68. The nucleic acid of embodiment 61, wherein the nucleic acid sequence is 100% identical to the nucleotide sequence selected from SEQ ID NO: 18, 19, 20, 21, 22, 23, 47, 82 or 98.
69. A vector comprising the nucleic acid of embodiment 61 to 68.
70. An AAV capsid protein encoded by the nucleic acid of embodiment 61 to 68.
71. The AAV capsid protein of embodiment 70, wherein the protein comprises the amino acid sequence of SEQ ID NO: 3, 30, 31, 32, 33, 34, 49 or 84.
72. An AAV viral vector comprising the AAV capsid protein encoded by the nucleic acid of embodiment 61 to 68 and an AAV vector, wherein the AAV vector comprises a heterologous nucleic acid.
73. The AAV viral vector of embodiment 72, wherein the heterologous nucleic acid is operably linked to a constitutive promoter.
74. The AAV viral vector of embodiment 72 or 73, wherein the heterologous nucleic acid encodes a polypeptide.
75. The AAV viral vector of embodiment 72 or 73, wherein the heterologous nucleic acid encodes an antisense RNA, microRNA, or RNAi.
76. The AAV viral vector of embodiment 72 to 75, wherein the AAV capsid protein comprises the amino acid sequence of SEQ ID NO: 3, 30, 31, 32, 33, 34, 49 or 84.
77. A nucleic acid encoding an AAV capsid protein comprising a VP1 portion, a VP2 portion and a VP3 portion, wherein the VP3 portion comprises variable regions (VR) I to IX wherein:

(a) VR-I comprises amino acid sequence SASTGAS (SEQ ID NO: 52)

(b) VR-II comprises amino acid sequence DNNGVK (SEQ ID NO. 54);

(c) VR-III comprises amino acid sequence NDGS (SEQ ID NO. 55);

(d) VR-IV comprises amino acid sequence INGSGQNQQT (SEQ ID NO. 56);

(e) VR-V comprises amino acid sequence RVSTTTGQNNNSNFAWTA (SEQ ID NO. 57);

(f) VR-VI comprises amino acid sequence HKEGEDRFFPLSG (SEQ ID NO. 58);

(g) VR-VII comprises amino acid sequence KQNAARDNADYSDV (SEQ ID NO. 59);

(h) VR-VIII comprises amino acid sequence ADNLQQQNTAPQI (SEQ ID NO. 60); and
(i) VR-IX comprises amino acid sequence NYYKSTSVDF (SEQ ID NO. 61).
78. The nucleic acid of embodiment 77, wherein the VP3 portion further comprises one or more of:
(i) an asparagine (N) at amino acid 223;
(ii) an alanine (A) residue at amino acid 224;
(iii) a threonine (T) residue at amino acid 410;
(iv) a histidine residue at amino acid 724; and
(v) a proline (P) residue at amino acid 734, wherein amino acid positions in the AAV capsid protein are numbered with respect to amino acid positions in the amino acid sequence of SEQ ID NO: 3.
79. The nucleic acid of embodiment 77 or 78, wherein the VP3 portion has the sequence of SEQ ID NO: 41.
80. The nucleic acid of embodiment 77 to 79, wherein the VP1 and VP2 portion of the encoded AAV capsid amino acid sequence is at least 95% identical to the amino acid sequence of the VP1 and VP2 portion of SEQ ID NO: 3, 31, 32, 33 or 34.
81. The nucleic acid of embodiment 77 to 80, wherein the encoded AAV capsid amino acid sequence is at least 99% identical to the amino acid sequence of SEQ ID NO: 3, 31, 32, 33 or 34.
82. The nucleic acid of embodiment 77 to 81, wherein the nucleic acid sequence is at least 99% identical to the nucleotide sequence selected from SEQ ID NO: 18, 20, 21, 22 or 23.
83. The nucleic acid of embodiment 77 to 82, wherein the nucleic acid sequence is 100% identical to the nucleotide sequence selected from SEQ ID NO: 18, 20, 21, 22 or 23.
84. A vector comprising the nucleic acid of embodiments 57 to 83.
85. An AAV capsid protein encoded by the nucleic acid of embodiments 77 to 83.
86. The AAV capsid protein of embodiment 85, wherein the protein comprises the amino acid sequence of SEQ ID NO: 3, 31, 32, 33 or 34.
87. An AAV viral vector comprising the AAV capsid protein encoded by the nucleic acid of embodiment 88 and an AAV vector, wherein the AAV vector comprises a heterologous nucleic acid.
88. The AAV viral vector of embodiment 87, wherein the heterologous nucleic acid is operably linked to a constitutive promoter.
89. The AAV viral vector of embodiment 87 or 88, wherein the heterologous nucleic acid encodes a polypeptide.
90. The AAV viral vector of embodiment 87 or 88, wherein the heterologous nucleic acid encodes an antisense RNA, microRNA, or RNAi.
91. The AAV viral vector of embodiment 87 to 90, wherein the AAV capsid protein comprises the amino acid sequence of SEQ ID NO: 3, 31, 32, 33 or 34.
92. A nucleic acid encoding an AAV capsid protein comprising a VP1 portion, a VP2 portion and a VP3 portion, wherein the VP1 portion comprises a leucine (L) residue at amino acid 129, wherein the VP2 portion comprises a threonine (T) or asparagine (N) residue at amino acid 157 and a lysine (K) or serine(S) residue at amino acid 162, and wherein the VP3 portion comprises asparagine (N) residue at amino acid 223, an alanine (A) residue at amino acid 224, a histidine (H) residue at amino acid 272, a threonine (T) residue at amino acid 410, a histidine (H) residue at amino acid 724 and a proline (P) residue at amino acid 734, wherein amino acid positions in the AAV capsid protein are numbered with respect to amino acid positions in the amino acid sequence of SEQ ID NO: 3.
93. The nucleic acid of embodiment 92, wherein the VP3 portion comprises variable regions (VR) I to IX wherein:
(a) VR-I comprises amino acid sequence SASTGAS (SEQ ID NO: 52);
(b) VR-II comprises amino acid sequence DNNGVK (SEQ ID NO. 54);
(c) VR-III comprises amino acid sequence NDGS (SEQ ID NO. 55);
(d) VR-IV comprises amino acid sequence INGSGQNQQT (SEQ ID NO. 56);
(e) VR-V comprises amino acid sequence RVSTTTGQNNNSNFAWTA (SEQ ID NO. 57);
(f) VR-VI comprises amino acid sequence HKEGEDRFFPLSG (SEQ ID NO. 58);
(g) VR-VII comprises amino acid sequence KQNAARDNADYSDV (SEQ ID NO. 59);
(h) VR-VIII comprises amino acid sequence ADNLQQQNTAPQI (SEQ ID NO. 60); and
(i) VR-IX comprises amino acid sequence NYYKSTSVDF (SEQ ID NO. 61).
94. The nucleic acid of embodiment 92 or 93, wherein the VP1 portion further comprises an aspartic acid (D) or alanine (A) residue at amino acid 24, wherein amino acid positions in the AAV capsid protein are numbered with respect to amino acid positions in the amino acid sequence of SEQ ID NO: 3.

95. The nucleic acid of embodiment 92 to 94, wherein the VP2 portion further comprises one or more of
(i) a proline (P) residue at amino acid 148;
(ii) an arginine (R) residue inserted at amino acid 152;
(iii) an arginine (R) residue at amino acid 168;
(iv) an isoleucine (I) residue at amino acid 189; and
(v) a serine(S) residue at amino acid 200,
wherein amino acid positions in the AAV capsid protein are numbered with respect to amino acid positions in the amino acid sequence of SEQ ID NO: 3.
96. The nucleic acid of embodiment 92 to 96, wherein the encoded AAV capsid amino acid sequence is at least 95% identical to the amino acid sequence of SEQ ID NO: 31, 32, 33 or 34.
97. The nucleic acid of embodiment 92, wherein the encoded AAV capsid amino acid sequence is at least 99% identical to the amino acid sequence of SEQ ID NO: 31, 32, 33 or 34.
98. The nucleic acid of embodiment 92, wherein the nucleic acid sequence is at least 99% identical to the nucleotide sequence selected from SEQ ID NO: 20, 21, 22 or 23.
99. The nucleic acid of embodiment 98, wherein the nucleic acid sequence is 100% identical to the nucleotide sequence selected from SEQ ID NO: 20, 21, 22 or 23.
100. A vector comprising the nucleic acid of embodiment 92 to 99.
101. An AAV capsid protein encoded by the nucleic acid of embodiment 92 to 99.
102. The AAV capsid protein of embodiment 101, wherein the protein comprises the amino acid sequence of SEQ ID NO: 31, 32, 33 or 34.
103. An AAV viral vector comprising the AAV capsid protein encoded by the nucleic acid of embodiment 101 or 102 and an AAV vector, wherein the AAV vector comprises a heterologous nucleic acid.
104. The AAV viral vector of embodiment 103, wherein the heterologous nucleic acid is operably linked to a constitutive promoter.
105. The AAV viral vector of embodiment 103 or 104, wherein the heterologous nucleic acid encodes a polypeptide, an antisense RNA, microRNA, or RNAi.

1.-28. (canceled)

29. A method of treating an eye disease or disorder in a subject in need thereof, comprising administering an adeno-associated virus (AAV) viral vector to an eye of the subject, wherein the AAV viral vector comprises an AAV capsid protein comprising an amino acid sequence of SEQ ID NO: 2.

30. The method of claim 29, wherein the AAV viral vector comprises an AAV vector comprising, in 5′ to 3′ orientation,
(a) a first AAV inverted terminal repeat,
(b) a promoter,
(c) a heterologous nucleic acid,
(d) a poly-A tail; and
(e) a second AAV inverted terminal repeat.

31. The method of claim 30, wherein the heterologous nucleic acid encodes a polypeptide.

32. The method of claim 31, wherein the polypeptide is retinoschisin 1 (RS1) or phosphodiesterase 6B (PDE6B).

33. The method of claim 30, wherein the heterologous nucleic acid comprises a gene selected from SPATA7, LRAT, TULP1, AIPL1, RPGR, AIPL1, ABCA4, CHM, MY07A, CDH23, USH2A, CLRN1, RS1, CYP4V2, CNGA3, CNGB3, GNAT2, PDE6C, PDE6H, and BEST-1.

34. The method of claim 30, wherein heterologous nucleic acid encodes an antisense RNA, microRNA, siRNA, or guide RNA (gRNA).

35. The method of claim 30, wherein the promoter is a constitutive promoter.

36. The method of claim 30, wherein the promoter is a Rous sarcoma virus (RSV) LTR promoter, a cytomegalovirus (CMV) promoter, an SV40 promoter, a dihydrofolate reductase promoter, a beta-actin promoter, a phosphoglycerol kinase (PGK) promoter, a U6 promoter, an H1 promoter, a CAG promoter, a hybrid chicken beta-actin promoter, an MeCP2 promoter, an EF1 promoter, a ubiquitous chicken β-actin hybrid (CBh) promoter, a U1a promoter, a U1b promoter, an MeCP2 promoter, an MeP418 promoter, an MeP426 promoter, a minimal MeCP2 promoter, a VMD2 promoter, an mRho promoter, EF1a promoter, Ubc promoter, human β-actin promoter, TRE promoter, Ac5 promoter, Polyhedrin promoter, CaMKIIa promoter, Gal1 promoter, TEF1 promoter, GDS promoter, ADH1 promoter, Ubi promoter, or α-1-antitrypsin (hAAT) promoter.

37. The method of claim 30, wherein the promoter is an eye-specific promoter.

38. The method of claim 37, wherein the eye-specific promoter is a VMD2 promoter, a PDE6b promoter, or a mRho promoter.

39. The method of claim 29, wherein the AAV viral vector is administered to the subject intra-ocularly, peri-ocularly, subretinally, or intravitreally.

40. The method of claim 29, wherein the AAV viral vector transduces one or more cell types selected from photoreceptors, retinal pigment epithelium, Müller glia cells, retinal ganglion cells and bipolar cells.

41. The method of claim 29, wherein the eye disease or disorder is macular degeneration, retinitis pigmentosa, Leber congenital amaurosis, cone-rod dystrophy, Stargardt disease, choroideremia, Usher syndrome, retinoschisis, Bietti's Crystalline Dystrophy, or achromatopsia.

42. The method of claim 41, wherein the retinitis pigmentosa is autosomal recessive, autosomal dominant, or X-linked.

43. The method of claim 29, wherein the eye disease or disorder is caused by a mutation in a bestrophin-1 (BEST-1) gene.

44. The method of claim 29, wherein the eye disease or disorder is vitelliform macular dystrophy, age-related macular degeneration, autosomal dominant vitreoretinochoroidopathy, glaucoma, or cataract.

45. The method of claim 29, wherein the eye disease or disorder is retinoschisis.

46. An AAV capsid protein comprising an amino acid sequence of SEQ ID NO: 2.

47. An AAV viral vector comprising the AAV capsid protein of claim 46.

48. A nucleic acid comprising a polynucleotide sequence encoding the AAV capsid protein of claim 46.

49. The nucleic acid of claim 48, wherein the polynucleotide sequence is at least 95% identical to SEQ ID NO: 15.


SEQUENCE LISTING (163 sequences)

SEQ ID NO: 1 - protein, 737 aa, synthetic construct
  REGION 1..737: note = AAV110 VPl
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDKDKFFPMSGVMIFGKESAGASNTALDNVMITDEEEIKATNPVATERFGTVAVNLQSSSTDPATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 2 - protein, 736 aa, synthetic construct
  REGION 1..736: note = AAV204 VP1
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDKDKFFPMSGVMIFGKESAGASNTALDNVMITDEEEIKATNPVATERFGTVAVNLQNSSTDPATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 3 - protein, 735 aa, synthetic construct
  REGION 1..735: note = AAV214 VPl
MAADGYLPDWLEDNLSEGIREWWALKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPFGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTDNNGVKTIANNLTSTVQVFTDSDYQLPYVLGSAHQGCLPPFPADVFMIPQYGYLTLNDGSQAVGRSSFYCLEYFPSQMLRTGNNFTFSYTFEDVPFHSSYAHSQSLDRLMNPLIDQYLYYLSKTINGSGQNQQTLKFSQGGPNTMANQAKNWLPGPCYRQQRVSTTTGQNNNSNFAWTAGTKYHLNGRNSLMNPGPAMASHKEGEDRFFPLSGSLIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPHPIGTRYLTRPL

SEQ ID NO: 4 - DNA, 4287 bp, synthetic construct
  misc_feature 1..4287: note = CFTRdeltaR
  source 1..4287: mol_type = other DNA
atgcagagaagccccctggagaaggcctctgtggtgagcaagctgttcttcagctggaccagacccatcctgagaaagggctacagacagagactggagctgtctgacatctaccagatcccctctgtggactctgctgacaacctgtctgagaagctggagagagagtgggacagagagctggccagcaagaagaaccccaagctgatcaatgccctgagaagatgcttcttctggagattcatgttctatggcatcttcctgtacctgggggaggtgaccaaggctgtgcagcccctgctgctgggcagaatcattgccagctatgaccctgacaacaaggaggagagaagcattgccatctacctgggcattggcctgtgcctgctgttcattgtgagaaccctgctgctgcaccctgccatctttggcctgcaccacattggcatgcagatgagaattgccatgttcagcctgatctacaagaagaccctgaagctgagcagcagagtgctggacaagatcagcattggccagctggtgagcctgctgagcaacaacctgaacaagtttgatgagggcctggccctggcccactttgtgtggattgcccccctgcaggtggccctgctgatgggcctgatctgggagctgctgcaggcctctgccttctgtggcctgggcttcctgattgtgctggccctgttccaggctggcctgggcagaatgatgatgaagtacagagaccagagagctggcaagatctctgagagactggtgatcacctctgagatgattgagaacatccagtctgtgaaggcctactgctgggaggaggccatggagaagatgattgagaacctgagacagacagagctgaagctgaccagaaaggctgcctatgtgagatacttcaacagctctgccttcttcttctctggcttctttgtggtgttcctgtctgtgctgccctatgccctgatcaagggcatcatcctgagaaagatcttcaccaccatcagcttctgcattgtgctgagaatggctgtgaccagacagttcccctgggctgtgcagacctggtatgacagcctgggggccatcaacaagatccaggacttcctgcagaagcaggagtacaagaccctggagtacaacctgaccaccacagaggtggtgatggagaatgtgacagccttctgggaggagggctttggggagctgtttgagaaggccaagcagaacaacaacaacagaaagaccagcaatggggatgacagcctgttcttcagcaacttcagcctgctgggcacccctgtgctgaaggacatcaacttcaagattgagagaggccagctgctggctgtggctggcagcacaggggctggcaagaccagcctgctgatgatgatcatgggggagctggagccctctgagggcaagatcaagcactctggcagaatcagcttctgcagccagttcagctggatcatgcctggcaccatcaaggagaacatcatctttggggtgagctatgatgagtacagatacagatctgtgatcaaggcctgccagctggaggaggacatcagcaagtttgctgagaaggacaacattgtgctgggggaggggggcatcaccctgtctgggggccagagagccagaatcagcctggccagagctgtgtacaaggatgctgacctgtacctgctggacagcccctttggctacctggatgtgctgacagagaaggagatctttgagagctgtgtgtgcaagctgatggccaacaagaccagaatcctggtgaccagcaagatggagcacctgaagaaggctgacaagatcctgatcctgcatgagggcagcagctacttctatggcaccttctctgagctgcagaacctgcagcctgacttcagcagcaagctgatgggctgtgacagctttgaccagttctctgctgagagaagaaacagcatcctgacagagaccctgcacagattcagcctggagggggatgcccctgtgagctggacagagaccaagaagcagagcttcaagcagacaggggagtttggggagaagagaaagaacagcatcctgaaccccatcaacagcaccctgcaggccagaagaagacagtctgtgctgaacctgatgacccactctgtgaaccagggccagaacatccacagaaagaccacagccagcaccagaaaggtgagcctggccccccaggccaacctgacagagctggacatctacagcagaagactgagccaggagacaggcctggagatctctgaggagatcaatgaggaggacctgaaggagtgcttctttgatgacatggagagcatccctgctgtgaccacctggaacacctacctgagatacatcacagtgcacaagagcctgatctttgtgctgatctggtgcctggtgatcttcctggctgaggtggctgccagcctggtggtgctgtggctgctgggcaacacccccctgcaggacaagggcaacagcacccacagcagaaacaacagctatgctgtgatcatcaccagcaccagcagctactatgtgttctacatctatgtgggggtggctgacaccctgctggccatgggcttcttcagaggcctgcccctggtgcacaccctgatcacagtgagcaagatcctgcaccacaagatgctgcactctgtgctgcaggcccccatgagcaccctgaacaccctgaaggctgggggcatcctgaacagattcagcaaggacattgccatcctggatgacctgctgcccctgaccatctttgacttcatccagctgctgctgattgtgattggggccattgctgtggtggctgtgctgcagccctacatctttgtggccacagtgcctgtgattgtggccttcatcatgctgagagcctacttcctgcagaccagccagcagctgaagcagctggagtctgagggcagaagccccatcttcacccacctggtgaccagcctgaagggcctgtggaccctgagagcctttggcagacagccctactttgagaccctgttccacaaggccctgaacctgcacacagccaactggttcctgtacctgagcaccctgagatggttccagatgagaattgagatgatctttgtgatcttcttcattgctgtgaccttcatcagcatcctgaccacaggggagggggagggcagagtgggcatcatcctgaccctggccatgaacatcatgagcaccctgcagtgggctgtgaacagcagcattgatgtggacagcctgatgagatctgtgagcagagtgttcaagttcattgacatgcccacagagggcaagcccaccaagagcaccaagccctacaagaatggccagctgagcaaggtgatgatcattgagaacagccatgtgaagaaggatgacatctggccctctgggggccagatgacagtgaaggacctgacagccaagtacacagaggggggcaatgccatcctggagaacatcagcttcagcatcagccctggccagagagtgggcctgctgggcagaacaggctctggcaagagcaccctgctgtctgccttcctgagactgctgaacacagagggggagatccagattgatggggtgagctgggacagcatcaccctgcagcagtggagaaaggcctttggggtgatcccccagaaggtgttcatcttctctggcaccttcagaaagaacctggacccctatgagcagtggtctgaccaggagatctggaaggtggctgatgaggtgggcctgagatctgtgattgagcagttccctggcaagctggactttgtgctggtggatgggggctgtgtgctgagccatggccacaagcagctgatgtgcctggccagatctgtgctgagcaaggccaagatcctgctgctggatgagccctctgcccacctggaccctgtgacctaccagatcatcagaagaaccctgaagcaggcctttgctgactgcacagtgatcctgtgtgagcacagaattgaggccatgctggagtgccagcagttcctggtgattgaggagaacaaggtgagacagtatgacagcatccagaagctgctgaatgagagaagcctgttcagacaggccatcagcccctctgacagagtgaagctgttcccccacagaaacagcagcaagtgcaagagcaagccccagattgctgccctgaaggaggagaccgaggaggaggtgcaggacaccagactgtaa

SEQ ID NO: 5 - DNA, 2859 bp, synthetic construct
  misc_feature 1..2859: note = GAA
  source 1..2859: mol_type = other DNA
atgggagtccgccacccgccctgctcacatcgcctgcttgctgtctgtgccctcgtgtcacttgctaccgccgcgctgcttggtcacattctgctgcacgactttttactagttccgagggaactgtcgggatccagccccgtgctcgaggaaactcaccccgcgcaccaacagggggcgtccaggccgggaccgcgcgacgcccaggcccacccgggccggcctcgggccgtgccaactcagtgcgatgtgccgccgaactcccgcttcgactgtgcgcctgacaaggccataacccaggaacagtgcgaagcacgcggctgctgctatattccggcgaagcagggcttgcagggtgcccaaatgggtcagccttggtgcttctttcccccgtcgtacccctcgtacaagctggagaacctgagcagcagcgaaatggggtacaccgccactctgacccggacgaccccgaccttcttcccgaaagacatcctgaccctgcggctggatgtgatgatggaaactgagaacagactgcacttcactatcaaggaccccgcgaaccgcagatatgaggtgccactggaaacccctcatgtgcattcccgggccccatcccctctgtactcggtggaattctccgaagaacccttcggggtcattgtgcgccggcagcttgatggccgggtcctgctcaacaccaccgtggcaccccttttcttcgctgaccagttcctccagctgagcacctcgctgccgagccagtacatcaccggactggccgagcacctctcccctctgatgctgtccactagctggactaggatcactctgtggaaccgggatctggcccctaccccgggcgcgaacctgtacggatcgcaccccttctacctggccctcgaggacggaggctccgcccacggagtgttcctgctgaactccaacgctatggacgtggtgctccagccgtcccctgcactgtcctggcggagcacagggggtattctggatgtctacatcttcctcggcccggagccaaagtccgtggtgcaacagtatctggatgtcgtgggttacccattcatgccgccatactggggccttggcttccacctgtgccgctggggatacagctccaccgccatcactagacaggtcgtggaaaacatgactagagcccacttccccctcgatgtccagtggaatgacctggactacatggattccagacgcgacttcactttcaacaaggatggattcagagatttccccgctatggtccaagaactgcaccagggtggccggcggtacatgatgattgtggaccccgccatttcaagctccggaccagcgggctcgtaccggccctacgacgaaggtttgcgccgcggcgtgttcatcactaacgaaaccggccagccactgattgggaaggtctggcctggaagcaccgcgttcccggacttcactaacccaacggccttggcgtggtgggaggacatggtggccgaattccacgaccaagtcccattcgacggaatgtggatcgacatgaacgagcccagcaacttcatccgaggctccgaggacggctgccctaacaacgaacttgagaaccctccgtacgtgcctggcgtcgtcggcggaacactgcaggccgctacgatctgtgcctcatcgcatcagttcctgtcaacccactacaacctccataatctgtacggcctcaccgaagccatcgcctcccaccgggccctggtcaaggcccgggggactaggcccttcgtgattagccggagcactttcgccggacacggaagatacgccggacattggaccggcgacgtgtggtcatcgtgggagcagctcgcctcctccgtccccgaaatcctgcagttcaatctcctgggagtccccctcgtgggcgcggacgtgtgcggattcctgggcaatacctctgaggagctgtgcgtgagatggacccagctgggggcgttctaccccttcatgcggaaccacaactcactgctgtccctgcctcaagagccgtactcattctccgagccggcacaacaggccatgcgaaaggctctgaccctccgctatgcgctcttgccccacctctacactctgtttcaccaagcccatgtcgcgggcgaaacagtggccagaccactctttctggaattcccaaaggactcctcaacctggactgtggatcatcagctgctctggggagaggcactgctgatcaccccggtgctccaagccggaaaggcggaagtgaccggatacttccctctcggtacttggtacgacctccaaaccgtgccggtcgaggccctgggcagcttgcctccgccgccggctgccccgcgggagcctgcaatccactccgaggggcaatgggtgaccctccctgcaccactggacaccatcaacgtgcacctccgggccggctacatcatcccgctgcaaggaccgggtctgactaccaccgaatcccggcagcagcccatggcactggccgtggccctgaccaagggaggggaagcacggggagaactcttttgggacgatggagaatccctggaagtgctcgagcggggagcctacactcaagtcatctttcttgcccgcaacaacaccatcgtgaacgaattggtccgcgtgacctccgagggggccggactccagctgcaaaaagtgaccgtgctgggggtggcaaccgccccgcaacaagtgttgtctaacggagtgccggtgtccaacttcacctactcccctgataccaaagttctagatatttgcgtgagcctgctgatgggagaacagttcctggtgtcctggtgctga

SEQ ID NO: 6 - DNA, 2859 bp, synthetic construct; identical to SEQ ID NO: 5
  misc_feature 1..2859: note = GAA codon-optimized nucleotide sequence 1 (GAA 15)
  source 1..2859: mol_type = other DNA

SEQ ID NO: 7 - DNA, 2859 bp, synthetic construct
  misc_feature 1..2859: note = GAA Codon-optimized 2 (GAA21)
  source 1..2859: mol_type = other DNA
atgggagttagacaccctccatgtagccacagactgctggccgtgtgtgctctggtgtctctggctacagctgccctgctgggacatatcctgctgcacgacttcttactagttcccagagagctgtccggcagcagccctgtgctggaagaaacacaccctgcacatcagcagggcgcctctagacctggacctagagatgctcaggcccatcctggcagacctagagctgtgcccacacagtgtgacgtgccacctaacagcagattcgactgcgcccctgacaaggccatcacacaagagcagtgtgaagccagaggctgctgctacatccctgccaaacaaggactgcagggcgctcagatgggacagccctggtgcttcttcccaccatcttaccccagctacaagctggaaaacctgagcagcagcgagatgggctacaccgccacactgaccagaaccacacctacattcttcccgaaggacatcctgacactgcggctggacgtgatgatggaaaccgagaaccggctgcacttcaccatcaaggaccccgccaatcggagatacgaggtgccactggaaacccctcacgtgcactctagagccccatctccactgtacagcgtggaattcagcgaggaacccttcggcgtgatcgtgcggagacagctggatggaagagtgctgctgaacaccacagtggcccctctgttcttcgccgaccagtttctgcagctgtccaccagcctgcctagccagtatatcacaggcctggccgagcacctgtctccactgatgctgtctaccagctggacccggatcaccctgtggaacagggatcttgctcctacacctggcgccaacctgtacggctctcaccctttttatctggccctggaagatggcggatctgcccacggtgtctttctgctgaactccaacgccatggacgtggtgctgcagccatctcctgctctgtcttggagaagcacaggcggcatcctggacgtgtacatctttctgggccccgagcctaagagcgtggtgcagcagtatctggacgtcgtgggctaccccttcatgcctccttattggggcctgggcttccacctgtgcagatggggatacagcagcaccgccatcaccagacaggtggtggaaaacatgacccgggctcacttcccactggatgtgcagtggaacgacctggactacatggacagcagacgggacttcaccttcaacaaggacggcttcagagacttccccgccatggtgcaagaactgcaccaaggcggcagacggtacatgatgatcgtggatccagccatcagctctagcggccctgccggctcttacagaccttacgatgagggcctgagaagaggcgtgttcatcaccaacgagacaggccagcctctgatcggcaaagtgtggcctggcagcacagcctttccagacttcacaaaccccaccgctctggcttggtgggaagatatggtggccgagtttcacgatcaggtgcccttcgacggcatgtggatcgacatgaacgagcccagcaacttcatccggggcagcgaggatggctgccccaacaacgaactggaaaatcctccttacgtgcccggcgttgtcggcggaacacttcaggccgctacaatctgtgccagcagccaccagttcctcagcacccactacaacctgcacaatctgtatggcctgaccgaggccattgccagccatagagccctggttaaggccaggggcaccagacctttcgtgatcagcagaagcaccttcgccggccacggcagatatgccggacattggacaggcgacgtgtggtctagttgggagcagctggctagcagcgtgccagagatcctgcagttcaatctgctgggcgtgccactcgtgggagccgatgtttgtggcttcctgggcaacacctccgaggaactgtgtgtgcgttggacacagctgggcgccttctatcccttcatgagaaaccacaacagccttctcagcctgccacaagagccctacagcttctctgagcctgcacagcaggccatgagaaaggccctgactctgagatacgctctgctgccccacctgtacaccctgtttcaccaggctcatgtggccggggagacagtggctagacctctgttcctggaattccccaaggacagctccacctggaccgtggatcatcagctgctgtggggagaagccctgctcatcacacctgttctgcaggccggaaaggccgaagtgaccggctattttcctctcggcacttggtacgacctgcagaccgtgcctgttgaggctctgggatctcttcctccacctcctgccgctcctagagagcctgccattcactctgaaggccagtgggttaccctgcctgctcctctggacaccatcaacgtgcacctgagagctggctacatcatccctctgcaaggccctggcctgacaaccaccgaatctagacagcagcccatggctctggccgtggctttgacaaaaggcggagaggctagaggcgagctgttctgggatgatggcgagagcctggaagtgctggaacggggcgcttatacccaagtgatcttcctggccagaaacaacaccatcgtgaacgaactcgtgcgcgtgaccagtgaaggtgctggactgcaactgcagaaagtgaccgtgctcggagtggccacagcacctcagcaggttctgtctaatggcgtgcccgtgtccaacttcacatacagccccgacaccaaggtcctggacatctgtgtgtcactgctgatgggcgagcagttcctggtgtcctggtgttga

SEQ ID NO: 8 - protein, 952 aa, Homo sapiens
  REGION 1..952: note = Acid Alpha-Glucosidase (GAA)
MGVRHPPCSHRLLAVCALVSLATAALLGHILLHDFLLVPRELSGSSPVLEETHPAHQQGASRPGPRDAQAHPGRPRAVPTQCDVPPNSRFDCAPDKAITQEQCEARGCCYIPAKQGLQGAQMGQPWCFFPPSYPSYKLENLSSSEMGYTATLTRTTPTFFPKDILTLRLDVMMETENRLHFTIKDPANRRYEVPLETPHVHSRAPSPLYSVEFSEEPFGVIVRRQLDGRVLLNTTVAPLFFADQFLQLSTSLPSQYITGLAEHLSPLMLSTSWTRITLWNRDLAPTPGANLYGSHPFYLALEDGGSAHGVFLLNSNAMDVVLQPSPALSWRSTGGILDVYIFLGPEPKSVVQQYLDVVGYPFMPPYWGLGFHLCRWGYSSTAITRQVVENMTRAHFPLDVQWNDLDYMDSRRDFTFNKDGFRDFPAMVQELHQGGRRYMMIVDPAISSSGPAGSYRPYDEGLRRGVFITNETGQPLIGKVWPGSTAFPDFTNPTALAWWEDMVAEFHDQVPFDGMWIDMNEPSNFIRGSEDGCPNNELENPPYVPGVVGGTLQAATICASSHQFLSTHYNLHNLYGLTEAIASHRALVKARGTRPFVISRSTFAGHGRYAGHWTGDVWSSWEQLASSVPEILQFNLLGVPLVGADVCGFLGNTSEELCVRWTQLGAFYPFMRNHNSLLSLPQEPYSFSEPAQQAMRKALTLRYALLPHLYTLFHQAHVAGETVARPLFLEFPKDSSTWTVDHQLLWGEALLITPVLQAGKAEVTGYFPLGTWYDLQTVPVEALGSLPPPPAAPREPAIHSEGQWVTLPAPLDTINVHLRAGYIIPLQGPGLTTTESRQQPMALAVALTKGGEARGELFWDDGESLEVLERGAYTQVIFLARNNTIVNELVRVTSEGAGLQLQKVTVLGVATAPQQVLSNGVPVSNFTYSPDTKVLDICVSLLMGEQFLVSWC

SEQ ID NO: 9 - DNA, 1290 bp, synthetic construct
  misc_feature 1..1290: note = GLA
  source 1..1290: mol_type = other DNA
atgcagctgaggaacccagaactacatctgggctgcgcgcttgcgcttcgcttcctggccctcgtttcctgggacatccctggggctagagcactggacaatggattggcaaggacgcctaccatgggctggctgcactgggagcgcttcatgtgcaaccttgactgccaggaagagccagattcctgcatcagtgagaagctcttcatggagatggcagagctcatggtctcagaaggctggaaggatgcaggttatgagtacctctgcattgatgactgttggatggctccccaaagagattcagaaggcagacttcaggcagaccctcagcgctttcctcatgggattcgccagctagctaattatgttcacagcaaaggactgaagctagggatttatgcagatgttggaaataaaacctgcgcaggcttccctgggagttttggatactacgacattgatgcccagacctttgctgactggggagtagatctgctaaaatttgatggttgttactgtgacagtttggaaaatttggcagatggttataagcacatgtccttggccctgaataggactggcagaagcattgtgtactcctgtgagtggcctctttatatgtggccctttcaaaagcccaattatacagaaatccgacagtactgcaatcactggcgaaattttgctgacattgatgattcctggaaaagtataaagagtatcttggactggacatcttttaaccaggagagaattgttgatgttgctggaccagggggttggaatgacccagatatgttagtgattggcaactttggcctcagctggaatcagcaagtaactcagatggccctctgggctatcatggctgctcctttattcatgtctaatgacctccgacacatcagccctcaagccaaagctctccttcaggataaggacgtaattgccatcaatcaggaccccttgggcaagcaagggtaccagcttagacagggagacaactttgaagtgtgggaacgacctctctcaggcttagcctgggctgtagctatgataaaccggcaggagattggtggacctcgctcttataccatcgcagttgcttccctgggtaaaggagtggcctgtaatcctgcctgcttcatcacacagctcctccctgtgaaaaggaagctagggttctatgaatggacttcaaggttaagaagtcacataaatcccacaggcactgttttgcttcagctagaaaatacaatgcagatgtcattaaaagacttactttaa

SEQ ID NO: 10 - DNA, 1290 bp, synthetic construct
  misc_feature 1..1290: note = GLA codon-optimized
  source 1..1290: mol_type = other DNA
atgcagctgagaaatcctgaactgcacctgggctgtgccctggctctgagatttctggctctggtgtcctgggacattcctggcgctagagccctggataatggcctggccagaacacctacaatgggctggctgcactgggagagattcatgtgcaacctggactgccaagaggaacccgacagctgcatcagcgagaagctgttcatggaaatggccgagctgatggtgtccgaaggctggaaggatgccggctacgagtacctgtgcatcgacgattgctggatggcccctcagagagattctgagggcagactgcaggccgatcctcagagatttcctcacggaatccggcagctggccaactacgtgcactctaagggactgaagctgggcatctacgccgacgtgggcaacaagacatgtgccggctttccaggcagcttcggctactacgatatcgacgcccagacctttgccgattggggcgtcgacctgctgaagttcgatggctgctactgcgacagcctggaaaacctggccgacggctacaaacacatgtctctggccctgaaccggaccggcagatctatcgtgtactcttgcgagtggcccctgtacatgtggcccttccagaagcctaactacaccgagatcagacagtactgcaaccactggcggaacttcgccgacatcgatgacagctggaagtccatcaagagcatcctggactggaccagcttcaatcaagagcggatcgtggatgtggctggcccaggcggatggaacgatcctgatatgctggtcatcggcaacttcggcctgagctggaatcagcaagtgacccagatggccctgtgggccattatggccgctcctctgttcatgagcaacgacctgagacacatcagccctcaggccaaggctctgctgcaggataaggacgtgatcgccatcaaccaggatcctctgggcaagcagggctatcagctgagacagggcgacaatttcgaagtgtgggaaagacctctgagcggcctggcttgggccgtcgccatgatcaatagacaagagatcggcggaccccggtcctatacaattgccgtggcttctctcggaaaaggcgtggcctgcaatcctgcctgctttatcacacagctgctccccgtgaagagaaagctgggcttttacgagtggaccagcagactgagatcccacatcaaccccacaggcactgttctgctgcaactggaaaacacaatgcagatgagcctgaaggacctgctgtag

SEQ ID NO: 11 - protein, 429 aa, synthetic construct
  REGION 1..429: note = GLA
MQLRNPELHLGCALALRFLALVSWDIPGARALDNGLARTPTMGWLHWERFMCNLDCQEEPDSCISEKLFMEMAELMVSEGWKDAGYEYLCIDDCWMAPQRDSEGRLQADPQRFPHGIRQLANYVHSKGLKLGIYADVGNKTCAGFPGSFGYYDIDAQTFADWGVDLLKFDGCYCDSLENLADGYKHMSLALNRTGRSIVYSCEWPLYMWPFQKPNYTEIRQYCNHWRNFADIDDSWKSIKSILDWTSFNQERIVDVAGPGGWNDPDMLVIGNFGLSWNQQVTQMALWAIMAAPLFMSNDLRHISPQAKALLQDKDVIAINQDPLGKQGYQLRQGDNFEVWERPLSGLAWAVAMINRQEIGGPRSYTIAVASLGKGVACNPACFITQLLPVKRKLGFYEWTSRLRSHINPTGTVLLQLENTMQMSLKDLL

SEQ ID NO: 12 - DNA, 1317 bp, synthetic construct
  misc_feature 1..1317: note = CLN3
  source 1..1317: mol_type = other DNA
atgggaggctgtgcaggctcgcggcggcgcttttcggattccgagggggaggagaccgtcccggagccccggctccctctgttggaccatcagggcgcgcattggaagaacgcggtgggcttctggctgctgggcctttgcaacaacttctcttatgtggtgatgctgagtgccgcccacgacatccttagccacaagaggacatcgggaaaccagagccatgtggacccaggcccaacgccgatcccccacaacagctcatcacgatttgactgcaactctgtctctacggctgctgtgctcctggcggacatcctccccacactcgtcatcaaattgttggctcctcttggccttcacctgctgccctacagcccccgggttctcgtcagtgggatttgtgctgctggaagcttcgtcctggttgccttttctcattctgtggggaccagcctgtgtggtgtggtcttcgctagcatctcatcaggccttggggaggtcaccttcctctccctcactgccttctaccccagggccgtgatctcctggtggtcctcagggactgggggagctgggctgctgggggccctgtcctacctgggcctcacccaggccggcctctcccctcagcagaccctgctgtccatgctgggtatccctgccctgctgctggccagctatttcttgttgctcacatctcctgaggcccaggaccctggaggggaagaagaagcagagagcgcagcccggcagcccctcataagaaccgaggccccggagtcgaagccaggctccagctccagcctctcccttcgggaaaggtggacagtgttcaagggtctgctgtggtacattgttcccttggtcgtagtttactttgccgagtatttcattaaccagggactttttgaactcctctttttctggaacacttccctgagtcacgctcagcaataccgctggtaccagatgctgtaccaggctggcgtctttgcctcccgctcttctctccgctgctgtcgcatccgtttcacctgggccctggccctgctgcagtgcctcaacctggtgttcctgctggcagacgtgtggttcggctttctgccaagcatctacctcgtcttcctgatcattctgtatgaggggctcctgggaggcgcagcctacgtgaacaccttccacaacatcgccctggagaccagtgatgagcaccgggagtttgcaatggcggccacctgcatctctgacacactggggatctccctgtcggggctcctggctttgcctctgcatgacttcctctgccagctctcctga

SEQ ID NO: 13 - DNA, 1318 bp, synthetic construct
  misc_feature 1..1318: note = CLN3 codon-optimized
  source 1..1318: mol_type = other DNA
atgggaggatgtgctgggtcaagaagacggtttagcgattccgaaggagaggagactgtgcctgagccaagactgcccctgctggatcaccagggagcacactggaagaacgcagtgggattctggctgctgggcctgtgcaacaacttcagctacgtggtcatgctgtccgccgcccacgacatcctgtcccacaagcggacctccggcaatcagtctcacgtggaccccggccctacaccaatcccccacaacagcagcagccggttcgactgtaattccgtgtctaccgcagccgtgctgctggcagacatcctgcccaccctggtcatcaagctgctggcaccactgggcctgcacctgctgccttattctccaagggtgctggtgagcggcatctgcgcagcaggcagcttcgtgctggtggcctttagccactccgtgggcacctctctgtgcggagtggtgtttgcaagcatcagctccggcctgggagaggtgaccttcctgagcctgacagccttttaccctcgcgccgtgatctcctggtggtctagcggcacaggaggagcaggcctgctgggcgccctgtcctatctgggcctgacccaggcaggcctgtccccacagcagacactgctgtctatgctgggcatccctgccctgctgctggcaagctacttcctgctgctgacctccccagaggcacaggaccccggaggagaggaggaggccgagagcgccgcaaggcagccactgatcaggaccgaggcaccagagtccaagcctggctcctctagctccctgtctctgcgggagagatggacagtgttcaagggcctgctgtggtacatcgtgcccctggtggtggtgtacttcgccgagtacttcatcaaccagggcctgtttgagctgctgttcttttggaatacctctctgagccacgcccagcagtaccggtggtatcagatgctgtatcaggcaggcgtgttcgcctcccggtctagcctgagatgctgtcggatcagattcacctgggcactggccctgctgcagtgcctgaacctggtgttcctgctggccgacgtgtggttcggctttctgccctctatctacctggtgtttctgatcatcctgtatgagggcctgctgggaggagcagcctatgtgaacaccttccacaatatcgccctggagacatctgacgagcacagagagtttgctatggccgccacctgtatcagcgatacactgggcatctctctgagcggactgctggctctgcctctgcatgactttctgtgccagctgagttaat

SEQ ID NO: 14 - protein, 438 aa, Homo sapiens
  REGION 1..438: note = Ceroid Lipofuscinosis, Neuronal 3 (CLN3)
MGGCAGSRRRFSDSEGEETVPEPRLPLLDHQGAHWKNAVGFWLLGLCNNFSYVVMLSAAHDILSHKRTSGNQSHVDPGPTPIPHNSSSRFDCNSVSTAAVLLADILPTLVIKLLAPLGLHLLPYSPRVLVSGICAAGSFVLVAFSHSVGTSLCGVVFASISSGLGEVTFLSLTAFYPRAVISWWSSGTGGAGLLGALSYLGLTQAGLSPQQTLLSMLGIPALLLASYFLLLTSPEAQDPGGEEEAESAARQPLIRTEAPESKPGSSSSLSLRERWTVFKGLLWYIVPLVVVYFAEYFINQGLFELLFFWNTSLSHAQQYRWYQMLYQAGVFASRSSLRCCRIRFTWALALLQCLNLVFLLADVWFGFLPSIYLVFLIILYEGLLGGAAYVNTFHNIALETSDEHREFAMAATCISDTLGISLSGLLALPLHDFLCQLS

SEQ ID NO: 15 - DNA, 2211 bp, synthetic construct
  misc_feature 1..2211: note = AAV204 VP1
  source 1..2211: mol_type = other DNA
atggctgccgatggttatcttccagattggctcgaggacaacctctctgagggcattcgcgagtggtgggacttgaaacctggagccccgaagcccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaaacgtccggtagagcagtcaccacaagagccagactcctcctcgggcatcggcaagacaggccagcagcccgctaaaaagagactcaattttggtcagactggcgactcagagtcagtccccgacccacaacctctcggagaacctccagcaacccccgctgctgtgggacctactacaatggcttcaggcggtggcgcaccaatggcggacaataacgaaggcgccgacggagtgggtaatgcctcaggaaattggcattgcgattccacatggctgggcgacagagtcatcaccaccagcacccgaacatgggccttgcccacctataacaaccacctctacaagcaaatctccagtgcttcaacgggggccagcaacgacaaccactacttcggctacagcaccccctgggggtattttgatttcaacagattccactgccatttctcaccacgtgactggcagcgactcatcaacaacaattggggattccggcccaagagactcaacttcaagctcttcaacatccaagtcaaggaggtcacgacgaatgatggcgtcacgaccatcgctaataaccttaccagcacggttcaagtcttctcggactcggagtaccagttgccgtacgtcctcggctctgcgcaccagggctgcctccctccgttcccggcggacgtgttcatgattccgcagtacggctacctaacgctcaacaatggcagccaggcagtgggacggtcatccttttactgcctggaatatttcccatcgcagatgctgagaacgggcaataactttaccttcagctacaccttcgaggacgtgcctttccacagcagctacgcgcacagccagagcctggaccggctgatgaatcctctcatcgaccagtacctgtattacctgaacagaactcagaatcagtccggaagtgcccaaaacaaggacttgctgtttagccgggggtctccagctggcatgtctgttcagcccaaaaactggctacctggaccctgttaccggcagcagcgcgtttctaaaacaaaaacagacaacaacaacagcaactttacctggacaggtgcttcaaaatataaccttaatgggcgtgaatctataatcaaccctggcactgctatggcctcacacaaagacgacaaagacaagttctttcccatgagcggtgtcatgatttttggaaaggagagcgccggagcttcaaacactgcattggacaatgtcatgatcacagacgaagaggaaatcaaagccactaaccccgtggccaccgaaagatttgggactgtggcagtcaatctccagaacagcagcacagaccctgcgaccggagatgtgcatgttatgggagccttacctggaatggtgtggcaagacagagacgtatacctgcagggtcctatttgggccaaaattcctcacacggatggacactttcacccgtctcctctcatgggcggctttggacttaagcacccgcctcctcagatcctcatcaaaaacacgcctgttcctgcgaatcctccggcagagttttcggctacaaagtttgcttcattcatcacccagtattccacaggacaagtgagcgtggagattgaatgggagctgcagaaagaaaacagcaaacgctggaatcccgaagtgcagtatacatctaactatgcaaaatctgccaacgttgatttcactgtagacaacaatggactttatactgagcctcgccccattggcacccgttacctcacccgtcccctgtaa

SEQ ID NO: 16 - DNA, 1605 bp, synthetic construct
  misc_feature 1..1605: note = AAV204 VP3
  source 1..1605: mol_type = other DNA
atggcttcaggcggtggcgcaccaatggcggacaataacgaaggcgccgacggagtgggtaatgcctcaggaaattggcattgcgattccacatggctgggcgacagagtcatcaccaccagcacccgaacatgggccttgcccacctataacaaccacctctacaagcaaatctccagtgcttcaacgggggccagcaacgacaaccactacttcggctacagcaccccctgggggtattttgatttcaacagattccactgccatttctcaccacgtgactggcagcgactcatcaacaacaattggggattccggcccaagagactcaacttcaagctcttcaacatccaagtcaaggaggtcacgacgaatgatggcgtcacgaccatcgctaataaccttaccagcacggttcaagtcttctcggactcggagtaccagttgccgtacgtcctcggctctgcgcaccagggctgcctccctccgttcccggcggacgtgttcatgattccgcagtacggctacctaacgctcaacaatggcagccaggcagtgggacggtcatccttttactgcctggaatatttcccatcgcagatgctgagaacgggcaataactttaccttcagctacaccttcgaggacgtgcctttccacagcagctacgcgcacagccagagcctggaccggctgatgaatcctctcatcgaccagtacctgtattacctgaacagaactcagaatcagtccggaagtgcccaaaacaaggacttgctgtttagccgggggtctccagctggcatgtctgttcagcccaaaaactggctacctggaccctgttaccggcagcagcgcgtttctaaaacaaaaacagacaacaacaacagcaactttacctggacaggtgcttcaaaatataaccttaatgggcgtgaatctataatcaaccctggcactgctatggcctcacacaaagacgacaaagacaagttctttcccatgagcggtgtcatgatttttggaaaggagagcgccggagcttcaaacactgcattggacaatgtcatgatcacagacgaagaggaaatcaaagccactaaccccgtggccaccgaaagatttgggactgtggcagtcaatctccagaacagcagcacagaccctgcgaccggagatgtgcatgttatgggagccttacctggaatggtgtggcaagacagagacgtatacctgcagggtcctatttgggccaaaattcctcacacggatggacactttcacccgtctcctctcatgggcggctttggacttaagcacccgcctcctcagatcctcatcaaaaacacgcctgttcctgcgaatcctccggcagagttttcggctacaaagtttgcttcattcatcacccagtattccacaggacaagtgagcgtggagattgaatgggagctgcagaaagaaaacagcaaacgctggaatcccgaagtgcagtatacatctaactatgcaaaatctgccaacgttgatttcactgtagacaacaatggactttatactgagcctcgccccattggcacccgttacctcacccgtcccctgtaa

SEQ ID NO: 17 - protein, 534 aa, synthetic construct
  REGION 1..534: note = AAV204 VP3
MASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDKDKFFPMSGVMIFGKESAGASNTALDNVMITDEEEIKATNPVATERFGTVAVNLQNSSTDPATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 18 - DNA, 2208 bp, synthetic construct
  misc_feature 1..2208: note = ITB102 214 (AAV214) VP1
  source 1..2208: mol_type = other DNA
atggctgccgatggttatcttccagattggctcgaggacaacctctctgagggcattcgcgagtggtgggcgctgaaacctggagccccgaagcccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagcgggttctcgaaccttttggtctggttgaggaaggtgctaagacggctcctggaaagaaacgtccggtagagcagtcgccacaagagccagactcctcctcgggcatcggcaagacaggccagcagcccgctaaaaagagactcaattttggtcagactggcgactcagagtcagtccccgacccacaacctctcggagaacctccagcaacccccgctgctgtgggacctactacaatggcttcaggcggtggcgcaccaatggcggacaataacgaaggcgccgacggagtgggtaatgcctcaggaaattggcattgcgattccacatggctgggcgacagagtcatcaccaccagcacccgcacctgggccttgcccacctacaataaccacctctacaagcaaatctccagtgcttcaacgggggccagcaacgacaaccactacttcggctacagcaccccctgggggtattttgacttcaacagattccactgccacttttcaccacgtgactggcaaagactcatcaacaacaactggggattccgacccaagagactcaacttcaagctctttaacattcaagtcaaagaggttacggacaacaatggagtcaagaccatcgccaataaccttaccagcacggtccaggtcttcacggactcagactatcagctcccgtacgtcctcggctctgcgcaccagggctgcctccctccgttcccggcggacgtgttcatgattccgcagtacggctacctaacgctcaacgacggcagccaggcagtgggacggtcatccttttactgcctggaatatttcccatcgcagatgctgagaacgggcaacaactttaccttcagctacacctttgaggacgttcctttccacagcagctacgctcacagccagagtctggaccgtctcatgaatcctctgattgaccagtacctgtactacttgtctaagactatcaacggatccggccagaatcagcagactctgaagttcagccaaggtgggcctaatacaatggccaatcaggcaaagaactggctgccaggaccctgttaccgccaacaacgcgtctcaacgacaaccgggcaaaacaacaatagcaactttgcctggactgctgggaccaaataccatctgaatggaagaaattcattgatgaatcctggccccgctatggcatcccacaaagagggcgaggaccgtttttttcccctgtccgggtccctgatttttggcaaacaaaatgctgccagagacaatgcggattacagcgatgtcatgctcaccagcgaggaagaaatcaaaaccactaaccctgtggctacagaggaatacggtatcgtggcagataacttgcagcagcaaaacacggctcctcaaattggaactgtcaacagccagggggccttacccggtatggtctggcagaaccgggacgtgtacctgcagggtcccatctgggccaagattcctcacacggacggcaacttccacccgtctccgctgatgggcggctttggcctgaaacatcctccgcctcagatcctgatcaagaacacgcctgtacctgcggatcctccgaccaccttcaaccagtcaaagctgaactctttcatcacgcaatacagcaccggacaggtcagcgtggaaattgaatgggagctgcagaaggaaaacagcaagcgctggaaccccgagatccagtacacctccaactactacaaatctacaagtgtggactttgctgttaatacagaaggcgtgtactctgaaccccaccccattggcacccgttacctcacccgtcccctgtaa

SEQ ID NO: 19 - DNA, 2211 bp, synthetic construct
  misc_feature 1..2211: note = AAV214-A VP1
  source 1..2211: mol_type = other DNA
atggctgccgatggttatcttccagattggctcgaggacaacctctctgagggcattcgcgagtggtgggcgctgaaacctggagccccgaagcccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagcgggttctcgaaccttttggtctggttgaggaaggtgctaagacggctcctggaaagaaacgtccggtagagcagtcgccacaagagccagactcctcctcgggcatcggcaagacaggccagcagcccgctaaaaagagactcaattttggtcagactggcgactcagagtcagtccccgacccacaacctctcggagaacctccagcaacccccgctgctgtgggacctactacaatggcttcaggcggtggcgcaccaatggcggacaataacgaaggcgccgacggagtgggtaatgcctcaggaaattggcattgcgattccacatggctgggcgacagagtcatcaccaccagcacccgcacctgggccttgcccacctacaataaccacctctacaagcaaatctccaacagcacatctggaggatcttcaaatgacaacgcctacttcggctacagcaccccctgggggtattttgacttcaacagattccactgccacttttcaccacgtgactggcaaagactcatcaacaacaactggggattccgacccaagagactcaacttcaagctctttaacattcaagtcaaagaggttacggacaacaatggagtcaagaccatcgccaataaccttaccagcacggtccaggtcttcacggactcagactatcagctcccgtacgtcctcggctctgcgcaccagggctgcctccctccgttcccggcggacgtgttcatgattccgcagtacggctacctaacgctcaacgacggcagccaggcagtgggacggtcatccttttactgcctggaatatttcccatcgcagatgctgagaacgggcaacaactttaccttcagctacacctttgaggacgttcctttccacagcagctacgctcacagccagagtctggaccgtctcatgaatcctctgattgaccagtacctgtactacttgtctaagactatcaacggatccggccagaatcagcagactctgaagttcagccaaggtgggcctaatacaatggccaatcaggcaaagaactggctgccaggaccctgttaccgccaacaacgcgtctcaacgacaaccgggcaaaacaacaatagcaactttgcctggactgctgggaccaaataccatctgaatggaagaaattcattgatgaatcctggccccgctatggcatcccacaaagagggcgaggaccgtttttttcccctgtccgggtccctgatttttggcaaacaaaatgctgccagagacaatgcggattacagcgatgtcatgctcaccagcgaggaagaaatcaaaaccactaaccctgtggctacagaggaatacggtatcgtggcagataacttgcagcagcaaaacacggctcctcaaattggaactgtcaacagccagggggccttacccggtatggtctggcagaaccgggacgtgtacctgcagggtcccatctgggccaagattcctcacacggacggcaacttccacccgtctccgctgatgggcggctttggcctgaaacatcctccgcctcagatcctgatcaagaacacgcctgtacctgcggatcctccgaccaccttcaaccagtcaaagctgaactctttcatcacgcaatacagcaccggacaggtcagcgtggaaattgaatgggagctgcagaaggaaaacagcaagcgctggaaccccgagatccagtacacctccaactactacaaatctacaagtgtggactttgctgttaatacagaaggcgtgtactctgaaccccaccccattggcacccgttacctcacccgtcccctgtaa

SEQ ID NO: 20 - DNA, 2211 bp, synthetic construct
  misc_feature 1..2211: note = AAV214e VP1
  source 1..2211: mol_type = other DNA
atggctgccgatggttatcttccagattggctcgaggacaacctctctgagggcattcgcgagtggtgggacttgaaacctggagccccgaaacccaaagccaaccagcaaaagcaggacgacggccggggtctggtgcttcctggctacaagtacctcggacccttcaacggactcgacaagggggagcccgtcaacgcggcggatgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagagaccggtagagccatcaccccagcgttctccagactcctctacgggcatcggcaagaaaggccaacagcccgccagaaaaagactcaattttggtcagactggcgactcagagtcagtccccgacccacaacctctcggagaacctccagcaacccccgctgctgtgggacctactacaatggcttcaggcggtggcgcaccaatggcagacaataacgaaggcgccgacggagtgggtaatgcctcaggaaattggcattgcgattccacatggctgggcgacagagtcatcaccaccagcacccgcacctgggccttgcccacctacaataaccacctctacaagcaaatctccagtgcttcaacgggggccagcaacgacaaccactacttcggctacagcaccccctgggggtattttgacttcaacagattccactgccacttttcaccacgtgactggcaaagactcatcaacaacaactggggattccgacccaagagactcaacttcaagctctttaacattcaagtcaaagaggttacggacaacaatggagtcaagaccatcgccaataaccttaccagcacggtccaggtcttcacggactcagactatcagctcccgtacgtcctcggctctgcgcaccagggctgcctccctccgttcccggcggacgtgttcatgattccgcagtacggctacctaacgctcaacgacggcagccaggcagtgggacggtcatccttttactgcctggaatatttcccatcgcagatgctgagaacgggcaacaactttaccttcagctacacctttgaggacgttcctttccacagcagctacgctcacagccagagtctggaccgtctcatgaatcctctgattgaccagtacctgtactacttgtctaagactatcaacggatccggccagaatcagcagactctgaagttcagccaaggtgggcctaatacaatggccaatcaggcaaagaactggctgccaggaccctgttaccgccaacaacgcgtctcaacgacaaccgggcaaaacaacaatagcaactttgcctggactgctgggaccaaataccatctgaatggaagaaattcattgatgaatcctggccccgctatggcatcccacaaagagggcgaggaccgtttttttcccctgtccgggtccctgatttttggcaaacaaaatgctgccagagacaatgcggattacagcgatgtcatgctcaccagcgaggaagaaatcaaaaccactaaccctgtggctacagaggaatacggtatcgtggcagataacttgcagcagcaaaacacggctcctcaaattggaactgtcaacagccagggggccttacccggtatggtctggcagaaccgggacgtgtacctgcagggtcccatctgggccaagattcctcacacggacggcaacttccacccgtctccgctgatgggcggctttggcctgaaacatcctccgcctcagatcctgatcaagaacacgcctgtacctgcggatcctccgaccaccttcaaccagtcaaagctgaactctttcatcacgcaatacagcaccggacaggtcagcgtggaaattgaatgggagctgcagaaggaaaacagcaagcgctggaaccccgagatccagtacacctccaactactacaaatctacaagtgtggactttgctgttaatacagaaggcgtgtactctgaaccccaccccattggcacccgttacctcacccgtcccctgtaa

SEQ ID NO: 21 - DNA, 2211 bp, synthetic construct
  misc_feature 1..2211: note = AAV214e8 VP1
  source 1..2211: mol_type = other DNA
atggctgccgatggttatcttccagattggctcgaggacaacctctctgagggcattcgcgagtggtgggcgctgaaacctggagccccgaagcccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctgcaggcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagagaccggtagagccatcaccccagcgttctccagactcctctacgggcatcggcaagaaaggccaacagcccgccagaaaaagactcaattttggtcagactggcgactcagagtcagttccagaccctcaacctctcggagaacctccagcagcgccctctggtgtgggacctaatacaatggcttcaggcggtggcgcaccaatggcggacaataacgaaggcgccgacggagtgggtaatgcctcaggaaattggcattgcgattccacatggctgggcgacagagtcatcaccaccagcacccgcacctgggccttgcccacctacaataaccacctctacaagcaaatctccagtgcttcaacgggggccagcaacgacaaccactacttcggctacagcaccccctgggggtattttgacttcaacagattccactgccacttttcaccacgtgactggcaaagactcatcaacaacaactggggattccgacccaagagactcaacttcaagctctttaacattcaagtcaaagaggttacggacaacaatggagtcaagaccatcgccaataaccttaccagcacggtccaggtcttcacggactcagactatcagctcccgtacgtcctcggctctgcgcaccagggctgcctccctccgttcccggcggacgtgttcatgattccgcagtacggctacctaacgctcaacgacggcagccaggcagtgggacggtcatccttttactgcctggaatatttcccatcgcagatgctgagaacgggcaacaactttaccttcagctacacctttgaggacgttcctttccacagcagctacgctcacagccagagtctggaccgtctcatgaatcctctgattgaccagtacctgtactacttgtctaagactatcaacggatccggccagaatcagcagactctgaagttcagccaaggtgggcctaatacaatggccaatcaggcaaagaactggctgccaggaccctgttaccgccaacaacgcgtctcaacgacaaccgggcaaaacaacaatagcaactttgcctggactgctgggaccaaataccatctgaatggaagaaattcattgatgaatcctggccccgctatggcatcccacaaagagggcgaggaccgtttttttcccctgtccgggtccctgatttttggcaaacaaaatgctgccagagacaatgcggattacagcgatgtcatgctcaccagcgaggaagaaatcaaaaccactaaccctgtggctacagaggaatacggtatcgtggcagataacttgcagcagcaaaacacggctcctcaaattggaactgtcaacagccagggggccttacccggtatggtctggcagaaccgggacgtgtacctgcagggtcccatctgggccaagattcctcacacggacggcaacttccacccgtctccgctgatgggcggctttggcctgaaacatcctccgcctcagatcctgatcaagaacacgcctgtacctgcggatcctccgaccaccttcaaccagtcaaagctgaactctttcatcacgcaatacagcaccggacaggtcagcgtggaaattgaatgggagctgcagaaggaaaacagcaagcgctggaaccccgagatccagtacacctccaactactacaaatctacaagtgtggactttgctgttaatacagaaggcgtgtactctgaaccccaccccattggcacccgttacctcacccgtcccctgtaa

SEQ ID NO: 22 - DNA, 2208 bp, synthetic construct
  misc_feature 1..2208: note = AAV214e9 VP1
  source 1..2208: mol_type = other DNA
atggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctccccaggaaccggactcctccgcgggtattggcaaatcgggtgcacagcccgctaaaaagagactcaatttcggtcagactggcgacacagagtcagtcccagaccctcaaccaatcggagaacctcccgcagccccctctggtgtgggatctcttacaatggcttcaggcggtggcgcaccaatggcggacaataacgaaggcgccgacggagtgggtaatgcctcaggaaattggcattgcgattccacatggctgggcgacagagtcatcaccaccagcacccgcacctgggccttgcccacctacaataaccacctctacaagcaaatctccagtgcttcaacgggggccagcaacgacaaccactacttcggctacagcaccccctgggggtattttgacttcaacagattccactgccacttttcaccacgtgactggcaaagactcatcaacaacaactggggattccgacccaagagactcaacttcaagctctttaacattcaagtcaaagaggttacggacaacaatggagtcaagaccatcgccaataaccttaccagcacggtccaggtcttcacggactcagactatcagctcccgtacgtcctcggctctgcgcaccagggctgcctccctccgttcccggcggacgtgttcatgattccgcagtacggctacctaacgctcaacgacggcagccaggcagtgggacggtcatccttttactgcctggaatatttcccatcgcagatgctgagaacgggcaacaactttaccttcagctacacctttgaggacgttcctttccacagcagctacgctcacagccagagtctggaccgtctcatgaatcctctgattgaccagtacctgtactacttgtctaagactatcaacggatccggccagaatcagcagactctgaagttcagccaaggtgggcctaatacaatggccaatcaggcaaagaactggctgccaggaccctgttaccgccaacaacgcgtctcaacgacaaccgggcaaaacaacaatagcaactttgcctggactgctgggaccaaataccatctgaatggaagaaattcattgatgaatcctggccccgctatggcatcccacaaagagggcgaggaccgtttttttcccctgtccgggtccctgatttttggcaaacaaaatgctgccagagacaatgcggattacagcgatgtcatgctcaccagcgaggaagaaatcaaaaccactaaccctgtggctacagaggaatacggtatcgtggcagataacttgcagcagcaaaacacggctcctcaaattggaactgtcaacagccagggggccttacccggtatggtctggcagaaccgggacgtgtacctgcagggtcccatctgggccaagattcctcacacggacggcaacttccacccgtctccgctgatgggcggctttggcctgaaacatcctccgcctcagatcctgatcaagaacacgcctgtacctgcggatcctccgaccaccttcaaccagtcaaagctgaactctttcatcacgcaatacagcaccggacaggtcagcgtggaaattgaatgggagctgcagaaggaaaacagcaagcgctggaaccccgagatccagtacacctccaactactacaaatctacaagtgtggactttgctgttaatacagaaggcgtgtactctgaaccccaccccattggcacccgttacctcacccgtcccctgtaa

SEQ ID NO: 23 - DNA, 2211 bp, synthetic construct
  misc_feature 1..2211: note = AAV214e10 VP1
  source 1..2211: mol_type = other DNA
atggctgccgatggttatcttccagattggctcgaggacaacctctctgagggcattcgcgagtggtgggacttgaaacctggagccccgaaacccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagagaccggtagagccatcaccccagcgttctccagactcctctacgggcatcggcaagaaaggccagcagcccgcgaaaaagagactcaactttgggcagactggcgactcagagtcagtgcccgaccctcaaccaatcggagaaccccccgcaggcccctctggtctgggatctggtacaatggcttcaggcggtggcgcaccaatggcggacaataacgaaggcgccgacggagtgggtaatgcctcaggaaattggcattgcgattccacatggctgggcgacagagtcatcaccaccagcacccgcacctgggccttgcccacctacaataaccacctctacaagcaaatctccagtgcttcaacgggggccagcaacgacaaccactacttcggctacagcaccccctgggggtattttgacttcaacagattccactgccacttttcaccacgtgactggcaaagactcatcaacaacaactggggattccgacccaagagactcaacttcaagctctttaacattcaagtcaaagaggttacggacaacaatggagtcaagaccatcgccaataaccttaccagcacggtccaggtcttcacggactcagactatcagctcccgtacgtcctcggctctgcgcaccagggctgcctccctccgttcccggcggacgtgttcatgattccgcagtacggctacctaacgctcaacgacggcagccaggcagtgggacggtcatccttttactgcctggaatatttcccatcgcagatgctgagaacgggcaacaactttaccttcagctacacctttgaggacgttcctttccacagcagctacgctcacagccagagtctggaccgtctcatgaatcctctgattgaccagtacctgtactacttgtctaagactatcaacggatccggccagaatcagcagactctgaagttcagccaaggtgggcctaatacaatggccaatcaggcaaagaactggctgccaggaccctgttaccgccaacaacgcgtctcaacgacaaccgggcaaaacaacaatagcaactttgcctggactgctgggaccaaataccatctgaatggaagaaattcattgatgaatcctggccccgctatggcatcccacaaagagggcgaggaccgtttttttcccctgtccgggtccctgatttttggcaaacaaaatgctgccagagacaatgcggattacagcgatgtcatgctcaccagcgaggaagaaatcaaaaccactaaccctgtggctacagaggaatacggtatcgtggcagataacttgcagcagcaaaacacggctcctcaaattggaactgtcaacagccagggggccttacccggtatggtctggcagaaccgggacgtgtacctgcagggtcccatctgggccaagattcctcacacggacggcaacttccacccgtctccgctgatgggcggctttggcctgaaacatcctccgcctcagatcctgatcaagaacacgcctgtacctgcggatcctccgaccaccttcaaccagtcaaagctgaactctttcatcacgcaatacagcaccggacaggtcagcgtggaaattgaatgggagctgcagaaggaaaacagcaagcgctggaaccccgagatccagtacacctccaactactacaaatctacaagtgtggactttgctgttaatacagaaggcgtgtactctgaaccccaccccattggcacccgttacctcacccgtcccctgtaa

SEQ ID NO: 24 - DNA, 1602 bp, synthetic construct
  misc_feature 1..1602: note = ITB102 214 (AAV214) VP3
  source 1..1602: mol_type = other DNA
atggcttcaggcggtggcgcaccaatggcggacaataacgaaggcgccgacggagtgggtaatgcctcaggaaattggcattgcgattccacatggctgggcgacagagtcatcaccaccagcacccgcacctgggccttgcccacctacaataaccacctctacaagcaaatctccagtgcttcaacgggggccagcaacgacaaccactacttcggctacagcaccccctgggggtattttgacttcaacagattccactgccacttttcaccacgtgactggcaaagactcatcaacaacaactggggattccgacccaagagactcaacttcaagctctttaacattcaagtcaaagaggttacggacaacaatggagtcaagaccatcgccaataaccttaccagcacggtccaggtcttcacggactcagactatcagctcccgtacgtcctcggctctgcgcaccagggctgcctccctccgttcccggcggacgtgttcatgattccgcagtacggctacctaacgctcaacgacggcagccaggcagtgggacggtcatccttttactgcctggaatatttcccatcgcagatgctgagaacgggcaacaactttaccttcagctacacctttgaggacgttcctttccacagcagctacgctcacagccagagtctggaccgtctcatgaatcctctgattgaccagtacctgtactacttgtctaagactatcaacggatccggccagaatcagcagactctgaagttcagccaaggtgggcctaatacaatggccaatcaggcaaagaactggctgccaggaccctgttaccgccaacaacgcgtctcaacgacaaccgggcaaaacaacaatagcaactttgcctggactgctgggaccaaataccatctgaatggaagaaattcattgatgaatcctggccccgctatggcatcccacaaagagggcgaggaccgtttttttcccctgtccgggtccctgatttttggcaaacaaaatgctgccagagacaatgcggattacagcgatgtcatgctcaccagcgaggaagaaatcaaaaccactaaccctgtggctacagaggaatacggtatcgtggcagataacttgcagcagcaaaacacggctcctcaaattggaactgtcaacagccagggggccttacccggtatggtctggcagaaccgggacgtgtacctgcagggtcccatctgggccaagattcctcacacggacggcaacttccacccgtctccgctgatgggcggctttggcctgaaacatcctccgcctcagatcctgatcaagaacacgcctgtacctgcggatcctccgaccaccttcaaccagtcaaagctgaactctttcatcacgcaatacagcaccggacaggtcagcgtggaaattgaatgggagctgcagaaggaaaacagcaagcgctggaaccccgagatccagtacacctccaactactacaaatctacaagtgtggactttgctgttaatacagaaggcgtgtactctgaaccccaccccattggcacccgttacctcacccgtcccctgtaa

SEQ ID NO: 25 - DNA, 1605 bp, synthetic construct
  misc_feature 1..1605: note = AAV214-A VP3
  source 1..1605: mol_type = other DNA
atggcttcaggcggtggcgcaccaatggcggacaataacgaaggcgccgacggagtgggtaatgcctcaggaaattggcattgcgattccacatggctgggcgacagagtcatcaccaccagcacccgcacctgggccttgcccacctacaataaccacctctacaagcaaatctccaacagcacatctggaggatcttcaaatgacaacgcctacttcggctacagcaccccctgggggtattttgacttcaacagattccactgccacttttcaccacgtgactggcaaagactcatcaacaacaactggggattccgacccaagagactcaacttcaagctctttaacattcaagtcaaagaggttacggacaacaatggagtcaagaccatcgccaataaccttaccagcacggtccaggtcttcacggactcagactatcagctcccgtacgtcctcggctctgcgcaccagggctgcctccctccgttcccggcggacgtgttcatgattccgcagtacggctacctaacgctcaacgacggcagccaggcagtgggacggtcatccttttactgcctggaatatttcccatcgcagatgctgagaacgggcaacaactttaccttcagctacacctttgaggacgttcctttccacagcagctacgctcacagccagagtctggaccgtctcatgaatcctctgattgaccagtacctgtactacttgtctaagactatcaacggatccggccagaatcagcagactctgaagttcagccaaggtgggcctaatacaatggccaatcaggcaaagaactggctgccaggaccctgttaccgccaacaacgcgtctcaacgacaaccgggcaaaacaacaatagcaactttgcctggactgctgggaccaaataccatctgaatggaagaaattcattgatgaatcctggccccgctatggcatcccacaaagagggcgaggaccgtttttttcccctgtccgggtccctgatttttggcaaacaaaatgctgccagagacaatgcggattacagcgatgtcatgctcaccagcgaggaagaaatcaaaaccactaaccctgtggctacagaggaatacggtatcgtggcagataacttgcagcagcaaaacacggctcctcaaattggaactgtcaacagccagggggccttacccggtatggtctggcagaaccgggacgtgtacctgcagggtcccatctgggccaagattcctcacacggacggcaacttccacccgtctccgctgatgggcggctttggcctgaaacatcctccgcctcagatcctgatcaagaacacgcctgtacctgcggatcctccgaccaccttcaaccagtcaaagctgaactctttcatcacgcaatacagcaccggacaggtcagcgtggaaattgaatgggagctgcagaaggaaaacagcaagcgctggaaccccgagatccagtacacctccaactactacaaatctacaagtgtggactttgctgttaatacagaaggcgtgtactctgaaccccaccccattggcacccgttacctcacccgtcccctgtaa

SEQ ID NO: 26 - DNA, 1602 bp, synthetic construct
  misc_feature 1..1602: note = AAV214e VP3
  source 1..1602: mol_type = other DNA
atggcttcaggcggtggcgcaccaatggcagacaataacgaaggcgccgacggagtgggtaatgcctcaggaaattggcattgcgattccacatggctgggcgacagagtcatcaccaccagcacccgcacctgggccttgcccacctacaataaccacctctacaagcaaatctccagtgcttcaacgggggccagcaacgacaaccactacttcggctacagcaccccctgggggtattttgacttcaacagattccactgccacttttcaccacgtgactggcaaagactcatcaacaacaactggggattccgacccaagagactcaacttcaagctctttaacattcaagtcaaagaggttacggacaacaatggagtcaagaccatcgccaataaccttaccagcacggtccaggtcttcacggactcagactatcagctcccgtacgtcctcggctctgcgcaccagggctgcctccctccgttcccggcggacgtgttcatgattccgcagtacggctacctaacgctcaacgacggcagccaggcagtgggacggtcatccttttactgcctggaatatttcccatcgcagatgctgagaacgggcaacaactttaccttcagctacacctttgaggacgttcctttccacagcagctacgctcacagccagagtctggaccgtctcatgaatcctctgattgaccagtacctgtactacttgtctaagactatcaacggatccggccagaatcagcagactctgaagttcagccaaggtgggcctaatacaatggccaatcaggcaaagaactggctgccaggaccctgttaccgccaacaacgcgtctcaacgacaaccgggcaaaacaacaatagcaactttgcctggactgctgggaccaaataccatctgaatggaagaaattcattgatgaatcctggccccgctatggcatcccacaaagagggcgaggaccgtttttttcccctgtccgggtccctgatttttggcaaacaaaatgctgccagagacaatgcggattacagcgatgtcatgctcaccagcgaggaagaaatcaaaaccactaaccctgtggctacagaggaatacggtatcgtggcagataacttgcagcagcaaaacacggctcctcaaattggaactgtcaacagccagggggccttacccggtatggtctggcagaaccgggacgtgtacctgcagggtcccatctgggccaagattcctcacacggacggcaacttccacccgtctccgctgatgggcggctttggcctgaaacatcctccgcctcagatcctgatcaagaacacgcctgtacctgcggatcctccgaccaccttcaaccagtcaaagctgaactctttcatcacgcaatacagcaccggacaggtcagcgtggaaattgaatgggagctgcagaaggaaaacagcaagcgctggaaccccgagatccagtacacctccaactactacaaatctacaagtgtggactttgctgttaatacagaaggcgtgtactctgaaccccaccccattggcacccgttacctcacccgtcccctgtaa

SEQ ID NO: 27 - DNA, 1602 bp, synthetic construct; identical to SEQ ID NO: 24
  misc_feature 1..1602: note = AAV214e8 VP3
  source 1..1602: mol_type = other DNA

SEQ ID NO: 28 - DNA, 1602 bp, synthetic construct; identical to SEQ ID NO: 24
  misc_feature 1..1602: note = AAV214e9 VP3
  source 1..1602: mol_type = other DNA

SEQ ID NO: 29 - DNA, 1602 bp, synthetic construct; identical to SEQ ID NO: 24
  misc_feature 1..1602: note = AAV214e10 VP3
  source 1..1602: mol_type = other DNA

SEQ ID NO: 30 - protein, 736 aa, synthetic construct
  REGION 1..736: note = AAV214A VP1
MAADGYLPDWLEDNLSEGIREWWALKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPFGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHQGCLPPFPADVFMIPQYGYLTLNDGSQAVGRSSFYCLEYFPSQMLRTGNNFTFSYTFEDVPFHSSYAHSQSLDRLMNPLIDQYLYYLSKTINGSGQNQQTLKFSQGGPNTMANQAKNWLPGPCYRQQRVSTTTGQNNNSNFAWTAGTKYHLNGRNSLMNPGPAMASHKEGEDRFFPLSGSLIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPHPIGTRYLTRPL

SEQ ID NO: 31 - protein, 736 aa, synthetic construct
  REGION 1..736: note = AAV214e VP1
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTDNNGVKTIANNLTSTVQVFTDSDYQLPYVLGSAHQGCLPPFPADVFMIPQYGYLTLNDGSQAVGRSSFYCLEYFPSQMLRTGNNFTFSYTFEDVPFHSSYAHSQSLDRLMNPLIDQYLYYLSKTINGSGQNQQTLKFSQGGPNTMANQAKNWLPGPCYRQQRVSTTTGQNNNSNFAWTAGTKYHLNGRNSLMNPGPAMASHKEGEDRFFPLSGSLIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPHPIGTRYLTRPL

SEQ ID NO: 32 - protein, 736 aa, synthetic construct
  REGION 1..736: note = AAV214e8 VP1
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTDNNGVKTIANNLTSTVQVFTDSDYQLPYVLGSAHQGCLPPFPADVFMIPQYGYLTLNDGSQAVGRSSFYCLEYFPSQMLRTGNNFTFSYTFEDVPFHSSYAHSQSLDRLMNPLIDQYLYYLSKTINGSGQNQQTLKFSQGGPNTMANQAKNWLPGPCYRQQRVSTTTGQNNNSNFAWTAGTKYHLNGRNSLMNPGPAMASHKEGEDRFFPLSGSLIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPHPIGTRYLTRPL

SEQ ID NO: 33 - protein, 735 aa, synthetic construct
  REGION 1..735: note = AAV214e9 VP1
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTDNNGVKTIANNLTSTVQVFTDSDYQLPYVLGSAHQGCLPPFPADVFMIPQYGYLTLNDGSQAVGRSSFYCLEYFPSQMLRTGNNFTFSYTFEDVPFHSSYAHSQSLDRLMNPLIDQYLYYLSKTINGSGQNQQTLKFSQGGPNTMANQAKNWLPGPCYRQQRVSTTTGQNNNSNFAWTAGTKYHLNGRNSLMNPGPAMASHKEGEDRFFPLSGSLIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPHPIGTRYLTRPL

SEQ ID NO: 34 - protein, 736 aa, synthetic construct
  REGION 1..736: note = AAV214e10 VP1
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEPSPQRSPDSSTGIGKKGQQPAKKRLNFGQTGDSESVPDPQPIGEPPAGPSGLGSGTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTDNNGVKTIANNLTSTVQVFTDSDYQLPYVLGSAHQGCLPPFPADVFMIPQYGYLTLNDGSQAVGRSSFYCLEYFPSQMLRTGNNFTFSYTFEDVPFHSSYAHSQSLDRLMNPLIDQYLYYLSKTINGSGQNQQTLKFSQGGPNTMANQAKNWLPGPCYRQQRVSTTTGQNNNSNFAWTAGTKYHLNGRNSLMNPGPAMASHKEGEDRFFPLSGSLIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPHPIGTRYLTRPL

SEQ ID NO: 35 - protein, 598 aa, synthetic construct
  REGION 1..598: note = AAV214 VP2
MAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTDNNGVKTIANNLTSTVQVFTDSDYQLPYVLGSAHQGCLPPFPADVFMIPQYGYLTLNDGSQAVGRSSFYCLEYFPSQMLRTGNNFTFSYTFEDVPFHSSYAHSQSLDRLMNPLIDQYLYYLSKTINGSGQNQQTLKFSQGGPNTMANQAKNWLPGPCYRQQRVSTTTGQNNNSNFAWTAGTKYHLNGRNSLMNPGPAMASHKEGEDRFFPLSGSLIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPHPIGTRYLTRPL

SEQ ID NO: 36 - protein, 599 aa, synthetic construct
  REGION 1..599: note = AAV214A VP2
MAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHQGCLPPFPADVFMIPQYGYLTLNDGSQAVGRSSFYCLEYFPSQMLRTGNNFTFSYTFEDVPFHSSYAHSQSLDRLMNPLIDQYLYYLSKTINGSGQNQQTLKFSQGGPNTMANQAKNWLPGPCYRQQRVSTTTGQNNNSNFAWTAGTKYHLNGRNSLMNPGPAMASHKEGEDRFFPLSGSLIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPHPIGTRYLTRPL

SEQ ID NO: 37 - protein, 599 aa, synthetic construct
  REGION 1..599: note = AAV214e VP2
MAPGKKRPVEPSPQRSPDSSTGIGKKGQQPARKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTDNNGVKTIANNLTSTVQVFTDSDYQLPYVLGSAHQGCLPPFPADVFMIPQYGYLTLNDGSQAVGRSSFYCLEYFPSQMLRTGNNFTFSYTFEDVPFHSSYAHSQSLDRLMNPLIDQYLYYLSKTINGSGQNQQTLKFSQGGPNTMANQAKNWLPGPCYRQQRVSTTTGQNNNSNFAWTAGTKYHLNGRNSLMNPGPAMASHKEGEDRFFPLSGSLIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPHPIGTRYLTRPL

SEQ ID NO: 38 - protein, 599 aa, synthetic construct
  REGION 1..599: note = AAV214e8 VP2
MAPGKKRPVEPSPQRSPDSSTGIGKKGQQPARKRLNFGQTGDSESVPDPQPLGEPPAAPSGVGPNTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTDNNGVKTIANNLTSTVQVFTDSDYQLPYVLGSAHQGCLPPFPADVFMIPQYGYLTLNDGSQAVGRSSFYCLEYFPSQMLRTGNNFTFSYTFEDVPFHSSYAHSQSLDRLMNPLIDQYLYYLSKTINGSGQNQQTLKFSQGGPNTMANQAKNWLPGPCYRQQRVSTTTGQNNNSNFAWTAGTKYHLNGRNSLMNPGPAMASHKEGEDRFFPLSGSLIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPHPIGTRYLTRPL

SEQ ID NO: 39 - protein, 598 aa, synthetic construct
  REGION 1..598: note = AAV214e9 VP2
MAPGKKRPVEQSPQEPDSSAGIGKSGAQPAKKRLNFGQTGDTESVPDPQPIGEPPAAPSGVGSLTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTDNNGVKTIANNLTSTVQVFTDSDYQLPYVLGSAHQGCLPPFPADVFMIPQYGYLTLNDGSQAVGRSSFYCLEYFPSQMLRTGNNFTFSYTFEDVPFHSSYAHSQSLDRLMNPLIDQYLYYLSKTINGSGQNQQTLKFSQGGPNTMANQAKNWLPGPCYRQQRVSTTTGQNNNSNFAWTAGTKYHLNGRNSLMNPGPAMASHKEGEDRFFPLSGSLIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPHPIGTRYLTRPL

SEQ ID NO: 40 - protein, 599 aa, synthetic construct
  REGION 1..599: note = AAV214e10 VP2
MAPGKKRPVEPSPQRSPDSSTGIGKKGQQPAKKRLNFGQTGDSESVPDPQPIGEPPAGPSGLGSGTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTDNNGVKTIANNLTSTVQVFTDSDYQLPYVLGSAHQGCLPPFPADVFMIPQYGYLTLNDGSQAVGRSSFYCLEYFPSQMLRTGNNFTFSYTFEDVPFHSSYAHSQSLDRLMNPLIDQYLYYLSKTINGSGQNQQTLKFSQGGPNTMANQAKNWLPGPCYRQQRVSTTTGQNNNSNFAWTAGTKYHLNGRNSLMNPGPAMASHKEGEDRFFPLSGSLIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPHPIGTRYLTRPL

SEQ ID NO: 41 - protein, 533 aa, synthetic construct
  REGION 1..533: note = AAV214 VP3
MASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTDNNGVKTIANNLTSTVQVFTDSDYQLPYVLGSAHQGCLPPFPADVFMIPQYGYLTLNDGSQAVGRSSFYCLEYFPSQMLRTGNNFTFSYTFEDVPFHSSYAHSQSLDRLMNPLIDQYLYYLSKTINGSGQNQQTLKFSQGGPNTMANQAKNWLPGPCYRQQRVSTTTGQNNNSNFAWTAGTKYHLNGRNSLMNPGPAMASHKEGEDRFFPLSGSLIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPHPIGTRYLTRPL

SEQ ID NO: 42 - protein, 534 aa, synthetic construct
  REGION 1..534: note = AAV214A VP3
MASGGGAPMADNNEGADGVGNASGNWHCDSTWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHQGCLPPFPADVFMIPQYGYLTLNDGSQAVGRSSFYCLEYFPSQMLRTGNNFTFSYTFEDVPFHSSYAHSQSLDRLMNPLIDQYLYYLSKTINGSGQNQQTLKFSQGGPNTMANQAKNWLPGPCYRQQRVSTTTGQNNNSNFAWTAGTKYHLNGRNSLMNPGPAMASHKEGEDRFFPLSGSLIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPHPIGTRYLTRPL

SEQ ID NO: 43 - protein, 533 aa, synthetic construct; identical to SEQ ID NO: 41
  REGION 1..533: note = AAV214E VP3

SEQ ID NO: 44 - protein, 533 aa, synthetic construct; identical to SEQ ID NO: 41
  REGION 1..533: note = AAV214E8 VP3

SEQ ID NO: 45 - protein, 533 aa, synthetic construct; identical to SEQ ID NO: 41
  REGION 1..533: note = AAV214E9 VP3

SEQ ID NO: 46 - protein, 533 aa, synthetic construct; identical to SEQ ID NO: 41
  REGION 1..533: note = AAV214E10 VP3

SEQ ID NO: 47 - DNA, 2211 bp, synthetic construct
  misc_feature 1..2211: note = ITB102 45 VP1
  source 1..2211: mol_type = other DNA
atggctgccgatggttatcttccagattggctcgaggacaacctctctgagggcattcgcgagtggtgggcgctgaaacctggagccccgaagcccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagcgggttctcgaaccttttggtctggttgaggaaggtgctaagacggctcctggaaagaaacgtccggtagagcagtcgccacaagagccagactcctcctcgggcatcggcaagacaggccagcagcccgctaaaaagagactcaattttggtcagactggcgactcagagtcagtccccgacccacaacctctcggagaacctccagcaacccccgctgctgtgggacctactacaatggcttcaggcggtggcgcaccaatggcggacaataacgaaggcgccgacggagtgggtaatgcctcaggaaattggcattgcgattccacatggctgggcgacagagtcatcaccaccagcacccgcacctgggccttgcccacctacaataaccacctctacaagcaaatctccagtgcttcaacgggggccagcaacgacaaccactacttcggctacagcaccccctgggggtattttgacttcaacagattccactgccacttttcaccacgtgactggcaaagactcatcaacaacaactggggattccgacccaagagactcaacttcaagctctttaacattcaagtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacggctacctaacgctcaacaatggcagccaggcagtgggacggtcatccttttactgcctggaatatttcccatcgcagatgctgagaacgggcaacaactttaccttcagctacacctttgaggacgttcctttccacagcagctacgctcacagccagagtctggaccggctgatgaatcctctgattgaccagtacctgtactacttgtctcggactcaaacaacaggaggcacggcaaatacgcagactctgggcttcagccaaggtgggcctaatacaatggccaatcaggcaaagaactggctgccaggaccctgttaccgccaacaacgcgtctcaacgacaaccgggcaaaacaacaatagcaactttgcctggactgctgggaccaaataccatctgaatggaagaaattcattgatgaatcctggccccgctatggcatcccacaaagagggcgaggaccgtttttttcccctgtccgggtccctgatttttggcaaacaaggcactggcagagacaatgtggatgccgacaaagtcatgatcaccaacgaggaagaaatcaaaaccactaaccctgtggctacagaggaatacggtatcgtggcagataacttgcagcagcaaaacacggctcctcaaattggaactgtcaacagccagggggccttacccggtatggtctggcagaaccgggacgtgtacctgcagggtcccatctgggccaagattcctcacacggacggcaacttccacccgtctccgctgatgggcggctttggcctgaaacatcctccgcctcagatcctgatcaagaacacgcctgtacctgcggatcctccgaccaccttcaaccagtcaaagctgaactctttcatcacgcaatacagcaccggacaggtcagcgtggaaattgaatgggagctgcagaaggaaaacagcaagcgctggaaccccgagatccagtacacctccaactactacaaatctacaagtgtggactttgctgttaatacagaaggcgtgtactctgaaccccaccccattggcacccgttacctcacccgtcccctgtaa

SEQ ID NO: 48 - DNA, 1605 bp, synthetic construct
  misc_feature 1..1605: note = ITB102 45 VP3
  source 1..1605: mol_type = other DNA
atggcttcaggcggtggcgcaccaatggcggacaataacgaaggcgccgacggagtgggtaatgcctcaggaaattggcattgcgattccacatggctgggcgacagagtcatcaccaccagcacccgcacctgggccttgcccacctacaataaccacctctacaagcaaatctccagtgcttcaacgggggccagcaacgacaaccactacttcggctacagcaccccctgggggtattttgacttcaacagattccactgccacttttcaccacgtgactggcaaagactcatcaacaacaactggggattccgacccaagagactcaacttcaagctctttaacattcaagtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacggctacctaacgctcaacaatggcagccaggcagtgggacggtcatccttttactgcctggaatatttcccatcgcagatgctgagaacgggcaacaactttaccttcagctacacctttgaggacgttcctttccacagcagctacgctcacagccagagtctggaccggctgatgaatcctctgattgaccagtacctgtactacttgtctcggactcaaacaacaggaggcacggcaaatacgcagactctgggcttcagccaaggtgggcctaatacaatggccaatcaggcaaagaactggctgccaggaccctgttaccgccaacaacgcgtctcaacgacaaccgggcaaaacaacaatagcaactttgcctggactgctgggaccaaataccatctgaatggaagaaattcattgatgaatcctggccccgctatggcatcccacaaagagggcgaggaccgtttttttcccctgtccgggtccctgatttttggcaaacaaggcactggcagagacaatgtggatgccgacaaagtcatgatcaccaacgaggaagaaatcaaaaccactaaccctgtggctacagaggaatacggtatcgtggcagataacttgcagcagcaaaacacggctcctcaaattggaactgtcaacagccagggggccttacccggtatggtctggcagaaccgggacgtgtacctgcagggtcccatctgggccaagattcctcacacggacggcaacttccacccgtctccgctgatgggcggctttggcctgaaacatcctccgcctcagatcctgatcaagaacacgcctgtacctgcggatcctccgaccaccttcaaccagtcaaagctgaactctttcatcacgcaatacagcaccggacaggtcagcgtggaaattgaatgggagctgcagaaggaaaacagcaagcgctggaaccccgagatccagtacacctccaactactacaaatctacaagtgtggactttgctgttaatacagaaggcgtgtactctgaaccccaccccattggcacccgttacctcacccgtcccctgtaa

SEQ ID NO: 49 - protein, 736 aa, synthetic construct
  REGION 1..736: note = ITB102 45 VP1
MAADGYLPDWLEDNLSEGIREWWALKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPFGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNNGSQAVGRSSFYCLEYFPSQMLRTGNNFTFSYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLMNPGPAMASHKEGEDRFFPLSGSLIFGKQGTGRDNVDADKVMITNEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPHPIGTRYLTRPL

SEQ ID NO: 50 - protein, 599 aa, synthetic construct
  REGION 1..599: note = ITB102 45 VP2
MAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNNGSQAVGRSSFYCLEYFPSQMLRTGNNFTFSYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLMNPGPAMASHKEGEDRFFPLSGSLIFGKQGTGRDNVDADKVMITNEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPHPIGTRYLTRPL

SEQ ID NO: 51 - protein, 534 aa, synthetic construct
  REGION 1..534: note = ITB102 45 VP3
MASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNNGSQAVGRSSFYCLEYFPSQMLRTGNNFTFSYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLMNPGPAMASHKEGEDRFFPLSGSLIFGKQGTGRDNVDADKVMITNEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPHPIGTRYLTRPL

SEQ ID NO: 52 - protein, 7 aa, synthetic construct
  REGION 1..7: note = VR-I
SASTGAS

SEQ ID NO: 53 - protein, 8 aa, synthetic construct
  REGION 1..8: note = VR-I
NSTSGGSS

SEQ ID NO: 54 - protein, 6 aa, synthetic construct
  REGION 1..6: note = VR-II
DNNGVK

SEQ ID NO: 55 - protein, 4 aa, synthetic construct
  REGION 1..4: note = VR-III
NDGS

SEQ ID NO: 56 - protein, 10 aa, synthetic construct
  REGION 1..10: note = VR-IV
INGSGQNQQT

SEQ ID NO: 57 - protein, 18 aa, synthetic construct
  REGION 1..18: note = VR-V
RVSTTTGQNNNSNFAWTA

SEQ ID NO: 58 - protein, 13 aa, synthetic construct
  REGION 1..13: note = VR-VI
HKEGEDRFFPLSG

SEQ ID NO: 59 - protein, 14 aa, synthetic construct
  REGION 1..14: note = VR-VII
KQNAARDNADYSDV

SEQ ID NO: 60 - protein, 13 aa, synthetic construct
  REGION 1..13: note = VR-VIII
ADNLQQQNTAPQI

SEQ ID NO: 61 - protein, 10 aa, synthetic construct
  REGION 1..10: note = VR-IX
NYYKSTSVDF

SEQ ID NO: 62 - DNA, 2211 bp, synthetic construct
  misc_feature 1..2211: note = AAV6 VP1
  source 1..2211: mol_type = other DNA
atggctgccgatggttatcttccagattggctcgaggacaacctctctgagggcattcgcgagtggtgggacttgaaacctggagccccgaaacccaaagccaaccagcaaaagcaggacgacggccggggtctggtgcttcctggctacaagtacctcggacccttcaacggactcgacaagggggagcccgtcaacgcggcggatgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagagggttctcgaaccttttggtctggttgaggaaggtgctaagacggctcctggaaagaaacgtccggtagagcagtcgccacaagagccagactcctcctcgggcattggcaagacaggccagcagcccgctaaaaagagactcaattttggtcagactggcgactcagagtcagtccccgacccacaacctctcggagaacctccagcaacccccgctgctgtgggacctactacaatggcttcaggcggtggcgcaccaatggcagacaataacgaaggcgccgacggagtgggtaatgcctcaggaaattggcattgcgattccacatggctgggcgacagagtcatcaccaccagcacccgaacatgggccttgcccacctataacaaccacctctacaagcaaatctccagtgcttcaacgggggccagcaacgacaaccactacttcggctacagcaccccctgggggtattttgatttcaacagattccactgccatttctcaccacgtgactggcagcgactcatcaacaacaattggggattccggcccaagagactcaacttcaagctcttcaacatccaagtcaaggaggtcacgacgaatgatggcgtcacgaccatcgctaataaccttaccagcacggttcaagtcttctcggactcggagtaccagttgccgtacgtcctcggctctgcgcaccagggctgcctccctccgttcccggcggacgtgttcatgattccgcagtacggctacctaacgctcaacaatggcagccaggcagtgggacggtcatccttttactgcctggaatatttcccatcgcagatgctgagaacgggcaataactttaccttcagctacaccttcgaggacgtgcctttccacagcagctacgcgcacagccagagcctggaccggctgatgaatcctctcatcgaccagtacctgtattacctgaacagaactcagaatcagtccggaagtgcccaaaacaaggacttgctgtttagccgggggtctccagctggcatgtctgttcagcccaaaaactggctacctggaccctgttaccggcagcagcgcgtttctaaaacaaaaacagacaacaacaacagcaactttacctggactggtgcttcaaaatataaccttaatgggcgtgaatctataatcaaccctggcactgctatggcctcacacaaagacgacaaagacaagttctttcccatgagcggtgtcatgatttttggaaaggagagcgccggagcttcaaacactgcattggacaatgtcatgatcacagacgaagaggaaatcaaagccactaaccccgtggccaccgaaagatttgggactgtggcagtcaatctccagagcagcagcacagaccctgcgaccggagatgtgcatgttatgggagccttacctggaatggtgtggcaagacagagacgtatacctgcagggtcctatttgggccaaaattcctcacacggatggacactttcacccgtctcctctcatgggcggctttggacttaagcacccgcctcctcagatcctcatcaaaaacacgcctgttcctgcgaatcctccggcagagttttcggctacaaagtttgcttcattcatcacccagtattccacaggacaagtgagcgtggagattgaatgggagctgcagaaagaaaacagcaaacgctggaatcccgaagtgcagtatacatctaactatgcaaaatctgccaacgttgatttcactgtggacaacaatggactttatactgagcctcgccccattggcacccgttacctcacccgtcccctgtaa

SEQ ID NO: 63 - protein, 736 aa, synthetic construct
  REGION 1..736: note = AAV6 VP1
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPFGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDKDKFFPMSGVMIFGKESAGASNTALDNVMITDEEEIKATNPVATERFGTVAVNLQSSSTDPATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 64 - protein, 599 aa, synthetic construct
  REGION 1..599: note = AAV6 VP2
TAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDKDKFFPMSGVMIFGKESAGASNTALDNVMITDEEEIKATNPVATERFGTVAVNLQSSSTDPATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 65 - protein, 534 aa, synthetic construct
  REGION 1..534: note = AAV6 VP3
MASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDKDKFFPMSGVMIFGKESAGASNTALDNVMITDEEEIKATNPVATERFGTVAVNLQSSSTDPATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 66 - DNA, 2217 bp, synthetic construct
  misc_feature 1..2217: note = AAV8 VP1
  source 1..2217: mol_type = other DNA
atggctgccgatggttatcttccagattggctcgaggacaacctctctgagggcattcgcgagtggtgggcgctgaaacctggagccccgaagcccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctgcaggcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagagaccggtagagccatcaccccagcgttctccagactcctctacgggcatcggcaagaaaggccaacagcccgccagaaaaagactcaattttggtcagactggcgactcagagtcagttccagaccctcaacctctcggagaacctccagcagcgccctctggtgtgggacctaatacaatggctgcaggcggtggcgcaccaatggcagacaataacgaaggcgccgacggagtgggtagttcctcgggaaattggcattgcgattccacatggctgggcgacagagtcatcaccaccagcacccgaacctgggccctgcccacctacaacaaccacctctacaagcaaatctccaacgggacatcgggaggagccaccaacgacaacacctacttcggctacagcaccccctgggggtattttgactttaacagattccactgccacttttcaccacgtgactggcagcgactcatcaacaacaactggggattccggcccaagagactcagcttcaagctcttcaacatccaggtcaaggaggtcacgcagaatgaaggcaccaagaccatcgccaataacctcaccagcaccatccaggtgtttacggactcggagtaccagctgccgtacgttctcggctctgcccaccagggctgcctgcctccgttcccggcggacgtgttcatgattccccagtacggctacctaacactcaacaacggtagtcaggccgtgggacgctcctccttctactgcctggaatactttccttcgcagatgctgagaaccggcaacaacttccagtttacttacaccttcgaggacgtgcctttccacagcagctacgcccacagccagagcttggaccggctgatgaatcctctgattgaccagtacctgtactacttgtctcggactcaaacaacaggaggcacggcaaatacgcagactctgggcttcagccaaggtgggcctaatacaatggccaatcaggcaaagaactggctgccaggaccctgttaccgccaacaacgcgtctcaacgacaaccgggcaaaacaacaatagcaactttgcctggactgctgggaccaaataccatctgaatggaagaaattcattggctaatcctggcatcgctatggcaacacacaaagacgacgaggagcgtttttttcccagtaacgggatcctgatttttggcaaacaaaatgctgccagagacaatgcggattacagcgatgtcatgctcaccagcgaggaagaaatcaaaaccactaaccctgtggctacagaggaatacggtatcgtggcagataacttgcagcagcaaaacacggctcctcaaattggaactgtcaacagccagggggccttacccggtatggtctggcagaaccgggacgtgtacctgcagggtcccatctgggccaagattcctcacacggacggcaacttccacccgtctccgctgatgggcggctttggcctgaaacatcctccgcctcagatcctgatcaagaacacgcctgtacctgcggatcctccgaccaccttcaaccagtcaaagctgaactctttcatcacgcaatacagcaccggacaggtcagcgtggaaattgaatgggagctgcagaaggaaaacagcaagcgctggaaccccgagatccagtacacctccaactactacaaatctacaagtgtggactttgctgttaatacagaaggcgtgtactctgaaccccgccccattggcacccgttacctcacccgtaatctgtaa

SEQ ID NO: 67 - protein, 738 aa, synthetic construct
  REGION 1..738: note = AAV8 VP1
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 68 - protein, 601 aa, synthetic construct
  REGION 1..601: note = AAV8 VP2
MAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 69 - protein, 535 aa, synthetic construct
  REGION 1..535: note = AAV8 VP3
MAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 70 - DNA, 2214 bp, synthetic construct
  misc_feature 1..2214: note = AAV9 VP1
  source 1..2214: mol_type = other DNA
atggctgccgatggttatcttccagattggctcgaggacaacctctctgagggcattcgcgagtggtgggcgctgaaacctggagccccgaagcccaaagccaaccagcaaaagcaggacgacggccggggtctggtgcttcctggctacaagtacctcggacccttcaacggactcgacaagggggagcccgtcaacgcggcggacgcagcggccctcgagcacggcaaggcctacgaccagcagctgcaggcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagagaccggtagagccatcaccccagcgttctccagactcctctacgggcatcggcaagaaaggccaacagcccgccagaaaaagactcaattttggtcagactggcgactcagagtcagttccagaccctcaacctctcggagaacctccagcagcgccctctggtgtgggacctaatacaatggctgcaggcggtggcgcaccaatggcagacaataacgaaggcgccgacggagtgggtaattcctcgggaaattggcattgcgattccacatggctgggggacagagtcatcaccaccagcacccgaacctgggcattgcccacctacaacaaccacctctacaagcaaatctccaatggaacatcgggaggaagcaccaacgacaacacctactttggctacagcaccccctgggggtattttgacttcaacagattccactgccacttctcaccacgtgactggcagcgactcatcaacaacaactggggattccggccaaagagactcaacttcaagctgttcaacatccaggtcaaggaggttacgacgaacgaaggcaccaagaccatcgccaataaccttaccagcaccgtccaggtctttacggactcggagtaccagctaccgtacgtcctaggctctgcccaccaaggatgcctgccaccgtttcctgcagacgtcttcatggttcctcagtacggctacctgacgctcaacaatggaagtcaagcgttaggacgttcttctttctactgtctggaatacttcccttctcagatgctgagaaccggcaacaactttcagttcagctacactttcgaggacgtgcctttccacagcagctacgcacacagccagagtctagatcgactgatgaaccccctcatcgaccagtacctatactacctggtcagaacacagacaactggaactgggggaactcaaactttggcattcagccaagcaggccctagctcaatggccaatcaggctagaaactgggtacccgggccttgctaccgtcagcagcgcgtctccacaaccaccaaccaaaataacaacagcaactttgcgtggacgggagctgctaaattcaagctgaacgggagagactcgctaatgaatcctggcgtggctatggcatcgcacaaagacgacgaggaccgcttctttccatcaagtggcgttctcatatttggcaagcaaggagccgggaacgatggagtcgactacagccaggtgctgattacagatgaggaagaaattaaagccaccaaccctgtagccacagaggaatacggagcagtggccatcaacaaccaggccgctaacacgcaggcgcaaactggacttgtgcataaccagggagttattcctggtatggtctggcagaaccgggacgtgtacctgcagggccctatttgggctaaaatacctcacacagatggcaactttcacccgtctcctctgatgggtggatttggactgaaacacccacctccacagattctaattaaaaatacaccagtgccggcagatcctcctcttaccttcaatcaagccaagctgaactctttcatcacgcagtacagcacgggacaagtcagcgtggaaatcgagtgggagctgcagaaagaaaacagcaagcgctggaatccagagatccagtatacttcaaactactacaaatctacaaatgtggactttgctgtcaataccaaaggtgtttactctgagcctcgccccattggtactcgttacctcacccgtaatttgtaa

SEQ ID NO: 71 - protein, 736 aa, synthetic construct
  REGION 1..736: note = AAV9 VP1
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 72 - protein, 599 aa, synthetic construct
  REGION 1..599: note = AAV9 VP2
MAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 73 - protein, 534 aa, synthetic construct
  REGION 1..534: note = AAV9 VP3
MASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 74 - protein, 6 aa, synthetic construct
  REGION 1..6: note = VRII-204/AAV6
TNDGVK

SEQ ID NO: 75 - protein, 4 aa, synthetic construct
  REGION 1..4: note = VRIII-204/AAV6
NNGS

SEQ ID NO: 76 - protein, 11 aa, synthetic construct
  REGION 1..11: note = VRIV-204/AAV6
QNQSGSAQNKD

SEQ ID NO: 77 - protein, 18 aa, synthetic construct
  REGION 1..18: note = VRV-204/AAV6
RVSKTKTDNNNSNFTWTG

SEQ ID NO: 78 - protein, 13 aa, synthetic construct
  REGION 1..13: note = VRVI-204/AAV6
HKDDKDKFFPMSG

SEQ ID NO: 79 - protein, 14 aa, synthetic construct
  REGION 1..14: note = VRVII-204/AAV6
KESAGASNTALDNV

SEQ ID NO: 80 - protein, 13 aa, synthetic construct
  REGION 1..13: note = VRVIII-204/AAV6
AVNLQNSSTDPAT

SEQ ID NO: 81 - protein, 10 aa, synthetic construct
  REGION 1..10: note = VRIX-204/AAV6
NYAKSANVDF

SEQ ID NO: 82 - DNA, 2211 bp, synthetic construct
  misc_feature 1..2211: note = AAV214-AB VP1
  source 1..2211: mol_type = other DNA
atggctgccgatggttatcttccagattggctcgaggacaacctctctgagggcattcgcgagtggtgggcgctgaaacctggagccccgaagcccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagcgggttctcgaaccttttggtctggttgaggaaggtgctaagacggctcctggaaagaaacgtccggtagagcagtcgccacaagagccagactcctcctcgggcatcggcaagacaggccagcagcccgctaaaaagagactcaattttggtcagactggcgactcagagtcagtccccgacccacaacctctcggagaacctccagcaacccccgctgctgtgggacctactacaatggcttcaggcggtggcgcaccaatggcggacaataacgaaggcgccgacggagtgggtaatgcctcaggaaattggcattgcgattccacatggctgggcgacagagtcatcaccaccagcacccgcacctgggccttgcccacctacaataaccacctctacaagcaaatctccagcagcacatctggaggatcttcaaatgacaacgcctacttcggctacagcaccccctgggggtattttgacttcaacagattccactgccacttttcaccacgtgactggcaaagactcatcaacaacaactggggattccgacccaagagactcaacttcaagctctttaacattcaagtcaaagaggttacggacaacaatggagtcaagaccatcgccaataaccttaccagcacggtccaggtcttcacggactcagactatcagctcccgtacgtcctcggctctgcgcaccagggctgcctccctccgttcccggcggacgtgttcatgattccgcagtacggctacctaacgctcaacgacggcagccaggcagtgggacggtcatccttttactgcctggaatatttcccatcgcagatgctgagaacgggcaacaactttaccttcagctacacctttgaggacgttcctttccacagcagctacgctcacagccagagtctggaccgtctcatgaatcctctgattgaccagtacctgtactacttgtctaagactatcaacggatccggccagaatcagcagactctgaagttcagccaaggtgggcctaatacaatggccaatcaggcaaagaactggctgccaggaccctgttaccgccaacaacgcgtctcaacgacaaccgggcaaaacaacaatagcaactttgcctggactgctgggaccaaataccatctgaatggaagaaattcattgatgaatcctggccccgctatggcatcccacaaagagggcgaggaccgtttttttcccctgtccgggtccctgatttttggcaaacaaaatgctgccagagacaatgcggattacagcgatgtcatgctcaccagcgaggaagaaatcaaaaccactaaccctgtggctacagaggaatacggtatcgtggcagataacttgcagcagcaaaacacggctcctcaaattggaactgtcaacagccagggggccttacccggtatggtctggcagaaccgggacgtgtacctgcagggtcccatctgggccaagattcctcacacggacggcaacttccacccgtctccgctgatgggcggctttggcctgaaacatcctccgcctcagatcctgatcaagaacacgcctgtacctgcggatcctccgaccaccttcaaccagtcaaagctgaactctttcatcacgcaatacagcaccggacaggtcagcgtggaaattgaatgggagctgcagaaggaaaacagcaagcgctggaaccccgagatccagtacacctccaactactacaaatctacaagtgtggactttgctgttaatacagaaggcgtgtactctgaaccccaccccattggcacccgttacctcacccgtcccctgtaa

SEQ ID NO: 83 - DNA, 1605 bp, synthetic construct
  misc_feature 1..1605: note = AAV214-AB VP3
  source 1..1605: mol_type = other DNA
atggcttcaggcggtggcgcaccaatggcggacaataacgaaggcgccgacggagtgggtaatgcctcaggaaattggcattgcgattccacatggctgggcgacagagtcatcaccaccagcacccgcacctgggccttgcccacctacaataaccacctctacaagcaaatctccagcagcacatctggaggatcttcaaatgacaacgcctacttcggctacagcaccccctgggggtattttgacttcaacagattccactgccacttttcaccacgtgactggcaaagactcatcaacaacaactggggattccgacccaagagactcaacttcaagctctttaacattcaagtcaaagaggttacggacaacaatggagtcaagaccatcgccaataaccttaccagcacggtccaggtcttcacggactcagactatcagctcccgtacgtcctcggctctgcgcaccagggctgcctccctccgttcccggcggacgtgttcatgattccgcagtacggctacctaacgctcaacgacggcagccaggcagtgggacggtcatccttttactgcctggaatatttcccatcgcagatgctgagaacgggcaacaactttaccttcagctacacctttgaggacgttcctttccacagcagctacgctcacagccagagtctggaccgtctcatgaatcctctgattgaccagtacctgtactacttgtctaagactatcaacggatccggccagaatcagcagactctgaagttcagccaaggtgggcctaatacaatggccaatcaggcaaagaactggctgccaggaccctgttaccgccaacaacgcgtctcaacgacaaccgggcaaaacaacaatagcaactttgcctggactgctgggaccaaataccatctgaatggaagaaattcattgatgaatcctggccccgctatggcatcccacaaagagggcgaggaccgtttttttcccctgtccgggtccctgatttttggcaaacaaaatgctgccagagacaatgcggattacagcgatgtcatgctcaccagcgaggaagaaatcaaaaccactaaccctgtggctacagaggaatacggtatcgtggcagataacttgcagcagcaaaacacggctcctcaaattggaactgtcaacagccagggggccttacccggtatggtctggcagaaccgggacgtgtacctgcagggtcccatctgggccaagattcctcacacggacggcaacttccacccgtctccgctgatgggcggctttggcctgaaacatcctccgcctcagatcctgatcaagaacacgcctgtacctgcggatcctccgaccaccttcaaccagtcaaagctgaactctttcatcacgcaatacagcaccggacaggtcagcgtggaaattgaatgggagctgcagaaggaaaacagcaagcgctggaaccccgagatccagtacacctccaactactacaaatctacaagtgtggactttgctgttaatacagaaggcgtgtactctgaaccccaccccattggcacccgttacctcacccgtcccctgtaa

SEQ ID NO: 84 - protein, 736 aa, synthetic construct
  REGION 1..736: note = AAV214AB VP1
MAADGYLPDWLEDNLSEGIREWWALKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPFGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHQGCLPPFPADVFMIPQYGYLTLNDGSQAVGRSSFYCLEYFPSQMLRTGNNFTFSYTFEDVPFHSSYAHSQSLDRLMNPLIDQYLYYLSKTINGSGQNQQTLKFSQGGPNTMANQAKNWLPGPCYRQQRVSTTTGQNNNSNFAWTAGTKYHLNGRNSLMNPGPAMASHKEGEDRFFPLSGSLIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPHPIGTRYLTRPL

SEQ ID NO: 85 - protein, 599 aa, synthetic construct
  REGION 1..599: note = AAV214AB VP2
MAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHQGCLPPFPADVFMIPQYGYLTLNDGSQAVGRSSFYCLEYFPSQMLRTGNNFTFSYTFEDVPFHSSYAHSQSLDRLMNPLIDQYLYYLSKTINGSGQNQQTLKFSQGGPNTMANQAKNWLPGPCYRQQRVSTTTGQNNNSNFAWTAGTKYHLNGRNSLMNPGPAMASHKEGEDRFFPLSGSLIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPHPIGTRYLTRPL

SEQ ID NO: 86 - protein, 534 aa, synthetic construct
  REGION 1..534: note = AAV214AB VP3
MASGGGAPMADNNEGADGVGNASGNWHCDSTWLGDRVITTSTRTWALPTYNNHLYKQISSSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHQGCLPPFPADVFMIPQYGYLTLNDGSQAVGRSSFYCLEYFPSQMLRTGNNFTFSYTFEDVPFHSSYAHSQSLDRLMNPLIDQYLYYLSKTINGSGQNQQTLKFSQGGPNTMANQAKNWLPGPCYRQQRVSTTTGQNNNSNFAWTAGTKYHLNGRNSLMNPGPAMASHKEGEDRFFPLSGSLIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPHPIGTRYLTRPL

SEQ ID NO: 87 - protein, 8 aa, synthetic construct
  REGION 1..8: note = AAV214AB VR-1 amino acid
SSTSGGSS

SEQ ID NO: 88 - DNA, 6719 bp, synthetic construct
  misc_feature 1..6719: note = pA-CF1
  source 1..6719: mol_type = other DNA
tcctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcatggaggcggtactatgtagatgagaattcaggagcaaactgggaaaagcaactgcttccaaatatttgtgatttttacagtgtagttttggaaaaactcttagcctaccaattcttctaagtgttttaaaatgtgggagccagtacacatgaagttatagagtgttttaatgaggcttaaatatttaccgtaactatgaaatgctacgcatatcatgctgttcaggctccgtggccacgcaactcataccggtagtactcgccaccatgcagagaagccccctggagaaggcctctgtggtgagcaagctgttcttcagctggaccagacccatcctgagaaagggctacagacagagactggagctgtctgacatctaccagatcccctctgtggactctgctgacaacctgtctgagaagctggagagagagtgggacagagagctggccagcaagaagaaccccaagctgatcaatgccctgagaagatgcttcttctggagattcatgttctatggcatcttcctgtacctgggggaggtgaccaaggctgtgcagcccctgctgctgggcagaatcattgccagctatgaccctgacaacaaggaggagagaagcattgccatctacctgggcattggcctgtgcctgctgttcattgtgagaaccctgctgctgcaccctgccatctttggcctgcaccacattggcatgcagatgagaattgccatgttcagcctgatctacaagaagaccctgaagctgagcagcagagtgctggacaagatcagcattggccagctggtgagcctgctgagcaacaacctgaacaagtttgatgagggcctggccctggcccactttgtgtggattgcccccctgcaggtggccctgctgatgggcctgatctgggagctgctgcaggcctctgccttctgtggcctgggcttcctgattgtgctggccctgttccaggctggcctgggcagaatgatgatgaagtacagagaccagagagctggcaagatctctgagagactggtgatcacctctgagatgattgagaacatccagtctgtgaaggcctactgctgggaggaggccatggagaagatgattgagaacctgagacagacagagctgaagctgaccagaaaggctgcctatgtgagatacttcaacagctctgccttcttcttctctggcttctttgtggtgttcctgtctgtgctgccctatgccctgatcaagggcatcatcctgagaaagatcttcaccaccatcagcttctgcattgtgctgagaatggctgtgaccagacagttcccctgggctgtgcagacctggtatgacagcctgggggccatcaacaagatccaggacttcctgcagaagcaggagtacaagaccctggagtacaacctgaccaccacagaggtggtgatggagaatgtgacagccttctgggaggagggctttggggagctgtttgagaaggccaagcagaacaacaacaacagaaagaccagcaatggggatgacagcctgttcttcagcaacttcagcctgctgggcacccctgtgctgaaggacatcaacttcaagattgagagaggccagctgctggctgtggctggcagcacaggggctggcaagaccagcctgctgatgatgatcatgggggagctggagccctctgagggcaagatcaagcactctggcagaatcagcttctgcagccagttcagctggatcatgcctggcaccatcaaggagaacatcatctttggggtgagctatgatgagtacagatacagatctgtgatcaaggcctgccagctggaggaggacatcagcaagtttgctgagaaggacaacattgtgctgggggaggggggcatcaccctgtctgggggccagagagccagaatcagcctggccagagctgtgtacaaggatgctgacctgtacctgctggacagcccctttggctacctggatgtgctgacagagaaggagatctttgagagctgtgtgtgcaagctgatggccaacaagaccagaatcctggtgaccagcaagatggagcacctgaagaaggctgacaagatcctgatcctgcatgagggcagcagctacttctatggcaccttctctgagctgcagaacctgcagcctgacttcagcagcaagctgatgggctgtgacagctttgaccagttctctgctgagagaagaaacagcatcctgacagagaccctgcacagattcagcctggagggggatgcccctgtgagctggacagagaccaagaagcagagcttcaagcagacaggggagtttggggagaagagaaagaacagcatcctgaaccccatcaacagcaccctgcaggccagaagaagacagtctgtgctgaacctgatgacccactctgtgaaccagggccagaacatccacagaaagaccacagccagcaccagaaaggtgagcctggccccccaggccaacctgacagagctggacatctacagcagaagactgagccaggagacaggcctggagatctctgaggagatcaatgaggaggacctgaaggagtgcttctttgatgacatggagagcatccctgctgtgaccacctggaacacctacctgagatacatcacagtgcacaagagcctgatctttgtgctgatctggtgcctggtgatcttcctggctgaggtggctgccagcctggtggtgctgtggctgctgggcaacacccccctgcaggacaagggcaacagcacccacagcagaaacaacagctatgctgtgatcatcaccagcaccagcagctactatgtgttctacatctatgtgggggtggctgacaccctgctggccatgggcttcttcagaggcctgcccctggtgcacaccctgatcacagtgagcaagatcctgcaccacaagatgctgcactctgtgctgcaggcccccatgagcaccctgaacaccctgaaggctgggggcatcctgaacagattcagcaaggacattgccatcctggatgacctgctgcccctgaccatctttgacttcatccagctgctgctgattgtgattggggccattgctgtggtggctgtgctgcagccctacatctttgtggccacagtgcctgtgattgtggccttcatcatgctgagagcctacttcctgcagaccagccagcagctgaagcagctggagtctgagggcagaagccccatcttcacccacctggtgaccagcctgaagggcctgtggaccctgagagcctttggcagacagccctactttgagaccctgttccacaaggccctgaacctgcacacagccaactggttcctgtacctgagcaccctgagatggttccagatgagaattgagatgatctttgtgatcttcttcattgctgtgaccttcatcagcatcctgaccacaggggagggggagggcagagtgggcatcatcctgaccctggccatgaacatcatgagcaccctgcagtgggctgtgaacagcagcattgatgtggacagcctgatgagatctgtgagcagagtgttcaagttcattgacatgcccacagagggcaagcccaccaagagcaccaagccctacaagaatggccagctgagcaaggtgatgatcattgagaacagccatgtgaagaaggatgacatctggccctctgggggccagatgacagtgaaggacctgacagccaagtacacagaggggggcaatgccatcctggagaacatcagcttcagcatcagccctggccagagagtgggcctgctgggcagaacaggctctggcaagagcaccctgctgtctgccttcctgagactgctgaacacagagggggagatccagattgatggggtgagctgggacagcatcaccctgcagcagtggagaaaggcctttggggtgatcccccagaaggtgttcatcttctctggcaccttcagaaagaacctggacccctatgagcagtggtctgaccaggagatctggaaggtggctgatgaggtgggcctgagatctgtgattgagcagttccctggcaagctggactttgtgctggtggatgggggctgtgtgctgagccatggccacaagcagctgatgtgcctggccagatctgtgctgagcaaggccaagatcctgctgctggatgagccctctgcccacctggaccctgtgacctaccagatcatcagaagaaccctgaagcaggcctttgctgactgcacagtgatcctgtgtgagcacagaattgaggccatgctggagtgccagcagttcctggtgattgaggagaacaaggtgagacagtatgacagcatccagaagctgctgaatgagagaagcctgttcagacaggccatcagcccctctgacagagtgaagctgttcccccacagaaacagcagcaagtgcaagagcaagccccagattgctgccctgaaggaggagaccgaggaggaggtgcaggacaccagactgtaaataaaatacgaaatggatctgaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaattaattaaggcgatgaacggtaatcgtaaaactagcatgtcaatcatatgtaccccggttgataatcagaaaagccccaaaaacaggaagattgtataagcattaattaatttaaatacatggacatgtcagaattggttaattggttgtaacactgacccctatttgtttatttttctaaatacattcaaatatgtatccgctcatgagacaataaccctgataaatgcttcaataatattgaaaaaggaagaatatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgccacttccgaccatcaagcattttatccgtactcctgatgatgcatggttactcaccactgcgatccccggaaaaacagcgttccaggtattagaagaatatcctgattcaggtgaaaatattgttgatgcgctggcagtgttcctgcgccggttgcactcgattcctgtttgtaattgtccttttaacagcgatcgcgtatttcgcctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaaaagcagagcattacgctgacttgacgggacggcgcaagctcatgaccaaaatcccttaacgtgagttacgcgcgcgtcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttagcccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgtttaaaccatg

SEQ ID NO: 89 - DNA, 6751 bp, synthetic construct
  misc_feature 1..6751: note = pA-CF3
  source 1..6751: mol_type = other DNA
tcctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcatggaggcggtactatgtagatgagaattcaggagcaaactgggaaaagcaactgcttccaaatatttgtgatttttacagtgtagttttggaaaaactcttagcctaccaattcttctaagtgttttaaaatgtgggagccagtacacatgaagttatagagtgttttaatgaggcttaaatatttaccgtaactatgaaatgctacgcatatcatgctgttcaggctccgtggccacgcaactcataccggtagtactcgccaccatgcagagaagccccctggagaaggcctctgtggtgagcaagctgttcttcagctggaccagacccatcctgagaaagggctacagacagagactggagctgtctgacatctaccagatcccctctgtggactctgctgacaacctgtctgagaagctggagagagagtgggacagagagctggccagcaagaagaaccccaagctgatcaatgccctgagaagatgcttcttctggagattcatgttctatggcatcttcctgtacctgggggaggtgaccaaggctgtgcagcccctgctgctgggcagaatcattgccagctatgaccctgacaacaaggaggagagaagcattgccatctacctgggcattggcctgtgcctgctgttcattgtgagaaccctgctgctgcaccctgccatctttggcctgcaccacattggcatgcagatgagaattgccatgttcagcctgatctacaagaagaccctgaagctgagcagcagagtgctggacaagatcagcattggccagctggtgagcctgctgagcaacaacctgaacaagtttgatgagggcctggccctggcccactttgtgtggattgcccccctgcaggtggccctgctgatgggcctgatctgggagctgctgcaggcctctgccttctgtggcctgggcttcctgattgtgctggccctgttccaggctggcctgggcagaatgatgatgaagtacagagaccagagagctggcaagatctctgagagactggtgatcacctctgagatgattgagaacatccagtctgtgaaggcctactgctgggaggaggccatggagaagatgattgagaacctgagacagacagagctgaagctgaccagaaaggctgcctatgtgagatacttcaacagctctgccttcttcttctctggcttctttgtggtgttcctgtctgtgctgccctatgccctgatcaagggcatcatcctgagaaagatcttcaccaccatcagcttctgcattgtgctgagaatggctgtgaccagacagttcccctgggctgtgcagacctggtatgacagcctgggggccatcaacaagatccaggacttcctgcagaagcaggagtacaagaccctggagtacaacctgaccaccacagaggtggtgatggagaatgtgacagccttctgggaggagggctttggggagctgtttgagaaggccaagcagaacaacaacaacagaaagaccagcaatggggatgacagcctgttcttcagcaacttcagcctgctgggcacccctgtgctgaaggacatcaacttcaagattgagagaggccagctgctggctgtggctggcagcacaggggctggcaagaccagcctgctgatgatgatcatgggggagctggagccctctgagggcaagatcaagcactctggcagaatcagcttctgcagccagttcagctggatcatgcctggcaccatcaaggagaacatcatctttggggtgagctatgatgagtacagatacagatctgtgatcaaggcctgccagctggaggaggacatcagcaagtttgctgagaaggacaacattgtgctgggggaggggggcatcaccctgtctgggggccagagagccagaatcagcctggccagagctgtgtacaaggatgctgacctgtacctgctggacagcccctttggctacctggatgtgctgacagagaaggagatctttgagagctgtgtgtgcaagctgatggccaacaagaccagaatcctggtgaccagcaagatggagcacctgaagaaggctgacaagatcctgatcctgcatgagggcagcagctacttctatggcaccttctctgagctgcagaacctgcagcctgacttcagcagcaagctgatgggctgtgacagctttgaccagttctctgctgagagaagaaacagcatcctgacagagaccctgcacagattcagcctggagggggatgcccctgtgagctggacagagaccaagaagcagagcttcaagcagacaggggagtttggggagaagagaaagaacagcatcctgaaccccatcaacagcaccctgcaggccagaagaagacagtctgtgctgaacctgatgacccactctgtgaaccagggccagaacatccacagaaagaccacagccagcaccagaaaggtgagcctggccccccaggccaacctgacagagctggacatctacagcagaagactgagccaggagacaggcctggagatctctgaggagatcaatgaggaggacctgaaggagtgcttctttgatgacatggagagcatccctgctgtgaccacctggaacacctacctgagatacatcacagtgcacaagagcctgatctttgtgctgatctggtgcctggtgatcttcctggctgaggtggctgccagcctggtggtgctgtggctgctgggcaacacccccctgcaggacaagggcaacagcacccacagcagaaacaacagctatgctgtgatcatcaccagcaccagcagctactatgtgttctacatctatgtgggggtggctgacaccctgctggccatgggcttcttcagaggcctgcccctggtgcacaccctgatcacagtgagcaagatcctgcaccacaagatgctgcactctgtgctgcaggcccccatgagcaccctgaacaccctgaaggctgggggcatcctgaacagattcagcaaggacattgccatcctggatgacctgctgcccctgaccatctttgacttcatccagctgctgctgattgtgattggggccattgctgtggtggctgtgctgcagccctacatctttgtggccacagtgcctgtgattgtggccttcatcatgctgagagcctacttcctgcagaccagccagcagctgaagcagctggagtctgagggcagaagccccatcttcacccacctggtgaccagcctgaagggcctgtggaccctgagagcctttggcagacagccctactttgagaccctgttccacaaggccctgaacctgcacacagccaactggttcctgtacctgagcaccctgagatggttccagatgagaattgagatgatctttgtgatcttcttcattgctgtgaccttcatcagcatcctgaccacaggggagggggagggcagagtgggcatcatcctgaccctggccatgaacatcatgagcaccctgcagtgggctgtgaacagcagcattgatgtggacagcctgatgagatctgtgagcagagtgttcaagttcattgacatgcccacagagggcaagcccaccaagagcaccaagccctacaagaatggccagctgagcaaggtgatgatcattgagaacagccatgtgaagaaggatgacatctggccctctgggggccagatgacagtgaaggacctgacagccaagtacacagaggggggcaatgccatcctggagaacatcagcttcagcatcagccctggccagagagtgggcctgctgggcagaacaggctctggcaagagcaccctgctgtctgccttcctgagactgctgaacacagagggggagatccagattgatggggtgagctgggacagcatcaccctgcagcagtggagaaaggcctttggggtgatcccccagaaggtgttcatcttctctggcaccttcagaaagaacctggacccctatgagcagtggtctgaccaggagatctggaaggtggctgatgaggtgggcctgagatctgtgattgagcagttccctggcaagctggactttgtgctggtggatgggggctgtgtgctgagccatggccacaagcagctgatgtgcctggccagatctgtgctgagcaaggccaagatcctgctgctggatgagccctctgcccacctggaccctgtgacctaccagatcatcagaagaaccctgaagcaggcctttgctgactgcacagtgatcctgtgtgagcacagaattgaggccatgctggagtgccagcagttcctggtgattgaggagaacaaggtgagacagtatgacagcatccagaagctgctgaatgagagaagcctgttcagacaggccatcagcccctctgacagagtgaagctgttcccccacagaaacagcagcaagtgcaagagcaagccccagattgctgccctgaaggaggagaccgaggaggaggtgcaggacaccagactgtaaataaatatctttattttcattacatctgtgtgttggttttttgtgtggatctgaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaattaattaaggcgatgaacggtaatcgtaaaactagcatgtcaatcatatgtaccccggttgataatcagaaaagccccaaaaacaggaagattgtataagcattaattaatttaaatacatggacatgtcagaattggttaattggttgtaacactgacccctatttgtttatttttctaaatacattcaaatatgtatccgctcatgagacaataaccctgataaatgcttcaataatattgaaaaaggaagaatatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgccacttccgaccatcaagcattttatccgtactcctgatgatgcatggttactcaccactgcgatccccggaaaaacagcgttccaggtattagaagaatatcctgattcaggtgaaaatattgttgatgcgctggcagtgttcctgcgccggttgcactcgattcctgtttgtaattgtccttttaacagcgatcgcgtatttcgcctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaaaagcagagcattacgctgacttgacgggacggcgcaagctcatgaccaaaatcccttaacgtgagttacgcgcgcgtcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttagcccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgtttaaaccatg

SEQ ID NO: 90 - DNA, 6603 bp, synthetic construct
  misc_feature 1..6603: note = pA-CF5
  source 1..6603: mol_type = other DNA
tcctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcaatatttgcatgtcgctatgtgttctgggaaatcaccataaacgtgaaatgtctttggatttgggaatcttcgaagttctgtatgagaccacagatctccaccggtagtactcgccaccatgcagagaagccccctggagaaggcctctgtggtgagcaagctgttcttcagctggaccagacccatcctgagaaagggctacagacagagactggagctgtctgacatctaccagatcccctctgtggactctgctgacaacctgtctgagaagctggagagagagtgggacagagagctggccagcaagaagaaccccaagctgatcaatgccctgagaagatgcttcttctggagattcatgttctatggcatcttcctgtacctgggggaggtgaccaaggctgtgcagcccctgctgctgggcagaatcattgccagctatgaccctgacaacaaggaggagagaagcattgccatctacctgggcattggcctgtgcctgctgttcattgtgagaaccctgctgctgcaccctgccatctttggcctgcaccacattggcatgcagatgagaattgccatgttcagcctgatctacaagaagaccctgaagctgagcagcagagtgctggacaagatcagcattggccagctggtgagcctgctgagcaacaacctgaacaagtttgatgagggcctggccctggcccactttgtgtggattgcccccctgcaggtggccctgctgatgggcctgatctgggagctgctgcaggcctctgccttctgtggcctgggcttcctgattgtgctggccctgttccaggctggcctgggcagaatgatgatgaagtacagagaccagagagctggcaagatctctgagagactggtgatcacctctgagatgattgagaacatccagtctgtgaaggcctactgctgggaggaggccatggagaagatgattgagaacctgagacagacagagctgaagctgaccagaaaggctgcctatgtgagatacttcaacagctctgccttcttcttctctggcttctttgtggtgttcctgtctgtgctgccctatgccctgatcaagggcatcatcctgagaaagatcttcaccaccatcagcttctgcattgtgctgagaatggctgtgaccagacagttcccctgggctgtgcagacctggtatgacagcctgggggccatcaacaagatccaggacttcctgcagaagcaggagtacaagaccctggagtacaacctgaccaccacagaggtggtgatggagaatgtgacagccttctgggaggagggctttggggagctgtttgagaaggccaagcagaacaacaacaacagaaagaccagcaatggggatgacagcctgttcttcagcaacttcagcctgctgggcacccctgtgctgaaggacatcaacttcaagattgagagaggccagctgctggctgtggctggcagcacaggggctggcaagaccagcctgctgatgatgatcatgggggagctggagccctctgagggcaagatcaagcactctggcagaatcagcttctgcagccagttcagctggatcatgcctggcaccatcaaggagaacatcatctttggggtgagctatgatgagtacagatacagatctgtgatcaaggcctgccagctggaggaggacatcagcaagtttgctgagaaggacaacattgtgctgggggaggggggcatcaccctgtctgggggccagagagccagaatcagcctggccagagctgtgtacaaggatgctgacctgtacctgctggacagcccctttggctacctggatgtgctgacagagaaggagatctttgagagctgtgtgtgcaagctgatggccaacaagaccagaatcctggtgaccagcaagatggagcacctgaagaaggctgacaagatcctgatcctgcatgagggcagcagctacttctatggcaccttctctgagctgcagaacctgcagcctgacttcagcagcaagctgatgggctgtgacagctttgaccagttctctgctgagagaagaaacagcatcctgacagagaccctgcacagattcagcctggagggggatgcccctgtgagctggacagagaccaagaagcagagcttcaagcagacaggggagtttggggagaagagaaagaacagcatcctgaaccccatcaacagcaccctgcaggccagaagaagacagtctgtgctgaacctgatgacccactctgtgaaccagggccagaacatccacagaaagaccacagccagcaccagaaaggtgagcctggccccccaggccaacctgacagagctggacatctacagcagaagactgagccaggagacaggcctggagatctctgaggagatcaatgaggaggacctgaaggagtgcttctttgatgacatggagagcatccctgctgtgaccacctggaacacctacctgagatacatcacagtgcacaagagcctgatctttgtgctgatctggtgcctggtgatcttcctggctgaggtggctgccagcctggtggtgctgtggctgctgggcaacacccccctgcaggacaagggcaacagcacccacagcagaaacaacagctatgctgtgatcatcaccagcaccagcagctactatgtgttctacatctatgtgggggtggctgacaccctgctggccatgggcttcttcagaggcctgcccctggtgcacaccctgatcacagtgagcaagatcctgcaccacaagatgctgcactctgtgctgcaggcccccatgagcaccctgaacaccctgaaggctgggggcatcctgaacagattcagcaaggacattgccatcctggatgacctgctgcccctgaccatctttgacttcatccagctgctgctgattgtgattggggccattgctgtggtggctgtgctgcagccctacatctttgtggccacagtgcctgtgattgtggccttcatcatgctgagagcctacttcctgcagaccagccagcagctgaagcagctggagtctgagggcagaagccccatcttcacccacctggtgaccagcctgaagggcctgtggaccctgagagcctttggcagacagccctactttgagaccctgttccacaaggccctgaacctgcacacagccaactggttcctgtacctgagcaccctgagatggttccagatgagaattgagatgatctttgtgatcttcttcattgctgtgaccttcatcagcatcctgaccacaggggagggggagggcagagtgggcatcatcctgaccctggccatgaacatcatgagcaccctgcagtgggctgtgaacagcagcattgatgtggacagcctgatgagatctgtgagcagagtgttcaagttcattgacatgcccacagagggcaagcccaccaagagcaccaagccctacaagaatggccagctgagcaaggtgatgatcattgagaacagccatgtgaagaaggatgacatctggccctctgggggccagatgacagtgaaggacctgacagccaagtacacagaggggggcaatgccatcctggagaacatcagcttcagcatcagccctggccagagagtgggcctgctgggcagaacaggctctggcaagagcaccctgctgtctgccttcctgagactgctgaacacagagggggagatccagattgatggggtgagctgggacagcatcaccctgcagcagtggagaaaggcctttggggtgatcccccagaaggtgttcatcttctctggcaccttcagaaagaacctggacccctatgagcagtggtctgaccaggagatctggaaggtggctgatgaggtgggcctgagatctgtgattgagcagttccctggcaagctggactttgtgctggtggatgggggctgtgtgctgagccatggccacaagcagctgatgtgcctggccagatctgtgctgagcaaggccaagatcctgctgctggatgagccctctgcccacctggaccctgtgacctaccagatcatcagaagaaccctgaagcaggcctttgctgactgcacagtgatcctgtgtgagcacagaattgaggccatgctggagtgccagcagttcctggtgattgaggagaacaaggtgagacagtatgacagcatccagaagctgctgaatgagagaagcctgttcagacaggccatcagcccctctgacagagtgaagctgttcccccacagaaacagcagcaagtgcaagagcaagccccagattgctgccctgaaggaggagaccgaggaggaggtgcaggacaccagactgtaaataaatatctttattttcattacatctgtgtgttggttttttgtgtggatctgaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaattaattaaggcgatgaacggtaatcgtaaaactagcatgtcaatcatatgtaccccggttgataatcagaaaagccccaaaaacaggaagattgtataagcattaattaatttaaatacatggacatgtcagaattggttaattggttgtaacactgacccctatttgtttatttttctaaatacattcaaatatgtatccgctcatgagacaataaccctgataaatgcttcaataatattgaaaaaggaagaatatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgccacttccgaccatcaagcattttatccgtactcctgatgatgcatggttactcaccactgcgatccccggaaaaacagcgttccaggtattagaagaatatcctgattcaggtgaaaatattgttgatgcgctggcagtgttcctgcgccggttgcactcgattcctgtttgtaattgtccttttaacagcgatcgcgtatttcgcctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaaaagcagagcattacgctgacttgacgggacggcgcaagctcatgaccaaaatcccttaacgtgagttacgcgcgcgtcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttagcccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgtttaaaccatg

SEQ ID NO: 91 - DNA, 7519 bp, synthetic construct
  misc_feature 1..7519: note = pA-CF7
  source 1..7519: mol_type = other DNA
tcctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcaatatttgcatgtcgctatgtgttctgggaaatcaccataaacgtgaaatgtctttggatttgggaatcttcgaagttctgtatgagaccacagatctccaccggtagtactcgccaccatgcagagaagccccctggagaaggcctctgtggtgagcaagctgttcttcccccctggagaaggcctctgtggtgagcaagctgttcttcagctggaccagacccatcctgagaaagggctacagacagagactggagctgtctgacatctaccagatcccctctgtggactctgctgacaacctgtctgagaagctggagagagagtgggacagagagctggccagcaagaagaaccccaagctgatcaatgccctgagaagatgcttcttctggagattcatgttctatggcatcttcctgtacctgggggaggtgaccaaggctgtgcagcccctgctgctgggcagaatcattgccagctatgacccagcccctgctgctgggcagaatcattgccagctatgaccctgacaacaaggaggagagaagcattgccatctacctgggcattggcctgtgcctgctgttcattgtgagaaccctgctgctgcaccctgccatctttggcctgcaccacattggcatgcagatgagaattgccatgttcagcctgatctacaagaagaccctgaagctgagcagcagagtgctggacaagatcagcattggccagctggtgagcctgctgagcaacaacctgaacaagtttgatgagggcctggccctggcccactttgtgtggattgcttgatgagggcctggccctggcccactttgtgtggattgcccccctgcaggtggccctgctgatgggcctgatctgggagctgctgcaggcctctgccttctgtggcctgggcttcctgattgtgctggccctgttccaggctggcctgggcagaatgatgatgaagtacagagaccagagagctggcaagatctctgagagactggtgatcacctctgagatgattgagaacatccagtctgtgaaggcctactgctgggaggaggccatggagaagatgattgagaacctgagacagacagagctgaagctgaccagaaaggctgcctatgtgagatacttcaacagctctgccttcttcttctctggcttctttgtggtgttcctgtctgtgctgcctctgccttcttcttctctggcttctttgtggtgttcctgtctgtgctgccctatgccctgatcaagggcatcatcctgagaaagatcttcaccaccatcagcttctgcattgtgctgagaatggctgtgaccagacagttcccctgggctgtgcagacctggtatgacagcctgggggccatcaacaagatccaggacttcctgcagaagcaggagtacaagaccctggagtacaacctgaccaccacagaggtggtgatggagaatgtgacagccttctgggaggagggctttggggagctgtttgagaaggccaagcagaacaacaacaacagaaagaccagcaatggggatgacagcctgttcttcagcaacttcagcctgctgggcacccctgtgcggatgacagcctgttcttcagcaacttcagcctgctgggcacccctgtgctgaaggacatcaacttcaagattgagagaggccagctgctggctgtggctggcagcacaggggctggcaagaccagcctgctgatgatgatcatgggggagctggagccctctgagggcaagatcaagcactctggcagaatcagcttctgcagccagttcagctggatcatgcctggcaccatcaaggagaacatcatctttggggtgagctatgatgagtacagatacagatctgtgatcaaggcctgccagctggaggaggacatcaagatctgtgatcaaggcctgccagctggaggaggacatcagcaagtttgctgagaaggacaacattgtgctgggggaggggggcatcaccctgtctgggggccagagagccagaatcagcctggccagagctgtgtacaaggatgctgacctgtacctgctggacagcccctttggctacctggatgtgctgacagagaaggagatctttgagagctgtgtgtgcaagctgatggccaacaagaccagaatcctggtgaccagcaagatggagcacctgaagaaggctgacaagatcctgatcctgcatgagggcagcagagaaggctgacaagatcctgatcctgcatgagggcagcagctacttctatggcaccttctctgagctgcagaacctgcagcctgacttcagcagcaagctgatgggctgtgacagctttgaccagttctctgctgagagaagaaacagcatcctgacagagaccctgcacagattcagcctggagggggatgcccctgtgagctggacagagaccaagaagcagagcttcaagcagacaggggagtttggggagaagagaaagaacagcatcctgaaccccatcaacagcatcagaaagttcagcattgtgcagaagacccatcaacagcatcagaaagttcagcattgtgcagaagacccccctgcagatgaatggcattgaggaggactctgatgagcccctggagagaagactgagcctggtgcctgactctgagcagggggaggccatcctgcccagaatctctgtgatcagcacaggccccaccctgcaggccagaagaagacagtctgtgctgaacctgatgacccactctgtgaaccagggccagaacatccaccactctgtgaaccagggccagaacatccacagaaagaccacagccagcaccagaaaggtgagcctggccccccaggccaacctgacagagctggacatctacagcagaagactgagccaggagacaggcctggagatctctgaggagatcaatgaggaggacctgaaggagtgcttctttgatgacatggagagcatccctgctgtgaccacctggaacacctacctgagatacatcacagtgcacaagagcctgatctttgtgctgatctggtgcctggtgatcttcctggctgaggtggctgccagcctggtggtgcgtgatcttcctggctgaggtggctgccagcctggtggtgctgtggctgctgggcaacacccccctgcaggacaagggcaacagcacccacagcagaaacaacagctatgctgtgatcatcaccagcaccagcagctactatgtgttctacatctatgtgggggtggctgacaccctgctggccatgggcttcttcagaggcctgcccctggtgcacaccctgatcacagtgagcaagatcctgcaccacaagatgctgcactctgtgctgcaggcccccatgagcaccctgaacaccctgaaggctgggggcatcctgaatgagcaccctgaacaccctgaaggctgggggcatcctgaacagattcagcaaggacattgccatcctggatgacctgctgcccctgaccatctttgacttcatccagctgctgctgattgtgattggggccattgctgtggtggctgtgctgcagccctacatctttgtggccacagtgcctgtgattgtggccttcatcatgctgagagcctacttcctgcagaccagccagcagctgaagcagctggagtctgagggcagaagccccatcttcacccacctggtgaccagcctgaagggcctgtggaccctgagagcccctggtgaccagcctgaagggcctgtggaccctgagagcctttggcagacagccctactttgagaccctgttccacaaggccctgaacctgcacacagccaactggttcctgtacctgagcaccctgagatggttccagatgagaattgagatgatctttgtgatcttcttcattgctgtgaccttcatcagcatcctgaccacaggggagggggagggcagagtgggcatcatcctgaccctggccatgaacatcatgagcaccctgcagtgggctgtgaacagcagcattgatgtggacagcctgatgagatctgtgagcagagtgttcaagttcattgacatgcccacagagggcaagcccaccaagagcaccaagccctacaagaatggccagctgcagagggcaagcccaccaagagcaccaagccctacaagaatggccagctgagcaaggtgatgatcattgagaacagccatgtgaagaaggatgacatctggccctctgggggccagatgacagtgaaggacctgacagccaagtacacagaggggggcaatgccatcctggagaacatcagcttcagcatcagccctggccagagagtgggcctgctgggcagaacaggctctggcaagagcaccctgctgtctgccttcctgagactgctgaacacagagggggagatccagattgatggggtgagctgggacagcatcaccctgcagcagtggagaaaggcctttggggtgatcccccagaaggtgttcatcttctctggcaccttcagaaagaacctgtgatcccccagaaggtgttcatcttctctggcaccttcagaaagaacctggacccctatgagcagtggtctgaccaggagatctggaaggtggctgatgaggtgggcctgagatctgtgattgagcagttccctggcaagctggactttgtgctggtggatgggggctgtgtgctgagccatggccacaagcagctgatgtgcctggccagatctgtgctgagcaaggccaagatcctgctgctggatgagccctctgcccacctggaccctgtgacctaccagatcatcagaagaaccctgaagcaggcctttgctgaaccagatcatcagaagaaccctgaagcaggcctttgctgactgcacagtgatcctgtgtgagcacagaattgaggccatgctggagtgccagcagttcctggtgattgaggagaacaaggtgagacagtatgacagcatccagaagctgctgaatgagagaagcctgttcagacaggccatcagcccctctgacagagtgaagctgttcccccacagaaacagcagcaagtgcaagagcaagccccagattgctgccctgaaggaggagaccgaggaggaggtgcaggacaccagactgtaaataaatatctttattttcattacatctgtgtgttggttttttgtgtggatctgaggaacccctagtgatggagttggccactccctctctgcgcgctcatctgaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaattaattaaggcgatgaacggtaatcgtaaaactagcatgtcaatcatatgtaccccggttgataatcagaaaagccccaaaaacaggaagattgtataagcattaattaatttaaatacatggacatgtcagaattggttaattggttgtaacactgacccctatttgtttatttttctaaatacattcaaatatgtatccgctcatgagacaataaccctgataaatgcttcaataatattgaaaaaggaagaatatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgccacttccgaccatcaagcattttatccgtactcctgatgatgcatggttactcaccactgcgatccccggaaaaacagcgttccaggtattagaagaatatcctgattcaggtgaaaatattgttgatgcgctggcagtgttcctgcgccggttgcactcgattcctgtttgtaattgtccttttaacagcgatcgcgtatttcgcctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaaaagcagagcattacgctgacttgacgggacggcgcaagctcatgaccaaaatcccttaacgtgagttacgcgcgcgtcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttagcccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggaccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgtttaaaccatg

SEQ ID NO: 92 - DNA, 11577 bp, synthetic construct
  misc_feature 1..11577: note = pHELPK plasmid DNA
  source 1..11577: mol_type = other DNA
ggtacccaactccatgcttaacagtccccaggtacagcccaccctgcgtcgcaaccaggaacagctctacagcttcctggagcgccactcgccctacttccgcagccacagtgcgcagattaggagcgccacttctttttgtcacttgaaaaacatgtaaaaataatgtactaggagacactttcaataaaggcaaatgtttttatttgtacactctcgggtgattatttaccccccacccttgccgtctgcgccgtttaaaaatcaaaggggttctgccgcgcatcgctatgcgccactggcagggacacgttgcgatactggtgtttagtgctccacttaaactcaggcacaaccatccgcggcagctcggtgaagttttcactccacaggctgcgcaccatcaccaacgcgtttagcaggtcgggcgccgatatcttgaagtcgcagttggggcctccgccctgcgcgcgcgagttgcgatacacagggttgcagcactggaacactatcagcgccgggtggtgcacgctggccagcacgctcttgtcggagatcagatccgcgtccaggtcctccgcgttgctcagggcgaacggagtcaactttggtagctgccttcccaaaaagggtgcatgcccaggctttgagttgcactcgcaccgtagtggcatcagaaggtgaccgtgcccggtctgggcgttaggatacagcgcctgcatgaaagccttgatctgcttaaaagccacctgagcctttgcgccttcagagaagaacatgccgcaagacttgccggaaaactgattggccggacaggccgcgtcatgcacgcagcaccttgcgtcggtgttggagatctgcaccacatttcggccccaccggttcttcacgatcttggccttgctagactgctccttcagcgcgcgctgcccgttttcgctcgtcacatccatttcaatcacgtgctccttatttatcataatgctcccgtgtagacacttaagctcgccttcgatctcagcgcagcggtgcagccacaacgcgcagcccgtgggctcgtggtgcttgtaggttacctctgcaaacgactgcaggtacgcctgcaggaatcgccccatcatcgtcacaaaggtcttgttgctggtgaaggtcagctgcaacccgcggtgctcctcgtttagccaggtcttgcatacggccgccagagcttccacttggtcaggcagtagcttgaagtttgcctttagatcgttatccacgtggtacttgtccatcaacgcgcgcgcagcctccatgcccttctcccacgcagacacgatcggcaggctcagcgggtttatcaccgtgctttcactttccgcttcactggactcttccttttcctcttgcgtccgcataccccgcgccactgggtcgtcttcattcagccgccgcaccgtgcgcttacctcccttgccgtgcttgattagcaccggtgggttgctgaaacccaccatttgtagcgccacatcttctctttcttcctcgctgtccacgatcacctctggggatggcgggcgctcgggcttgggagaggggcgcttctttttctttttggacgcaatggccaaatccgccgtcgaggtcgatggccgcgggctgggtgtgcgcggcaccagcgcatcttgtgacgagtcttcttcgtcctcggactcgagacgccgcctcagccgcttttttgggggcgcgcggggaggcggcggcgacggcgacggggacgacacgtcctccatggttggtggacgtcgcgccgcaccgcgtccgcgctcgggggtggtttcgcgctgctcctcttcccgactggccatttccttctcctataggcagaaaaagatcatggagtcagtcgagaaggaggacagcctaaccgccccctttgagttcgccaccaccgcctccaccgatgccgccaacgcgcctaccaccttccccgtcgaggcacccccgcttgaggaggaggaagtgattatcgagcaggacccaggttttgtaagcgaagacgacgaggatcgctcagtaccaacagaggataaaaagcaagaccaggacgacgcagaggcaaacgaggaacaagtcgggcggggggaccaaaggcatggcgactacctagatgtgggagacgacgtgctgttgaagcatctgcagcgccagtgcgccattatctgcgacgcgttgcaagagcgcagcgatgtgcccctcgccatagcggatgtcagccttgcctacgaacgccacctgttctcaccgcgcgtaccccccaaacgccaagaaaacggcacatgcgagcccaacccgcgcctcaacttctaccccgtatttgccgtgccagaggtgcttgccacctatcacatctttttccaaaactgcaagatacccctatcctgccgtgccaaccgcagccgagcggacaagcagctggccttgcggcagggcgctgtcatacctgatatcgcctcgctcgacgaagtgccaaaaatctttgagggtcttggacgcgacgagaaacgcgcggcaaacgctctgcaacaagaaaacagcgaaaatgaaagtcactgtggagtgctggtggaacttgagggtgacaacgcgcgcctagccgtgctgaaacgcagcatcgaggtcacccactttgcctacccggcacttaacctaccccccaaggttatgagcacagtcatgagcgagctgatcgtgcgccgtgcacgacccctggagagggatgcaaacttgcaagaacaaaccgaggagggcctacccgcagttggcgatgagcagctggcgcgctggcttgagacgcgcgagcctgccgacttggaggagcgacgcaagctaatgatggccgcagtgcttgttaccgtggagcttgagtgcatgcagcggttctttgctgacccggagatgcagcgcaagctagaggaaacgttgcactacacctttcgccagggctacgtgcgccaggcctgcaaaatttccaacgtggagctctgcaacctggtctcctaccttggaattttgcacgaaaaccgcctcgggcaaaacgtgcttcattccacgctcaagggcgaggcgcgccgcgactacgtccgcgactgcgtttacttatttctgtgctacacctggcaaacggccatgggcgtgtggcagcaatgcctggaggagcgcaacctaaaggagctgcagaagctgctaaagcaaaacttgaaggacctatggacggccttcaacgagcgctccgtggccgcgcacctggcggacattatcttccccgaacgcctgcttaaaaccctgcaacagggtctgccagacttcaccagtcaaagcatgttgcaaaactttaggaactttatcctagagcgttcaggaattctgcccgccacctgctgtgcgcttcctagcgactttgtgcccattaagtaccgtgaatgccctccgccgctttggggtcactgctaccttctgcagctagccaactaccttgcctaccactccgacatcatggaagacgtgagcggtgacggcctactggagtgtcactgtcgctgcaacctatgcaccccgcaccgctccctggtctgcaattcgcaactgcttagcgaaagtcaaattatcggtacctttgagctgcagggtccctcgcctgacgaaaagtccgcggctccggggttgaaactcactccggggctgtggacgtcggcttaccttcgcaaatttgtacctgaggactaccacgcccacgagattaggttctacgaagaccaatcccgcccgccaaatgcggagcttaccgcctgcgtcattacccagggccacatccttggccaattgcaagccatcaacaaagcccgccaagagtttctgctacgaaagggacggggggtttacctggacccccagtccggcgaggagctcaacccaatccccccgccgccgcagccctatcagcagccgcgggcccttgcttcccaggatggcacccaaaaagaagctgcagctgccgccgccgccacccacggacgaggaggaatactgggacagtcaggcagaggaggttttggacgaggaggaggagatgatggaagactgggacagcctagacgaagcttccgaggccgaagaggtgtcagacgaaacaccgtcaccctcggtcgcattcccctcgccggcgccccagaaattggcaaccgttcccagcatcgctacaacctccgctcctcaggcgccgccggcactgcctgttcgccgacccaaccgtagatgggacaccactggaaccagggccggtaagtctaagcagccgccgccgttagcccaagagcaacaacagcgccaaggctaccgctcgtggcgcgggcacaagaacgccatagttgcttgcttgcaagactgtgggggcaacatctccttcgcccgccgctttcttctctaccatcacggcgtggccttcccccgtaacatcctgcattactaccgtcatctctacagcccctactgcaccggcggcagcggcagcggcagcaacagcagcggtcacacagaagcaaaggcgaccggatagcaagactctgacaaagcccaagaaatccacagcggcggcagcagcaggaggaggagcgctgcgtctggcgcccaacgaacccgtatcgacccgcgagcttagaaataggatttttcccactctgtatgctatatttcaacaaagcaggggccaagaacaagagctgaaaataaaaaacaggtctctgcgctccctcacccgcagctgcctgtatcacaaaagcgaagatcagcttcggcgcacgctggaagacgcggaggctctcttcagcaaatactgcgcgctgactcttaaggactagtttcgcgccctttctcaaatttaagcgcgaaaactacgtcatctccagcggccacacccggcgccagcacctgtcgtcagcgccattatgagcaaggaaattcccacgccctacatgtggagttaccagccacaaatgggacttgcggctggagctgcccaagactactcaacccgaataaactacatgagcgcgggaccccacatgatatcccgggtcaacggaatccgcgcccaccgaaaccgaattctcctcgaacaggcggctattaccaccacacctcgtaataaccttaatccccgtagttggcccgctgccctggtgtaccaggaaagtcccgctcccaccactgtggtacttcccagagacgcccaggccgaagttcagatgactaactcaggggcgcagcttgcgggcggctttcgtcacagggtgcggtcgcccgggcgttttagggcggagtaacttgcatgtattgggaattgtagtttttttaaaatgggaagtgacgtatcgtgggaaaacggaagtgaagatttgaggaagttgtgggttttttggctttcgtttctgggcgtaggttcgcgtgcggttttctgggtgttttttgtggactttaaccgttacgtcattttttagtcctatatatactcgctctgtacttggccctttttacactgtgactgattgagctggtgccgtgtcgagtggtgttttttaataggtttttttactggtaaggctgactgttatggctgccgctgtggaagcgctgtatgttgttctggagcgggagggtgctattttgcctaggcaggagggtttttcaggtgtttatgtgtttttctctcctattaattttgttatacctcctatgggggctgtaatgttgtctctacgcctgcgggtatgtattcccccgggctatttcggtcgctttttagcactgaccgatgttaaccaacctgatgtgtttaccgagtcttacattatgactccggacatgaccgaggaactgtcggtggtgctttttaatcacggtgaccagtttttttacggtcacgccggcatggccgtagtccgtcttatgcttataagggttgtttttcctgttgtaagacaggcttctaatgtttaaatgtttttttttttgttattttattttgtgtttaatgcaggaacccgcagacatgtttgagagaaaaatggtgtctttttctgtggtggttccggaacttacctgcctttatctgcatgagcatgactacgatgtgcttgcttttttgcgcgaggctttgcctgattttttgagcagcaccttgcattttatatcgccgcccatgcaacaagcttacataggggctacgctggttagcatagctccgagtatgcgtgtcataatcagtgtgggttcttttgtcatggttcctggcggggaagtggccgcgctggtccgtgcagacctgcacgattatgttcagctggccctgcgaagggacctacgggatcgcggtatttttgttaatgttccgcttttgaatcttatacaggtctgtgaggaacctgaatttttgcaatcatgattcgctgcttgaggctgaaggtggagggcgctctggagcagatttttacaatggccggacttaatattcgggatttgcttagagacatattgataaggtggcgagatgaaaattatttgggcatggttgaaggtgctggaatgtttatagaggagattcaccctgaagggtttagcctttacgtccacttggacgtgagggcagtttgccttttggaagccattgtgcaacatcttacaaatgccattatctgttctttggctgtagagtttgaccacgccaccggaggggagcgcgttcacttaatagatcttcattttgaggttttggataatcttttggaataaaaaaaaaaaaacatggttcttccagctcttcccgctcctcccgtgtgtgactcgcagaacgaatgtgtaggttggctgggtgtggcttattctgcggtggtggatgttatcagggcagcggcgcatgaaggagtttacatagaacccgaagccagggggcgcctggatgctttgagagagtggatatactacaactactacacagagcgagctaagcgacgagaccggagacgcagatctgtttgtcacgcccgcacctggttttgcttcaggaaatatgactacgtccggcgttccatttggcatgacactacgaccaacacgatctcggttgtctcggcgcactccgtacagtagggatcgcctacctccttttgagacagagacccgcgctaccatactggaggatcatccgctgctgcccgaatgtaacactttgacaatgcacaacgtgagttacgtgcgaggtcttccctgcagtgtgggatttacgctgattcaggaatgggttgttccctgggatatggttctgacgcgggaggagcttgtaatcctgaggaagtgtatgcacgtgtgcctgtgttgtgccaacattgatatcatgacgagcatgatgatccatggttacgagtcctgggctctccactgtcattgttccagtcccggttccctgcagtgcatagccggcgggcaggttttggccagctggtttaggatggtggtggatggcgccatgtttaatcagaggtttatatggtaccgggaggtggtgaattacaacatgccaaaagaggtaatgtttatgtccagcgtgtttatgaggggtcgccacttaatctacctgcgcttgtggtatgatggccacgtgggttctgtggtccccgccatgagctttggatacagcgccttgcactgtgggattttgaacaatattgtggtgctgtgctgcagttactgtgctgatttaagtgagatcagggtgcgctgctgtgcccggaggacaaggcgtctcatgctgcgggcggtgcgaatcatcgctgaggagaccactgccatgttgtattcctgcaggacggagcggcggcggcagcagtttattcgcgcgctgctgcagcaccaccgccctatcctgatgcacgattatgactctacccccatgtaggcgtggacttccccttcgccgcccgttgagcaaccgcaagttggacagcagcctgtggctcagcagctggacagcgacatgaacttaagcgagctgcccggggagtttattaatatcactgatgagcgtttggctcgacaggaaaccgtgtggaatataacacctaagaatatgtctgttacccatgatatgatgctttttaaggccagccggggagaaaggactgtgtactctgtgtgttgggagggaggtggcaggttgaatactagggttctgtgagtttgattaaggtacggtgatcaatataagctatgtggtggtggggctatactactgaatgaaaaatgacttgaaattttctgcaattgaaaaataaacacgttgaaacataacatgcaacaggttcacgattctttattcctgggcaatgtaggagaaggtgtaagagttggtagcaaaagtttcagtggtgtattttccactttcccaggaccatgtaaaagacatagagtaagtgcttacctcgctagtttctgtggattcactagaatcgatgtaggatgttgcccctcctgacgcggtaggagaaggggagggtgccctgcatgtctgccgctgctcttgctcttgccgctgctgaggaggggggcgcatctgccgcagcaccggatgcatctgggaaaagcaaaaaaggggctcgtccctgtttccggaggaatttgcaagcggggtcttgcatgacggggaggcaaacccccgttcgccgcagtccggccggcccgagactcgaaccgggggtcctgcgactcaacccttggaaaataaccctccggctacagggagcgagccacttaatgctttcgctttccagcctaaccgcttacgccgcgcgcggccagtggccaaaaaagctagcgcagcagccgccgcgcctggaaggaagccaaaaggagcgctcccccgttgtctgacgtcgcacacctgggttcgacacgcgggcggtaaccgcatggatcacggcggacggccggatccggggttcgaaccccggtcgtccgccatgatacccttgcgaatttatccaccagaccacggaagagtgcccgcttacaggctctccttttgcacggtctagagcgtcaacgactgcgcacgcctcaccggccagagcgtcccgaccatggagcactttttgccgctgcgcaacatctggaaccgcgtccgcgactttccgcgcgcctccaccaccgccgccggcatcacctggatgtccaggtacatctacggattacgtcgacgtttaaaccatatgatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccagggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaacaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgatggatcc

SEQ ID NO: 93 - DNA, 4443 bp, synthetic construct
  misc_feature 1..4443: note = CFTR
  source 1..4443: mol_type = other DNA
atgcagagaagccccctggagaaggcctctgtggtgagcaagctgttcttcagctggaccagacccatcctgagaaagggctacagacagagactggagctgtctgacatctaccagatcccctctgtggactctgctgacaacctgtctgagaagctggagagagagtgggacagagagctggccagcaagaagaaccccaagctgatcaatgccctgagaagatgcttcttctggagattcatgttctatggcatcttcctgtacctgggggaggtgaccaaggctgtgcagcccctgctgctgggcagaatcattgccagctatgaccctgacaacaaggaggagagaagcattgccatctacctgggcattggcctgtgcctgctgttcattgtgagaaccctgctgctgcaccctgccatctttggcctgcaccacattggcatgcagatgagaattgccatgttcagcctgatctacaagaagaccctgaagctgagcagcagagtgctggacaagatcagcattggccagctggtgagcctgctgagcaacaacctgaacaagtttgatgagggcctggccctggcccactttgtgtggattgcccccctgcaggtggccctgctgatgggcctgatctgggagctgctgcaggcctctgccttctgtggcctgggcttcctgattgtgctggccctgttccaggctggcctgggcagaatgatgatgaagtacagagaccagagagctggcaagatctctgagagactggtgatcacctctgagatgattgagaacatccagtctgtgaaggcctactgctgggaggaggccatggagaagatgattgagaacctgagacagacagagctgaagctgaccagaaaggctgcctatgtgagatacttcaacagctctgccttcttcttctctggcttctttgtggtgttcctgtctgtgctgccctatgccctgatcaagggcatcatcctgagaaagatcttcaccaccatcagcttctgcattgtgctgagaatggctgtgaccagacagttcccctgggctgtgcagacctggtatgacagcctgggggccatcaacaagatccaggacttcctgcagaagcaggagtacaagaccctggagtacaacctgaccaccacagaggtggtgatggagaatgtgacagccttctgggaggagggctttggggagctgtttgagaaggccaagcagaacaacaacaacagaaagaccagcaatggggatgacagcctgttcttcagcaacttcagcctgctgggcacccctgtgctgaaggacatcaacttcaagattgagagaggccagctgctggctgtggctggcagcacaggggctggcaagaccagcctgctgatgatgatcatgggggagctggagccctctgagggcaagatcaagcactctggcagaatcagcttctgcagccagttcagctggatcatgcctggcaccatcaaggagaacatcatctttggggtgagctatgatgagtacagatacagatctgtgatcaaggcctgccagctggaggaggacatcagcaagtttgctgagaaggacaacattgtgctgggggaggggggcatcaccctgtctgggggccagagagccagaatcagcctggccagagctgtgtacaaggatgctgacctgtacctgctggacagcccctttggctacctggatgtgctgacagagaaggagatctttgagagctgtgtgtgcaagctgatggccaacaagaccagaatcctggtgaccagcaagatggagcacctgaagaaggctgacaagatcctgatcctgcatgagggcagcagctacttctatggcaccttctctgagctgcagaacctgcagcctgacttcagcagcaagctgatgggctgtgacagctttgaccagttctctgctgagagaagaaacagcatcctgacagagaccctgcacagattcagcctggagggggatgcccctgtgagctggacagagaccaagaagcagagcttcaagcagacaggggagtttggggagaagagaaagaacagcatcctgaaccccatcaacagcatcagaaagttcagcattgtgcagaagacccccctgcagatgaatggcattgaggaggactctgatgagcccctggagagaagactgagcctggtgcctgactctgagcagggggaggccatcctgcccagaatctctgtgatcagcacaggccccaccctgcaggccagaagaagacagtctgtgctgaacctgatgacccactctgtgaaccagggccagaacatccacagaaagaccacagccagcaccagaaaggtgagcctggccccccaggccaacctgacagagctggacatctacagcagaagactgagccaggagacaggcctggagatctctgaggagatcaatgaggaggacctgaaggagtgcttctttgatgacatggagagcatccctgctgtgaccacctggaacacctacctgagatacatcacagtgcacaagagcctgatctttgtgctgatctggtgcctggtgatcttcctggctgaggtggctgccagcctggtggtgctgtggctgctgggcaacacccccctgcaggacaagggcaacagcacccacagcagaaacaacagctatgctgtgatcatcaccagcaccagcagctactatgtgttctacatctatgtgggggtggctgacaccctgctggccatgggcttcttcagaggcctgcccctggtgcacaccctgatcacagtgagcaagatcctgcaccacaagatgctgcactctgtgctgcaggcccccatgagcaccctgaacaccctgaaggctgggggcatcctgaacagattcagcaaggacattgccatcctggatgacctgctgcccctgaccatctttgacttcatccagctgctgctgattgtgattggggccattgctgtggtggctgtgctgcagccctacatctttgtggccacagtgcctgtgattgtggccttcatcatgctgagagcctacttcctgcagaccagccagcagctgaagcagctggagtctgagggcagaagccccatcttcacccacctggtgaccagcctgaagggcctgtggaccctgagagcctttggcagacagccctactttgagaccctgttccacaaggccctgaacctgcacacagccaactggttcctgtacctgagcaccctgagatggttccagatgagaattgagatgatctttgtgatcttcttcattgctgtgaccttcatcagcatcctgaccacaggggagggggagggcagagtgggcatcatcctgaccctggccatgaacatcatgagcaccctgcagtgggctgtgaacagcagcattgatgtggacagcctgatgagatctgtgagcagagtgttcaagttcattgacatgcccacagagggcaagcccaccaagagcaccaagccctacaagaatggccagctgagcaaggtgatgatcattgagaacagccatgtgaagaaggatgacatctggccctctgggggccagatgacagtgaaggacctgacagccaagtacacagaggggggcaatgccatcctggagaacatcagcttcagcatcagccctggccagagagtgggcctgctgggcagaacaggctctggcaagagcaccctgctgtctgccttcctgagactgctgaacacagagggggagatccagattgatggggtgagctgggacagcatcaccctgcagcagtggagaaaggcctttggggtgatcccccagaaggtgttcatcttctctggcaccttcagaaagaacctggacccctatgagcagtggtctgaccaggagatctggaaggtggctgatgaggtgggcctgagatctgtgattgagcagttccctggcaagctggactttgtgctggtggatgggggctgtgtgctgagccatggccacaagcagctgatgtgcctggccagatctgtgctgagcaaggccaagatcctgctgctggatgagccctctgcccacctggaccctgtgacctaccagatcatcagaagaaccctgaagcaggcctttgctgactgcacagtgatcctgtgtgagcacagaattgaggccatgctggagtgccagcagttcctggtgattgaggagaacaaggtgagacagtatgacagcatccagaagctgctgaatgagagaagcctgttcagacaggccatcagcccctctgacagagtgaagctgttcccccacagaaacagcagcaagtgcaagagcaagccccagattgctgccctgaaggaggagaccgaggaggaggtgcaggacaccagactgtaa

SEQ ID NO: 94 - protein, 1480 aa, synthetic construct
  REGION 1..1480: note = CFTR protein
MQRSPLEKASVVSKLFFSWTRPILRKGYRQRLELSDIYQIPSVDSADNLSEKLEREWDRELASKKNPKLINALRRCFFWRFMFYGIFLYLGEVTKAVQPLLLGRIIASYDPDNKEERSIAIYLGIGLCLLFIVRTLLLHPAIFGLHHIGMQMRIAMFSLIYKKTLKLSSRVLDKISIGQLVSLLSNNLNKFDEGLALAHFVWIAPLQVALLMGLIWELLQASAFCGLGFLIVLALFQAGLGRMMMKYRDQRAGKISERLVITSEMIENIQSVKAYCWEEAMEKMIENLRQTELKLTRKAAYVRYFNSSAFFFSGFFVVFLSVLPYALIKGIILRKIFTTISFCIVLRMAVTRQFPWAVQTWYDSLGAINKIQDFLQKQEYKTLEYNLTTTEVVMENVTAFWEEGFGELFEKAKQNNNNRKTSNGDDSLFFSNFSLLGTPVLKDINFKIERGQLLAVAGSTGAGKTSLLMMIMGELEPSEGKIKHSGRISFCSQFSWIMPGTIKENIIFGVSYDEYRYRSVIKACQLEEDISKFAEKDNIVLGEGGITLSGGQRARISLARAVYKDADLYLLDSPFGYLDVLTEKEIFESCVCKLMANKTRILVTSKMEHLKKADKILILHEGSSYFYGTFSELQNLQPDFSSKLMGCDSFDQFSAERRNSILTETLHRFSLEGDAPVSWTETKKQSFKQTGEFGEKRKNSILNPINSIRKFSIVQKTPLQMNGIEEDSDEPLERRLSLVPDSEQGEAILPRISVISTGPTLQARRRQSVLNLMTHSVNQGQNIHRKTTASTRKVSLAPQANLTELDIYSRRLSQETGLEISEEINEEDLKECFFDDMESIPAVTTWNTYLRYITVHKSLIFVLIWCLVIFLAEVAASLVVLWLLGNTPLQDKGNSTHSRNNSYAVIITSTSSYYVFYIYVGVADTLLAMGFFRGLPLVHTLITVSKILHHKMLHSVLQAPMSTLNTLKAGGILNRFSKDIAILDDLLPLTIFDFIQLLLIVIGAIAVVAVLQPYIFVATVPVIVAFIMLRAYFLQTSQQLKQLESEGRSPIFTHLVTSLKGLWTLRAFGRQPYFETLFHKALNLHTANWFLYLSTLRWFQMRIEMIFVIFFIAVTFISILTTGEGEGRVGIILTLAMNIMSTLQWAVNSSIDVDSLMRSVSRVFKFIDMPTEGKPTKSTKPYKNGQLSKVMIIENSHVKKDDIWPSGGQMTVKDLTAKYTEGGNAILENISFSISPGQRVGLLGRTGSGKSTLLSAFLRLLNTEGEIQIDGVSWDSITLQQWRKAFGVIPQKVFIFSGTFRKNLDPYEQWSDQEIWKVADEVGLRSVIEQFPGKLDFVLVDGGCVLSHGHKQLMCLARSVLSKAKILLLDEPSAHLDPVTYQIIRRTLKQAFADCTVILCEHRIEAMLECQQFLVIEENKVRQYDSIQKLLNERSLFRQAISPSDRVKLFPHRNSSKCKSKPQIAALKEETEEEVQDTRL

SEQ ID NO: 95 - protein, 1428 aa, synthetic construct
  REGION 1..1428: note = CFTRdeltaR protein
MQRSPLEKASVVSKLFFSWTRPILRKGYRQRLELSDIYQIPSVDSADNLSEKLEREWDRELASKKNPKLINALRRCFFWRFMFYGIFLYLGEVTKAVQPLLLGRIIASYDPDNKEERSIAIYLGIGLCLLFIVRTLLLHPAIFGLHHIGMQMRIAMFSLIYKKTLKLSSRVLDKISIGQLVSLLSNNLNKFDEGLALAHFVWIAPLQVALLMGLIWELLQASAFCGLGFLIVLALFQAGLGRMMMKYRDQRAGKISERLVITSEMIENIQSVKAYCWEEAMEKMIENLRQTELKLTRKAAYVRYFNSSAFFFSGFFVVFLSVLPYALIKGIILRKIFTTISFCIVLRMAVTRQFPWAVQTWYDSLGAINKIQDFLQKQEYKTLEYNLTTTEVVMENVTAFWEEGFGELFEKAKQNNNNRKTSNGDDSLFFSNFSLLGTPVLKDINFKIERGQLLAVAGSTGAGKTSLLMMIMGELEPSEGKIKHSGRISFCSQFSWIMPGTIKENIIFGVSYDEYRYRSVIKACQLEEDISKFAEKDNIVLGEGGITLSGGQRARISLARAVYKDADLYLLDSPFGYLDVLTEKEIFESCVCKLMANKTRILVTSKMEHLKKADKILILHEGSSYFYGTFSELQNLQPDFSSKLMGCDSFDQFSAERRNSILTETLHRFSLEGDAPVSWTETKKQSFKQTGEFGEKRKNSILNPINSTLQARRRQSVLNLMTHSVNQGQNIHRKTTASTRKVSLAPQANLTELDIYSRRLSQETGLEISEEINEEDLKECFFDDMESIPAVTTWNTYLRYITVHKSLIFVLIWCLVIFLAEVAASLVVLWLLGNTPLQDKGNSTHSRNNSYAVIITSTSSYYVFYIYVGVADTLLAMGFFRGLPLVHTLITVSKILHHKMLHSVLQAPMSTLNTLKAGGILNRFSKDIAILDDLLPLTIFDFIQLLLIVIGAIAVVAVLQPYIFVATVPVIVAFIMLRAYFLQTSQQLKQLESEGRSPIFTHLVTSLKGLWTLRAFGRQPYFETLFHKALNLHTANWFLYLSTLRWFQMRIEMIFVIFFIAVTFISILTTGEGEGRVGIILTLAMNIMSTLQWAVNSSIDVDSLMRSVSRVFKFIDMPTEGKPTKSTKPYKNGQLSKVMIIENSHVKKDDIWPSGGQMTVKDLTAKYTEGGNAILENISFSISPGQRVGLLGRTGSGKSTLLSAFLRLLNTEGEIQIDGVSWDSITLQQWRKAFGVIPQKVFIFSGTFRKNLDPYEQWSDQEIWKVADEVGLRSVIEQFPGKLDFVLVDGGCVLSHGHKQLMCLARSVLSKAKILLLDEPSAHLDPVTYQIIRRTLKQAFADCTVILCEHRIEAMLECQQFLVIEENKVRQYDSIQKLLNERSLFRQAISPSDRVKLFPHRNSSKCKSKPQIAALKEETEEEVQDTRL

SEQ ID NO: 96 - DNA, 250 bp, synthetic construct
  misc_feature 1..250: note = Mouse U1a promoter sequence
  source 1..250: mol_type = other DNA
atggaggcggtactatgtagatgagaattcaggagcaaactgggaaaagcaactgcttccaaatatttgtgatttttacagtgtagttttggaaaaactcttagcctaccaattcttctaagtgttttaaaatgtgggagccagtacacatgaagttatagagtgttttaatgaggcttaaatatttaccgtaactatgaaatgctacgcatatcatgctgttcaggctccgtggccacgcaactcatac

SEQ ID NO: 97 - DNA, 101 bp, synthetic construct
  misc_feature 1..101: note = Polymerase III H1 mutant promoter sequence
  source 1..101: mol_type = other DNA
aatatttgcatgtcgctatgtgttctgggaaatcaccataaacgtgaaatgtctttggatttgggaatcttcgaagttctgtatgagaccacagatctcca

SEQ ID NO: 98 - DNA, 2214 bp, synthetic construct
  misc_feature 1..2214: note = AAV110 DNA
  source 1..2214: mol_type = other DNA
atggctgccgatggttatcttccagattggctcgaggacaacctctctgagggcattcgcgagtggtgggacttgaaacctggagccccgaaacccaaagccaaccagcaaaagcaggacgacggccggggtctggtgcttcctggctacaagtacctcggacccttcaacggactcgacaagggggagcccgtcaacgcggcggatgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagagaccggtagagccatcaccccagcgttctccagactcctctacgggcatcggcaagaaaggccaacagcccgccagaaaaagactcaattttggtcagactggcgactcagagtcagtccccgacccacaacctctcggagaacctccagcaacccccgctgctgtgggacctactacaatggcttcaggcggtggcgcaccaatggcagacaataacgaaggcgccgacggagtgggtaatgcctcaggaaattggcattgcgattccacatggctgggcgacagagtcatcaccaccagcacccgaacatgggccttgcccacctataacaaccacctctacaagcaaatctccagtgcttcaacgggggccagcaacgacaaccactacttcggctacagcaccccctgggggtattttgatttcaacagattccactgccatttctcaccacgtgactggcagcgactcatcaacaacaattggggattccggcccaagagactcaacttcaagctcttcaacatccaagtcaaggaggtcacgacgaatgatggcgtcacgaccatcgctaataaccttaccagcacggttcaagtcttctcggactcggagtaccagttgccgtacgtcctcggctctgcgcaccagggctgcctccctccgttcccggcggacgtgttcatgattccgcagtacggctacctaacgctcaacaatggcagccaggcagtgggacggtcatccttttactgcctggaatatttcccatcgcagatgctgagaacgggcaataactttaccttcagctacaccttcgaggacgtgcctttccacagcagctacgcgcacagccagagcctggaccggctgatgaatcctctcatcgaccagtacctgtattacctgaacagaactcagaatcagtccggaagtgcccaaaacaaggacttgctgtttagccgggggtctccagctggcatgtctgttcagcccaaaaactggctacctggaccctgttaccggcagcagcgcgtttctaaaacaaaaacagacaacaacaacagcaactttacctggactggtgcttcaaaatataaccttaatgggcgtgaatctataatcaaccctggcactgctatggcctcacacaaagacgacaaagacaagttctttcccatgagcggtgtcatgatttttggaaaggagagcgccggagcttcaaacactgcattggacaatgtcatgatcacagacgaagaggaaatcaaagccactaaccccgtggccaccgaaagatttgggactgtggcagtcaatctccagagcagcagcacagaccctgcgaccggagatgtgcatgttatgggagccttacctggaatggtgtggcaagacagagacgtatacctgcagggtcctatttgggccaaaattcctcacacggatggacactttcacccgtctcctctcatgggcggctttggacttaagcacccgcctcctcagatcctcatcaaaaacacgcctgttcctgcgaatcctccggcagagttttcggctacaaagtttgcttcattcatcacccagtattccacaggacaagtgagcgtggagattgaatgggagctgcagaaagaaaacagcaaacgctggaatcccgaagtgcagtatacatctaactatgcaaaatctgccaacgttgatttcactgtggacaacaatggactttatactgagcctcgccccattggcacccgttacctcacccgtcccctgtaa

SEQ ID NO: 99 - DNA, 1509 bp, Homo sapiens
  misc_feature 1..1509: note = REGION - Sulfoglucosamine sulfohydrolase (SGSH)
  source 1..1509: mol_type = other DNA
atgagctgccccgtgcccgcctgctgcgcgctgctgctagtcctggggctctgccgggcgcgtccccggaacgcactgctgctcctcgcggatgacggaggctttgagagtggcgcgtacaacaacagcgccatcgccaccccgcacctggacgccttggcccgccgcagcctcctctttcgcaatgccttcacctcggtcagcagctgctctcccagccgcgccagcctcctcactggcctgccccagcatcagaatgggatgtacgggctgcaccaggacgtgcaccacttcaactccttcgacaaggtgcggagcctgccgctgctgctcagccaagctggtgtgcgcacaggcatcatcgggaagaagcacgtggggccggagaccgtgtacccgtttgactttgcgtacacggaggagaatggctccgtcctccaggtggggcggaacatcactagaattaagctgctcgtccggaaattcctgcagactcaggatgaccagcctttcttcctctacgtcgccttccacgacccccaccgctgtgggcactcccagccccagtacggaaccttctgtgagaagtttggcaacggagagagcggcatgggtcgtatcccagactggaccccccaggcctacgacccactggacgtgctggtgccttacttcgtccccaacaccccggcagcccgagccgacctggccgctcagtacaccaccgtcggccgcatggaccaaggagttggactggtgctccaggagctgcgtgacgccggtgtcctgaacgacacactggtgatcttcacgtccgacaacgggatccccttccccagcggcaggaccaacctgtactggccgggcactgctgaacccttactggtgtcatccccggagcacccaaaacgctggggccaagtcagcgaggcctacgtgagcctcctagacctcacgcccaccatcttggattggttctcgatcccgtaccccagctacgccatctttggctcgaagaccatccacctcactggccggtccctcctgccggcgctggaggccgagcccctctgggccaccgtctttggcagccagagccaccacgaggtcaccatgtcctaccccatgcgctccgtgcagcaccggcacttccgcctcgtgcacaacctcaacttcaagatgccctttcccatcgaccaggacttctacgtctcacccaccttccaggacctcctgaaccgcaccacagctggtcagcccacgggctggtacaaggacctccgtcattactactaccgggcgcgctgggagctctacgaccggagccgggacccccacgagacccagaacctggccaccgacccgcgctttgctcagcttctggagatgcttcgggaccagctggccaagtggcagtgggagacccacgacccctgggtgtgcgcccccgacggcgtcctggaggagaagctctctccccagtgccagcccctccacaatgagctgtga

SEQ ID NO: 100 - DNA, 1509 bp, synthetic construct
  misc_feature 1..1509: note = Codon optimized CO1-SGSH
  source 1..1509: mol_type = other DNA
atgagctgtcctgttccagcctgttgtgccctgctgctggttctgggactgtgcagagccagacctaggaacgctctgctgctgctcgctgacgatggcggatttgagagcggcgcctacaacaacagcgccattgccacacctcacctggatgccctggccagaagaagcctgctgttcagaaacgccttcaccagcgtgtccagctgcagcccttctagagctagcctgctgacaggactgccccagcaccagaatgggatgtatggcctgcaccaggacgtgcaccacttcaacagcttcgacaaagtgcggagcctgcctctgcttctgtctcaagccggcgtcagaacaggcatcatcggcaagaaacacgtgggccccgagacagtgtaccccttcgatttcgcctacaccgaagagaacggcagcgtgctgcaagtgggcagaaacatcacccggatcaagctgctcgtgcggaagttcctgcagacccaggacgaccagcctttcttcctgtacgtggccttccacgatcctcacagatgcggccatagccagcctcagtacggcaccttctgcgagaagtttggcaacggcgagagcggcatgggcagaatccctgattggacccctcaggcctacgatcccctggatgtgctggtgccttacttcgtgcctaacacaccagccgccagagccgatctggccgctcagtatacaaccgtgggaagaatggaccaaggcgtcggcctggttctgcaagagcttagagatgccggcgtgctgaacgacaccctggtcatctttaccagcgacaacggcatcccctttccatctggccggaccaatctgtactggcctggaacagctgagcccctgctggtgtctagccctgagcaccctaagagatggggccaagtgtctgaggcctacgtgtccctgctggatctgacccctaccatcctggactggttcagcatcccctatcctagctacgccatcttcggcagcaagaccatccacctgaccggcagatctctgctgccagctctggaagctgaacctctgtgggccacagtgtttggcagccagtctcaccacgaagtgacaatgagctaccccatgcggagcgtgcagcacagacacttcagactggtgcacaacctgaacttcaagatgccctttccaatcgaccaggacttctatgtgtccccaaccttccaggacctgctgaacagaaccacagccggccaacctaccggctggtacaaggacctgcggcactactactatagagccagatgggagctgtacgaccggtccagagatccccacgagacacagaacctggccaccgatcctagattcgcccagctgctggaaatgctgagagatcagctggccaagtggcagtgggagacacacgatccttgggtctgcgctcctgatggcgtgctggaagagaagctgtcccctcagtgtcagcccctgcacaacgagctttaa

SEQ ID NO: 101 - DNA, 1596 bp, synthetic construct
  misc_feature 1..1596: note = Codon optimized + GET CO1-SGSH-GET
  source 1..1596: mol_type = other DNA
atgagctgtcctgttccagcctgttgtgccctgctgctggttctgggactgtgcagagccagacctaggaacgctctgctgctgctcgctgacgatggcggatttgagagcggcgcctacaacaacagcgccattgccacacctcacctggatgccctggccagaagaagcctgctgttcagaaacgccttcaccagcgtgtccagctgcagcccttctagagctagcctgctgacaggactgccccagcaccagaatgggatgtatggcctgcaccaggacgtgcaccacttcaacagcttcgacaaagtgcggagcctgcctctgcttctgtctcaagccggcgtcagaacaggcatcatcggcaagaaacacgtgggccccgagacagtgtaccccttcgatttcgcctacaccgaagagaacggcagcgtgctgcaagtgggcagaaacatcacccggatcaagctgctcgtgcggaagttcctgcagacccaggacgaccagcctttcttcctgtacgtggccttccacgatcctcacagatgcggccatagccagcctcagtacggcaccttctgcgagaagtttggcaacggcgagagcggcatgggcagaatccctgattggacccctcaggcctacgatcccctggatgtgctggtgccttacttcgtgcctaacacaccagccgccagagccgatctggccgctcagtatacaaccgtgggaagaatggaccaaggcgtcggcctggttctgcaagagcttagagatgccggcgtgctgaacgacaccctggtcatctttaccagcgacaacggcatcccctttccatctggccggaccaatctgtactggcctggaacagctgagcccctgctggtgtctagccctgagcaccctaagagatggggccaagtgtctgaggcctacgtgtccctgctggatctgacccctaccatcctggactggttcagcatcccctatcctagctacgccatcttcggcagcaagaccatccacctgaccggcagatctctgctgccagctctggaagctgaacctctgtgggccacagtgtttggcagccagtctcaccacgaagtgacaatgagctaccccatgcggagcgtgcagcacagacacttcagactggtgcacaacctgaacttcaagatgccctttccaatcgaccaggacttctatgtgtccccaaccttccaggacctgctgaacagaaccacagccggccaacctaccggctggtacaaggacctgcggcactactactatagagccagatgggagctgtacgaccggtccagagatccccacgagacacagaacctggccaccgatcctagattcgcccagctgctggaaatgctgagagatcagctggccaagtggcagtgggagacacacgatccttgggtctgcgctcctgatggcgtgctggaagagaagctgtcccctcagtgtcagcccctgcacaacgagctgcggcgtcgtcggcgaagaagaagaaagcgcaagaaaaaaggcaaaggcctgggcaagaagcgggacccctgtctgagaaagtacaaataa

SEQ ID NO: 102 - DNA, 1509 bp, synthetic construct
  misc_feature 1..1509: note = Codon optimized CO2-SGSH
  source 1..1509: mol_type = other DNA
atgagctgccctgtgcctgcctgctgtgccctgctgctggtgctgggcctgtgcagagccagacctaggaatgccctgctgctgctggctgatgatgggggctttgagagtggggcctacaacaacagtgccattgccaccccccacctggatgccctggccagaagaagcctgctgttcagaaatgccttcaccagtgtgagcagctgcagccccagcagagccagcctgctgacaggcctgccccagcaccagaatggcatgtatggcctgcaccaggatgtgcaccacttcaacagctttgacaaggtgagaagcctgcccctgctgctgagccaggctggggtgagaacaggcatcattggcaagaagcatgtgggccctgagacagtgtacccctttgactttgcctacacagaggagaatggcagtgtgctgcaggtgggcagaaacatcaccagaatcaagctgctggtgagaaagttcctgcagacccaggatgaccagcccttcttcctgtatgtggccttccatgacccccacagatgtggccacagccagccccagtatggcaccttctgtgagaagtttggcaatggggagagtggcatgggcagaatccctgactggaccccccaggcctatgaccccctggatgtgctggtgccctactttgtgcccaacacccctgctgccagagctgacctggctgcccagtacaccacagtgggcagaatggaccagggggtgggcctggtgctgcaggagctgagagatgctggggtgctgaatgacaccctggtgatcttcaccagtgacaatggcatccccttccccagtggcagaaccaacctgtactggcctggcacagctgagcccctgctggtgagcagccctgagcaccccaagagatggggccaggtgagtgaggcctatgtgagcctgctggacctgacccccaccatcctggactggttcagcatcccctaccccagctatgccatctttggcagcaagaccatccacctgacaggcagaagcctgctgcctgccctggaggctgagcccctgtgggccacagtgtttggcagccagagccaccatgaggtgaccatgagctaccccatgagaagtgtgcagcacagacacttcagactggtgcacaacctgaacttcaagatgcccttccccattgaccaggacttctatgtgagccccaccttccaggacctgctgaacagaaccacagctggccagcccacaggctggtacaaggacctgagacactactactacagagccagatgggagctgtatgacagaagcagagacccccatgagacccagaacctggccacagaccccagatttgcccagctgctggagatgctgagagaccagctggccaagtggcagtgggagacccatgacccctgggtgtgtgcccctgatggggtgctggaggagaagctgagcccccagtgccagcccctgcacaatgagctgtga

SEQ ID NO: 103 - DNA, 921 bp, synthetic construct
  misc_feature 1..921: note = Codon optimized Ceroid Lipofuscinosis, Neuronal, 1 (CLN1)
  source 1..921: mol_type = other DNA
atggcttctccggggtgtctgtggctgctggcagtggcactccttccctggacttgcgccagccgggctctgcagcacctcgaccctccagcccctcttccactggtgatttggcacggaatgggtgattcctgctgtaatcccctgtcaatgggagccatcaagaagatggtggagaagaagatccctggaatctacgtgctgtcactggagattggaaagaccctgatggaggacgtcgagaactccttcttcctcaatgtcaactctcaagtgaccaccgtctgccaggccctggccaaggacccgaagctgcagcaggggtataatgctatggggttcagccagggaggacagttccttcgggctgtggcccaacgctgccctagcccacccatgatcaacctgatctcagtgggtggccagcatcagggcgtgttcggacttccccggtgtcccggggaatcctctcatatctgcgacttcatccgcaaaactctcaatgcaggcgcttattcaaaggtcgtccaagagaggctggtgcaagccgagtactggcacgatcccattaaggaggacgtgtacagaaatcactcaatctttctggccgacattaaccaggagaggggaattaacgaatcatataagaagaatctcatggccctcaaaaagttcgtcatggtgaagttccttaacgatagcattgtggacccagtggacagcgaatggttcggattttaccgctcaggccaggcaaaagaaaccatccctctccaagagacttctctttacacccaagacagacttgggcttaaggaaatggataacgctggtcagctggtgttcctcgccaccgaaggtgaccatctgcagctcagcgaagagtggttctacgctcatatcatcccgtttcttggttga

SEQ ID NO: 104 - DNA, 885 bp, Homo sapiens
  misc_feature 1..885: note = REGION - Survival Motor Neuron 1 (SMN1)
  source 1..885: mol_type = other DNA
atggcgatgagcagcggcggcagtggtggcggcgtcccggagcaggaggattccgtgctgttccggcgcggcacaggccagagcgatgattctgacatttgggatgatacagcactgataaaagcatatgataaagctgtggcttcatttaagcatgctctaaagaatggtgacatttgtgaaacttcgggtaaaccaaaaaccacacctaaaagaaaacctgctaagaagaataaaagccaaaagaagaatactgcagcttccttacaacagtggaaagttggggacaaatgttctgccatttggtcagaagacggttgcatttacccagctaccattgcttcaattgattttaagagagaaacctgtgttgtggtttacactggatatggaaatagagaggagcaaaatctgtccgatctactttccccaatctgtgaagtagctaataatatagaacagaatgctcaagagaatgaaaatgaaagccaagtttcaacagatgaaagtgagaactccaggtctcctggaaataaatcagataacatcaagcccaaatctgctccatggaactcttttctccctccaccaccccccatgccagggccaagactgggaccaggaaagccaggtctaaaattcaatggcccaccaccgccaccgccaccaccaccaccccacttactatcatgctggctgcctccatttccttctggaccaccaataattcccccaccacctcccatatgtccagattctcttgatgatgctgatgctttgggaagtatgttaatttcatggtacatgagtggctatcatactggctattatatgggttttagacaaaatcaaaaagaaggaaggtgctcacattccttaaattaa

SEQ ID NO: 105 - DNA, 885 bp, synthetic construct
  misc_feature 1..885: note = Codon optimized CO1-SMN1
  source 1..885: mol_type = other DNA
atggcgatgtctagtggtggatctggtggcggcgtgcccgagcaagaagatagcgtcctgttcagaagaggcaccggccagagcgacgacagcgacatctgggatgatacagccctgatcaaggcctacgacaaggccgtggccagctttaagcacgccctgaagaacggcgatatctgcgagacaagcggcaagcccaagaccacacctaagagaaagcccgccaagaagaacaagagccagaagaagaataccgccgccagcctgcagcagtggaaagtgggcgataagtgcagcgccatttggagcgaggacggctgtatctaccctgccacaatcgccagcatcgacttcaagcgggaaacctgcgtggtggtgtacacaggctacggcaacagagaggaacagaacctgagcgacctgctgtccccaatttgcgaggtggccaacaacatcgagcagaacgcccaagagaacgagaacgagtcccaggtgtccaccgacgagagcgagaatagcagaagccccggcaacaagagcgacaacatcaagcctaagagcgccccttggaacagcttcctgcctcctcctccaccaatgcctggacctagactcggacctggaaagcccggcctgaagttcaatggacctccaccaccgccaccacctccgcctccacatcttctgtcttgttggctgcctccatttcctagcggccctccaatcatcccgccacctccacctatctgccccgacagtctggatgatgctgatgccctgggctccatgctgatctcttggtacatgagcggctaccacaccggctactacatgggcttcagacagaaccagaaagagggccgttgcagccacagcctgaactga

SEQ ID NO: 106 - DNA, 885 bp, synthetic construct
  misc_feature 1..885: note = Codon optimized CO2-SMN1
  source 1..885: mol_type = other DNA
atggccatgagcagtgggggcagtggaggaggggtgcctgagcaggaggacagtgtgctgttcagaagaggcacaggccagagtgatgacagtgacatctgggatgacacagccctgatcaaggcctatgacaaggctgtggccagcttcaagcatgccctgaagaatggggacatctgtgagaccagtggcaagcccaagaccacccccaagagaaagcctgccaagaagaacaagagccagaagaagaacacagctgccagcctgcagcagtggaaggtgggagacaagtgcagtgccatctggagtgaggatggctgcatctaccctgccaccattgccagcattgacttcaagagagagacctgtgtggtggtgtacacaggctatggcaacagagaggagcagaacctgagtgacctgctgagccccatctgtgaggtggccaacaacattgagcagaatgcccaggagaatgagaatgagagccaggtgagcacagatgagagtgagaacagcagaagccctggcaacaagagtgacaacatcaagcccaagagtgccccttggaacagcttcctgccacccccaccacccatgcctggccccagactgggccctggcaagcctggcctgaagttcaatggcccaccaccccctcctccaccaccccctccccacctgctgagctgctggctgccccccttccccagtggcccacccatcatcccacctcccccacccatctgccctgacagcctggatgatgctgatgccctgggcagcatgctgatcagctggtacatgagtggctaccacacaggctactacatgggcttcagacagaaccagaaggagggcagatgcagccacagcctgaactga

SEQ ID NO: 107 - DNA, 1548 bp, Homo sapiens
  misc_feature 1..1548: note = REGION - Tissue Non-specific Alkaline Phosphatase (TNALP)
  source 1..1548: mol_type = other DNA
atgatttcaccattcttagtactggccattggcacctgccttactaactcactagtgccagagaaagagaaagaccccaagtactggcgagaccaagcgcaagagacactgaaatatgccctggagcttcagaagctcaacaccaacgtggctaagaatgtcatcatgttcctgggagatgggatgggtgtctccacagtgacggctgcccgcatcctcaagggtcagctccaccacaaccctggggaggagaccaggctggagatggacaagttccccttcgtggccctctccaagacgtacaacaccaatgcccaggtccctgacagcgccggcaccgccaccgcctacctgtgtggggtgaaggccaatgagggcaccgtgggggtaagcgcagccactgagcgttcccggtgcaacaccacccaggggaacgaggtcacctccatcctgcgctgggccaaggacgctgggaaatctgtgggcattgtgaccaccacgagagtgaaccatgccacccccagcgccgcctacgcccactcggctgaccgggactggtactcagacaacgagatgccccctgaggccttgagccagggctgtaaggacatcgcctaccagctcatgcataacatcagggacattgacgtgatcatggggggtggccggaaatacatgtaccccaagaataaaactgatgtggagtatgagagtgacgagaaagccaggggcacgaggctggacggcctggacctcgttgacacctggaagagcttcaaaccgagatacaagcactcccacttcatctggaaccgcacggaactcctgacccttgacccccacaatgtggactacctattgggtctcttcgagccaggggacatgcagtacgagctgaacaggaacaacgtgacggacccgtcactctccgagatggtggtggtggccatccagatcctgcggaagaaccccaaaggcttcttcttgctggtggaaggaggcagaattgaccacgggcaccatgaaggaaaagccaagcaggccctgcatgaggcggtggagatggaccgggccatcgggcaggcaggcagcttgacctcctcggaagacactctgaccgtggtcactgcggaccattcccacgtcttcacatttggtggatacaccccccgtggcaactctatctttggtctggcccccatgctgagtgacacagacaagaagcccttcactgccatcctgtatggcaatgggcctggctacaaggtggtgggcggtgaacgagagaatgtctccatggtggactatgctcacaacaactaccaggcgcagtctgctgtgcccctgcgccacgagacccacggcggggaggacgtggccgtcttctccaagggccccatggcgcacctgctgcacggcgtccacgagcagaactacgtcccccacgtgatggcgtatgcagcctgcatcggggccaacctcggccactgtgctcctgccagctcggcaggatccgatgatgacgacgacgatgacgatgatgattga

SEQ ID NO: 108 - DNA, 1548 bp, synthetic construct
  misc_feature 1..1548: note = Codon optimized, CO1-TNALP contains D10 tag at C end
  source 1..1548: mol_type = other DNA
atgatctctccatttctggtgctggccatcggcacctgtctgaccaactcactagtgcccgagaaagagaaggaccccaagtactggcgcgatcaggcccaagagacactgaagtacgccctggaactgcagaaactgaacaccaacgtggccaagaacgtgatcatgttcctcggcgacggcatgggcgtgtccacagttacagccgccagaatcctgaagggccagctgcaccataatcctggcgaagagacacggctggaaatggacaagttcccattcgtggccctgagcaagacctacaacaccaatgctcaggtgcccgattctgccggaacagccacagcttatctgtgcggcgtgaaggccaatgagggcaccgttggagtgtctgccgccaccgaaagatcccggtgcaataccacacagggcaacgaagtgaccagcatcctgagatgggccaaagacgccggcaagtctgtgggcatcgtgaccaccaccagagtgaaccacgccacacctagcgccgcctatgctcactctgccgacagagactggtacagcgacaacgagatgcctcctgaggctctgtctcagggctgcaaggatatcgcctaccagctgatgcacaacatccgggacattgatgtgatcatgggcggaggccggaagtacatgtatcccaagaacaagaccgacgtcgagtacgagagcgacgagaaggccagaggcacaagactggatggcctggacctggtggatacctggaagtccttcaagccccggtacaagcacagccacttcatctggaaccggaccgagctgctgacactggaccctcacaatgtggactacctgctgggcctgttcgagcccggcgatatgcagtacgagctgaaccggaacaacgtgacagaccccagcctgagcgagatggtggttgtggccattcagatcctgcggaagaaccccaagggattcttcctgctggtggaaggcggcaggatcgatcacggacaccatgagggaaaagccaagcaggccctgcacgaggccgtcgaaatggatagagccattggccaggccggcagcctgacaagctctgaggatacactgaccgtggtcaccgccgatcacagccacgtgttcacattcggcggctacacccctagaggcaacagcatctttggactggcccctatgctgagcgacaccgacaagaagcctttcaccgccatcctgtacggcaacggccctggctataaggttgtcggaggcgagagggaaaacgtgtccatggtggattacgcccacaacaactaccaggctcagagcgccgtgcctctgagacacgaaacacacggcggagaagatgtggccgtgttcagcaagggccccatggctcatctgctgcatggcgtgcacgagcagaattacgtgccacacgtgatggcctacgccgcctgtattggagccaatctgggacattgtgcccctgccagtagcgccggatccgacgatgatgacgacgacgatgacgatgactga

SEQ ID NO: 109 - DNA, 1548 bp, synthetic construct
  misc_feature 1..1548: note = Codon optimized, CO2-TNALP contains D10 tag at C end
  source 1..1548: mol_type = other DNA
atgatcagccccttcctggtgctggccattggcacctgcctgaccaacagcctggtgcctgagaaggagaaggaccccaagtactggagagaccaggcccaggagaccctgaagtatgccctggagctgcagaagctgaacaccaatgtggccaagaatgtgatcatgttcctgggggatggcatgggggtgagcacagtgacagctgccagaatcctgaagggccagctgcaccacaaccctggggaggagaccagactggagatggacaagttcccctttgtggccctgagcaagacctacaacaccaatgcccaggtgcctgacagtgctggcacagccacagcctacctgtgtggggtgaaggccaatgagggcacagtgggggtgagtgctgccacagagagaagcagatgcaacaccacccagggcaatgaggtgaccagcatcctgagatgggccaaggatgctggcaagagtgtgggcattgtgaccaccaccagagtgaaccatgccacccccagtgctgcctatgcccacagtgctgacagagactggtacagtgacaatgagatgccccctgaggccctgagccagggctgcaaggacattgcctaccagctgatgcacaacatcagagacattgatgtgatcatgggggggggcagaaagtacatgtaccccaagaacaagacagatgtggagtatgagagtgatgagaaggccagaggcaccagactggatggcctggacctggtggacacctggaagagcttcaagcccagatacaagcacagccacttcatctggaacagaacagagctgctgaccctggacccccacaatgtggactacctgctgggcctgtttgagcctggggacatgcagtatgagctgaacagaaacaatgtgacagaccccagcctgagtgagatggtggtggtggccatccagatcctgagaaagaaccccaagggcttcttcctgctggtggaggggggcagaattgaccatggccaccatgagggcaaggccaagcaggccctgcatgaggctgtggagatggacagagccattggccaggctggcagcctgaccagcagtgaggacaccctgacagtggtgacagctgaccacagccatgtgttcacctttgggggctacacccccagaggcaacagcatctttggcctggcccccatgctgagtgacacagacaagaagcccttcacagccatcctgtatggcaatggccctggctacaaggtggtggggggggagagagagaatgtgagcatggtggactatgcccacaacaactaccaggcccagagtgctgtgcccctgagacatgagacccatgggggggaggatgtggctgtgttcagcaagggccccatggcccacctgctgcatggggtgcatgagcagaactatgtgccccatgtgatggcctatgctgcctgcattggggccaacctgggccactgtgcccctgccagcagtgctggatccgatgatgatgatgatgatgatgatgatgactga

SEQ ID NO: 110 - DNA, 636 bp, Homo sapiens
  misc_feature 1..636: note = REGION - Glial Cell Derived Neurotrophic Factor (GDNF)
  source 1..636: mol_type = other DNA
atgaagttatgggatgtcgtggctgtctgcctggtgctgctccacaccgcgtccgccttcccgctgcccgccggcaagaggcctcccgaggcgcccgccgaagaccgctccctcggccgccgccgcgcgcccttcgcgctgagcagtgactcaaatatgccagaggattatcctgatcagttcgatgatgtcatggattttattcaagccaccattaaaagactgaaaaggtcaccagataaacaaatggcagtgcttcctagaagagagcggaatcggcaggctgcagctgccaacccagagaattccagaggaaaaggtcggagaggccagaggggcaaaaaccggggttgtgtcttaactgcaatacatttaaatgtcactgacttgggtctgggctatgaaaccaaggaggaactgatttttaggtactgcagcggctcttgcgatgcagctgagacaacgtacgacaaaatattgaaaaacttatccagaaatagaaggctggtgagtgacaaagtagggcaggcatgttgcagacccatcgcctttgatgatgacctgtcgtttttagatgataacctggtttaccatattctaagaaagcattccgctaaaaggtgtggatgtatctaa

SEQ ID NO: 111 - DNA, 1611 bp, Homo sapiens
  misc_feature 1..1611: note = REGION - Tissue Glucosyl Ceramidase beta (GBA1)
  source 1..1611: mol_type = other DNA
atggagttttcaagtccttccagagaggaatgtcccaagcctttgagtagggtaagcatcatggctggcagcctcacaggattgcttctacttcaggcagtgtcgtgggcatcaggtgcccgcccctgcatccctaaaagcttcggctacagctcggtggtgtgtgtctgcaatgccacatactgtgactcctttgaccccccgacctttcctgcccttggtaccttcagccgctatgagagtacacgcagtgggcgacggatggagctgagtatggggcccatccaggctaatcacacgggcacaggcctgctactgaccctgcagccagaacagaagttccagaaagtgaagggatttggaggggccatgacagatgctgctgctctcaacatccttgccctgtcaccccctgcccaaaatttgctacttaaatcgtacttctctgaagaaggaatcggatataacatcatccgggtaccaatggccagctgtgacttctccatccgcacctacacctatgcagacacccctgatgatttccagttgcacaacttcagcctcccagaggaagataccaagctcaagatacccctgattcaccgagccctgcagttggcccagcgtcccgtttcactccttgccagcccctggacatcacccacttggctcaagaccaatggagcggtgaatgggaaggggtcactcaagggacagcccggagacatctaccaccagacctgggccagatactttgtgaagttcctggatgcctatgctgagcacaagttacagttctgggcagtgacagctgaaaatgagccttctgctgggctgttgagtggataccccttccagtgcctgggcttcacccctgaacatcagcgagacttcattgcccgtgacctaggtcctaccctcgccaacagtactcaccacaatgtccgcctactcatgctggatgaccaacgcttgctgctgccccactgggcaaaggtggtactgacagacccagaagcagctaaatatgttcatggcattgctgtacattggtacctggactttctggctccagccaaagccaccctaggggagacacaccgcctgttccccaacaccatgctctttgcctcagaggcctgtgtgggctccaagttctgggagcagagtgtgcggctaggctcctgggatcgagggatgcagtacagccacagcatcatcacgaacctcctgtaccatgtggtcggctggaccgactggaaccttgccctgaaccccgaaggaggacccaattgggtgcgtaactttgtcgacagtcccatcattgtagacatcaccaaggacacgttttacaaacagcccatgttctaccaccttggccacttcagcaagttcattcctgagggctcccagagagtggggctggttgccagtcagaagaacgacctggacgcagtggcactgatgcatcccgatggctctgctgttgtggtcgtgctaaaccgctcctctaaggatgtgcctcttaccatcaaggatcctgctgtgggcttcctggagacaatctcacctggctactccattcacacctacctgtggcgtcgccagtga

SEQ ID NO: 112 - DNA, 1611 bp, synthetic construct
  misc_feature 1..1611: note = Codon optimized CO1-GBA1
  source 1..1611: mol_type = other DNA
atggagttcagcagccccagcagagaggagtgccccaagcccctgagcagagtgagcatcatggctggcagcctgacaggcctgctgctgctgcaggctgtgagctgggccagtggggccagaccctgcatccccaagagctttggctacagcagtgtggtgtgtgtgtgcaatgccacctactgtgacagctttgacccccccaccttccctgccctgggcaccttcagcagatatgagagcaccagaagtggcagaagaatggagctgagcatgggccccatccaggccaaccacacaggcacaggcctgctgctgaccctgcagcctgagcagaagttccagaaggtgaagggctttgggggggccatgacagatgctgctgccctgaacatcctggccctgagcccccctgcccagaacctgctgctgaagagctacttcagtgaggagggcattggctacaacatcatcagagtgccaatggccagctgtgacttcagcatcagaacctacacctatgctgacacccctgatgacttccagctgcacaacttcagcctgcctgaggaggacaccaagctgaagatccccctgatccacagagccctgcagctggcccagagacctgtgagcctgctggccagcccctggaccagccccacctggctgaagaccaatggggctgtgaatggcaagggcagcctgaagggccagcctggggacatctaccaccagacctgggccagatactttgtgaagttcctggatgcctatgctgagcacaagctgcagttctgggctgtgacagctgagaatgagcccagtgctggcctgctgagtggctaccccttccagtgcctgggcttcacccctgagcaccagagagacttcattgccagagacctgggccccaccctggccaacagcacccaccacaatgtgagactgctgatgctggatgaccagagactgctgctgccccactgggccaaggtggtgctgacagaccctgaggctgccaagtatgtgcatggcattgctgtgcactggtacctggacttcctggcccctgccaaggccaccctgggggagacccacagactgttccccaacaccatgctgtttgccagtgaggcctgtgtgggcagcaagttctgggagcagagtgtgagactgggcagctgggacagaggcatgcagtacagccacagcatcatcaccaacctgctgtaccatgtggtgggctggacagactggaacctggccctgaaccctgaggggggccccaactgggtgagaaactttgtggacagccccatcattgtggacatcaccaaggacaccttctacaagcagcccatgttctaccacctgggccacttcagcaagttcatccctgagggcagccagagagtgggcctggtggccagccagaagaatgacctggatgctgtggccctgatgcaccctgatggcagtgctgtggtggtggtgctgaacagaagcagcaaggatgtgcccctgaccatcaaggaccctgctgtgggcttcctggagaccatcagccctggctacagcatccacacctacctgtggagaagacagtga

SEQ ID NO: 113 - DNA, 1611 bp, synthetic construct
  misc_feature 1..1611: note = Codon optimized CO2-GBA1
  source 1..1611: mol_type = other DNA
atggagtttagcagccctagcagagaggaatgccccaagcctctgagccgggtgtcaatcatggccggatctctgacaggactgctgctgcttcaggccgtgtcttgggcttctggcgctagaccttgcatccccaagagcttcggctacagcagcgtcgtgtgcgtgtgcaatgccacctactgcgacagcttcgaccctcctacctttcctgctctgggcaccttcagcagatacgagagcaccagatccggcagacggatggaactgagcatgggacccatccaggccaatcacacaggcactggcctgctgctgacactgcagcctgagcagaaattccagaaagtgaaaggcttcggcggagccatgacagatgccgccgctctgaatatcctggctctgtctccaccagctcagaacctgctgctcaagagctacttcagcgaggaaggcatcggctacaacatcatccgggtgccaatggccagctgcgacttcagcatccggacctacacctacgccgacacacccgacgatttccagctgcacaacttcagcctgcctgaagaggacaccaagctgaagatccctctgatccacagagccctgcagctggcacaaagacccgtttctctgctggctagcccctggacatctcccacctggctgaaaacaaatggcgccgtgaatggcaagggcagcctgaaaggccaacctggcgatatctaccaccagacctgggccagatacttcgtgaagttcctggacgcctatgccgagcacaagctgcagttttgggccgtgacagccgagaacgaaccttctgctggactgctgagcggctacccctttcagtgcctgggctttacacccgagcaccagcgggactttatcgccagagatctgggacccacactggccaatagcacccaccataatgtgcggctgctgatgctggacgaccagagactgcttctgccccactgggctaaagtggtgctgacagatcctgaggccgccaaatacgtgcacggaatcgccgtgcactggtatctggactttctggcccctgccaaggccacactgggagagacacacagactgttccccaacaccatgctgttcgccagcgaagcctgtgtgggcagcaagttttgggaacagagcgtgcggctcggcagctgggatagaggcatgcagtacagccacagcatcatcaccaacctgctgtaccacgtcgtcggctggaccgactggaatctggccctgaatcctgaaggcggccctaactgggtccgaaacttcgtggacagccccatcatcgtggacatcaccaaggacaccttctacaagcagcccatgttctaccacctgggacacttcagcaagttcatccccgagggctctcagcgcgttggactggtggccagccagaagaatgatctggacgccgtggctctgatgcaccctgatggatctgctgtggtggtggtcctgaaccgcagcagcaaagatgtgcccctgaccatcaaggatcccgccgtgggattcctggaaacaatcagccctggctactccatccacacctacctgtggcggagacagtga

SEQ ID NO: 114 - DNA, 1962 bp, Homo sapiens
  misc_feature 1..1962: note = REGION - Iduronidase alpha-L- (IDUA)
  source 1..1962: mol_type = other DNA
atgcgtcccctgcgcccccgcgccgcgctgctggcgctcctggcctcgctcctggccgcgcccccggtggccccggccgaggccccgcacctggtgcatgtggacgcggcccgcgcgctgtggcccctgcggcgcttctggaggagcacaggcttctgccccccgctgccacacagccaggctgaccagtacgtcctcagctgggaccagcagctcaacctcgcctatgtgggcgccgtccctcaccgcggcatcaagcaggtccggacccactggctgctggagcttgtcaccaccagggggtccactggacggggcctgagctacaacttcacccacctggacgggtacctggaccttctcagggagaaccagctcctcccagggtttgagctgatgggcagcgcctcgggccacttcactgactttgaggacaagcagcaggtgtttgagtggaaggacttggtctccagcctggccaggagatacatcggtaggtacggactggcgcatgtttccaagtggaacttcgagacgtggaatgagccagaccaccacgactttgacaacgtctccatgaccatgcaaggcttcctgaactactacgatgcctgctcggagggtctgcgcgccgccagccccgccctgcggctgggaggccccggcgactccttccacaccccaccgcgatccccgctgagctggggcctcctgcgccactgccacgacggtaccaacttcttcactggggaggcgggcgtgcggctggactacatctccctccacaggaagggtgcgcgcagctccatctccatcctggagcaggagaaggtcgtcgcgcagcagatccggcagctcttccccaagttcgcggacacccccatttacaacgacgaggcggacccgctggtgggctggtccctgccacagccgtggagggcggacgtgacctacgcggccatggtggtgaaggtcatcgcgcagcatcagaacctgctactggccaacaccacctccgccttcccctacgcgctcctgagcaacgacaatgccttcctgagctaccacccgcaccccttcgcgcagcgcacgctcaccgcgcgcttccaggtcaacaacacccgcccgccgcacgtgcagctgttgcgcaagccggtgctcacggccatggggctgctggcgctgctggatgaggagcagctctgggccgaagtgtcgcaggccgggaccgtcctggacagcaaccacacggtgggcgtcctggccagcgcccaccgcccccagggcccggccgacgcctggcgcgccgcggtgctgatctacgcgagcgacgacacccgcgcccaccccaaccgcagcgtcgcggtgaccctgcggctgcgcggggtgccccccggcccgggcctggtctacgtcacgcgctacctggacaacgggctctgcagccccgacggcgagtggcggcgcctgggccggcccgtcttccccacggcagagcagttccggcgcatgcgcgcggctgaggacccggtggccgcggcgccccgccccttacccgccggcggccgcctgaccctgcgccccgcgctgcggctgccgtcgcttttgctggtgcacgtgtgtgcgcgccccgagaagccgcccgggcaggtcacgcggctccgcgccctgcccctgacccaagggcagctggttctggtctggtcggatgaacacgtgggctccaagtgcctgtggacatacgagatccagttctctcaggacggtaaggcgtacaccccggtcagcaggaagccatcgaccttcaacctctttgtgttcagcccagacacaggtgctgtctctggctcctaccgagttcgagccctggactactgggcccgaccaggccccttctcggaccctgtgccgtacctggaggtccctgtgccaagagggcccccatccccgggcaatccatga

SEQ ID NO: 115 - DNA, 1962 bp, synthetic construct
  misc_feature 1..1962: note = Codon optimized CO1-IDUA
  source 1..1962: mol_type = other DNA
atgagacccctgagacccagagctgccctgctggccctgctggccagcctgctggctgccccccctgtggcccctgctgaggccccccaccttgtacatgtggatgctgccagagccctgtggcccctgagaagattctggagaagcacaggcttctgcccccccctgccccacagccaggctgaccagtatgtgctgagctgggaccagcagctgaacctggcctatgtgggggctgtgccccacagaggcatcaagcaggtgagaacccactggctgctggagctggtgaccaccagaggcagcacaggcagaggcctgagctacaacttcacccacctggatggctacctggacctgctgagagagaaccagctgctgcctggctttgagctgatgggcagtgccagtggccacttcacagactttgaggacaagcagcaggtgtttgagtggaaggacctggtgagcagcctggccagaagatacattggcagatatggcctggcccatgtgagcaagtggaactttgagacctggaatgagcctgaccaccatgactttgacaatgtgagcatgaccatgcagggcttcctgaactactatgatgcctgcagtgagggcctgagagctgccagccctgccctgagactggggggccctggggacagcttccacaccccccccagaagccccctgagctggggcctgctgagacactgccatgatggcaccaacttcttcacaggggaggctggggtgagactggactacatcagcctgcacagaaagggggccagaagcagcatcagcatcctggagcaggagaaggtggtggcccagcagatcagacagctgttccccaagtttgctgacacccccatctacaatgatgaggctgaccccctggtgggctggagcctgccccagccctggagagctgatgtgacctatgctgccatggtggtgaaggtgattgcccagcaccagaacctgctgctggccaacaccaccagtgccttcccctatgccctgctgagcaatgacaatgccttcctgagctaccacccccacccctttgcccagagaaccctgacagccagattccaggtgaacaacaccagacccccccatgtgcagctgctgagaaagcctgtgctgacagccatgggcctgctggccctgctggatgaggagcagctgtgggctgaggtgagccaggctggcacagtgctggacagcaaccacacagtgggggtgctggccagtgcccacagaccccagggccctgctgatgcctggagagctgctgtgctgatctatgccagtgatgacaccagagcccaccccaacagaagtgtggctgtgaccctgagactgagaggggtgccccctggccctggcctggtgtatgtgaccagatacctggacaatggcctgtgcagccctgatggggagtggagaagactgggcagacctgtgttccccacagctgagcagttcagaagaatgagagctgctgaggaccctgtggctgctgcccccagacccctgcctgctgggggcagactgaccctgagacctgccctgagactgcccagcctgctgctggtgcatgtgtgtgccagacctgagaagccccctggccaggtgaccagactgagagccctgcccctgacccagggccagctggtgctggtgtggagtgatgagcatgtgggcagcaagtgcctgtggacctatgagatccagttcagccaggatggcaaggcctacacccctgtgagcagaaagcccagcaccttcaacctgtttgtgttcagccctgacacaggggctgtgagtggcagctacagagtgagagccctggactactgggccagacctggccccttcagtgaccctgtgccctacctggaggtgcctgtgcccagaggcccccccagccctggcaacccctga

SEQ ID NO: 116 - DNA, 1578 bp, Homo sapiens
  misc_feature 1..1578: note = REGION - Cytochrome P450 family 4 subfamily V member 2 (CYP4V2)
  source 1..1578: mol_type = other DNA
atggcggggctctggctggggctcgtgtggcagaagctgctgctgtggggcgcggcgagtgccctttccctggccggcgccagtctggtcctgagcctgctgcagagggtggcgagctacgcgcggaaatggcagcagatgcggcccatccccacggtggcccgcgcctacccactggtgggccacgcgctgctgatgaagccggacgggcgagaattttttcagcagatcattgagtacacagaggaataccgccacatgccgctgctgaagctctgggtcgggccagtgcccatggtggccctttataatgcagaaaatgtggaggtaattttaactagttcaaagcaaattgacaaatcctctatgtacaagtttttagaaccatggcttggcctaggacttcttacaagtactggaaacaaatggcgctccaggagaaagatgttaacacccactttccattttaccattctggaagatttcttagatatcatgaatgaacaagcaaatatattggttaagaaacttgaaaaacacattaaccaagaagcatttaactgctttttttacatcactctttgtgccttagatatcatctgtgaaacagctatggggaagaatattggtgctcaaagtaatgatgattccgagtatgtccgtgcagtttatagaatgagtgagatgatatttcgaagaataaagatgccctggctttggcttgatctctggtatcttatgtttaaagaaggatgggaacacaaaaagagccttcagatcctacatacttttaccaacagtgtcatcgctgaacgggccaatgaaatgaacgccaatgaagactgtagaggtgatggcaggggctctgccccctccaaaaataaacgcagggcctttcttgacttgcttttaagtgtgactgatgacgaagggaacaggctaagtcatgaagatattcgagaagaagttgacaccttcatgtttgaggggcacgatacaactgcagctgcaataaactggtccttatacctgttgggttctaacccagaagtccagaaaaaagtggatcatgaattggatgacgtgtttgggaagtctgaccgtcccgctacagtagaagacctgaagaaacttcggtatctggaatgtgttattaaggagacccttcgcctttttccttctgttcctttatttgcccgtagtgttagtgaagattgtgaagtggcaggttacagagttctaaaaggcactgaagccgtcatcattccctatgcattgcacagagatccgagatacttccccaaccccgaggagttccagcctgagcggttcttccccgagaatgcacaagggcgccatccatatgcctacgtgcccttctctgctggccccaggaactgtataggtcaaaagtttgctgtgatggaagaaaagaccattctttcgtgcatcctgaggcacttttggatagaatccaaccagaaaagagaagagcttggtctagaaggacagttgattcttcgtccaagtaatggcatctggatcaagttgaagaggagaaatgcagatgaacgctaa

SEQ ID NO: 117 - DNA, 711 bp, Homo sapiens
  misc_feature 1..711: note = REGION - Retinoschisin 1 (RS1)
  source 1..711: mol_type = other DNA
atgagccgcaagatagaaggctttttgttattacttctctttggctatgaagccacattgggattatcgtctaccgaggatgaaggcgaggacccctggtatcaaaaagcatgcgatgaaggcgaggacccctggtatcaaaaagcatgcaagtgcgattgccaaggaggacccaatgctctgtggtctgcaggtgccacctccttggactgtataccagaatgcccatatcacaagcctctgggtttcgagtcaggggaggtcacaccggaccagatcacctgctctaacccggagcagtatgtgggctggtattcttcgtggactgcaaacaaggcccggctcaacagtcaaggctttgggtgtgcctggctctccaagttccaggacagtagccagtggttacagatagatctgaaggagatcaaagtgatttcagggatcctcacccaggggcgctgtgacatcgatgagtggatgaccaagtacagcgtgcagtacaggaccgatgagcgcctgaactggatttactacaaggaccagactggaaacaaccgggtcttctatggcaactcggaccgcacctccacggttcagaacctgctgcggccccccatcatctcccgcttcatccgcctcatcccgctgggctggcacgtccgcattgccatccggatggagctgctggagtgcgtcagcaagtgtgcctga

SEQ ID NO: 118 - DNA, 2565 bp, Homo sapiens
  misc_feature 1..2565: note = REGION - Phosphodiesterase 6B (PDE6B)
  source 1..2565: mol_type = other DNA
atgagcctcagtgaggagcaggcccggagctttctggaccagaaccccgattttgcccgccagtactttgggaagaaactgagccctgagaatgtggccgcggcctgcgaggacgggtgcccgccggactgcgacagcctccgggacctctgccaggtggaggagagcacggcgctgctggagctggtgcaggatatgcaggagagcatcaacatggagcgcgtggtcttcaaggtcctgcggcgcctctgcaccctgctgcaggccgaccgctgcagcctcttcatgtaccgccagcgcaacggcgtggccgagctggccaccaggcttttcagcgtgcagccggacagcgtcctggaggactgcctggtgccccccgactccgagatcgtcttcccactggacatcggggtcgtgggccacgtggctcagaccaaaaagatggtgaacgtcgaggacgtggccgagtgccctcacttcagctcatttgctgacgagctcactgactacaagacaaagaatatgctggccacacccatcatgaatggcaaagacgtcgtggcggtgatcatggcagtgaacaagctcaacggcccattcttcaccagcgaagacgaagatgtgttcttgaagtacctgaattttgccacgttgtacctgaaaatctatcacctgagctacctccacaactgcgagacgcgccgcggccaggtgctgctgtggtcggccaacaaggtgtttgaggagctgacggacatcgagaggcagttccacaaggccttctacacggtgcgggcctacctcaactgcgagcggtactccgtgggcctcctggacatgaccaaggagaaggaattttttgacgtgtggtctgtgctgatgggagagtcccagccgtactcgggcccacgcacgcctgatggccgggaaattgtcttctacaaagtgatcgactacatcctccacggcaaggaggagatcaaggtcattcccacaccctcagccgatcactgggccctggccagcggccttccaagctacgtggcagaaagcggctttatttgtaacatcatgaatgcttccgctgacgaaatgttcaaatttcaggaaggggccctggacgactccgggtggctcatcaagaatgtgctgtccatgcccatcgtcaacaagaaggaggagattgtgggagtcgccacattttacaacaggaaagacgggaagccctttgacgaacaggacgaggttctcatggagtccctgacacagttcctgggctggtcagtgatgaacaccgacacctacgacaagatgaacaagctggagaaccgcaaggacatcgcacaggacatggtcctttaccacgtgaagtgcgacagggacgagatccagctcatcctgccaaccagagcgcgcctggggaaggagcctgctgactgcgatgaggacgagctgggcgaaatcctgaaggaggagctgccagggcccaccacatttgacatctacgaattccacttctctgacctggagtgcaccgaactggacctggtcaaatgtggcatccagatgtactacgagctgggcgtggtccgaaagttccagatcccccaggaggtcctggtgcggttcctgttctccatcagcaaaggctaccggagaatcacctaccacaactggcgccacggcttcaacgtggcccagacgatgttcacgctgctcatgaccggcaaactgaagagctactacacggacctggaggccttcgccatggtgacagccggcctgtgccatgacatcgaccaccgcggcaccaacaacctgtaccagatgaagtcccagaaccccttggctaaactccacggctcctcgattttggagcggcaccacctggagtttgggaagttcctgctctcggaggagaccctgaacatctaccagaacctgaaccggcggcagcacgagcacgtgatccacctgatggacatcgccatcatcgccacggacctggccctgtacttcaagaagagagcgatgtttcagaagatcgtggatgagtccaagaactaccaggacaagaagagctgggtggagtacctgtccctggagacgacccggaaggagatcgtcatggccatgatgatgacagcctgcgacctgtctgccatcaccaagccctgggaagtccagagcaaggtcgcacttctcgtggctgctgagttctgggagcaaggtgacttggaaaggacagtcttggatcagcagcccattcctatgatggaccggaacaaggcggccgagctccccaagctgcaagtgggcttcatcgacttcgtgtgcacattcgtgtacaaggagttctctcgtttccacgaagagatcctgcccatgttcgaccgactgcagaacaataggaaagagtggaaggcgctggctgatgagtatgaggccaaagtgaaggctctggaggagaaggaggaggaggagagggtggcagccaagaaagtaggcacagaaatttgcaatggcggcccagcacccaagtcttcaacctgctgtatcctgtga

SEQ ID NO: 119 - DNA, 1497 bp, Homo sapiens
  misc_feature 1..1497: note = REGION - Methyl-CpG Binding Protein (MeCP2)
  source 1..1497: mol_type = other DNA
atggccgccgccgccgccgccgcgccgagcggaggaggaggaggaggcgaggaggagagactggaagaaaagtcagaagaccaggacctccagggcctcaaggacaaacccctcaagtttaaaaaggtgaagaaagataagaaagaagagaaagagggcaagcatgagcccgtgcagccatcagcccaccactctgctgagcccgcagaggcaggcaaagcagagacatcagaagggtcaggctccgccccggctgtgccggaagcttctgcctcccccaaacagcggcgctccatcatccgtgaccggggacccatgtatgatgaccccaccctgcctgaaggctggacacggaagcttaagcaaaggaaatctggccgctctgctgggaagtatgatgtgtatttgatcaatccccagggaaaagcctttcgctctaaagtggagttgattgcgtacttcgaaaaggtaggcgacacatccctggaccctaatgattttgacttcacggtaactgggagagggagcccctcccggcgagagcagaaaccacctaagaagcccaaatctcccaaagctccaggaactggcagaggccggggacgccccaaagggagcggcaccacgagacccaaggcggccacgtcagagggtgtgcaggtgaaaagggtcctggagaaaagtcctgggaagctccttgtcaagatgccttttcaaacttcgccagggggcaaggctgaggggggtggggccaccacatccacccaggtcatggtgatcaaacgccccggcaggaagcgaaaagctgaggccgaccctcaggccattcccaagaaacggggccgaaagccggggagtgtggtggcagccgctgccgccgaggccaaaaagaaagccgtgaaggagtcttctatccgatctgtgcaggagaccgtactccccatcaagaagcgcaagacccgggagacggtcagcatcgaggtcaaggaagtggtgaagcccctgctggtgtccaccctcggtgagaagagcgggaaaggactgaagacctgtaagagccctgggcggaaaagcaaggagagcagccccaaggggcgcagcagcagcgcctcctcaccccccaagaaggagcaccaccaccatcaccaccactcagagtccccaaaggcccccgtgccactgctcccacccctgcccccacctccacctgagcccgagagctccgaggaccccaccagcccccctgagccccaggacttgagcagcagcgtctgcaaagaggagaagatgcccagaggaggctcactggagagcgacggctgccccaaggagccagctaagactcagcccgcggttgccaccgccgccacggccgcagaaaagtacaaacaccgaggggagggagagcgcaaagacattgtttcatcctccatgccaaggccaaacagagaggagcctgtggacagccggacgcccgtgaccgagagagttagctag

SEQ ID NO: 120 - DNA, 2232 bp, Homo sapiens
  misc_feature 1..2232: note = REGION - N-acetyl-alpha-glucosaminidase (NAGLU)
  source 1..2232: mol_type = other DNA
atggaggcggtggcggtggccgcggcggtgggggtccttctcctggccggggccgggggcgcggcaggcgacgaggcccgggaggcggcggccgtgcgggcgctcgtggcccggctgctggggccaggccccgcggccgacttctccgtgtcggtggagcgcgctctggctgccaagccgggcttggacacctacagcctgggcggcggcggcgcggcgcgcgtgcgggtgcgcggctccacgggcgtggcggccgccgcggggctgcaccgctacctgcgcgacttctgtggctgccacgtggcctggtccggctctcagctgcgcctgccgcggccactgccagccgtgccgggggagctgaccgaggccacgcccaacaggtaccgctattaccagaatgtgtgcacgcaaagctactccttcgtgtggtgggactgggcccgctgggagcgagagatagactggatggcgctgaatggcatcaacctggcactggcctggagcggccaggaggccatctggcagcgggtgtacctggccttgggcctgacccaggcagagatcaatgagttctttactggtcctgccttcctggcctgggggcgaatgggcaacctgcacacctgggatggccccctgcccccctcctggcacatcaagcagctttacctgcagcaccgggtcctggaccagatgcgctccttcggcatgaccccagtgctgcctgcattcgcggggcatgttcccgaggctgtcaccagggtgttccctcaggtcaatgtcacgaagatgggcagttggggccactttaactgttcctactcctgctccttccttctggctccggaagaccccatattccccatcatcgggagcctcttcctgcgagagctgatcaaagagtttggcacagaccacatctatggggccgacactttcaatgagatgcagccaccttcctcagagccctcctaccttgccgcagccaccactgccgtctatgaggccatgactgcagtggatactgaggctgtgtggctgctccaaggctggctcttccagcaccagccgcagttctgggggcccgcccagatcagggctgtgctgggagctgtgccccgtggccgcctcctggttctggacctgtttgctgagagccagcctgtgtatacccgcactgcctccttccagggccagcccttcatctggtgcatgctgcacaactttgggggaaaccatggtctttttggagccctagaggctgtgaacggaggcccagaagctgcccgcctcttccccaactccaccatggtaggcacgggcatggcccccgagggcatcagccagaacgaagtggtctattccctcatggctgagctgggctggcgaaaggacccagtgccagatttggcagcctgggtgaccagctttgccgcccggcggtatggggtctcccacccggacgcaggggcagcgtggaggctactgctccggagtgtgtacaactgctccggggaggcctgcaggggccacaatcgtagcccgctggtcaggcggccgtccctacagatgaataccagcatctggtacaaccgatctgatgtgtttgaggcctggcggctgctgctcacatctgctccctccctggccaccagccccgccttccgctacgacctgctggacctcactcggcaggcagtgcaggagctggtcagcttgtactatgaggaggcaagaagcgcctacctgagcaaggagctggcctccctgttgagggctggaggcgtcctggcctatgagctgctgccggcactggacgaggtgctggctagtgacagccgcttcttgctgggcagctggctagagcaggcccgagcagcggcagtcagtgaggccgaggccgatttctacgagcagaacagccgctaccagctgaccttgtgggggccagaaggcaacatcctggactatgccaacaagcagctggcggggttggtggccaactactacacccctcgctggcggcttttcctggaggcgctggttgacagtgtggcccagggcatccctttccaacagcaccagtttgacaaaaatgtcttccaactggagcaggccttcgttctcagcaagcagaggtaccccagccagccgcgaggagacactgtggacctggccaagaagatcttcctcaaatattaccccggctgggtggccggctcttggtga

SEQ ID NO: 121 - DNA, 1317 bp, Homo sapiens; identical to SEQ ID NO: 12
  misc_feature 1..1317: note = REGION - Ceroid Lipofuscinosis, Neuronal 3 (CLN3)
  source 1..1317: mol_type = other DNA

SEQ ID NO: 122 - DNA, 1317 bp, synthetic construct
  misc_feature 1..1317: note = Codon optimized CO1-CLN3
  source 1..1317: mol_type = other DNA
atgggaggatgtgctgggtcaagaagacggtttagcgattccgaaggagaggagactgtgcctgagccaagactgcccctgctggatcaccagggagcacactggaagaacgcagtgggattctggctgctgggcctgtgcaacaacttcagctacgtggtcatgctgtccgccgcccacgacatcctgtcccacaagcggacctccggcaatcagtctcacgtggaccccggccctacaccaatcccccacaacagcagcagccggttcgactgtaattccgtgtctaccgcagccgtgctgctggcagacatcctgcccaccctggtcatcaagctgctggcaccactgggcctgcacctgctgccttattctccaagggtgctggtgagcggcatctgcgcagcaggcagcttcgtgctggtggcctttagccactccgtgggcacctctctgtgcggagtggtgtttgcaagcatcagctccggcctgggagaggtgaccttcctgagcctgacagccttttaccctcgcgccgtgatctcctggtggtctagcggcacaggaggagcaggcctgctgggcgccctgtcctatctgggcctgacccaggcaggcctgtccccacagcagacactgctgtctatgctgggcatccctgccctgctgctggcaagctacttcctgctgctgacctccccagaggcacaggaccccggaggagaggaggaggccgagagcgccgcaaggcagccactgatcaggaccgaggcaccagagtccaagcctggctcctctagctccctgtctctgcgggagagatggacagtgttcaagggcctgctgtggtacatcgtgcccctggtggtggtgtacttcgccgagtacttcatcaaccagggcctgtttgagctgctgttcttttggaatacctctctgagccacgcccagcagtaccggtggtatcagatgctgtatcaggcaggcgtgttcgcctcccggtctagcctgagatgctgtcggatcagattcacctgggcactggccctgctgcagtgcctgaacctggtgttcctgctggccgacgtgtggttcggctttctgccctctatctacctggtgtttctgatcatcctgtatgagggcctgctgggaggagcagcctatgtgaacaccttccacaatatcgccctggagacatctgacgagcacagagagtttgctatggccgccacctgtatcagcgatacactgggcatctctctgagcggactgctggctctgcctctgcatgactttctgtgccagctgagttaa

SEQ ID NO: 123 - DNA, 2859 bp, Homo sapiens
  misc_feature 1..2859: note = REGION - Acid Alpha-Glucosidase (GAA)
  source 1..2859: mol_type = other DNA
atgggagtgaggcacccgccctgctcccaccggctcctggccgtctgcgccctcgtgtccttggcaaccgctgcactcctggggcacatcctactccatgatttcctgctggttccccgagagctgagtggctcctccccagtcctggaggagactcacccagctcaccagcagggagccagcagaccagggccccgggatgcccaggcacaccccggccgtcccagagcagtgcccacacagtgcgacgtcccccccaacagccgcttcgattgcgcccctgacaaggccatcacccaggaacagtgcgaggcccgcggctgttgctacatccctgcaaagcaggggctgcagggagcccagatggggcagccctggtgcttcttcccacccagctaccccagctacaagctggagaacctgagctcctctgaaatgggctacacggccaccctgacccgtaccacccccaccttcttccccaaggacatcctgaccctgcggctggacgtgatgatggagactgagaaccgcctccacttcacgatcaaagatccagctaacaggcgctacgaggtgcccttggagaccccgcatgtccacagccgggcaccgtccccactctacagcgtggagttctccgaggagcccttcggggtgatcgtgcgccggcagctggacggccgcgtgctgctgaacacgacggtggcgcccctgttctttgcggaccagttccttcagctgtccacctcgctgccctcgcagtatatcacaggcctcgccgagcacctcagtcccctgatgctcagcaccagctggaccaggatcaccctgtggaaccgggaccttgcgcccacgcccggtgcgaacctctacgggtctcaccctttctacctggcgctggaggacggcgggtcggcacacggggtgttcctgctaaacagcaatgccatggatgtggtcctgcagccgagccctgcccttagctggaggtcgacaggtgggatcctggatgtctacatcttcctgggcccagagcccaagagcgtggtgcagcagtacctggacgttgtgggatacccgttcatgccgccatactggggcctgggcttccacctgtgccgctggggctactcctccaccgctatcacccgccaggtggtggagaacatgaccagggcccacttccccctggacgtccagtggaacgacctggactacatggactcccggagggacttcacgttcaacaaggatggcttccgggacttcccggccatggtgcaggagctgcaccagggcggccggcgctacatgatgatcgtggatcctgccatcagcagctcgggccctgccgggagctacaggccctacgacgagggtctgcggaggggggttttcatcaccaacgagaccggccagccgctgattgggaaggtatggcccgggtccactgccttccccgacttcaccaaccccacagccctggcctggtgggaggacatggtggctgagttccatgaccaggtgcccttcgacggcatgtggattgacatgaacgagccttccaacttcatcaggggctctgaggacggctgccccaacaatgagctggagaacccaccctacgtgcctggggtggttggggggaccctccaggcggccaccatctgtgcctccagccaccagtttctctccacacactacaacctgcacaacctctacggcctgaccgaagccatcgcctcccacagggcgctggtgaaggctcgggggacacgcccatttgtgatctcccgctcgacctttgctggccacggccgatacgccggccactggacgggggacgtgtggagctcctgggagcagctcgcctcctccgtgccagaaatcctgcagtttaacctgctgggggtgcctctggtcggggccgacgtctgcggcttcctgggcaacacctcagaggagctgtgtgtgcgctggacccagctgggggccttctaccccttcatgcggaaccacaacagcctgctcagtctgccccaggagccgtacagcttcagcgagccggcccagcaggccatgaggaaggccctcaccctgcgctacgcactcctcccccacctctacacactgttccaccaggcccacgtcgcgggggagaccgtggcccggcccctcttcctggagttccccaaggactctagcacctggactgtggaccaccagctcctgtggggggaggccctgctcatcaccccagtgctccaggccgggaaggccgaagtgactggctacttccccttgggcacatggtacgacctgcagacggtgccagtagaggcccttggcagcctcccacccccacctgcagctccccgtgagccagccatccacagcgaggggcagtgggtgacgctgccggcccccctggacaccatcaacgtccacctccgggctgggtacatcatccccctgcagggccctggcctcacaaccacagagtcccgccagcagcccatggccctggctgtggccctgaccaagggtggggaggcccgaggggagctgttctgggacgatggagagagcctggaagtgctggagcgaggggcctacacacaggtcatcttcctggccaggaataacacgatcgtgaatgagctggtacgtgtgaccagtgagggagctggcctgcagctgcagaaggtgactgtcctgggcgtggccacggcgccccagcaggtcctctccaacggtgtccctgtctccaacttcacctacagccccgacaccaaggtcctggacatctgtgtctcgctgttgatgggagagcagtttctcgtcagctggtgttag

SEQ ID NO: 124 - DNA, 2859 bp, synthetic construct; identical to SEQ ID NO: 5
  misc_feature 1..2859: note = Codon optimized CO1-GAA
  source 1..2859: mol_type = other DNA

SEQ ID NO: 125 - DNA, 2859 bp, synthetic construct; identical to SEQ ID NO: 7
  misc_feature 1..2859: note = Codon optimized CO2-GAA
  source 1..2859: mol_type = other DNA

SEQ ID NO: 126 - DNA, 2859 bp, synthetic construct
  misc_feature 1..2859: note = Codon optimized CO3-GAA
  source 1..2859: mol_type = other DNA
atgggggtgagacaccccccctgcagccacagactgctggctgtgtgtgccctggtgagcctggccacagctgccctgctgggccacatcctgctgcatgacttcctactagtgcccagagagctgagtggcagcagccctgtgctggaggagacccaccctgcccaccagcagggggccagcagacctggccccagagatgcccaggcccaccctggcagacccagagctgtgcccacccagtgtgatgtgccccccaacagcagatttgactgtgcccctgacaaggccatcacccaggagcagtgtgaggccagaggctgctgctacatccctgccaagcagggcctgcagggggcccagatgggccagccctggtgcttcttcccccccagctaccccagctacaagctggagaacctgagcagcagtgagatgggctacacagccaccctgaccagaaccacccccaccttcttccccaaggacatcctgaccctgagactggatgtgatgatggagacagagaacagactgcacttcaccatcaaggaccctgccaacagaagatatgaggtgcccctggagaccccccatgtgcacagcagagcccccagccccctgtacagtgtggagttcagtgaggagccctttggggtgattgtgagaagacagctggatggcagagtgctgctgaacaccacagtggcccccctgttctttgctgaccagttcctgcagctgagcaccagcctgcccagccagtacatcacaggcctggctgagcacctgagccccctgatgctgagcaccagctggaccagaatcaccctgtggaacagagacctggcccccacccctggggccaacctgtatggcagccaccccttctacctggccctggaggatgggggcagtgcccatggggtgttcctgctgaacagcaatgccatggatgtggtgctgcagcccagccctgccctgagctggagaagcacagggggcatcctggatgtgtacatcttcctgggccctgagcccaagagtgtggtgcagcagtacctggatgtggtgggctaccccttcatgcccccctactggggcctgggcttccacctgtgcagatggggctacagcagcacagccatcaccagacaggtggtggagaacatgaccagagcccacttccccctggatgtgcagtggaatgacctggactacatggacagcagaagagacttcaccttcaacaaggatggcttcagagacttccctgccatggtgcaggagctgcaccaggggggcagaagatacatgatgattgtggaccctgccatcagcagcagtggccctgctggcagctacagaccctatgatgagggcctgagaagaggggtgttcatcaccaatgagacaggccagcccctgattggcaaggtgtggcctggcagcacagccttccctgacttcaccaaccccacagccctggcctggtgggaggacatggtggctgagttccatgaccaggtgccctttgatggcatgtggattgacatgaatgagcccagcaacttcatcagaggcagtgaggatggctgccccaacaatgagctggagaaccccccctatgtgcctggggtggtggggggcaccctgcaggctgccaccatctgtgccagcagccaccagttcctgagcacccactacaacctgcacaacctgtatggcctgacagaggccattgccagccacagagccctggtgaaggccagaggcaccagaccctttgtgatcagcagaagcacctttgctggccatggcagatatgctggccactggacaggggatgtgtggagcagctgggagcagctggccagcagtgtgcctgagatcctgcagttcaacctgctgggggtgcccctggtgggggctgatgtgtgtggcttcctgggcaacaccagtgaggagctgtgtgtgagatggacccagctgggggccttctaccccttcatgagaaaccacaacagcctgctgagcctgccccaggagccctacagcttcagtgagcctgcccagcaggccatgagaaaggccctgaccctgagatatgccctgctgccccacctgtacaccctgttccaccaggcccatgtggctggggagacagtggccagacccctgttcctggagttccccaaggacagcagcacctggacagtggaccaccagctgctgtggggggaggccctgctgatcacccctgtgctgcaggctggcaaggctgaggtgacaggctacttccccctgggcacctggtatgacctgcagacagtgcctgtggaggccctgggcagcctgccccccccccctgctgcccccagagagcctgccatccacagtgagggccagtgggtgaccctgcctgcccccctggacaccatcaatgtgcacctgagagctggctacatcatccccctgcagggccctggcctgaccaccacagagagcagacagcagcccatggccctggctgtggccctgaccaaggggggggaggccagaggggagctgttctgggatgatggggagagcctggaggtgctggagagaggggcctacacccaggtgatcttcctggccagaaacaacaccattgtgaatgagctggtgagagtgaccagtgagggggctggcctgcagctgcagaaggtgacagtgctgggggtggccacagccccccagcaggtgctgagcaatggggtgcctgtgagcaacttcacctacagccctgacaccaaggtgctggacatctgtgtgagcctgctgatgggggagcagttcctggtgagctggtgctga

SEQ ID NO: 127 - DNA, 1290 bp, Homo sapiens; identical to SEQ ID NO: 9
  misc_feature 1..1290: note = REGION - Alpha-Galactosidase A (GLA)
  source 1..1290: mol_type = other DNA

SEQ ID NO: 128 - DNA, 1290 bp, synthetic construct; identical to SEQ ID NO: 10
  misc_feature 1..1290: note = Codon optimized CO1-GLA
  source 1..1290: mol_type = other DNA

SEQ ID NO: 129 - DNA, 1377 bp, synthetic construct
  misc_feature 1..1377: note = Codon optimized + GET, CO1-GLA-GET
  source 1..1377: mol_type = other DNA
atgcagctgagaaatcctgaactgcacctgggctgtgccctggctctgagatttctggctctggtgtcctgggacattcctggcgctagagccctggataatggcctggccagaacacctacaatgggctggctgcactgggagagattcatgtgcaacctggactgccaagaggaacccgacagctgcatcagcgagaagctgttcatggaaatggccgagctgatggtgtccgaaggctggaaggatgccggctacgagtacctgtgcatcgacgattgctggatggcccctcagagagattctgagggcagactgcaggccgatcctcagagatttcctcacggaatccggcagctggccaactacgtgcactctaagggactgaagctgggcatctacgccgacgtgggcaacaagacatgtgccggctttccaggcagcttcggctactacgatatcgacgcccagacctttgccgattggggcgtcgacctgctgaagttcgatggctgctactgcgacagcctggaaaacctggccgacggctacaaacacatgtctctggccctgaaccggaccggcagatctatcgtgtactcttgcgagtggcccctgtacatgtggcccttccagaagcctaactacaccgagatcagacagtactgcaaccactggcggaacttcgccgacatcgatgacagctggaagtccatcaagagcatcctggactggaccagcttcaatcaagagcggatcgtggatgtggctggcccaggcggatggaacgatcctgatatgctggtcatcggcaacttcggcctgagctggaatcagcaagtgacccagatggccctgtgggccattatggccgctcctctgttcatgagcaacgacctgagacacatcagccctcaggccaaggctctgctgcaggataaggacgtgatcgccatcaaccaggatcctctgggcaagcagggctatcagctgagacagggcgacaatttcgaagtgtgggaaagacctctgagcggcctggcttgggccgtcgccatgatcaatagacaagagatcggcggaccccggtcctatacaattgccgtggcttctctcggaaaaggcgtggcctgcaatcctgcctgctttatcacacagctgctccccgtgaagagaaagctgggcttttacgagtggaccagcagactgagatcccacatcaaccccacaggcactgttctgctgcaactggaaaacacaatgcagatgagcctgaaggacctgctgcggagaagaagaaggcgcagacgcaagcgcaagaagaaaggcaaaggcctcggcaagaagcgggacccctgtctgagaaagtacaagtaa

SEQ ID NO: 130 - DNA, 1290 bp, synthetic construct
  misc_feature 1..1290: note = Codon optimized CO2-GLA
  source 1..1290: mol_type = other DNA
atgcagctgagaaaccctgagctgcacctgggctgtgccctggccctgagattcctggccctggtgagctgggacatccctggggccagagccctggacaatgggctagccagaacccccaccatgggctggctgcactgggagagattcatgtgcaacctggactgccaggaggagcctgacagctgcatcagtgagaagctgttcatggagatggctgagctgatggtgagtgagggctggaaggatgctggctatgagtacctgtgcattgatgactgctggatggccccccagagagacagtgagggcagactgcaggctgacccccagagattcccccatggcatcagacagctggccaactatgtgcacagcaagggcctgaagctgggcatctatgctgatgtgggcaacaagacctgtgctggcttccctggcagctttggctactatgacattgatgcccagacctttgctgactggggggtggacctgctgaagtttgatggctgctactgtgacagcctggagaacctggctgatggctacaagcacatgagcctggccctgaacagaacaggcagaagcattgtgtacagctgtgagtggcccctgtacatgtggcccttccagaagcccaactacacagagatcagacagtactgcaaccactggagaaactttgctgacattgatgacagctggaagagcatcaagagcatcctggactggaccagcttcaaccaggagagaattgtggatgtggctggccctgggggctggaatgaccctgacatgctggtgattggcaactttggcctgagctggaaccagcaggtgacccagatggccctgtgggccatcatggctgcccccctgttcatgagcaatgacctgagacacatcagcccccaggccaaggccctgctgcaggacaaggatgtgattgccatcaaccaggaccccctgggcaagcagggctaccagctgagacagggggacaactttgaggtgtgggagagacccctgagtggcctggcctgggctgtggccatgatcaacagacaggagattgggggccccagaagctacaccattgctgtggccagcctgggcaagggggtggcctgcaaccctgcctgcttcatcacccagctgctgcctgtgaagagaaagctgggcttctatgagtggaccagcagactgagaagccacatcaaccccacaggcacagtgctgctgcagctggagaacaccatgcagatgagcctgaaggacctgctgtga

SEQ ID NO: 131 - DNA, 1290 bp, synthetic construct
  misc_feature 1..1290: note = Codon optimized CO3-GLA
  source 1..1290: mol_type = other DNA
atgcagctgagaaaccctgagctgcacctgggctgtgccctggccctgagattcctggccctggtgagctgggacatccctggggccagagccctggacaatgggctagccagaacccccaccatgggctggctgcactgggagagattcatgtgcaacctggactgccaggaggagcctgacagctgcatcagtgagaagctgttcatggagatggctgagctgatggtgagtgagggctggaaggatgctggctatgagtacctgtgcattgatgactgctggatggccccccagagagacagtgagggcagactgcaggctgacccccagagattcccccatggcatcagacagctggccaactatgtgcacagcaagggcctgaagctgggcatctatgctgatgtgggcaacaagacctgtgctggcttccctggcagctttggctactatgacattgatgcccagacctttgctgactggggggtggacctgctgaagtttgatggctgctactgtgacagcctggagaacctggctgatggctacaagcacatgagcctggccctgaacagaacaggcagaagcattgtgtacagctgtgagtggcccctgtacatgtggcccttccagaagcccaactacacagagatcagacagtactgcaaccactggagaaactttgctgacattgatgacagctggaagagcatcaagagcatcctggactggaccagcttcaaccaggagagaattgtggatgtggctggccctgggggctggaatgaccctgacatgctggtgattggcaactttggcctgagctggaaccagcaggtgacccagatggccctgtgggccatcatggctgcccccctgttcatgagcaatgacctgagacacatcagcccccaggccaaggccctgctgcaggacaaggatgtgattgccatcaaccaggaccccctgggcaagcagggctaccagctgagacagggggacaactttgaggtgtgggagagacccctgagtggcctggcctgggctgtggccatgatcaacagacaggagattgggggccccagaagctacaccattgctgtggcttccctgggtaaaggagtggcctgtaatcctgcctgcttcatcacacagctcctccctgtgaaaaggaagctagggttctatgaatggacttcaaggttaagaagtcacataaatcccacaggcactgttttgcttcagctagaaaatacaatgcagatgtcattaaaagacttactttaa

SEQ ID NO: 132 - DNA, 4287 bp, synthetic construct; identical to SEQ ID NO: 4
  misc_feature 1..4287: note = Codon optimized, Cystic Fibrosis Transmembrane Regulator deltaR (CFTRdeltaR) contains R domain deletion
  source 1..4287: mol_type = other DNA

SEQ ID NO: 133 - DNA, 4443 bp, synthetic construct; identical to SEQ ID NO: 93
  misc_feature 1..4443: note = Codon optimized, full length Cystic Fibrosis Transmembrane Regulator (CFTR)
  source 1..4443: mol_type = other DNA

SEQ ID NO: 134 - protein, 502 aa, Homo sapiens
  REGION 1..502: note = Sulfoglucosamine sulfohydrolase (SGSH)
MSCPVPACCALLLVLGLCRARPRNALLLLADDGGFESGAYNNSAIATPHLDALARRSLLFRNAFTSVSSCSPSRASLLTGLPQHQNGMYGLHQDVHHFNSFDKVRSLPLLLSQAGVRTGIIGKKHVGPETVYPFDFAYTEENGSVLQVGRNITRIKLLVRKFLQTQDDQPFFLYVAFHDPHRCGHSQPQYGTFCEKFGNGESGMGRIPDWTPQAYDPLDVLVPYFVPNTPAARADLAAQYTTVGRMDQGVGLVLQELRDAGVLNDTLVIFTSDNGIPFPSGRTNLYWPGTAEPLLVSSPEHPKRWGQVSEAYVSLLDLTPTILDWFSIPYPSYAIFGSKTIHLTGRSLLPALEAEPLWATVFGSQSHHEVTMSYPMRSVQHRHFRLVHNLNFKMPFPIDQDFYVSPTFQDLLNRTTAGQPTGWYKDLRHYYYRARWELYDRSRDPHETQNLATDPRFAQLLEMLRDQLAKWQWETHDPWVCAPDGVLEEKLSPQCQPLHNEL

SEQ ID NO: 135 - protein, 531 aa, synthetic construct
  REGION 1..531: note = Codon optimized + GET CO1-SGSH-GET
MSCPVPACCALLLVLGLCRARPRNALLLLADDGGFESGAYNNSAIATPHLDALARRSLLFRNAFTSVSSCSPSRASLLTGLPQHQNGMYGLHQDVHHFNSFDKVRSLPLLLSQAGVRTGIIGKKHVGPETVYPFDFAYTEENGSVLQVGRNITRIKLLVRKFLQTQDDQPFFLYVAFHDPHRCGHSQPQYGTFCEKFGNGESGMGRIPDWTPQAYDPLDVLVPYFVPNTPAARADLAAQYTTVGRMDQGVGLVLQELRDAGVLNDTLVIFTSDNGIPFPSGRTNLYWPGTAEPLLVSSPEHPKRWGQVSEAYVSLLDLTPTILDWFSIPYPSYAIFGSKTIHLTGRSLLPALEAEPLWATVFGSQSHHEVTMSYPMRSVQHRHFRLVHNLNFKMPFPIDQDFYVSPTFQDLLNRTTAGQPTGWYKDLRHYYYRARWELYDRSRDPHETQNLATDPRFAQLLEMLRDQLAKWQWETHDPWVCAPDGVLEEKLSPQCQPLHNELRRRRRRRRKRKKKGKGLGKKRDPCLRKYK

SEQ ID NO: 136 - protein, 306 aa, synthetic construct
  REGION 1..306: note = Codon optimized Ceroid Lipofuscinosis, Neuronal, 1 (CLN1)
MASPGCLWLLAVALLPWTCASRALQHLDPPAPLPLVIWHGMGDSCCNPLSMGAIKKMVEKKIPGIYVLSLEIGKTLMEDVENSFFLNVNSQVTTVCQALAKDPKLQQGYNAMGFSQGGQFLRAVAQRCPSPPMINLISVGGQHQGVFGLPRCPGESSHICDFIRKTLNAGAYSKVVQERLVQAEYWHDPIKEDVYRNHSIFLADINQERGINESYKKNLMALKKFVMVKFLNDSIVDPVDSEWFGFYRSGQAKETIPLQETSLYTQDRLGLKEMDNAGQLVFLATEGDHLQLSEEWFYAHIIPFLG

SEQ ID NO: 137 - protein, 294 aa, Homo sapiens
  REGION 1..294: note = Survival Motor Neuron 1 (SMN1)
MAMSSGGSGGGVPEQEDSVLFRRGTGQSDDSDIWDDTALIKAYDKAVASFKHALKNGDICETSGKPKTTPKRKPAKKNKSQKKNTAASLQQWKVGDKCSAIWSEDGCIYPATIASIDFKRETCVVVYTGYGNREEQNLSDLLSPICEVANNIEQNAQENENESQVSTDESENSRSPGNKSDNIKPKSAPWNSFLPPPPPMPGPRLGPGKPGLKFNGPPPPPPPPPPHLLSCWLPPFPSGPPIIPPPPPICPDSLDDADALGSMLISWYMSGYHTGYYMGFRQNQKEGRCSHSLN

SEQ ID NO: 138 - protein, 515 aa, Homo sapiens
  REGION 1..515: note = Tissue Non-specific Alkaline Phosphatase (TNALP)
MISPFLVLAIGTCLTNSLVPEKEKDPKYWRDQAQETLKYALELQKLNTNVAKNVIMFLGDGMGVSTVTAARILKGQLHHNPGEETRLEMDKFPFVALSKTYNTNAQVPDSAGTATAYLCGVKANEGTVGVSAATERSRCNTTQGNEVTSILRWAKDAGKSVGIVTTTRVNHATPSAAYAHSADRDWYSDNEMPPEALSQGCKDIAYQLMHNIRDIDVIMGGGRKYMYPKNKTDVEYESDEKARGTRLDGLDLVDTWKSFKPRYKHSHFIWNRTELLTLDPHNVDYLLGLFEPGDMQYELNRNNVTDPSLSEMVVVAIQILRKNPKGFFLLVEGGRIDHGHHEGKAKQALHEAVEMDRAIGQAGSLTSSEDTLTVVTADHSHVFTFGGYTPRGNSIFGLAPMLSDTDKKPFTAILYGNGPGYKVVGGERENVSMVDYAHNNYQAQSAVPLRHETHGGEDVAVFSKGPMAHLLHGVHEQNYVPHVMAYAACIGANLGHCAPASSAGSDDDDDDDDDD

SEQ ID NO: 139 - protein, 211 aa, Homo sapiens
  REGION 1..211: note = Glial Cell Derived Neurotrophic Factor (GDNF)
MKLWDVVAVCLVLLHTASAFPLPAGKRPPEAPAEDRSLGRRRAPFALSSDSNMPEDYPDQFDDVMDFIQATIKRLKRSPDKQMAVLPRRERNRQAAAANPENSRGKGRRGQRGKNRGCVLTAIHLNVTDLGLGYETKEELIFRYCSGSCDAAETTYDKILKNLSRNRRLVSDKVGQACCRPIAFDDDLSFLDDNLVYHILRKHSAKRCGCI

SEQ ID NO: 140 - protein, 536 aa, Homo sapiens
  REGION 1..536: note = Tissue Glucosyl Ceramidase beta (GBA1)
MEFSSPSREECPKPLSRVSIMAGSLTGLLLLQAVSWASGARPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASCDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQCLGFTPEHQRDFIARDLGPTLANSTHHNVRLLMLDDQRLLLPHWAKVVLTDPEAAKYVHGIAVHWYLDFLAPAKATLGETHRLFPNTMLFASEACVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWRRQ

SEQ ID NO: 141 - protein, 653 aa, Homo sapiens
  REGION 1..653: note = Iduronidase alpha-L- (IDUA)
MRPLRPRAALLALLASLLAAPPVAPAEAPHLVHVDAARALWPLRRFWRSTGFCPPLPHSQADQYVLSWDQQLNLAYVGAVPHRGIKQVRTHWLLELVTTRGSTGRGLSYNFTHLDGYLDLLRENQLLPGFELMGSASGHFTDFEDKQQVFEWKDLVSSLARRYIGRYGLAHVSKWNFETWNEPDHHDFDNVSMTMQGFLNYYDACSEGLRAASPALRLGGPGDSFHTPPRSPLSWGLLRHCHDGTNFFTGEAGVRLDYISLHRKGARSSISILEQEKVVAQQIRQLFPKFADTPIYNDEADPLVGWSLPQPWRADVTYAAMVVKVIAQHQNLLLANTTSAFPYALLSNDNAFLSYHPHPFAQRTLTARFQVNNTRPPHVQLLRKPVLTAMGLLALLDEEQLWAEVSQAGTVLDSNHTVGVLASAHRPQGPADAWRAAVLIYASDDTRAHPNRSVAVTLRLRGVPPGPGLVYVTRYLDNGLCSPDGEWRRLGRPVFPTAEQFRRMRAAEDPVAAAPRPLPAGGRLTLRPALRLPSLLLVHVCARPEKPPGQVTRLRALPLTQGQLVLVWSDEHVGSKCLWTYEIQFSQDGKAYTPVSRKPSTFNLFVFSPDTGAVSGSYRVRALDYWARPGPFSDPVPYLEVPVPRGPPSPGNP

SEQ ID NO: 142 - protein, 525 aa, Homo sapiens
  REGION 1..525: note = Cytochrome P450 family 4 subfamily V member 2 (CYP4V2)
MAGLWLGLVWQKLLLWGAASALSLAGASLVLSLLQRVASYARKWQQMRPIPTVARAYPLVGHALLMKPDGREFFQQIIEYTEEYRHMPLLKLWVGPVPMVALYNAENVEVILTSSKQIDKSSMYKFLEPWLGLGLLTSTGNKWRSRRKMLTPTFHFTILEDFLDIMNEQANILVKKLEKHINQEAFNCFFYITLCALDIICETAMGKNIGAQSNDDSEYVRAVYRMSEMIFRRIKMPWLWLDLWYLMFKEGWEHKKSLQILHTFTNSVIAERANEMNANEDCRGDGRGSAPSKNKRRAFLDLLLSVTDDEGNRLSHEDIREEVDTFMFEGHDTTAAAINWSLYLLGSNPEVQKKVDHELDDVFGKSDRPATVEDLKKLRYLECVIKETLRLFPSVPLFARSVSEDCEVAGYRVLKGTEAVIIPYALHRDPRYFPNPEEFQPERFFPENAQGRHPYAYVPFSAGPRNCIGQKFAVMEEKTILSCILRHFWIESNQKREELGLEGQLILRPSNGIWIKLKRRNADER

SEQ ID NO: 143 - protein, 236 aa, Homo sapiens
  REGION 1..236: note = Retinoschisin 1 (RS1)
MSRKIEGFLLLLLFGYEATLGLSSTEDEGEDPWYQKACDEGEDPWYQKACKCDCQGGPNALWSAGATSLDCIPECPYHKPLGFESGEVTPDQITCSNPEQYVGWYSSWTANKARLNSQGFGCAWLSKFQDSSQWLQIDLKEIKVISGILTQGRCDIDEWMTKYSVQYRTDERLNWIYYKDQTGNNRVFYGNSDRTSTVQNLLRPPIISRFIRLIPLGWHVRIAIRMELLECVSKCA

SEQ ID NO: 144 - protein, 854 aa, Homo sapiens
  REGION 1..854: note = Phosphodiesterase 6B (PDE6B)
MSLSEEQARSFLDQNPDFARQYFGKKLSPENVAAACEDGCPPDCDSLRDLCQVEESTALLELVQDMQESINMERVVFKVLRRLCTLLQADRCSLFMYRQRNGVAELATRLFSVQPDSVLEDCLVPPDSEIVFPLDIGVVGHVAQTKKMVNVEDVAECPHFSSFADELTDYKTKNMLATPIMNGKDVVAVIMAVNKLNGPFFTSEDEDVFLKYLNFATLYLKIYHLSYLHNCETRRGQVLLWSANKVFEELTDIERQFHKAFYTVRAYLNCERYSVGLLDMTKEKEFFDVWSVLMGESQPYSGPRTPDGREIVFYKVIDYILHGKEEIKVIPTPSADHWALASGLPSYVAESGFICNIMNASADEMFKFQEGALDDSGWLIKNVLSMPIVNKKEEIVGVATFYNRKDGKPFDEQDEVLMESLTQFLGWSVMNTDTYDKMNKLENRKDIAQDMVLYHVKCDRDEIQLILPTRARLGKEPADCDEDELGEILKEELPGPTTFDIYEFHFSDLECTELDLVKCGIQMYYELGVVRKFQIPQEVLVRFLFSISKGYRRITYHNWRHGFNVAQTMFTLLMTGKLKSYYTDLEAFAMVTAGLCHDIDHRGTNNLYQMKSQNPLAKLHGSSILERHHLEFGKFLLSEETLNIYQNLNRRQHEHVIHLMDIAIIATDLALYFKKRAMFQKIVDESKNYQDKKSWVEYLSLETTRKEIVMAMMMTACDLSAITKPWEVQSKVALLVAAEFWEQGDLERTVLDQQPIPMMDRNKAAELPKLQVGFIDFVCTFVYKEFSRFHEEILPMFDRLQNNRKEWKALADEYEAKVKALEEKEEEERVAAKKVGTEICNGGPAPKSSTCCIL

SEQ ID NO: 145 - protein, 498 aa, Homo sapiens [sequence omitted]

SEQ ID NO: 146 - protein, 743 aa, Homo sapiens
  REGION 1..743: note = N-acetyl-alpha-glucosaminidase (NAGLU)
MEAVAVAAAVGVLLLAGAGGAAGDEAREAAAVRALVARLLGPGPAADFSVSVERALAAKPGLDTYSLGGGGAARVRVRGSTGVAAAAGLHRYLRDFCGCHVAWSGSQLRLPRPLPAVPGELTEATPNRYRYYQNVCTQSYSFVWWDWARWEREIDWMALNGINLALAWSGQEAIWQRVYLALGLTQAEINEFFTGPAFLAWGRMGNLHTWDGPLPPSWHIKQLYLQHRVLDQMRSFGMTPVLPAFAGHVPEAVTRVFPQVNVTKMGSWGHFNCSYSCSFLLAPEDPIFPIIGSLFLRELIKEFGTDHIYGADTFNEMQPPSSEPSYLAAATTAVYEAMTAVDTEAVWLLQGWLFQHQPQFWGPAQIRAVLGAVPRGRLLVLDLFAESQPVYTRTASFQGQPFIWCMLHNFGGNHGLFGALEAVNGGPEAARLFPNSTMVGTGMAPEGISQNEVVYSLMAELGWRKDPVPDLAAWVTSFAARRYGVSHPDAGAAWRLLLRSVYNCSGEACRGHNRSPLVRRPSLQMNTSIWYNRSDVFEAWRLLLTSAPSLATSPAFRYDLLDLTRQAVQELVSLYYEEARSAYLSKELASLLRAGGVLAYELLPALDEVLASDSRFLLGSWLEQARAAAVSEAEADFYEQNSRYQLTLWGPEGNILDYANKQLAGLVANYYTPRWRLFLEALVDSVAQGIPFQQHQFDKNVFQLEQAFVLSKQRYPSQPRGDTVDLAKKIFLKYYPGWVAGSW

SEQ ID NO: 147 - skipped

SEQ ID NO: 148 - protein, 429 aa, Homo sapiens; identical to SEQ ID NO: 11
  REGION 1..429: note = Alpha-Galactosidase A (GLA)

SEQ ID NO: 149 - protein, 458 aa, synthetic construct
  REGION 1..458: note = Codon optimized + GET, CO1-GLA-GET
MQLRNPELHLGCALALRFLALVSWDIPGARALDNGLARTPTMGWLHWERFMCNLDCQEEPDSCISEKLFMEMAELMVSEGWKDAGYEYLCIDDCWMAPQRDSEGRLQADPQRFPHGIRQLANYVHSKGLKLGIYADVGNKTCAGFPGSFGYYDIDAQTFADWGVDLLKFDGCYCDSLENLADGYKHMSLALNRTGRSIVYSCEWPLYMWPFQKPNYTEIRQYCNHWRNFADIDDSWKSIKSILDWTSFNQERIVDVAGPGGWNDPDMLVIGNFGLSWNQQVTQMALWAIMAAPLFMSNDLRHISPQAKALLQDKDVIAINQDPLGKQGYQLRQGDNFEVWERPLSGLAWAVAMINRQEIGGPRSYTIAVASLGKGVACNPACFITQLLPVKRKLGFYEWTSRLRSHINPTGTVLLQLENTMQMSLKDLLRRRRRRRRKRKKKGKGLGKKRDPCLRKYK

SEQ ID NO: 150 - protein, 1428 aa, synthetic construct; identical to SEQ ID NO: 95
  REGION 1..1428: note = Codon optimized, Cystic Fibrosis Transmembrane Regulator deltaR (CFTRdeltaR) contains R domain deletion

SEQ ID NO: 151 - protein, 1480 aa, synthetic construct; identical to SEQ ID NO: 94
  REGION 1..1480: note = Codon optimized, full length Cystic Fibrosis Transmembrane Regulator (CFTR)

SEQ ID NO: 152 - DNA, 250 bp, synthetic construct; identical to SEQ ID NO: 96
  misc_feature 1..250: note = Mouse U1a promoter
  source 1..250: mol_type = other DNA

SEQ ID NO: 153 - DNA, 101 bp, synthetic construct; identical to SEQ ID NO: 97
  misc_feature 1..101: note = Polymerase III H1 mutant promoter
  source 1..101: mol_type = other DNA

SEQ ID NO: 154 - DNA, 701 bp, synthetic construct
  misc_feature 1..701: note = Chicken beta-actin hybrid promoter CBh (CBh promoter consists of CMV enhancer, CBA promoter, first CBA exon and partial intron)
  source 1..701: mol_type = other DNA
cgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagc

SEQ ID NO: 155 - DNA, 229 bp, synthetic construct
  misc_feature 1..229: note = MeCP2 min promoter sequence
  source 1..229: mol_type = other DNA
agctgaatggggtccgcctcttttccctgcctaaacagacaggaactcctgccaattgagggcgtcaccgctaaggctccgccccagcctgggctccacaaccaatgaagggtaatctcgacaaagagcaaggggtggggcgcgggcgcgcaggtgcagcagcacacaggctggtcgggagggcggggcgcgacgtctgccgtgcggggtcccggcatcggttgcgcgc

SEQ ID NO: 156 - DNA, 737 bp, synthetic construct
  misc_feature 1..737: note = MeCP2 promoter sequence
  source 1..737: mol_type = other DNA
tcaaaccatctgattcaacaatgcacgaccgatctcttatgggcttggcacacaccatctgcccattataaacgtctgcaaagaccaaggtttgatatgttgattttactgtcagccttaagagtgcgacatctgctaatttagtgtaataatacaatcagtagaccctttaaaacaagtcccttggcttggaacaacgccaggctcctcaacaggcaactttgctacttctacagaaaatgataataaagaaatgctggtgaagtcaaatgcttatcacaatggtgaactactcagcagggaggctctaataggcgccaagagcctagacttccttaagcgccagagtccacaagggcccagttaatcctcaacattcaaatgctgcccacaaaaccagcccctctgtgccctagccgcctcttttttccaagtgacagtagaactccaccaatccgcagctgaatggggtccgcctcttttccctgcctaaacagacaggaactcctgccaattgagggcgtcaccgctaaggctccgccccagcctgggctccacaaccaatgaagggtaatctcgacaaagagcaaggggtggggcgcgggcgcgcaggtgcagcagcacacaggctggtcgggagggcggggcgcgacgtctgccgtgcggggtcccggcatcggttgcgcgcgcgctccctcctctcggagagagggctgtggtaaaacccgtccggaaaa

SEQ ID NO: 157 - DNA, 418 bp, synthetic construct
  misc_feature 1..418: note = MeCP418 promoter sequence
  source 1..418: mol_type = other DNA
ataggcgccaagagcctagacttccttaagcgccagagtccacaagggcccagttaatcctcaacattcaaatgctgcccacaaaaccagcccctctgtgccctagccgcctcttttttccaagtgacagtagaactccaccaatccgcagctgaatggggtccgcctcttttccctgcctaaacagacaggaactcctgccaattgagggcgtcaccgctaaggctccgccccagcctgggctccacaaccaatgaagggtaatctcgacaaagagcaaggggtggggcgcgggcgcgcaggtgcagcagcacacaggctggtcgggagggcggggcgcgacgtctgccgtgcggggtcccggcatcggttgcgcgcgcgctccctcctctcggagagagggctgtggtaaaacccg

SEQ ID NO: 158 - DNA, 426 bp, synthetic construct
  misc_feature 1..426: note = MeCP426 promoter sequence
  source 1..426: mol_type = other DNA
ataggcgccaagagcctagacttccttaagcgccagagtccacaagggcccagttaatcctcaacattcaaatgctgcccacaaaaccagcccctctgtgccctagccgcctcttttttccaagtgacagtagaactccaccaatccgcagctgaatggggtccgcctcttttccctgcctaaacagacaggaactcctgccaattgagggcgtcaccgctaaggctccgccccagcctgggctccacaaccaatgaagggtaatctcgacaaagagcaaggggtggggcgcgggcgcgcaggtgcagcagcacacaggctggtcgggagggcggggcgcgacgtctgccgtgcggggtcccggcatcggttgcgcgcgcgctccctcctctcggagagagggctgtggtaaaacccgtccggaaa

SEQ ID NO: 159 - DNA, 400 bp, synthetic construct
  misc_feature 1..400: note = VMD2 promoter
  source 1..400: mol_type = other DNA
aattctgtcattttactagggtgatgaaattcccaagcaacaccatccttttcagataagggcactgaggctgagagaggagctgaaacctacccggggtcaccacacacaggtggcaaggctgggaccagaaaccaggactgttgactctggattttagggccatggtagagggggtgttgccctaaattccagccctggtctcagcccaacaccctccaagaagaaattagaggggccatggccaggctgtgctagccgttgcttctgagcagattacaagaagggactaagacaaggactcctttgtggaggtcctggcttagggagtcaagtgacggcggctcagcactcacgtgggcagtgccagcctctaagagtgggcaggggcactggccac

SEQ ID NO: 160 - DNA, 136 bp, synthetic construct
  misc_feature 1..136: note = PDE6b promoter
  source 1..136: mol_type = other DNA
cccatttgtaggagtgagtcagctgacccgcccccggggttcctaatctcactaagaaagactttgctgatgacagggtttcctgggagtccatgcgtgcctggagcagcagcgtctccagggacaggcagccacc

SEQ ID NO: 161 - DNA, 2035 bp, synthetic construct
  misc_feature 1..2035: note = mRho promoter
  source 1..2035: mol_type = other DNA
gcgccaatcagccgatgacttctaacaatactcttaactcacacagagcttgtctcactgagccaacaccctgtaccctcagctcagtgacggctttcaacctgtggggctgcctctgttacccaagtgagagagggccagtgctcccagaggtgaccttgtttgcccattctctccctgggtcagccagtgtttatctgttgtatacccagtccaccctgcaggctcacatcagagcctaggagatggctagtgtccccgcggagaccacgatgaagcttcccagctgtctcaagcacaagctggctgcagaggctgctgaggcactgctagctggggatgggggcagggtagatctggggctgaccaccagggtcagaatcagaacctccaccttgacctcattaacgctggtcttaatcaccaagccaagctccttaaactgctagtggccaactcccaggccctgacacacatacctgccctgtgttcccaaacaagacacctgcatggaaggaagggggttgcttttctaagcaaacatctaggaatcccgggtgcagtgtgaggagactaggcgagggagtactttaagggcctcaaggctcagagaggaatacttcttccctggttagcctcgtgcctaggctccagggtctttgtcctgcctggatacctatgtggcaaggggcatagcatttcccccaccatcagctcttagctcaaccttatcttctcggaaagactgcgcagtgtaacaacacagcagagacttttcttttgtcccctgtctacccctgtaactgctactcagaagcatctttctcacagggtactggcttcttgcatccagagttttttgtctccctcgggcccccagaatcaaattcttcctctgggactcagtggatgtttcacacacgtatcggcctgacagtcatcctggagcatcctacacaggggccatcacagctgcatgtcagaaatgctggcctcacatcctcagacaccaggcctagtgctggtcttcctcagactggcgtccccagcaggccagtaggatcatcttttagcctacagagttctgaagcctcagagccccaggtccctggtcatcttctctgcccctgagatttttccaagttgtatgccttctaggtaaggcaaaacttcttacgcccctcctcgtggcctccaggccccacatgctcacctgaataacctggcagcctgctccctcatgcagggaccacgtcctgctgcacccagcaggccatcccgtctccatagcccatggtcatccctccctggacaggaatgtgtctcctccccgggctgagtcttgctcaagctagaagcactccgaacagggttatgggcgcctcctccatctcccaagtggctggcttatgaatgtttaatgtacatgtgagtgaacaaattccaattgaacgcaacaaatagttatcgagccgctgagccggggggcggggggtgtgagactggaggcgatggacggagctgacggcacacacagctcagatctgtcaagtgagccattgtcagggcttggggactggataagtcagggggtctcctgggaagagatgggataggtgagttcaggaggagacattgtcaactggagccatgtggagaagtgaatttagggcccaaaggttccagtcgcagcctgaggccaccagactgacatggggaggaattcccagaggactctggggcagacaagatgagacaccctttcctttctttacctaagggcctccacccgatgtcaccttggcccctctgcaagccaattaggccccggtggcagcagtgggattagcgttagtatgatatctcgcggatgctgaatcagcctctggcttagggagagaaggtcactttataagggtctggggggggtcagtgcctggagttgcgctgtgggagccgtcagtggctgagctcgccaagcagccttggtctctgtctacgaagagcccgtggggcagcctcgagagccgcagcc

SEQ ID NO: 162 - DNA, 511 bp, synthetic construct
  misc_feature 1..511: note = CMV promoter
  source 1..511: mol_type = other DNA
ccgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagat

SEQ ID NO: 163 - DNA, 334 bp, synthetic construct
  misc_feature 1..334: note = UbC promoter
  source 1..334: mol_type = other DNA
ggcctccgcgccgggttttggcgcctcccgcgggcgcccccctcctcacggcgagcgctgccacgtcagacgaagggcgcagcgagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagct